(19) United States
(12) Patent Application Publication
(10) Pub. No.: US 2018/0245074 A1
(43) Pub. Date: Aug. 30, 2018

(54) TREATING HEPATITIS B VIRUS INFECTION USING CRISPR

(71) Applicant: PROTIVA BIOTHERAPEUTICS, INC., Burnaby (CA)

(72) Inventors: Amy C. H. LEE, Burnaby (CA); Nicholas D. WEBER, Burnaby (CA)

(73) Assignee: PROTIVA BIOTHERAPEUTICS, INC., Burnaby, BC (CA)

(21) Appl. No.: 15/570,111

(22) PCT Filed: Jun. 6, 2016
(86) PCT No.: PCT/US2016/036068

§ 371 Date: Oct. 27, 2017

Related U.S. Application Data
(60) Provisional application No. 62/171,153, filed on Jun. 4, 2015.

(51) Int. Cl. C12N 15/11; A61K 9/50; C12N 9/22; C12N 15/90; A61P 31/20

(52) U.S. Cl. CPC C12N 15/11; A61K 9/5015; C12N 9/22; C12N 15/907; A61K 38/00; C12N 2310/20; C12N 2320/32; C12N 2800/80; A61P 31/20

(57) ABSTRACT

The present invention provides compositions comprising therapeutic nucleic acids such as gRNA that target Hepatitis B virus (HBV) gene expression, lipid particles comprising one or more (e.g., a combination) of the therapeutic nucleic acids, and methods of delivering and/or administering the lipid particles (e.g., for treating HBV infection and/or HDV infection in humans).

### CROSS-REFERENCE TO RELATED APPLICATION(S)

This patent application claims the benefit of priority of U.S. application Ser. No. 62/171,153, filed Jun. 4, 2015, which application is herein incorporated by reference.

### BACKGROUND

Hepatitis B virus (abbreviated as “HBV”) is a member of the Hepadnavirus family. The virus particle (sometimes referred to as a virion) includes an outer lipid envelope and an icosahedral nucleocapsid core composed of protein. The nucleocapsid encloses the viral DNA and a DNA polymerase that has reverse transcriptase activity. The outer envelope contains embedded proteins that are involved in viral binding of, and entry into, susceptible cells, typically liver hepatocytes. In addition to the infectious viral particles, filamentous and spherical bodies lacking a core can be found in the serum of infected individuals. These particles are not infectious and are composed of the lipid and protein that forms part of the surface of the virion, which is called the surface antigen (HBsAg), and is produced in excess during the life cycle of the virus.

The genome of HBV is made of circular DNA, but it is unusual because the DNA is not fully double-stranded. One end of the full length strand is linked to the viral DNA polymerase. The genome is 3020-3320 nucleotides long (for the full-length strand) and 1700-2800 nucleotides long (for the shorter strand). The negative-sense (non-coding) is complementary to the viral mRNA. The viral DNA is found in the nucleus soon after infection of the cell. There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small are produced. The function of the protein coded for by gene X is not fully understood but it is associated with the development of liver cancer. Replication of HBV is a complex process. Although replication takes place in the liver, the virus spreads to the blood where viral proteins and antibodies against them are found in infected people. The structure, replication and biology of HBV is reviewed in D. Glebe and C. M. Bremer, Seminars in Liver Disease, Vol. 33, No. 2, pages 103-112 (2013).

Infection of humans with HBV can cause an infectious inflammatory illness of the liver. Infected individuals may not exhibit symptoms for many years. It is estimated that about a third of the world population has been infected at one point in their lives, including 350 million who are chronic carriers.

The virus is transmitted by exposure to infectious blood or body fluids. Perinatal infection can also be a major route of infection. The acute illness causes liver inflammation, vomiting, jaundice, and possibly death. Chronic hepatitis B may eventually cause cirrhosis and liver cancer.

Although most people who are infected with HBV clear the infection through the action of their immune system, some infected people suffer an aggressive course of infection (fulminant hepatitis); while others are chronically infected thereby increasing their chance of liver disease. Several medications are currently approved for treatment of HBV infection, but infected individuals respond with various degrees of success to these medications, and none of these medications clear the virus from the infected person.

Hepatitis D virus (HDV) is a small circular enveloped RNA virus that can propagate only in the presence of the hepatitis B virus (HBV). In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Thus, there is a continuing need for compositions and methods for the treatment of HBV infection in humans, as well as for the treatment of HBV/HDV infection in humans.

### BRIEF SUMMARY

Targeted genome editing using engineered nucleases has progressed from being a niche technology to a method used by many biological researchers. This adoption has been largely fueled by the emergence of the clustered, regularly interspaced, short palindromic repeat (CRISPR) technology, an important new approach for generating RNA-guided nucleases, such as Cas9, with customizable specificities. See, e.g., Sander et al., Nature Biotechnology, 32(4), 347-355, including Supplementary Information (2014).

As described more fully herein, provided herein are compositions and methods for utilizing clustered, regularly interspaced, short palindromic repeat (CRISPR) technology to treat HBV. The guide RNA (gRNA) described herein to be utilized in the CRISPR technology are designed to target specifically identified sequences of the HBV genome. The molecules of the invention are useful, for example, for the treatment of HBV infection and/or HDV infection when administered in a therapeutic amount to a human subject infected with HBV or HBV/HDV. More generally, the invention provides molecules that are capable of inhibiting or silencing HBV gene expression in vitro and in vivo.

The present invention also provides nucleic acid-lipid particles, and formulations thereof, wherein the lipid particles each include one or more (e.g., a cocktail) of the molecules described herein, a cationic lipid, and a non-cationic lipid, and optionally a conjugated lipid that inhibits aggregation of particles.

The present invention also provides a pharmaceutical composition comprising one or a cocktail of gRNA molecules that target HBV gene expression, and a pharmaceutically acceptable carrier. For example, the present invention provides pharmaceutical compositions that each include one, two, or three gRNA molecules that target HBV gene expression, e.g., gRNA molecules that target the sequences described in FIG. 1, FIG. 2, FIG. 3 or FIG. 4, e.g, that target a conserved sequence. With respect to formulations that include a cocktail of gRNAs encapsulated within lipid particles, the different gRNA molecules may be co-encapsulated in the same lipid particle, or each type of gRNA species present in the cocktail may be encapsulated in separate particles, or some gRNA species may be coencapsulated in the same particle while other gRNA species are encapsulated in different particles within the formulation. Typically, the gRNA molecules of the invention are fully encapsulated in the lipid particle. In certain embodiments, the lipid particles comprise both gRNA and an mRNA encoding a Cas9. In certain embodiments, one population lipid particles comprises the gRNA and another population of lipid particles comprises the mRNA encoding a Cas9, which lipid particles may be in the same composition or in different compositions, and may be administered concurrently or sequentially.

The nucleic acid-lipid particles of the invention are useful for the prophylactic or therapeutic delivery, into a human infected with HBV or HBV/HDV, of gRNA molecules that silence the expression of one or more HBV genes, thereby ameliorating at least one symptom of HBV infection and/or HDV infection in the human. In some embodiments, one or more of the gRNA molecules described herein are formulated into nucleic acid-lipid particles, and the particles are administered to a mammal (e.g., a human) requiring such treatment. In certain instances, a therapeutically effective amount of the nucleic acid-lipid particle can be administered to the mammal, (e.g., for treating HBV and/or HDV infection in a human being). The nucleic acid-lipid particles of the invention are particularly useful for targeting liver cells in humans which is the site of most HBV gene expression. Administration of the nucleic acid-lipid particle can be by any route known in the art, such as, e.g., oral, intranasal, intravenous, intraperitoneal, intramuscular, intra-articular, intralesional, intratracheal, subcutaneous, or intradermal. In particular embodiments, the nucleic acid-lipid particle is administered systemically, e.g., via enteral or parenteral routes of administration.

In some embodiments, downregulation of HBV gene expression is determined by detecting HBV RNA or protein levels in a biological sample from a mammal after nucleic acid-lipid particle administration. In other embodiments, downregulation of HBV gene expression is determined by detecting HBV mRNA or protein levels in a biological sample from a mammal after nucleic acid-lipid particle administration. In certain embodiments, downregulation of HBV gene expression is detected by monitoring symptoms associated with HBV infection in a mammal after particle administration. In certain embodiments, inactivating mutations (e.g., specific inactivating mutations) in HBV DNA are detected to determine the efficacy of CRISPR/Cas9 at inactivating HBV.

In another embodiment, the present invention provides methods for introducing a combination of gRNA and Cas9 to silence HBV gene expression in a living cell, the method comprising the step of contacting the cell with a nucleic acid-lipid particle of the invention, wherein the nucleic acid-lipid particle includes an gRNA that targets HBV, under conditions whereby the gRNA enters the cell with the Cas9 mRNA and silences the expression of a Hepatitis B virus gene within the cell.

In another embodiment, the present invention provides a method for ameliorating one or more symptoms associated with Hepatitis B virus and/or Hepatitis D virus infection in a human, the method including the step of administering to the human a therapeutically effective amount of a nucleic acid-lipid particle of the present invention. In some embodiments, the nucleic acid-lipid particles used in the methods of this aspect of the invention include one, two or three or more different gRNA.

In another embodiment, the present invention provides methods for silencing HBV gene expression in a mammal (e.g., a human) in need thereof, wherein the methods each include the step of administering to the mammal a nucleic acid-lipid particle of the present invention.

In another aspect, the present invention provides methods for treating and/or ameliorating one or more symptoms associated with HBV and/or HDV infection in a human, wherein the methods each include the step of administering to the human a therapeutically effective amount of a nucleic acid-lipid particle of the present invention.

Certain embodiments of the present invention provide compositions and methods for inhibiting the replication of HDV, and/or ameliorating one or more symptoms of HDV infection, by administering to an individual infected with HDV a therapeutically effective amount of one or more compositions or nucleic acid-particles of the present invention that inhibit the synthesis of HBV surface antigen.

In another aspect, the present invention provides methods for inhibiting the expression of HBV in a mammal in need thereof (e.g., a human infected with HBV or HBV/HDV), wherein the methods each include the step of administering to the mammal a therapeutically effective amount of a nucleic acid-lipid particle of the present invention.

In a further aspect, the present invention provides methods for treating HBV and/or HDV infection in a human, wherein the methods each include the step of administering to the human a therapeutically effective amount of a nucleic acid-lipid particle of the present invention.

In a further aspect, the present invention provides for use of a molecule of the present invention for inhibiting Hepatitis B virus gene expression in a living cell.

In a further aspect, the present invention provides for use of a pharmaceutical composition of the present invention for inhibiting Hepatitis B virus gene expression in a living cell.

The compositions of the invention are also useful, for example, in biological assays (e.g., in vivo or in vitro assays) for inhibiting the expression of one or more HBV genes and/or transcripts to investigate HBV and/or HDV replication and biology, and/or to investigate or modulate the function of one or more HBV genes or transcripts. For example, the molecules of the invention can be screened using a biological assay to identify molecules that inhibit replication of HBV and/or HDV and that are candidate therapeutic agents for the treatment of HBV and/or HDV infection in humans, and/or the amelioration of at least one symptom associated with HBV and/or HDV infection in a human.

Other objects, features, and advantages of the present invention will be apparent to one of skill in the art from the following detailed description and figures.

### BRIEF DESCRIPTION OF THE FIGURES

FIG. 1 depicts identified HBV CRISPR target sites (indicated as matches) for Cas9 from Streptococcus pyogenes (SP), which Cas9 recognizes the protospacer adjacent motif (PAM) (see Table 1.) Target sites conserved across the four HBV genomes searched (A-D) are also presented. The PAM sequences, which are included in the figures, will not be represented in the gRNA. The first 20 nucleotides of the target sites represent the ‘target sequence’ of the gRNA.

FIG. 2 depicts identified HBV CRISPR target sites (indicated as matches) for Cas9 from Neisseria meningitidis (NM), which Cas9 recognizes the protospacer adjacent motif (PAM) (see Table 1.) The PAM sequences, which are included in the figures, will not be represented in the gRNA. The first 20 nucleotides of the target sites represent the ‘target sequence’ of the gRNA.

FIG. 3 depicts identified HBV CRISPR target sites (indicated as matches) for Cas9 from Streptococcus thermophilus (ST), which Cas9 recognizes the protospacer adjacent motif (PAM) (see Table 1.) Target sites conserved across the four HBV genomes searched (A-D) are also presented. The PAM sequences, which are included in the figures, will not be represented in the gRNA. The first 20 nucleotides of the target sites represent the ‘target sequence’ of the gRNA.

FIG. 4 depicts identified HBV CRISPR target sites (indicated as matches) for Cas9 from Streptococcus aureus (SA), which Cas9 recognizes the protospacer adjacent motif (PAM) (see Table 1.) Target sites conserved across the four HBV genomes searched (A-D) are also presented. The PAM sequences, which are included in the figures, will not be represented in the gRNA. The first 20 nucleotides of the target sites represent the ‘target sequence’ of the gRNA.

### DETAILED DESCRIPTION

The therapy described herein advantageously provides significant new compositions and methods for treating HBV and HDV infection in human beings and the symptoms associated therewith. Embodiments of the present invention can be administered, for example, once per day, once per week, or once every several weeks (e.g., once every two, three, four, five or six weeks).

Furthermore, the nucleic acid-lipid particles described herein enable the effective delivery of a nucleic acid drug into target tissues and cells within the body. The presence of the lipid particle confers protection from nuclease degradation in the bloodstream, allows preferential accumulation in target tissue and provides a means of drug entry into the cells.

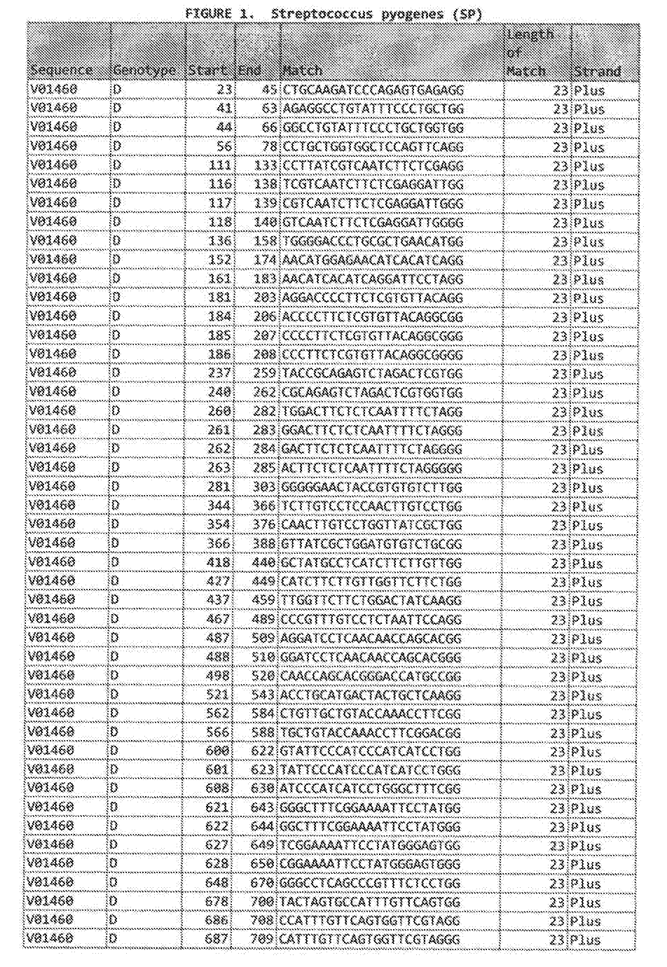

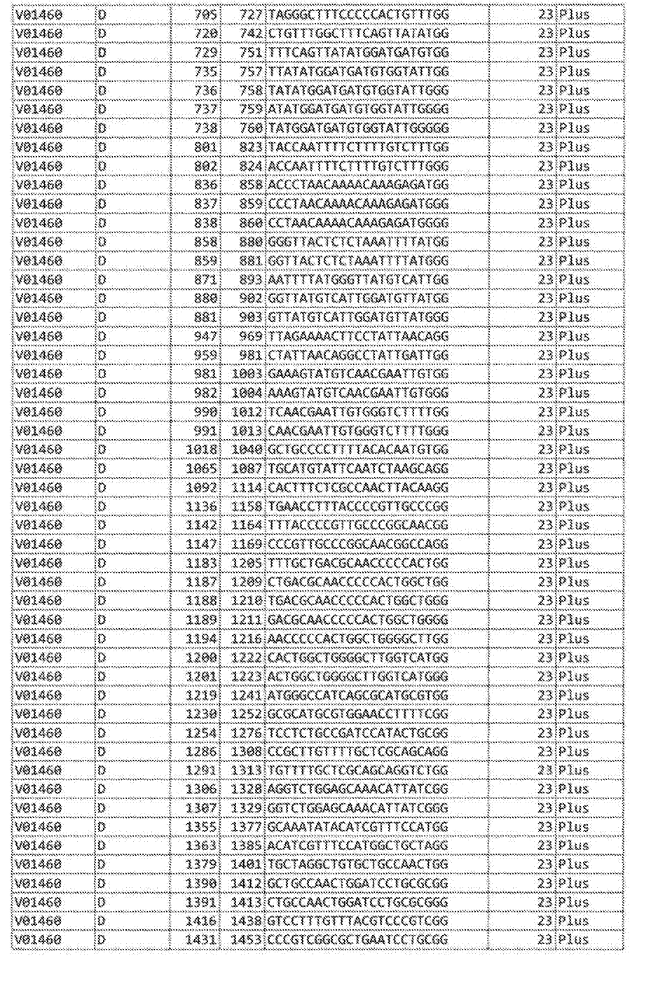

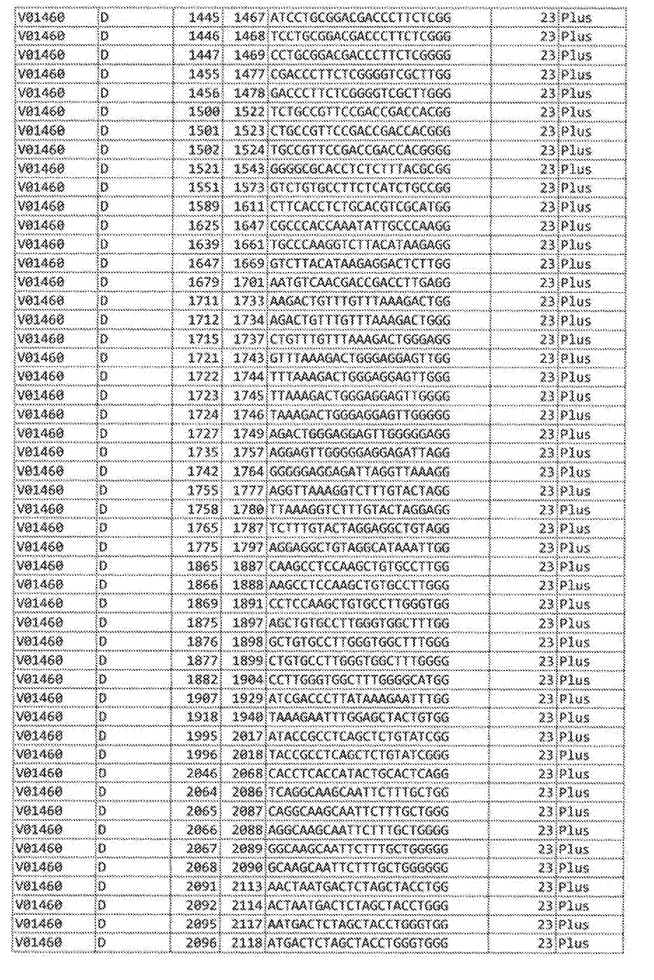

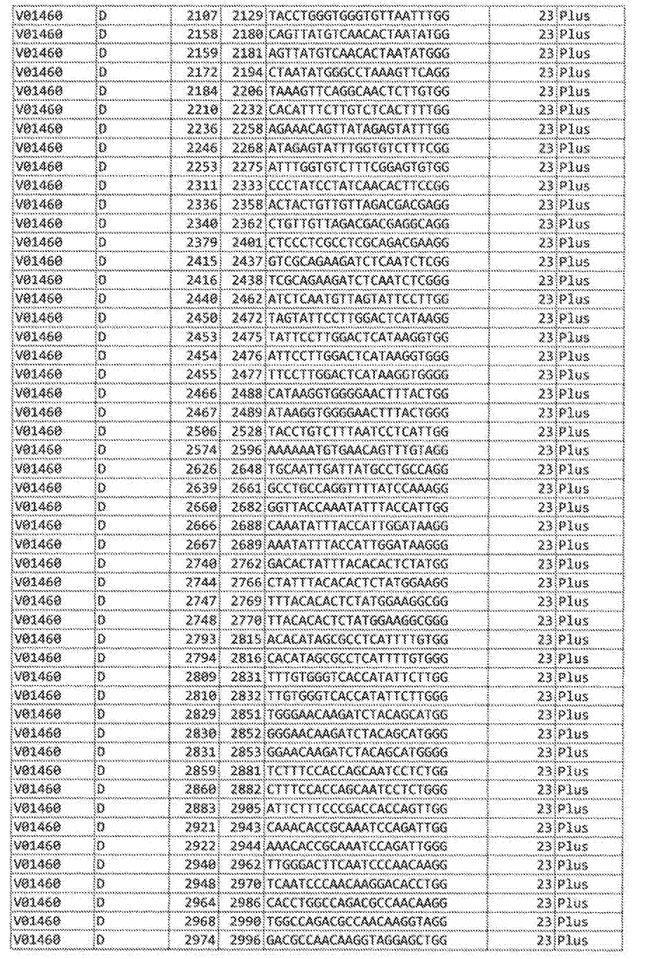

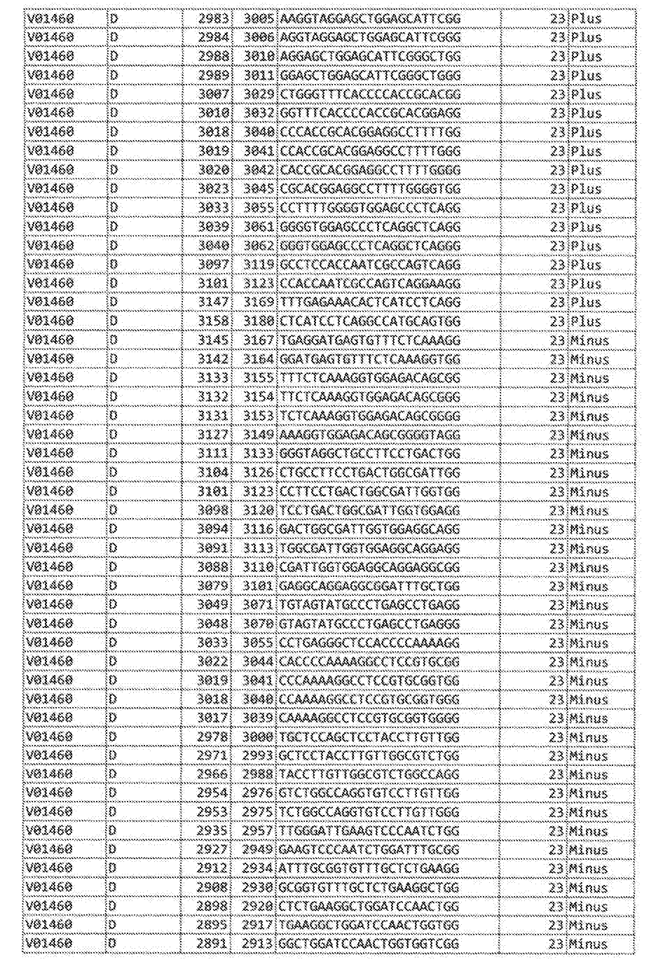

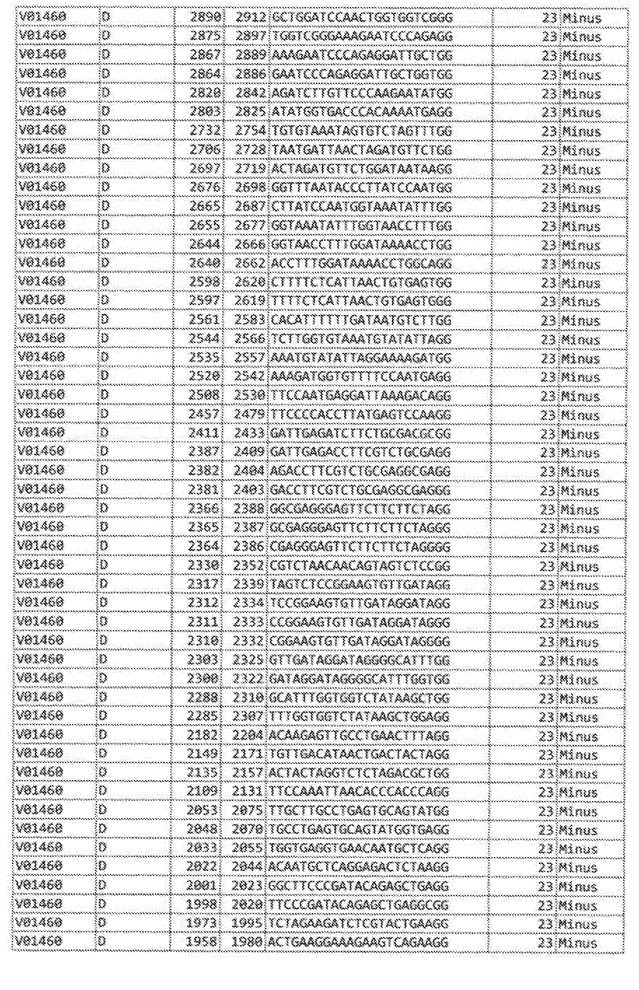

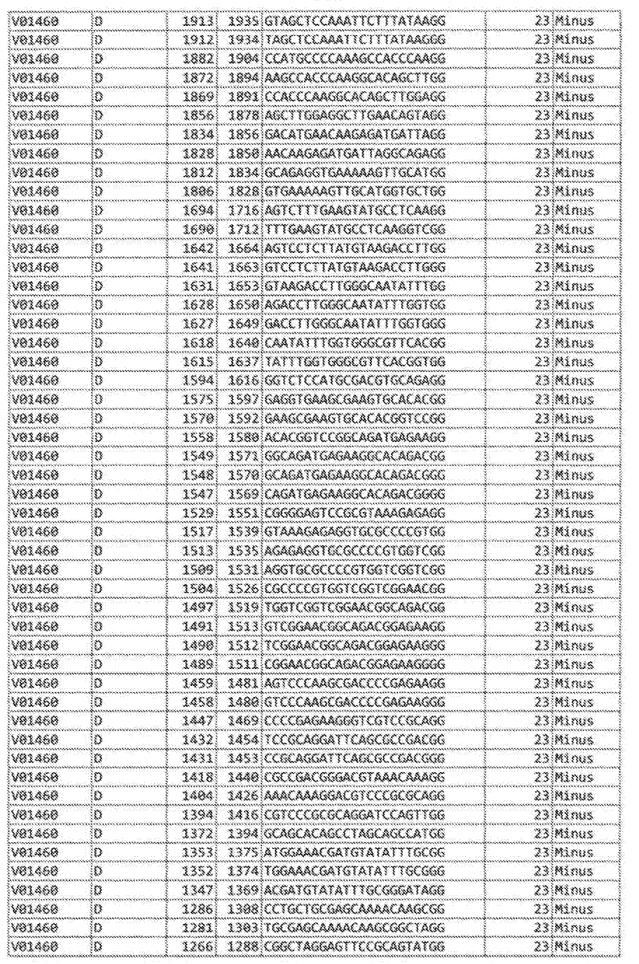

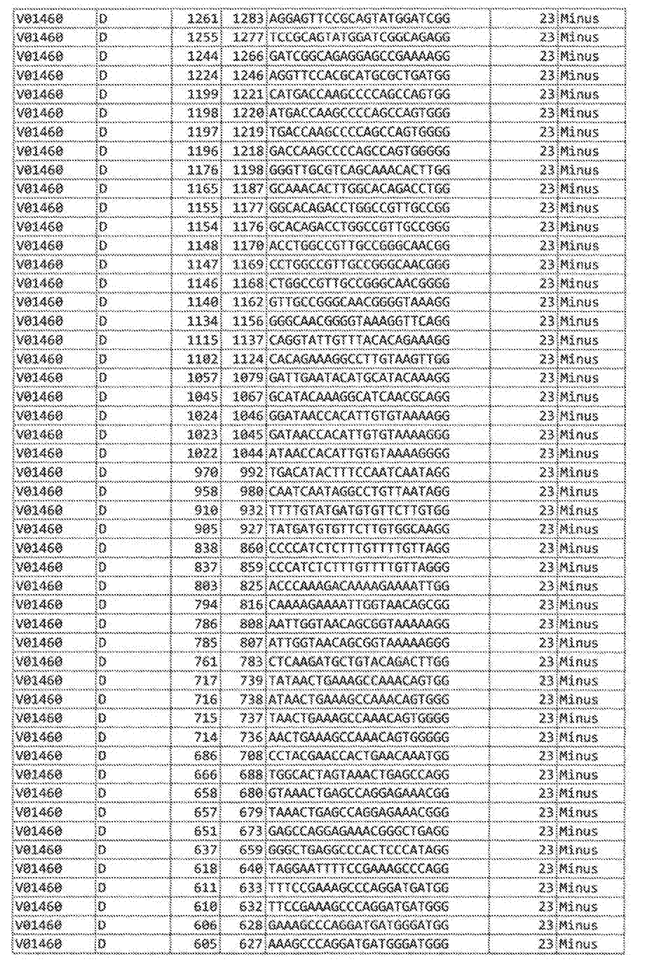

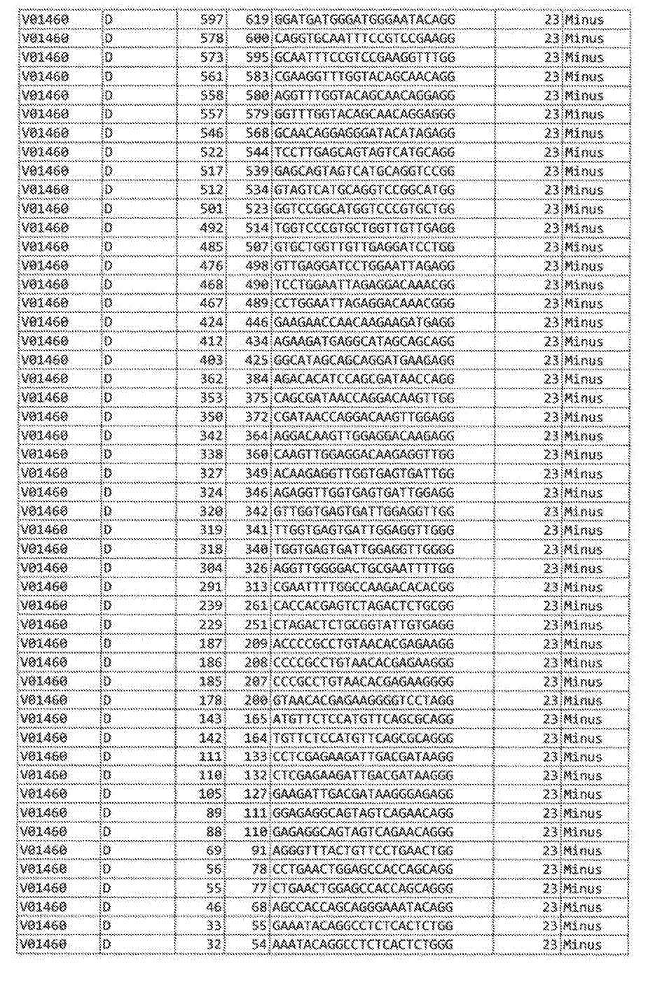

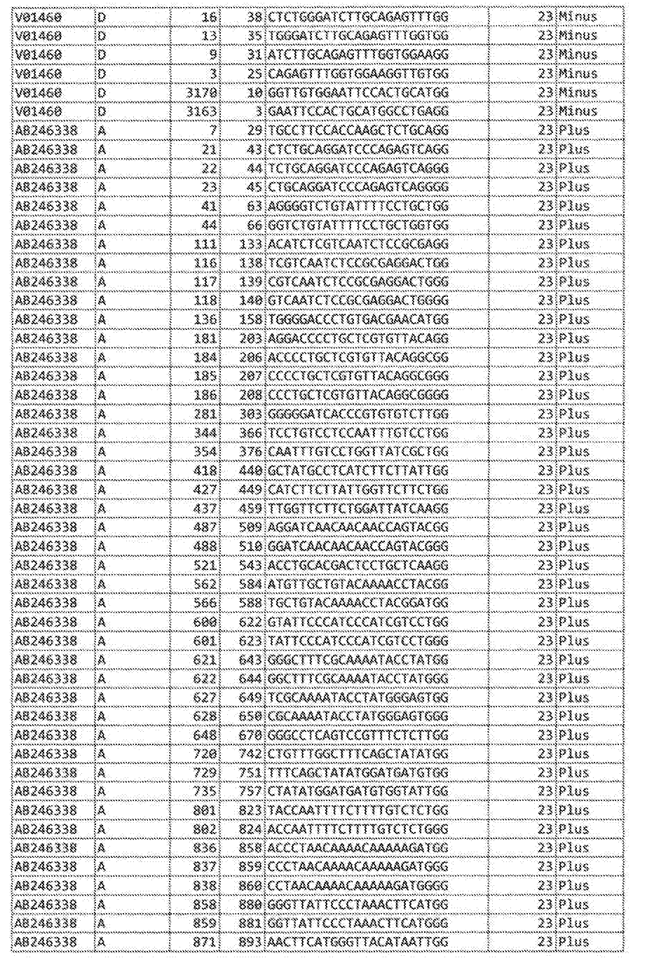

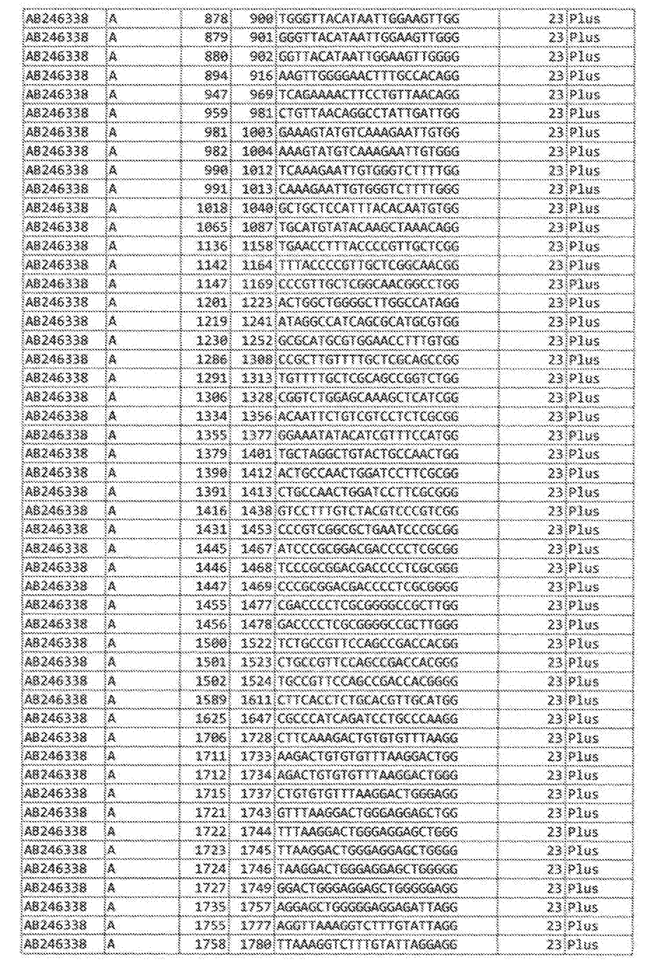

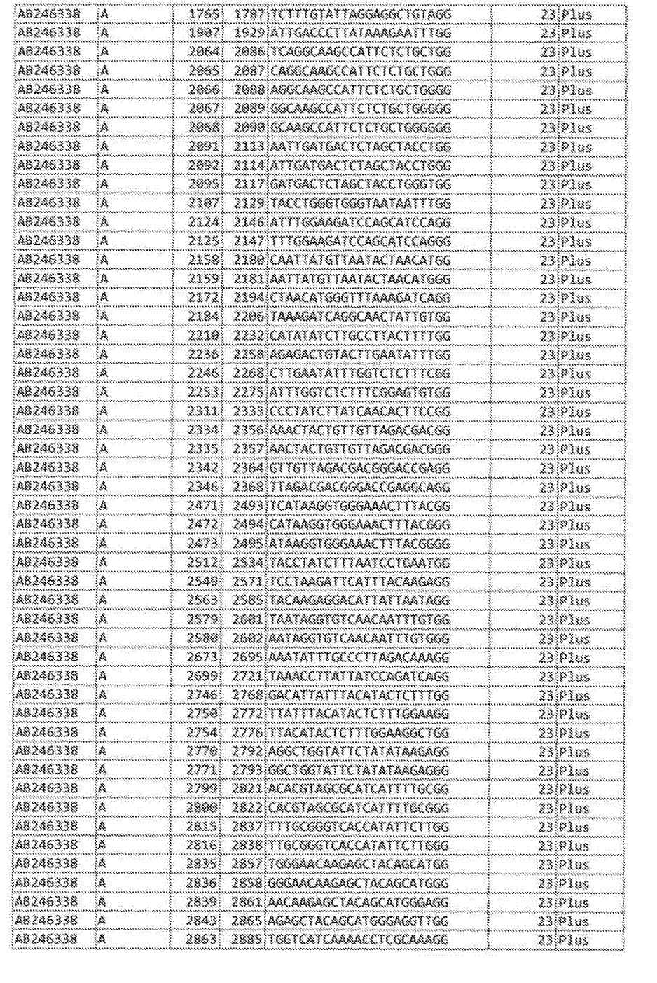

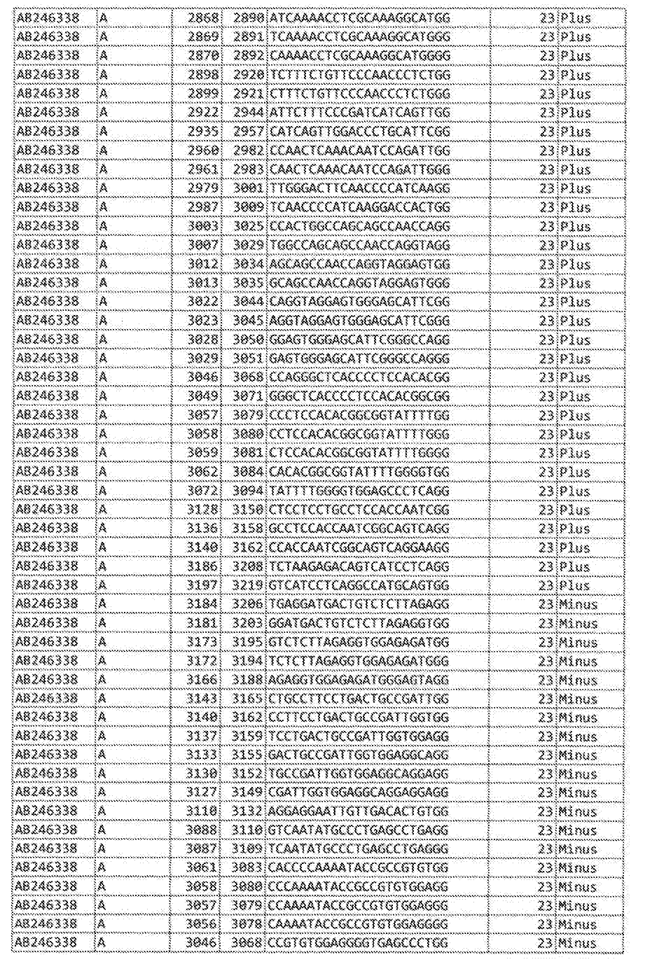

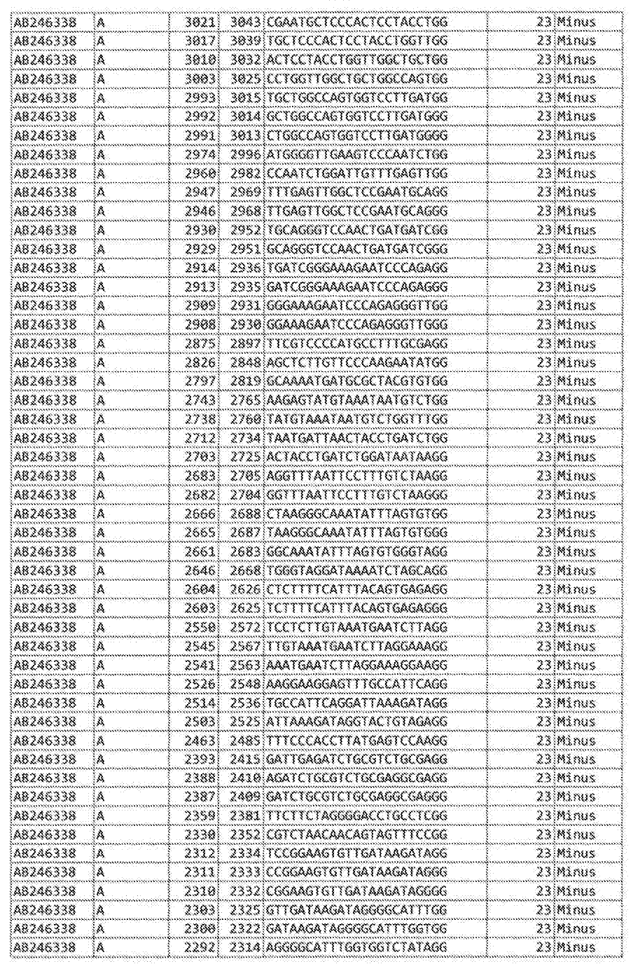

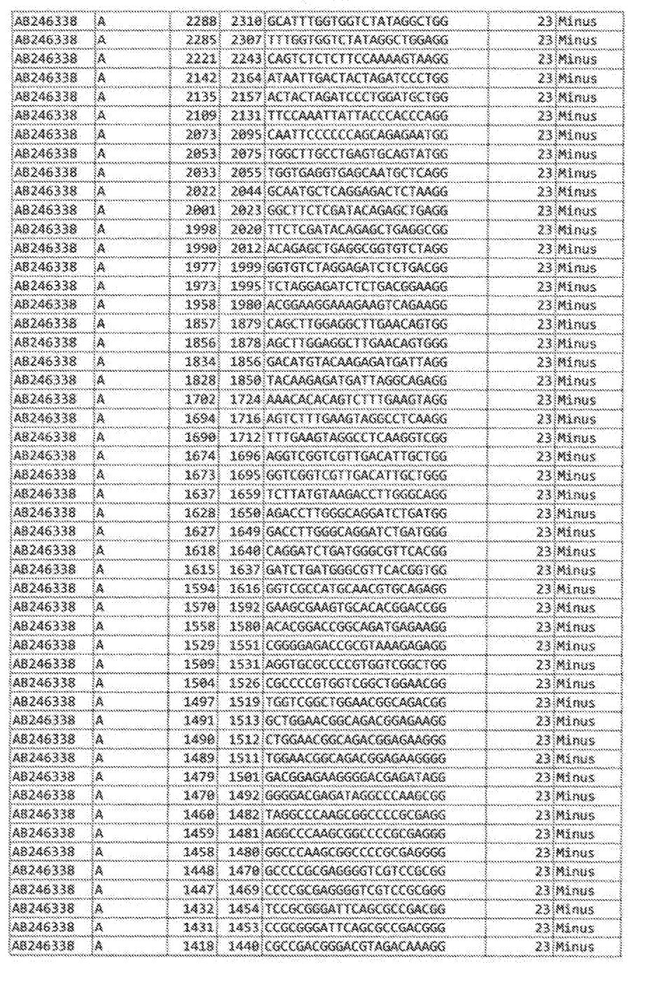

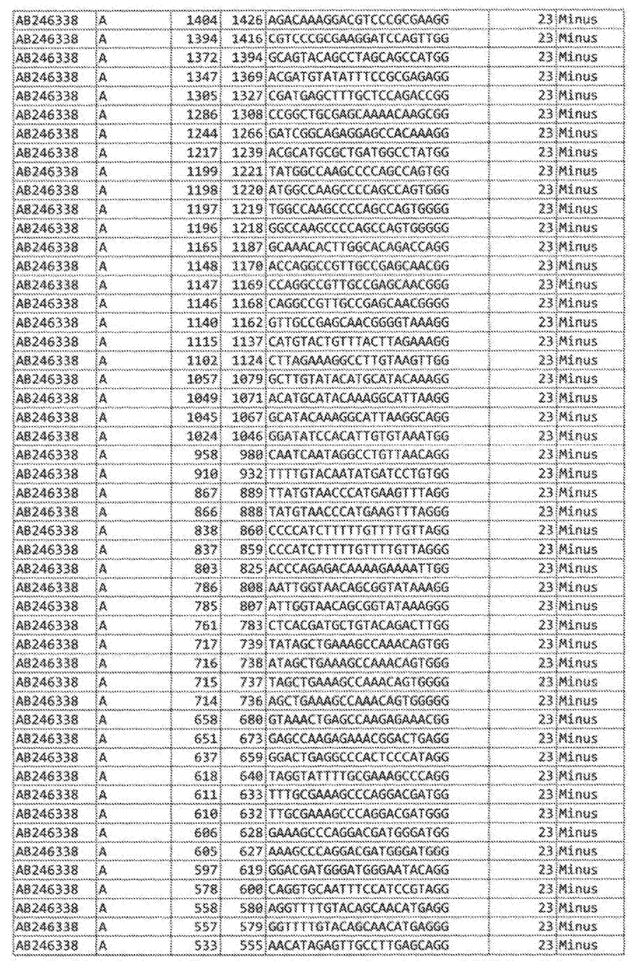

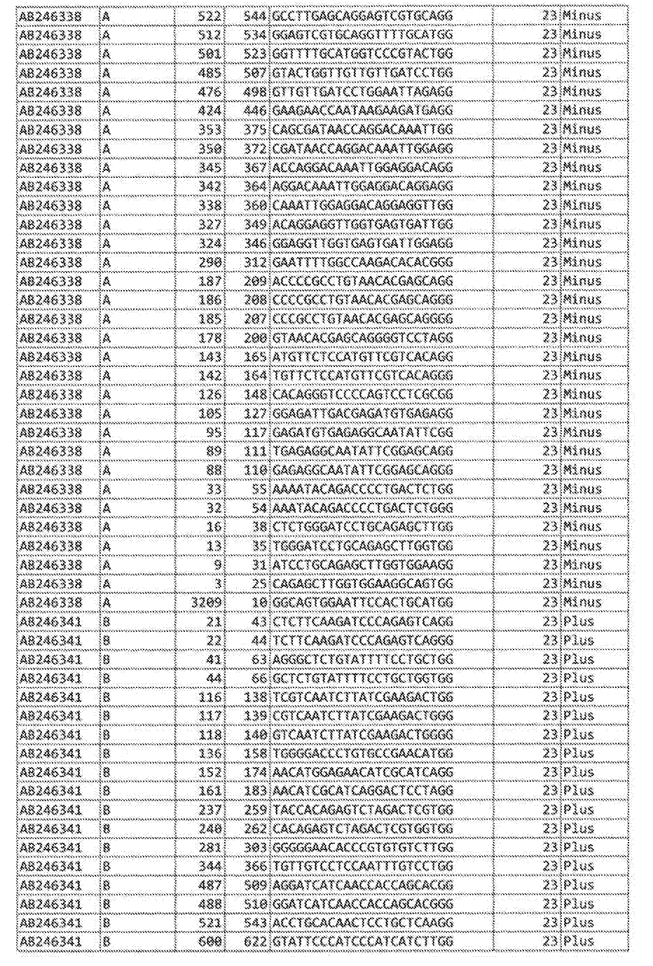

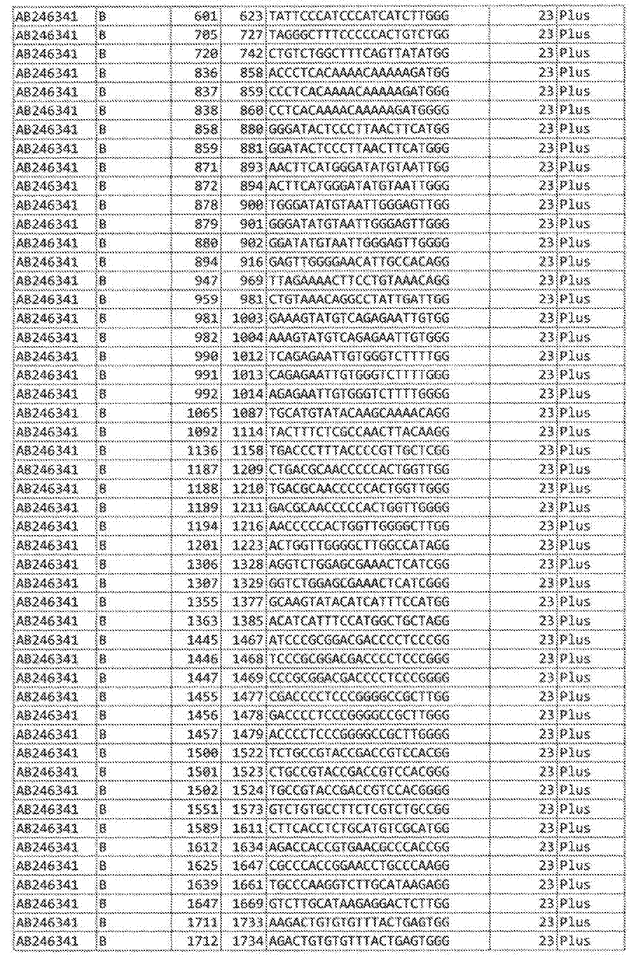

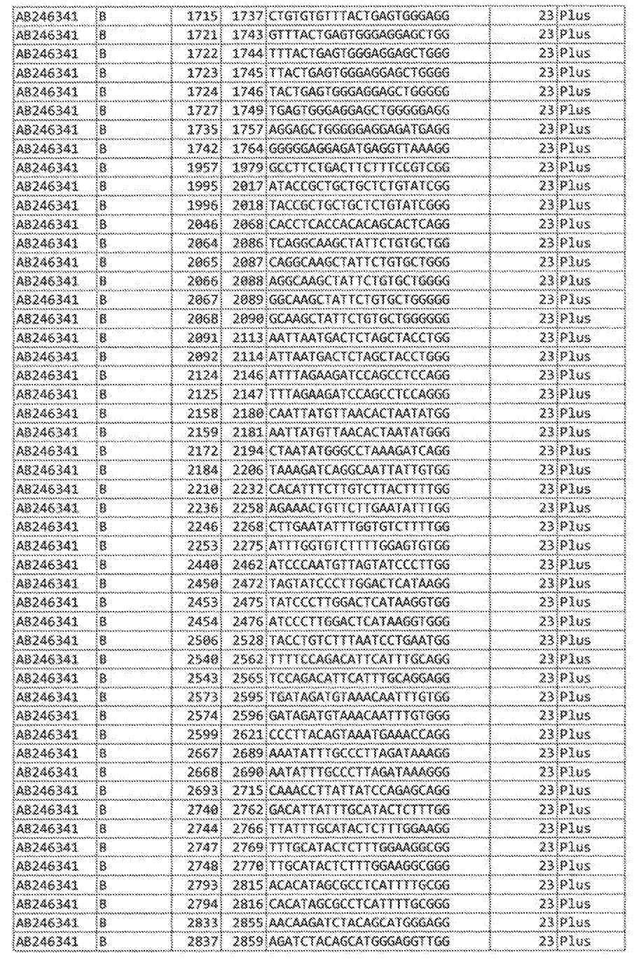

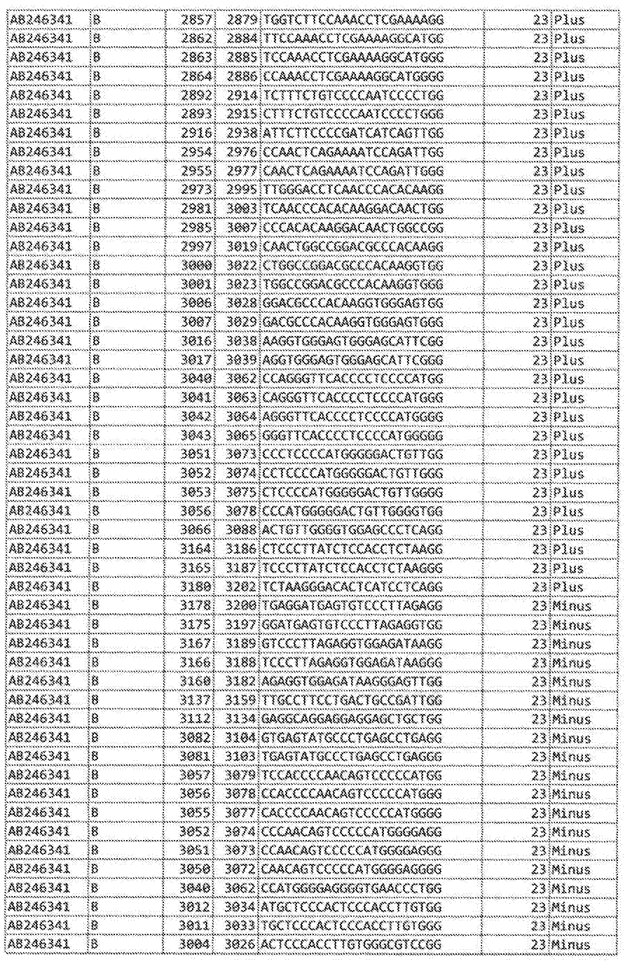

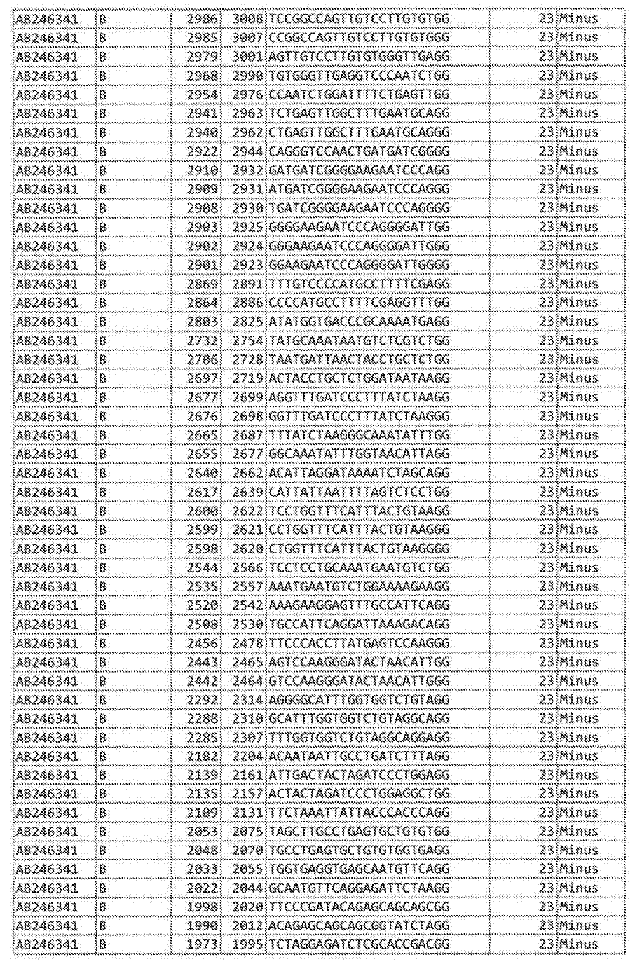

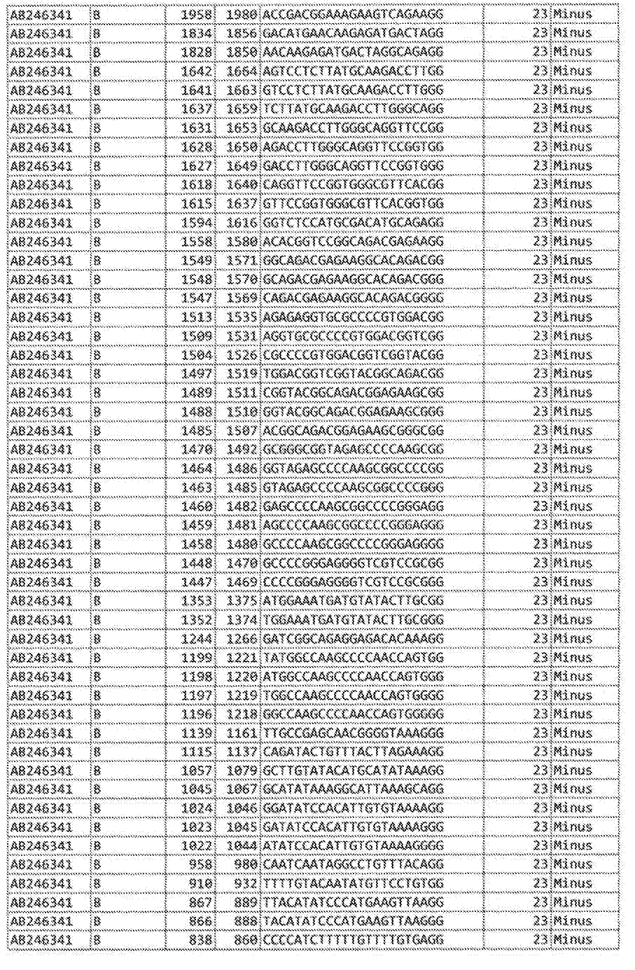

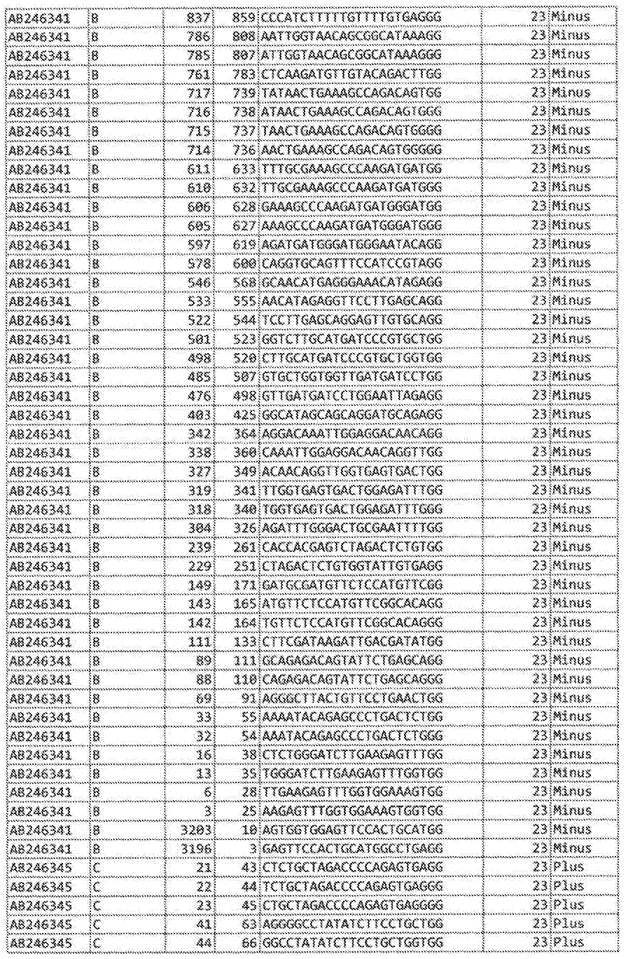

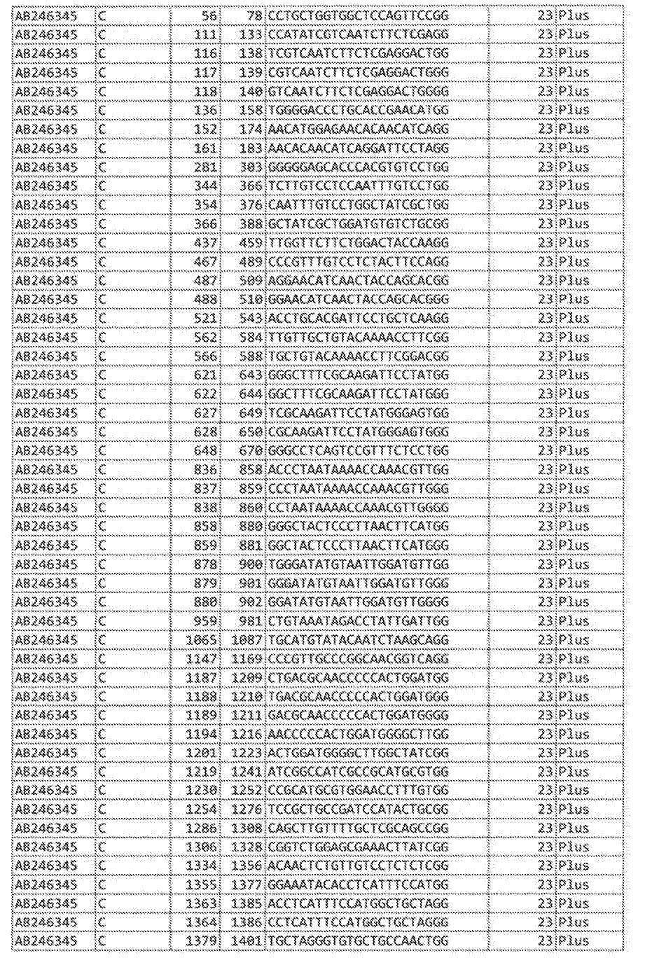

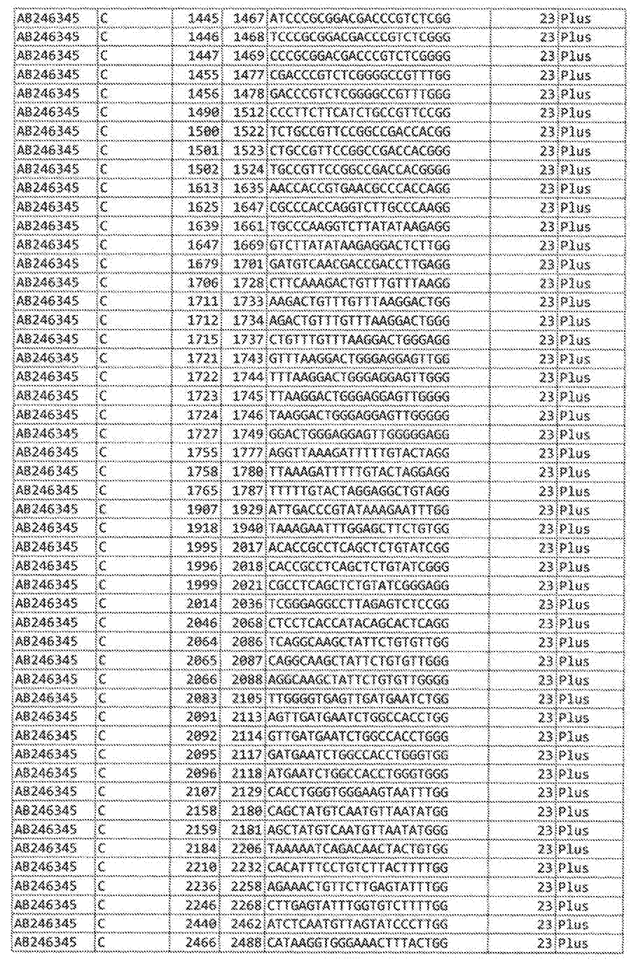

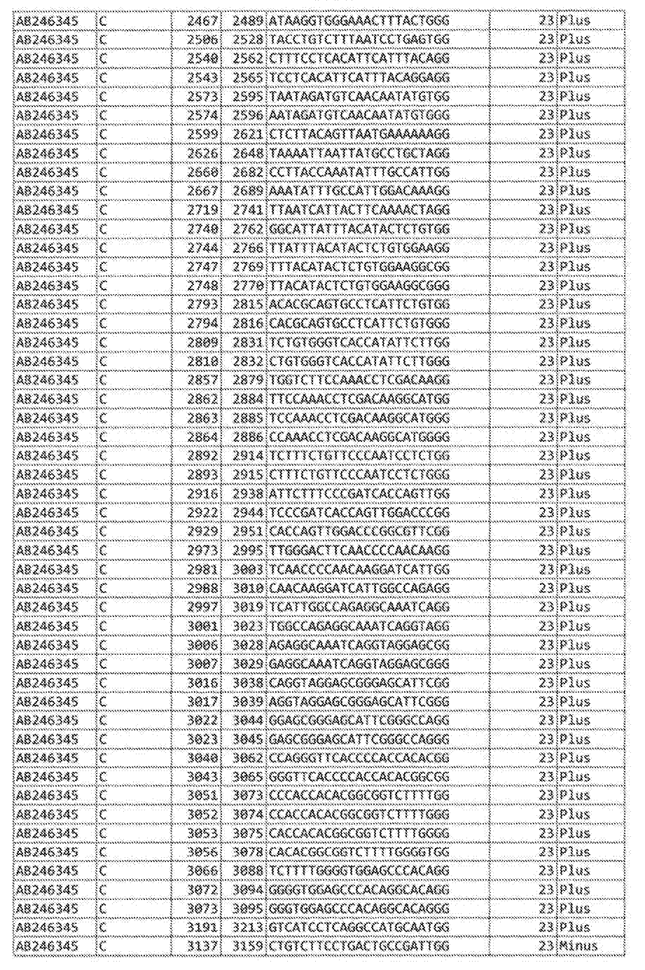

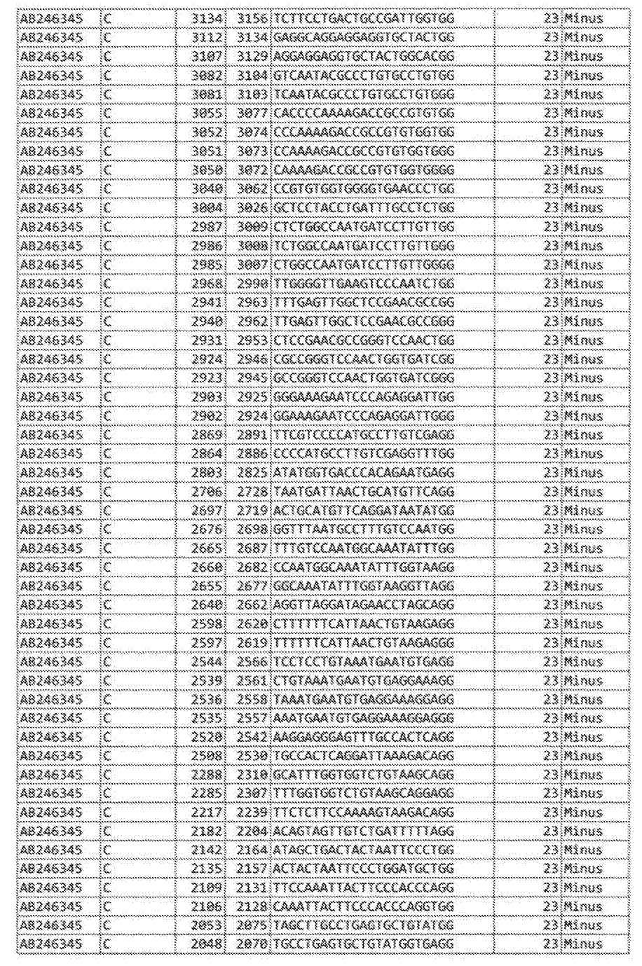

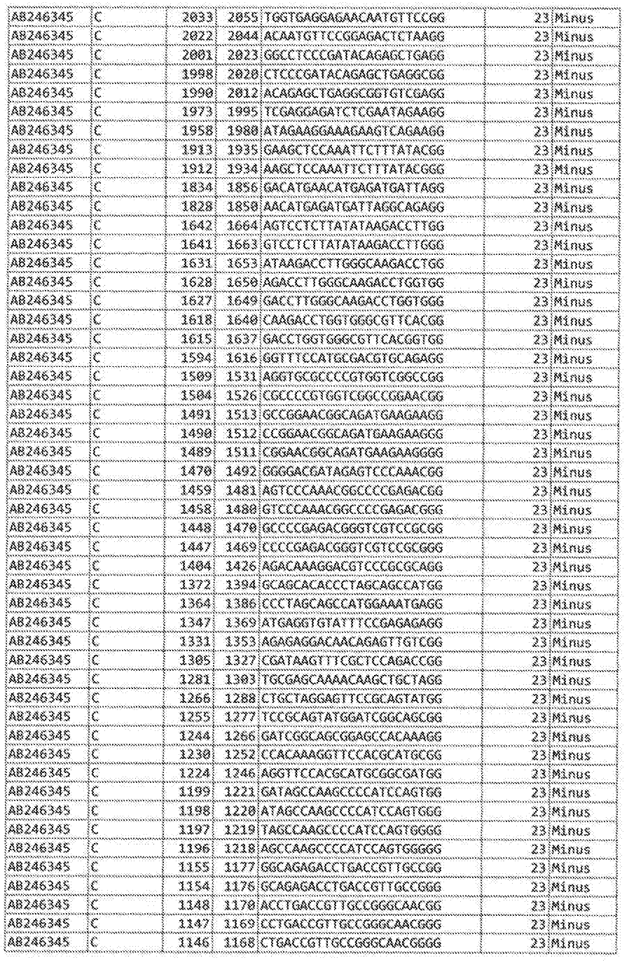

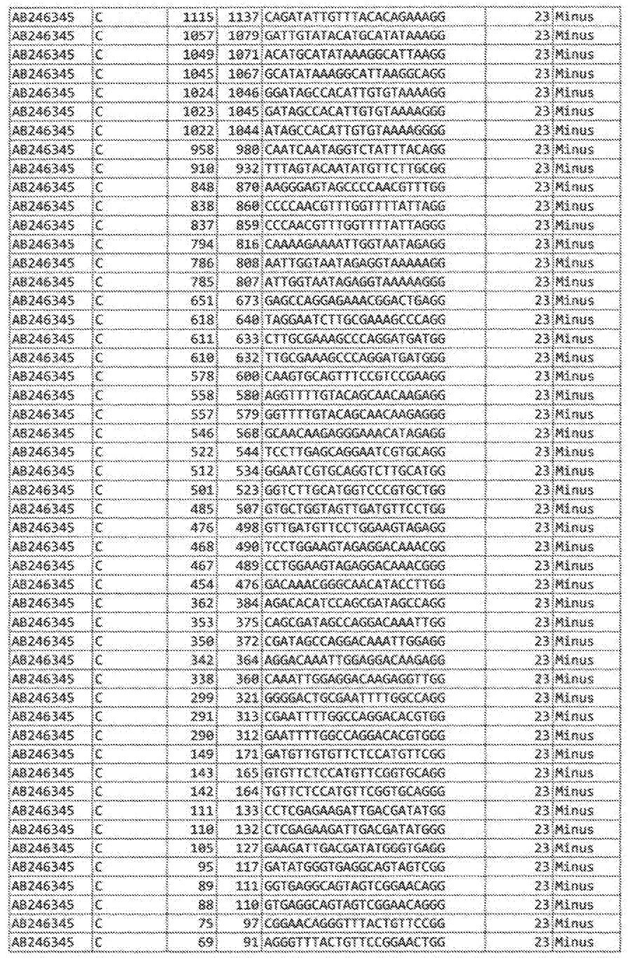

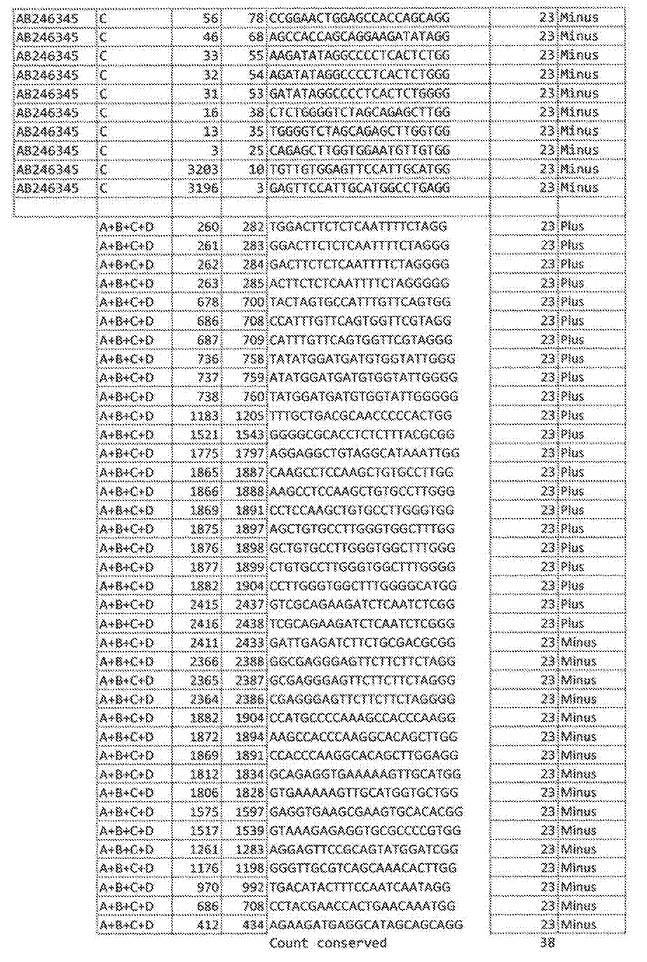

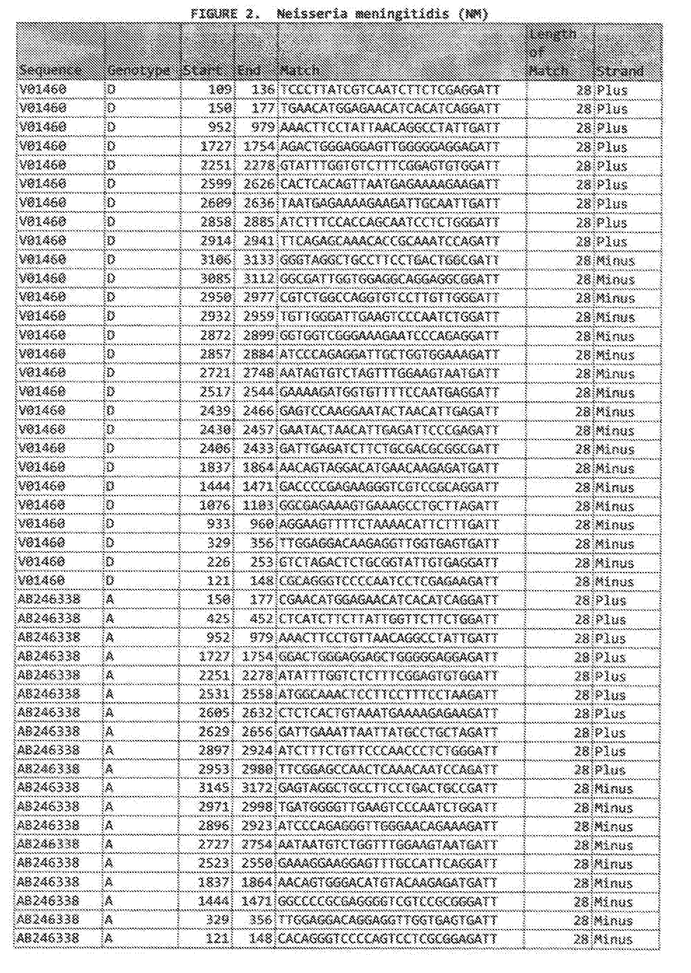

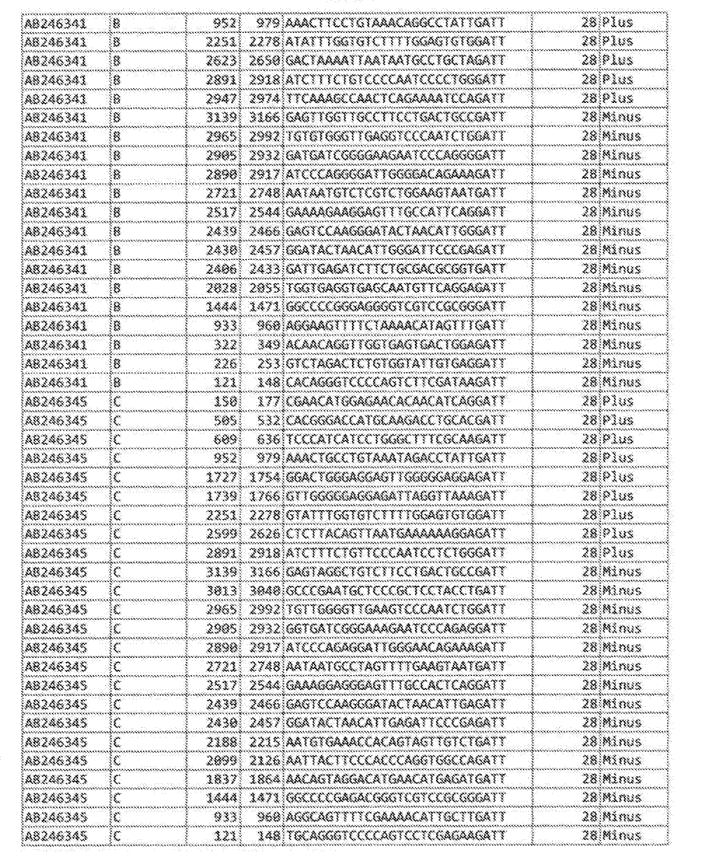

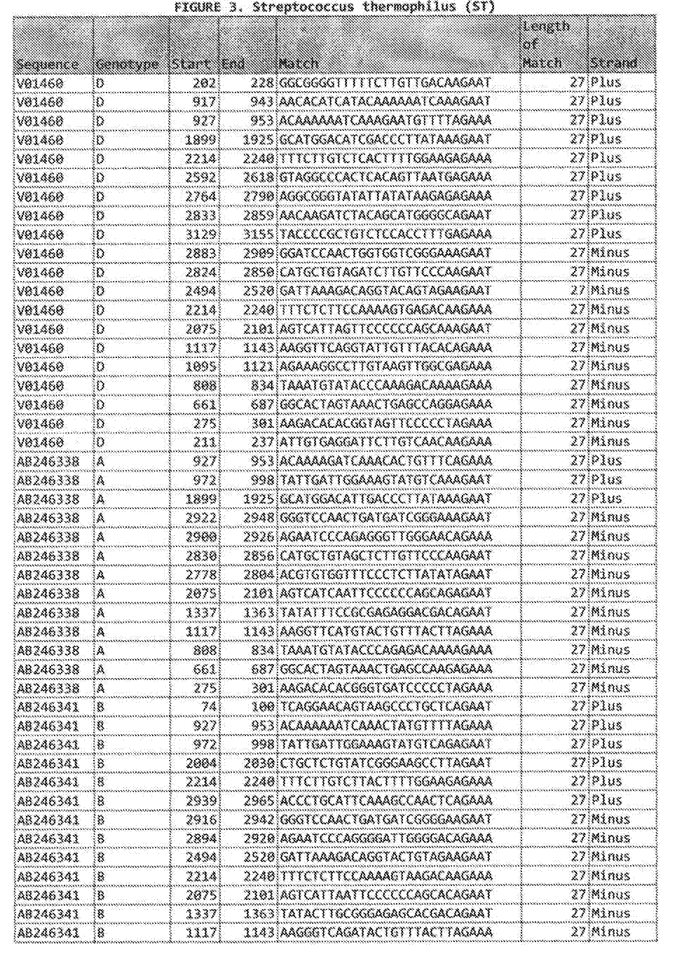

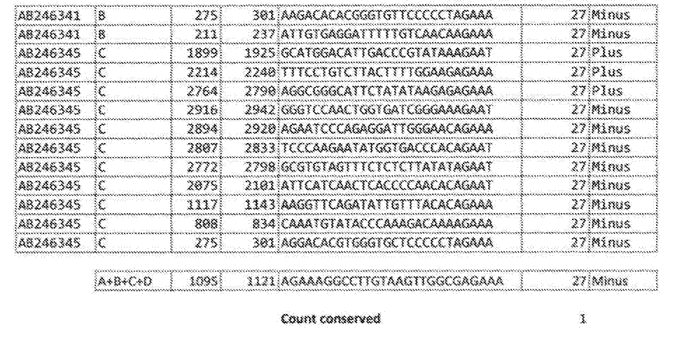

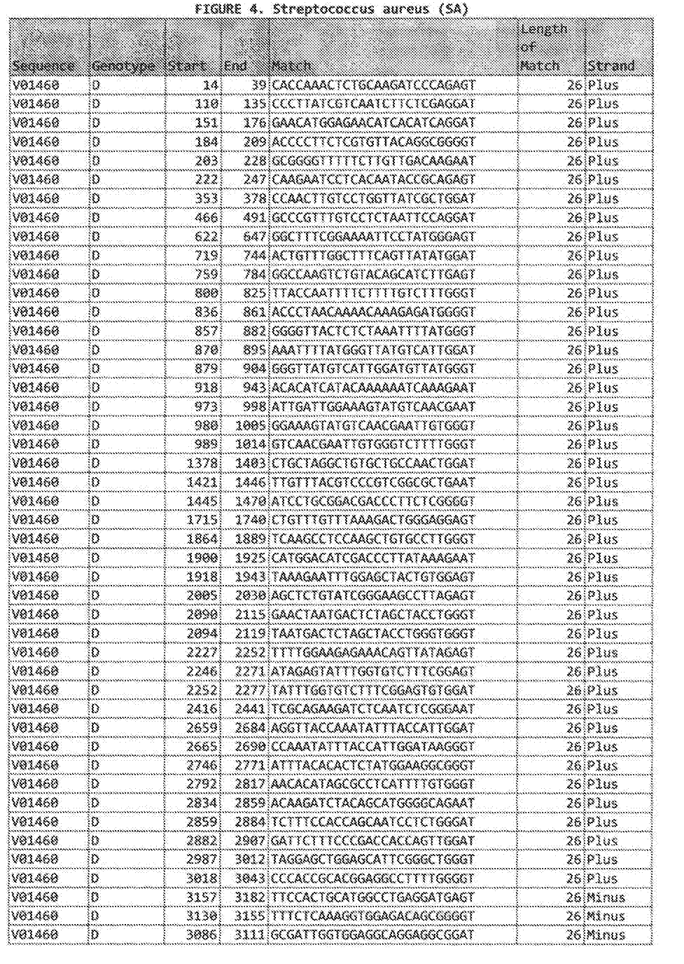

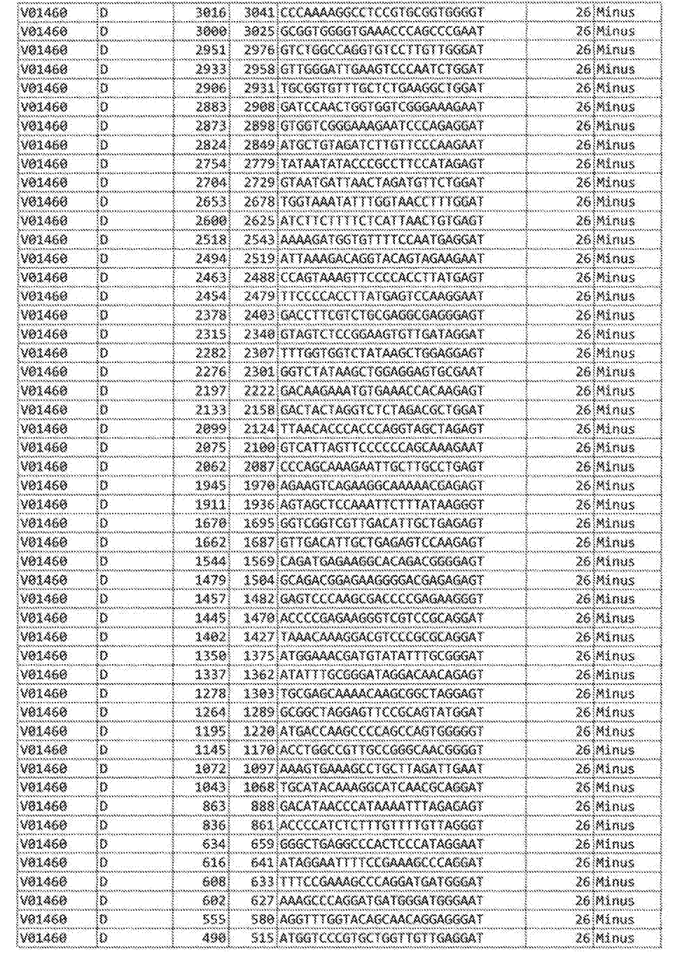

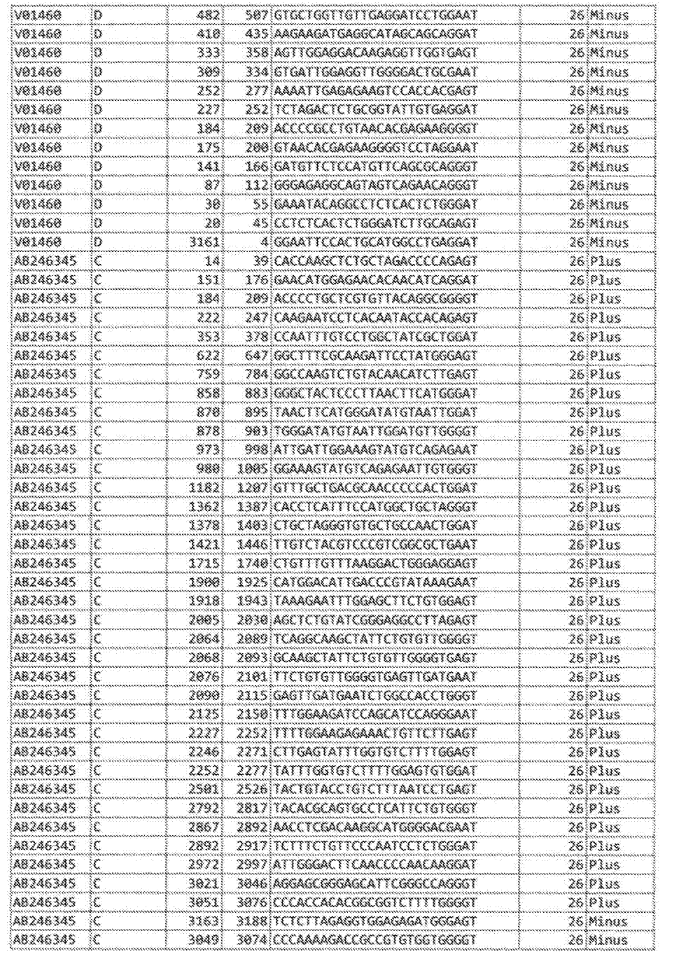

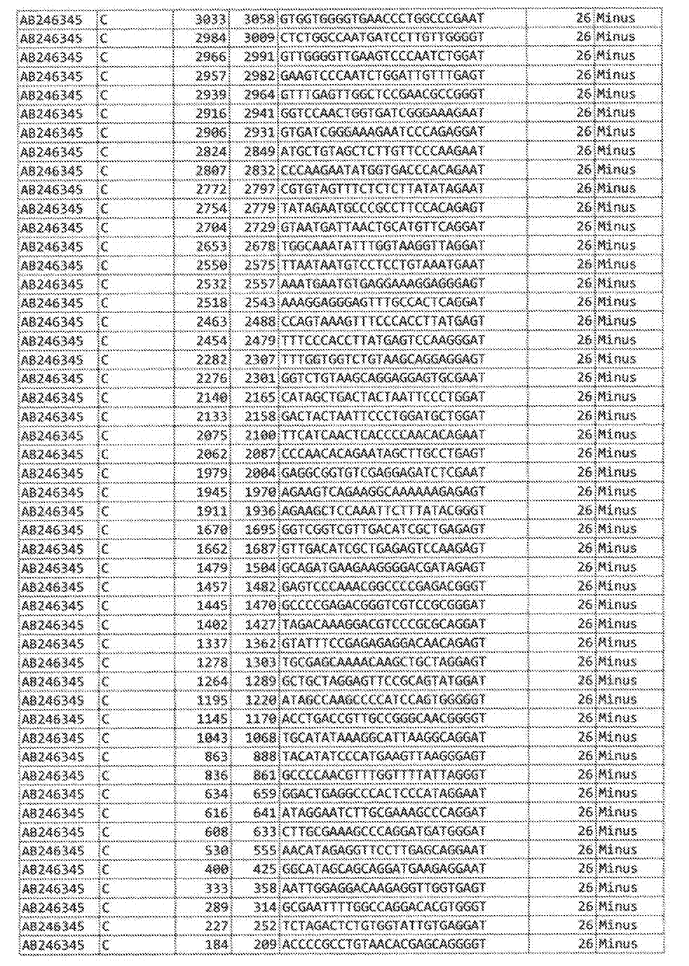

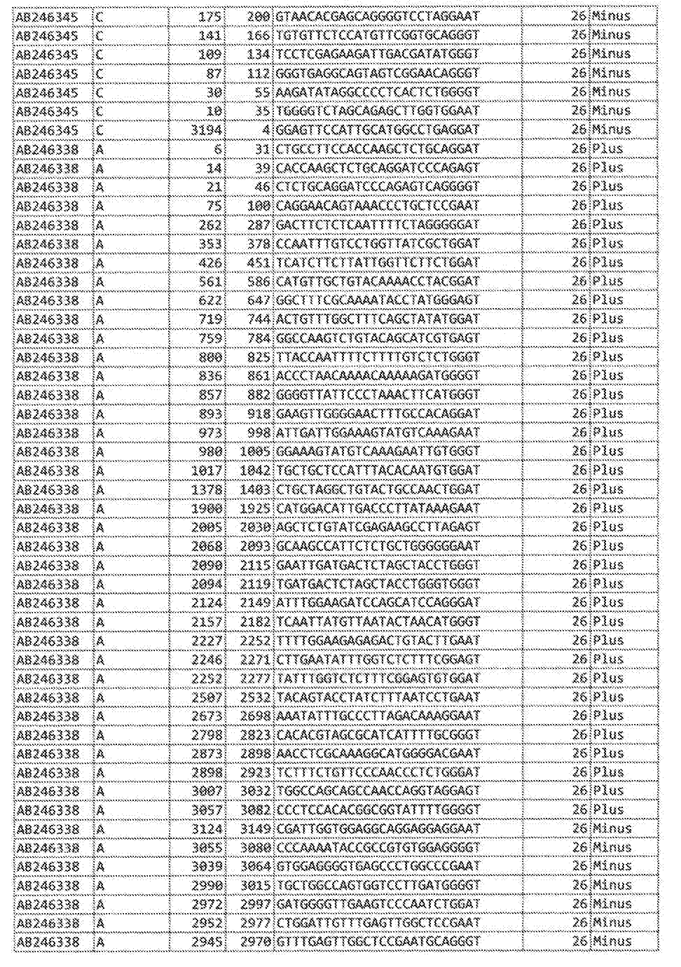

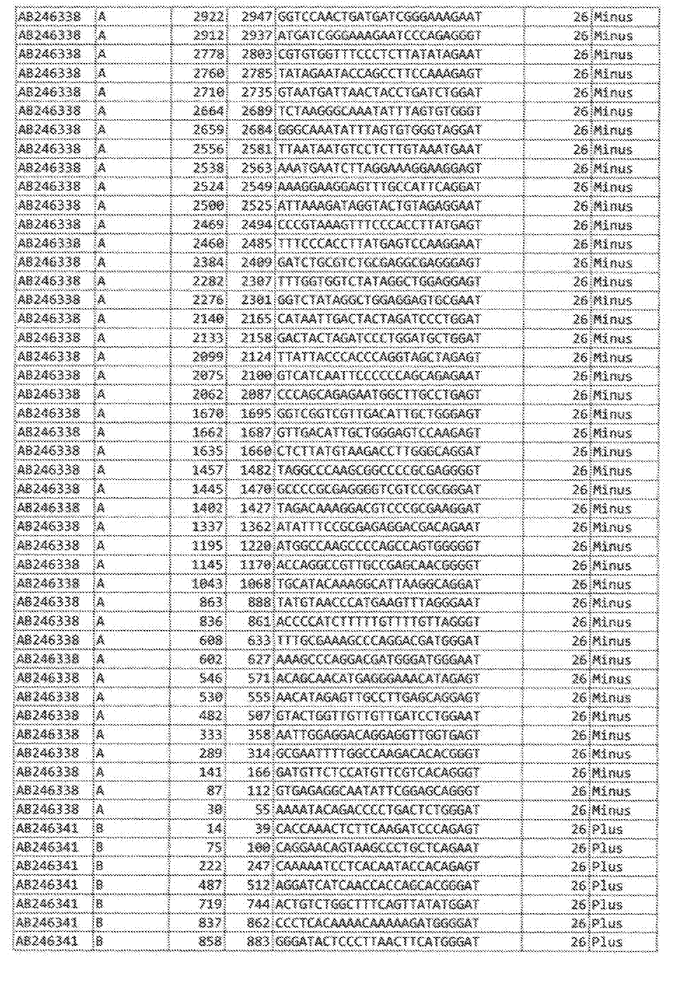

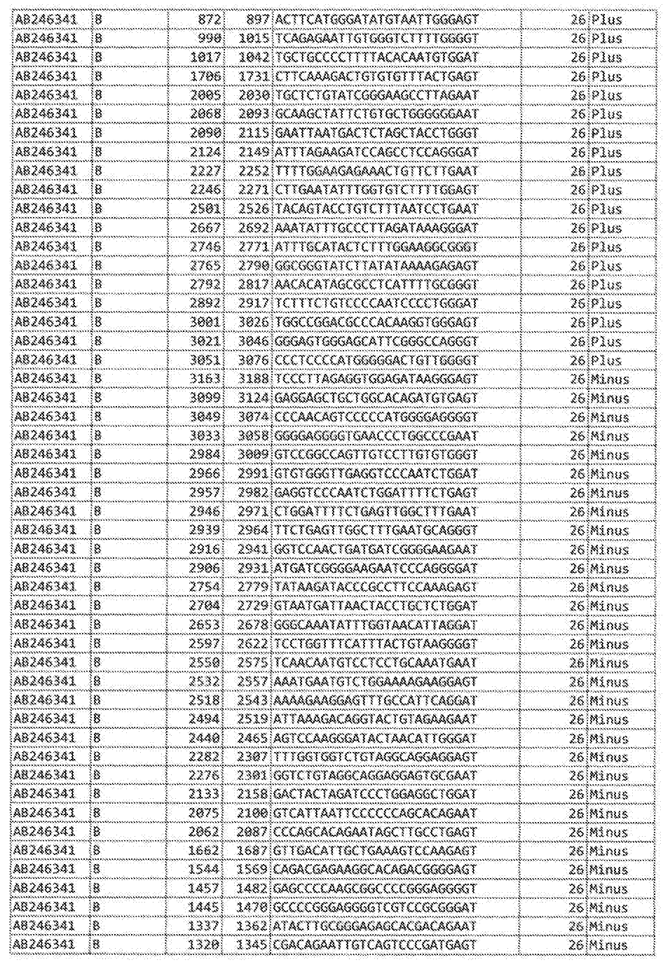

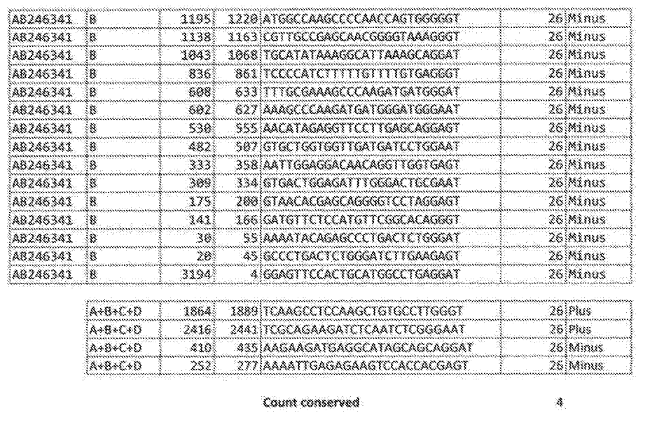

Accordingly, certain embodiments of the present invention provide a guide RNA (gRNA) sequence comprising a first sequence that corresponds to a target sequence described in FIG. 1, FIG. 2, FIG. 3 or FIG. 4 and a second sequence that is a tracer RNA sequence, e.g., located 3′ of the first sequence.

In certain embodiments, the target sequence is a conserved target sequence described in FIG. 1, FIG. 2, FIG. 3 or FIG. 4.

Certain embodiments provide a composition comprising a gRNA described herein and a mRNA sequence encoding a CRISPR associated protein 9 (Cas9).

Certain embodiments provide a nucleic acid-lipid particle comprising: (a) one or more gRNA described herein; (b) a cationic lipid; and (c) a non-cationic lipid.

In certain embodiments, the nucleic acid-lipid particle further comprises a mRNA sequence encoding a CRISPR associated protein 9 (Cas9).

Certain embodiments provide a pharmaceutical composition comprising a nucleic acid-lipid particle described herein and a pharmaceutically acceptable carrier, which composition optionally comprises a second nucleic acid-lipid particle comprising a mRNA sequence encoding a Cas9, which second nucleic acid-lipid particle does not comprise a gRNA.

Definitions

As used herein, the following terms have the meanings ascribed to them unless specified otherwise.

The term “Hepatitis B virus” (abbreviated as HBV) refers to a virus species of the genus Orthohepadnavirus, which is a part of the Hepadnaviridae family of viruses, and that is capable of causing liver inflammation in humans.

The term “Hepatitis D virus” (abbreviated as HDV) refers to a virus species of the genus Deltaviridae, which is capable of causing liver inflammation in humans.

Briefly, the CRISPR technology can be utilized by combining the CRISPR associated protein 9 (Cas9), an RNA-guided DNA endonuclease enzyme, with a guide RNA (gRNA) sequence that is designed to be utilized by the Cas9 to target a specific sequence of HBV. This combination functions to inhibit HBV expression.

An example of an open reading frame (ORF) for the Streptococcus pyogenes (SP) Cas9, found in a plasmid provided by the George Church Lab to addgene.org, is provided below. The mRNA encoding Cas9 will also generally include a polyA tail and other elements (e.g., including 5′ & 3′ UTR). Cas9 mRNA can also be purchased, e.g., from TriLink BioTechnologies, Inc. Other versions of this Cas9 mRNA may be used by varying the codons without changing the translated protein. Also the nature of the NLS (nuclear localization signal) can vary. The example provided below includes a SV40 NLS (PKKKRKV; SEQ ID NO:1) at the 3′ end. Further descriptions of CRISPR related proteins, and the expression thereof, can be found, e.g., in International Publication Number WO 2015/006747.

An Open Reading Frame for the Streptococcus
pyogenes (SP) Cas9
(SEQ ID NO: 2)
5′ ATGGACAAGAAGTACTCCATTGGGCTCGATATCGGCACAAACAGCGT
CGGCTGGGCCGTCATTACGGACGAGTACAAGGTGCCGAGCAAAAAATTCA
AAGTTCTGGGCAATACCGATCGCCACAGCATAAAGAAGAACCTCATTGGC
GCCCTCCTGTTCGACTCCGGGGAGACGGCCGAAGCCACGCGGCTCAAAAG
AACAGCACGGCGCAGATATACCCGCAGAAAGAATCGGATCTGCTACCTGC
AGGAGATCTTTAGTAATGAGATGGCTAAGGTGGATGACTCTTTCTTCCAT
AGGCTGGAGGAGTCCTTTTTGGTGGAGGAGGATAAAAAGCACGAGCGCCA
CCCAATCTTTGGCAATATCGTGGACGAGGTGGCGTACCATGAAAAGTACC
CAACCATATATCATCTGAGGAAGAAGCTTGTAGACAGTACTGATAAGGCT
GACTTGCGGTTGATCTATCTCGCGCTGGCGCATATGATCAAATTTCGGGG
ACACTTCCTCATCGAGGGGGACCTGAACCCAGACAACAGCGATGTCGACA
AACTCTTTATCCAACTGGTTCAGACTTACAATCAGCTTTTCGAAGAGAAC
CCGATCAACGCATCCGGAGTTGACGCCAAAGCAATCCTGAGCGCTAGGCT
GTCCAAATCCCGGCGGCTCGAAAACCTCATCGCACAGCTCCCTGGGGAGA
AGAAGAACGGCCTGTTTGGTAATCTTATCGCCCTGTCACTCGGGCTGACC
CCCAACTTTAAATCTAACTTCGACCTGGCCGAAGATGCCAAGCTTCAACT
GAGCAAAGACACCTACGATGATGATCTCGACAATCTGCTGGCCCAGATCG
GCGACCAGTACGCAGACCTTTTTTTGGCGGCAAAGAACCTGTCAGACGCC
ATTCTGCTGAGTGATATTCTGCGAGTGAACACGGAGATCACCAAAGCTCC
GCTGAGCGCTAGTATGATCAAGCGCTATGATGAGCACCACCAAGACTTGA
CTTTGCTGAAGGCCCTTGTCAGACAGCAACTGCCTGAGAAGTACAAGGAA
ATTTTCTICGATCAGTCTAAAAATGGCTACGCCGGATACATTGACGGCGG
AGCAAGCCAGGAGGAATTTTACAAATTTATTAAGCCCATCTTGGAAAAAA
TGGACGGCACCGAGGAGCTGCTGGTAAAGCTTAACAGAGAAGATCTGTTG
CGCAAACAGCGCACTTTCGACAATGGAAGCATCCCCCACCAGATTCACCT
GGGCGAACTGCACGCTATCCTCAGGCGGCAAGAGGATTTCTACCCCTTTT
TGAAAGATAACAGGGAAAAGATTGAGAAAATCCTCACATTTCGGATACCC
TACTATGTAGGCCCCCTCGCCCGGGGAAATTCCAGATTCGCGTGGATGAC
TCGCAAATCAGAAGAGACCATCACTCCCTGGAACTTCGAGGAAGTCGTGG
ATAAGGGGGCCTCTGCCCAGTCCTTCATCGAAAGGATGACTAACTTTGAT
AAAAATCTGCCTAACGAAAAGGTGCTTCCTAAACACTCTCTGCTGTACGA
GTACTTCACAGTTTATAACGAGCTCACCAAGGTCAAATACGTCACAGAAG
GGATGAGAAAGCCAGCATTCCTGTCTGGAGAGCAGAAGAAAGCTATCGTG
GACCTCCTCTTCAAGACGAACCGGAAAGTTACCGTGAAACAGCTCAAAGA
AGACTATTTCAAAAAGATTGAATGTTTCGACTCTGTTGAAATCAGCGGAG
TGGAGGATCGCTTCAACGCATCCCTGGGAACGTATCACGATCTCCTGAAA
ATCATTAAAGACAAGGACTTCCTGGACAATGAGGAGAACGAGGACATTCT
TGAGGACATTGTCCTCACCCTTACGTTGTTTGAAGATAGGGAGATGATTG
AAGAACGCTTGAAAACTTACGCTCATCTCTTCGACGACAAAGTCATGAAA
CAGCTCAAGAGGCGCCGATATACAGGATGGGGGCGGCTGTCAAGAAAACT
GATCAATGGGATCCGAGACAAGCAGAGTGGAAAGACAATCCTGGATTTTC
TTAAGTCCGATGGATTTGCCAACCGGAACTTCATGCAGTTGATCCATGAT
GACTCTCTCACCTTTAAGGAGGACATCCAGAAAGCACAAGTTTCTGGCCA
GGGGGACAGTCTTCACGAGCACATCGCTAATCTTGCAGGTAGCCCAGCTA
TCAAAAAGGGAATACTGCAGACCGTTAAGGTCGTGGATGAACTCGTCAAA
GTAATGGGAAGGCATAAGCCCGAGAATATCGTTATCGAGATGGCCCGAGA
GAACCAAACTACCCAGAAGGGACAGAAGAACAGTAGGGAAAGGATGAAGA
GGATTGAAGAGGGTATAAAAGAACTGGGGTCCCAAATCCTTAAGGAACAC
CCAGTTGAAAACACCCAGCTTCAGAATGAGAAGCTCTACCTGTACTACCT
GCAGAACGGCAGGGACATGTACGTGGATCAGGAACTGGACATCAATCGGC
TCTCCGACTACGACGTGGATCATATCGTGCCCCAGTCTTTTCTCAAAGAT
GATTCTATTGATAATAAAGTGTTGACAAGATCCGATAAAAATAGAGGGAA
GAGTGATAACGTCCCCTCAGAAGAAGTTGTCAAGAAAATGAAAAATTATT
GGCGGCAGCTGCTGAACGCCAAACTGATCACACAACGGAAGTTCGATAAT
CTGACTAAGGCTGAACGAGGTGGCCTGTCTGAGTTGGATAAAGCCGGCTT
CATCAAAAGGCAGCTTGTTGAGACACGCCAGATCACCAAGCACGTGGCCC
AAATTCTCGATTCACGCATGAACACCAAGTACGATGAAAATGACAAACTG
ATTCGAGAGGTGAAAGTTATTACTCTGAAGTCTAAGCTGGTCTCAGATTT
CAGAAAGGACTTTCAGTTTTATAAGGTGAGAGAGATCAACAATTACCACC
ATGCGCATGATGCCTACCTGAATGCAGTGGTAGGCACTGCACTTATCAAA
AAATATCCCAAGCTTGAATCTGAATTTGTTTACGGAGACTATAAAGTGTA
CGATGTTAGGAAAATGATCGCAAAGTCTGAGCAGGAAATAGGCAAGGCCA
CCGCTAAGTACTTCTTTTACAGCAATATTATGAATTTTTTCAAGACCGAG
ATTACACTGGCCAATGGAGAGATTCGGAAGCGACCACTTATCGAAACAAA
CGGAGAAACAGGAGAAATCGTGTGGGACAAGGGTAGGGATTTCGCGACAG
TCCGGAAGGTCCTGTCCATGCCGCAGGTGAACATCGTTAAAAAGACCGAA
GTACAGACCGGAGGCTTCTCCAAGGAAAGTATCCTCCCGAAAAGGAACAG
CGACAAGCTGATCGCACGCAAAAAAGATTGGGACCCCAAGAAATACGGCG
GATTCGATTCTCCTACAGTCGCTTACAGTGTACTGGTTGTGGCCAAAGTG
GAGAAAGGGAAGTCTAAAAAACTCAAAAGCGTCAAGGAACTGCTGGGCAT
CACAATCATGGAGCGATCAAGCTTCGAAAAAAACCCCATCGACTTTCTCG
AGGCGAAAGGATATAAAGAGGTCAAAAAAGACCTCATCATTAAGCTTCCC
AAGTACTCTCTCTTTGAGCTTGAAAACGGCCGGAAACGAATGCTCGCTAG
TGCGGGCGAGCTGCAGAAAGGTAACGAGCTGGCACTGCCCTCTAAATACG
TTAATTTCTTGTATCTGGCCAGCCACTATGAAAAGCTCAAAGGGTCTCCC
GAAGATAATGAGCAGAAGCAGCTGTTCGTGGAACAACACAAACACTACCT
TGATGAGATCATCGAGCAAATAAGCGAATTCTCCAAAAGAGTGATCCTCG
CCGACGCTAACCTCGATAAGGTGCTTTCTGCTTACAATAAGCACAGGGAT
AAGCCCATCAGGGAGCAGGCAGAAAACATTATCCACTTGTTTACTCTGAC
CAACTTGGGCGCGCCTGCAGCCTTCAAGTACTTCGACACCACCATAGACA
GAAAGCGGTACACCTCTACAAAGGAGGTCCTGGACGCCACACTGATTCAT
CAGTCAATTACGGGGCTCTATGAAACAAGAATCGACCTCTCTCAGCTCGG
TGGAGACAGCAGGGCTGACCCCAAGAAGAAGAGGAAGGTGTGA3′

CRISPR Guide RNA (gRNA)

Regarding gRNA described herein, the first 20 Ns in gRNA sequence below correspond to the target sequence (see FIGS. 1-4; and as described in the Figures, said sequence does not include the PAM sequence). The remaining portion of the gRNA sequence (the tracer RNA sequence) comprises sequences for guiding the gRNA into the Cas9. The gRNA scaffold below was described in Sander et al., Nature Biotechnology, 32(4), 347-355, including Supplementary Information (2014). As is described in Sander et al., different gRNA strategies (e.g., utilizing different tracer RNA sequences) have been tried with more or less success.

A gRNA Sequence
(SEQ ID NO: 3)
5′ NNNNNNNNNNNNNNNNNNNNGUUUUAGAGCUAGAAAUAGCAAGUUA
AAAUAAGGCUAGUCCGUUAUCAACUUGAAAAAGUGGCACCGAGUCGGUG
CUUUU3′

Other examples of tracer RNA sequences are provided below. (see, e.g., Ran et al., Nature, 520, 186-191 (2015))

A Tracer RNA for Streptococcus aureus (SA)
(SEQ ID NO: 4)
5′ GUUUUAGUACUCUGGAAACAGAAUCUACUAAAACAAGGCAAAAUGCC
GUGUUUAUCUCGUCAACUUGUUGGCGAGAUUUU3′
A Tracer RNA for Streptococcus thermophilus (ST)
(SEQ ID NO: 5)
5′ GUUUUUGUACUCGAAAGAAGCUACAAAGAUAAGGCUUCAUGCCGAAA
UCAACACCCUGUCAUUUUAUGGCAGGGUGUUUU3′

The tracer RNA sequence of the gRNA of the present invention in certain embodiments corresponds to one of the three tracer sequences described hereinabove. In certain embodiments, the tracer sequence is at least 90% identical to any one of the three tracer sequences (e.g., at least 90%, 91%, 92%, 93%, 94%, 95%, 96%, 97%, 98% or 99% identical).

The phrase “inhibiting expression of a target gene” refers to the ability to silence, reduce, or inhibit expression of a target gene (e.g., a gene within the HBV genome). To examine the extent of gene silencing, a test sample (e.g., a biological sample from an organism of interest expressing the target gene or a sample of cells in culture expressing the target gene) is contacted with a composition that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the composition. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an gRNA sequence that targets a different gene, a scrambled gRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art. An “effective amount” or “therapeutically effective amount” of a therapeutic nucleic acid is an amount sufficient to produce the desired effect, e.g., an inhibition of expression of a target sequence in comparison to the normal expression level detected in the absence of the nucleic acid. In particular embodiments, inhibition of expression of a target gene or target sequence is achieved when the value obtained relative to the control (e.g., buffer only, an gRNA sequence that targets a different gene, a scrambled gRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, in the context of this invention, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

In certain embodiments, a nucleic acid sequence may include at least one “unlocked nucleobase analogue” (UNA).

The invention encompasses isolated or substantially purified nucleic acid molecules and compositions containing those molecules. In the context of the present invention, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “unlocked nucleobase analogue” (abbreviated as “UNA”) refers to an acyclic nucleobase in which the C2′ and C3′ atoms of the ribose ring are not covalently linked. The term “unlocked nucleobase analogue” includes nucleobase analogues having the following structure identified as Structure A:

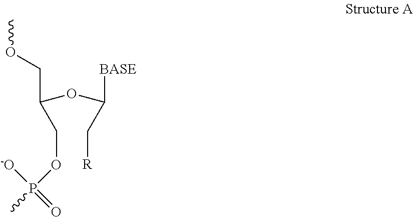

wherein R is hydroxyl, and Base is any natural or unnatural base such as, for example, adenine (A), cytosine (C), guanine (G) and thymine (T). UNA useful in the practice of the present invention include the molecules identified as acyclic 2′-3′-seco-nucleotide monomers in U.S. Pat. Ser. No. 8,314,227 which is incorporated by reference herein in its entirety.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid particle” includes a lipid formulation that can be used to deliver a therapeutic nucleic acid (e.g., gRNA) to a target site of interest (e.g., cell, tissue, organ, and the like). In preferred embodiments, the lipid particle of the invention is typically formed from a cationic lipid, a non-cationic lipid, and optionally a conjugated lipid that prevents aggregation of the particle. A lipid particle that includes a nucleic acid molecule (e.g., gRNA molecule) is referred to as a nucleic acid-lipid particle. Typically, the nucleic acid is fully encapsulated within the lipid particle, thereby protecting the nucleic acid from enzymatic degradation.

In certain instances, nucleic acid-lipid particles are extremely useful for systemic applications, as they can exhibit extended circulation lifetimes following intravenous (i.v.) injection, they can accumulate at distal sites (e.g., sites physically separated from the administration site), and they can mediate silencing of target gene expression at these distal sites. The nucleic acid may be complexed with a condensing agent and encapsulated within a lipid particle as set forth in PCT Publication No. WO 00/03683, the disclosure of which is herein incorporated by reference in its entirety for all purposes.

The lipid particles of the invention typically have a mean diameter of from about 30 nm to about 150 nm, from about 40 nm to about 150 nm, from about 50 rim to about 150 nm, from about 60 nm to about 130 nm, from about 70 rim to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 rim, 95 nm, 100 nm, 105 nm, 110 nm, 115 nm, 120 nm, 125 nm, 130 nm, 135 nm, 140 nm, 145 rim, or 150 nm, and are substantially non-toxic. In addition, nucleic acids, when present in the lipid particles of the present invention, are resistant in aqueous solution to degradation with a nuclease. Nucleic acid-lipid particles and their method of preparation are disclosed in, e.g., U.S. Patent Publication Nos. 20040142025 and 20070042031, the disclosures of which are herein incorporated by reference in their entirety for all purposes.

As used herein, “lipid encapsulated” can refer to a lipid particle that provides a therapeutic nucleic acid such as a gRNA, with full encapsulation, partial encapsulation, or both. In a preferred embodiment, the nucleic acid (e.g., gRNA) is fully encapsulated in the lipid particle (e.g., to form a nucleic acid-lipid particle).

The term “lipid conjugate” refers to a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used.

The term “amphipathic lipid” refers, in part, to any suitable material wherein the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “anionic lipid” refers to any lipid that is negatively charged at physiological pH. These lipids include, but are not limited to, phosphatidylglycerols, cardiolipins, diacyiphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

The terms “cationic lipid” and “amino lipid” are used interchangeably herein to include those lipids and salts thereof having one, two, three, or more fatty acid or fatty alkyl chains and a pH-titratable amino head group (e.g., an alkylamino or dialkylamino head group). The cationic lipid is typically protonated (i.e., positively charged) at a pH below the pKₐ of the cationic lipid and is substantially neutral at a pH above the pKₐ. The cationic lipids of the invention may also be termed titratable cationic lipids. In some embodiments, the cationic lipids comprise: a protonatable tertiary amine (e.g., pH-titratable) head group; C₁₈ alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, y-DLenDMA, DLin-K-DMA, DLin-K-C2-DMA (also known as DLin-C2K-DMA, XTC2, and C2K), DLin-K-C3-DMA, DLin-K-C4-DMA, DLen-C2K-DMA, y-DLen-C2K-DMA, DLin-M-C2-DMA (also known as MC2), and DLin-M-C3-DMA (also known as MC3).

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

The term “alkenyl” includes an alkyl, as defined above, containing at least one double bond between adjacent carbon atoms. Alkenyls include both cis and trans isomers. Representative straight chain and branched alkenyls include, but are not limited to, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

The term “alkynyl” includes any alkyl or alkenyl, as defined above, which additionally contains at least one triple bond between adjacent carbons. Representative straight chain and branched alkynyls include, without limitation, acetylenyl, propynyl, 1-butynyl, 2-butynyl, 1-pentynyl, 2-pentynyl, 3-methyl-1 butynyl, and the like.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

The term “heterocycle” includes a 5- to 7-membered monocyclic, or 7- to 10-membered bicyclic, heterocyclic ring which is either saturated, unsaturated, or aromatic, and which contains from 1 or 2 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include, but are not limited to, heteroaryls as defined below, as well as morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The terms “optionally substituted alkyl”, “optionally substituted alkenyl”, “optionally substituted alkynyl”, “optionally substituted acyl”, and “optionally substituted heterocycle” mean that, when substituted, at least one hydrogen atom is replaced with a substituent. In the case of an oxo substituent (═O), two hydrogen atoms are replaced. In this regard, substituents include, but are not limited to, oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}, wherein n is 0, 1, or 2, R^{x} and R^{y} are the same or different and are independently hydrogen, alkyl, or heterocycle, and each of the alkyl and heterocycle substituents may be further substituted with one or more of oxo, halogen, —OH, —CN, alkyl, —OR^{x}, heterocycle, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}. The term “optionally substituted,” when used before a list of substituents, means that each of the substituents in the list may be optionally substituted as described herein.

The term “halogen” includes fluoro, chloro, bromo, and iodo.

The term “fusogenic” refers to the ability of a lipid particle to fuse with the membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

The term “electron dense core”, when used to describe a lipid particle of the present invention, refers to the dark appearance of the interior portion of a lipid particle when visualized using cryo transmission electron microscopy (“cyroTEM”). Some lipid particles of the present invention have an electron dense core and lack a lipid bilayer structure. Some lipid particles of the present invention have an elctron dense core, lack a lipid bilayer structure, and have an inverse Hexagonal or Cubic phase structure. While not wishing to be bound by theory, it is thought that the non-bilayer lipid packing provides a 3-dimensional network of lipid cylinders with water and nucleic acid on the inside, i.e., essentially a lipid droplet interpenetrated with aqueous channels containing the nucleic acid.

“Distal site,” as used herein, refers to a physically separated site, which is not limited to an adjacent capillary bed, but includes sites broadly distributed throughout an organism.

“Serum-stable” in relation to nucleic acid-lipid particles means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of lipid particles that leads to a broad biodistribution of an active agent within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent directly to a target site within an organism. For example, an agent can be locally delivered by direct injection into a disease site, other target site, or a target organ such as the liver, heart, pancreas, kidney, and the like.

The term “virus particle load”, as used herein, refers to a measure of the number of virus particles (e.g., HBV and/or HDV) present in a bodily fluid, such as blood. For example, particle load may be expressed as the number of virus particles per milliliter of, e.g., blood. Particle load testing may be performed using nucleic acid amplification based tests, as well as non-nucleic acid-based tests (see, e.g., Puren et al., The Journal of Infectious Diseases, 201:S27-36 (2010)).

The term “mammal” refers to any mammalian species such as a human, mouse, rat, dog, cat, hamster, guinea pig, rabbit, livestock, and the like.

Description of Certain Embodiments

The present invention provides gRNA molecules that target the expression of one or more HBV genes, nucleic acid-lipid particles comprising one or more (e.g., a cocktail) of the gRNAs, and methods of delivering and/or administering the nucleic acid-lipid particles (e.g., for the treatment of HBV and/or HDV infection in humans). The gRNA molecules may be delivered concurrently with or sequentially with a mRNA molecule that encodes Cas9, thereby delivering components to utilize the CRISPR/Cas9 system to treat HBV and/or HDV infection in a human in need of such treatment. The Cas9 mRNA and gRNA may be present in the same nucleic acid-lipid particle, or they may be present in different nucleic acid-lipid particles.

In one aspect, the present invention provides gRNA molecules that target expression of one or more HBV genes. In certain instances, the present invention provides compositions comprising a combination (e.g., a cocktail, pool, or mixture) of gRNAs that target different regions of the HBV genome. In certain instances, the gRNA molecules of the invention are capable of inhibiting the replication of HBV and/or HDV in vitro or in vivo.

The present invention also provides a pharmaceutical composition comprising one or more (e.g., a cocktail) of the gRNAs described herein and a pharmaceutically acceptable carrier.

In certain embodiments, a composition described herein comprises one or more gRNA molecules, which silences expression of a Hepatitis B virus gene.

In another aspect, the present invention provides a nucleic acid-lipid particle that targets HBV gene expression. The nucleic acid-lipid particles typically comprise one or more (e.g., a cocktail) of the molecules described herein, a cationic lipid, and a non-cationic lipid. In certain instances, the nucleic acid-lipid particles further comprise a conjugated lipid that inhibits aggregation of particles. The nucleic acid-lipid particles may comprise one or more (e.g., a cocktail) of the molecules described herein, a cationic lipid, a non-cationic lipid, and a conjugated lipid that inhibits aggregation of particles.

In some embodiments, the gRNAs of the present invention are fully encapsulated in the nucleic acid-lipid particle. With respect to formulations comprising an gRNA cocktail, the different types of gRNA species present in the cocktail (e.g., gRNA compounds with different sequences) may be co-encapsulated in the same particle, or each type of gRNA species present in the cocktail may be encapsulated in a separate particle. The gRNA cocktail may be formulated in the particles described herein using a mixture of two, three or more individual gRNAs (each having a unique sequence) at identical, similar, or different concentrations or molar ratios. In one embodiment, a cocktail of gRNAs (corresponding to a plurality of gRNAs with different sequences) is formulated using identical, similar, or different concentrations or molar ratios of each gRNA species, and the different types of gRNAs are co-encapsulated in the same particle. In another embodiment, each type of gRNA species present in the cocktail is encapsulated in different particles at identical, similar, or different gRNA concentrations or molar ratios, and the particles thus formed (each containing a different gRNA payload) are administered separately (e.g., at different times in accordance with a therapeutic regimen), or are combined and administered together as a single unit dose (e.g., with a pharmaceutically acceptable carrier). The particles described herein are serum-stable, are resistant to nuclease degradation, and are substantially non-toxic to mammals such as humans.

The cationic lipid in the nucleic acid-lipid particles of the invention may comprise, e.g., one or more cationic lipids of Formula I-III described herein or any other cationic lipid species. In one embodiment, cationic lipid is a dialkyl lipid. In another embodiment, the cationic lipid is a trialkyl lipid. In one particular embodiment, the cationic lipid is selected from the group consisting of 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA; Compound (15)), 2,2-dilinoleyl-4-(2-dimethylaminoethyl)[1,3]-dioxolane (DLin-K-C2-DMA), 2,2-dilinoleyl-4-dimethylaminomethyl[1,3]-dioxolane (DLin-K-DMA), dilinoleylmethyl-3-dimethylaminopropionate (DLin-M-C2-DMA), (6Z,9Z,28Z,31Z)-heptatriaconta-6,9,28,31-tetraen-19-yl 4-(dimethylamino)butanoate (DLin-M-C3-DMA; Compound (7)), salts thereof, and mixtures thereof.

In another particular embodiment, the cationic lipid is selected from the group consisting of 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA; Compound (15)), 3-((6Z,9Z,28Z,31Z)-heptatriaconta-6,9,28,31-tetraen-19-yloxy)-N,N-dimethylpropan-1-amine (DLin-MP-DMA; Compound (8)), (6Z,9Z,28Z,31Z)-heptatriaconta-6,9,28,31-tetraen-19-yl 4-(dimethylamino)butanoate) (Compound (7)), (6Z,16Z)-12-((Z)-dec-4-enyl)docosa-6,16-dien-11-yl 5-(dimethylamino)pentanoate (Compound (13)), a salt thereof, or a mixture thereof.

In certain embodiments, the cationic lipid comprises from about 48 mol % to about 62 mol % of the total lipid present in the particle.

The non-cationic lipid in the nucleic acid-lipid particles of the present invention may comprise, e.g., one or more anionic lipids and/or neutral lipids. In some embodiments, the non-cationic lipid comprises one of the following neutral lipid components: (1) a mixture of a phospholipid and cholesterol or a derivative thereof; (2) cholesterol or a derivative thereof; or (3) a phospholipid. In certain preferred embodiments, the phospholipid comprises dipalmitoylphosphatidylcholine (DPPC), distearoylphosphatidylcholine (DSPC), or a mixture thereof. In a preferred embodiment, the non-cationic lipid is a mixture of DPPC and cholesterol. In a preferred embodiment, the non-cationic lipid is a mixture of DSPC and cholesterol.

In certain embodiments, the non-cationic lipid comprises a mixture of a phospholipid and cholesterol or a derivative thereof, wherein the phospholipid comprises from about 7 mol % to about 17 mol % of the total lipid present in the particle and the cholesterol or derivative thereof comprises from about 25 mol % to about 40 mol % of the total lipid present in the particle.

The lipid conjugate in the nucleic acid-lipid particles of the invention inhibits aggregation of particles and may comprise, e.g., one or more of the lipid conjugates described herein. In one particular embodiment, the lipid conjugate comprises a PEG-lipid conjugate. Examples of PEG-lipid conjugates include, but are not limited to, PEG-DAG conjugates, PEG-DAA conjugates, and mixtures thereof. In certain embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof. In certain embodiments, the PEG-lipid conjugate is a PEG-DAA conjugate. In certain embodiments, the PEG-DAA conjugate in the lipid particle may comprise a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, or mixtures thereof. In certain embodiments, wherein the PEG-DAA conjugate is a PEG-dimyristyloxypropyl (C₁₄) conjugate. In another embodiment, the PEG-DAA conjugate is a compound (66) (PEG-C-DMA) conjugate. In another embodiment, the lipid conjugate comprises a POZ-lipid conjugate such as a POZ-DAA conjugate.

In certain embodiments, the conjugated lipid that inhibits aggregation of particles comprises from about 0.5 mol % to about 3 mol % of the total lipid present in the particle.

In certain embodiments, the nucleic acid-lipid particle has a total lipid:gRNA mass ratio of from about 5:1 to about 15:1.

In certain embodiments, the nucleic acid-lipid particle has a median diameter of from about 30 nm to about 150 nm.

In certain embodiments, the nucleic acid-lipid particle has an electron dense core.

In some embodiments, the present invention provides nucleic acid-lipid particles comprising: (a) one or more (e.g., a cocktail) gRNA molecules described herein; (b) one or more cationic lipids or salts thereof comprising from about 50 mol % to about 85 mol % of the total lipid present in the particle; (c) one or more non-cationic lipids comprising from about 13 mol % to about 49.5 mol % of the total lipid present in the particle; and (d) one or more conjugated lipids that inhibit aggregation of particles comprising from about 0.5 mol % to about 2 mol % of the total lipid present in the particle.

In one aspect of this embodiment, the nucleic acid-lipid particle comprises: (a) one or more (e.g., a cocktail) gRNA molecules described herein; (b) a cationic lipid or a salt thereof comprising from about 52 mol % to about 62 mol % of the total lipid present in the particle; (c) a mixture of a phospholipid and cholesterol or a derivative thereof comprising from about 36 mol % to about 47 mol % of the total lipid present in the particle; and (d) a PEG-lipid conjugate comprising from about 1 mol % to about 2 mol % of the total lipid present in the particle. In one particular embodiment, the formulation is a four-component system comprising about 1.4 mol % PEG-lipid conjugate (e.g., PEG2000-C-DMA), about 57.1 mol % cationic lipid (e.g., DLin-K-C2-DMA) or a salt thereof, about 7.1 mol % DPPC (or DSPC), and about 34.3 mol % cholesterol (or derivative thereof).

In another aspect of this embodiment, the nucleic acid-lipid particle comprises: (a) one or more (e.g., a cocktail) gRNA molecules described herein; (b) a cationic lipid or a salt thereof comprising from about 56.5 mol % to about 66.5 mol % of the total lipid present in the particle; (c) cholesterol or a derivative thereof comprising from about 31.5 mol % to about 42.5 mol % of the total lipid present in the particle; and (d) a PEG-lipid conjugate comprising from about 1 mol % to about 2 mol % of the total lipid present in the particle. In one particular embodiment, the formulation is a three-component system which is phospholipid-free and comprises about 1.5 mol % PEG-lipid conjugate (e.g., PEG2000-C-DMA), about 61.5 mol % cationic lipid (e.g., DLin-K-C2-DMA) or a salt thereof, and about 36.9 mol % cholesterol (or derivative thereof).

Additional formulations are described in PCT Publication No. WO 09/127060 and published US patent application publication number US 2011/0071208 A1, the disclosures of which are herein incorporated by reference in their entirety for all purposes.

In other embodiments, the present invention provides nucleic acid-lipid particles comprising: (a) one or more (e.g., a cocktail) gRNA molecules described herein; (b) one or more cationic lipids or salts thereof comprising from about 2 mol % to about 50 mol % of the total lipid present in the particle; (c) one or more non-cationic lipids comprising from about 5 mol % to about 90 mol % of the total lipid present in the particle; and (d) one or more conjugated lipids that inhibit aggregation of particles comprising from about 0.5 mol % to about 20 mol % of the total lipid present in the particle.

In one aspect of this embodiment, the nucleic acid-lipid particle comprises: (a) one or more (e.g., a cocktail) gRNA molecules described herein; (b) a cationic lipid or a salt thereof comprising from about 30 mol % to about 50 mol % of the total lipid present in the particle; (c) a mixture of a phospholipid and cholesterol or a derivative thereof comprising from about 47 mol % to about 69 mol % of the total lipid present in the particle; and (d) a PEG-lipid conjugate comprising from about 1 mol % to about 3 mol % of the total lipid present in the particle. In one particular embodiment, the formulation is a four-component system which comprises about 2 mol % PEG-lipid conjugate (e.g., PEG2000-C-DMA), about 40 mol % cationic lipid (e.g., DLin-K-C2-DMA) or a salt thereof, about 10 mol % DPPC (or DSPC), and about 48 mol % cholesterol (or derivative thereof).

In further embodiments, the present invention provides nucleic acid-lipid particles comprising: (a) one or more (e.g., a cocktail) gRNA molecules described herein; (b) one or more cationic lipids or salts thereof comprising from about 50 mol % to about 65 mol % of the total lipid present in the particle; (c) one or more non-cationic lipids comprising from about 25 mol % to about 45 mol % of the total lipid present in the particle; and (d) one or more conjugated lipids that inhibit aggregation of particles comprising from about 5 mol % to about 10 mol % of the total lipid present in the particle.

In one aspect of this embodiment, the nucleic acid-lipid particle comprises: (a) one or more (e.g., a cocktail) gRNA molecules described herein; (b) a cationic lipid or a salt thereof comprising from about 50 mol % to about 60 mol % of the total lipid present in the particle; (c) a mixture of a phospholipid and cholesterol or a derivative thereof comprising from about 35 mol % to about 45 mol % of the total lipid present in the particle; and (d) a PEG-lipid conjugate comprising from about 5 mol % to about 10 mol % of the total lipid present in the particle. In certain instances, the non-cationic lipid mixture in the formulation comprises: (i) a phospholipid of from about 5 mol % to about 10 mol % of the total lipid present in the particle; and (ii) cholesterol or a derivative thereof of from about 25 mol % to about 35 mol % of the total lipid present in the particle. In one particular embodiment, the formulation is a four-component system which comprises about 7 mol % PEG-lipid conjugate (e.g., PEG750-C-DMA), about 54 mol % cationic lipid (e.g., DLin-K-C2-DMA) or a salt thereof, about 7 mol % DPPC (or DSPC), and about 32 mol % cholesterol (or derivative thereof).

In another aspect of this embodiment, the nucleic acid-lipid particle comprises: (a) one or more (e.g., a cocktail) gRNA molecules described herein; (b) a cationic lipid or a salt thereof comprising from about 55 mol % to about 65 mol % of the total lipid present in the particle; (c) cholesterol or a derivative thereof comprising from about 30 mol % to about 40 mol % of the total lipid present in the particle; and (d) a PEG-lipid conjugate comprising from about 5 mol % to about 10 mol % of the total lipid present in the particle. In one particular embodiment, the formulation is a three-component system which is phospholipid-free and comprises about 7 mol % PEG-lipid conjugate (e.g., PEG750-C-DMA), about 58 mol % cationic lipid (e.g., DLin-K-C2-DMA) or a salt thereof, and about 35 mol % cholesterol (or derivative thereof).

Additional embodiments of useful formulations are described in published US patent application publication number US 2011/0076335 A1, the disclosure of which is herein incorporated by reference in its entirety for all purposes.

In certain embodiments of the invention, the nucleic acid-lipid particle comprises: (a) one or more (e.g., a cocktail) gRNA molecules described herein; (b) a cationic lipid or a salt thereof comprising from about 48 mol % to about 62 mol % of the total lipid present in the particle; (c) a mixture of a phospholipid and cholesterol or a derivative thereof, wherein the phospholipid comprises about 7 mol % to about 17 mol % of the total lipid present in the particle, and wherein the cholesterol or derivative thereof comprises about 25 mol % to about 40 mol % of the total lipid present in the particle; and (d) a PEG-lipid conjugate comprising from about 0.5 mol % to about 3.0 mol % of the total lipid present in the particle. Exemplary lipid formulations A-Z of this aspect of the invention are included below.

Exemplary lipid formulation A includes the following components (wherein the percentage values of the components are mole percent): PEG-lipid (1.2%), cationic lipid (53.2%), phospholipid (9.3%), cholesterol (36.4%), wherein the actual amounts of the lipids present may vary by, e.g., ±5% (or e.g., ±4 mol %, ±3 mol %, ±2 mol %, 1 mol %,f 0.75 mol %, f 0.5 mol %, ±0.25 mol %, or ±0.1 mol %). For example, in one representative embodiment, the PEG-lipid is PEG-C-DMA (compound (66)) (1.2%), the cationic lipid is 1,2-dilinoleyloxy-N,N-dimethylaminopropane (DLinDMA) (53.2%), the phospholipid is DPPC (9.3%), and cholesterol is present at 36.4%, wherein the actual amounts of the lipids present may vary by, e.g,±5% (or e.g., ±4 mol %, ±3 mol %, ±2 mol %, 1 mol %, 0.75 mol %, 0.5 mol %, ±0.25 mol %, or ±0.1 mol %). Thus, certain embodiments of the invention provide a nucleic acid-lipid particle based on formulation A, which comprises one or more gRNA molecules described herein. For example, in certain embodiments, the nucleic acid lipid particle based on formulation A may comprise two different gRNA molecules, wherein a combination of the two different gRNA molecules is selected from any one of the combinations described herein. In certain other embodiments, the nucleic acid lipid particle based on formulation A may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation B which includes the following components (wherein the percentage values of the components are mole percent): PEG-lipid (0.8%), cationic lipid (59.7%), phospholipid (14.2%), cholesterol (25.3%), wherein the actual amounts of the lipids present may vary by, e.g., ±5% (or e.g., ±4 mol %, ±3 mol %, ±2 mol %, 1 mol %, 0.75 mol %, ±0.5 mol %, ±0.25 mol %, or ±0.1 mol %). For example, in one representative embodiment, the PEG-lipid is PEG-C-DOMG (compound (67)) (0.8%), the cationic lipid is 1,2-dilinoleyloxy-N,N-dimethylaminopropane (DLinDMA) (59.7%), the phospholipid is DSPC (14.2%), and cholesterol is present at 25.3%, wherein the actual amounts of the lipids present may vary by, e.g., ±5% (or e.g., ±4 mol %, ±3 mol %, ±2 mol %, 1 mol %, 0.75 mol %, 0.5 mol %, 0.25 mol %, or ±0.1 mol %). Thus, certain embodiments of the invention provide a nucleic acid-lipid particle based on formulation B, which comprises one or more gRNA molecules described herein. For example, in certain embodiments, the nucleic acid lipid particle based on formulation B may comprise two different gRNA molecules, wherein a combination of the two different gRNA molecules is selected from any one of the combinations described herein. In certain other embodiments, the nucleic acid lipid particle based on formulation B may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation C includes the following components (wherein the percentage values of the components are mole percent): PEG-lipid (1.9%), cationic lipid (52.5%), phospholipid (14.8%), cholesterol (30.8%), wherein the actual amounts of the lipids present may vary by, e.g., ±5% (or e.g., ±4 mol %, ±3 mol %, ±2 mol %, ±1 mol %, 0.75 mol %, ±0.5 mol %, ±0.25 mol %, or ±0.1 mol %). For example, in one representative embodiment, the PEG-lipid is PEG-C-DOMG (compound (67)) (1.9%), the cationic lipid is 1,2-di-γ-linolenyloxy-N,N-dimethylaminopropane (γ-DLenDMA; Compound (15)) (52.5%), the phospholipid is DSPC (14.8%), and cholesterol is present at 30.8%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation C, which comprises one or more gRNA molecules described herein. For example, in certain embodiments, the nucleic acid lipid particle based on formulation C may comprise two different gRNA molecules, wherein a combination of the two different gRNA molecules is selected from any one of the combinations described herein. In certain other embodiments, the nucleic acid lipid particle based on formulation C may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation D includes the following components (wherein the percentage values of the components are mole percent): PEG-lipid (0.7%), cationic lipid (60.3%), phospholipid (8.4%), cholesterol (30.5%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (0.7%), the cationic lipid is 3-((6Z,9Z,28Z,31Z)-heptatriaconta-6,9,28,31-tetraen-19-yloxy)-N,N-dimethylpropan-1-amine (DLin-MP-DMA; Compound (8) (60.3%), the phospholipid is DSPC (8.4%), and cholesterol is present at 30.5%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation D, which comprises one or more gRNA molecules described herein. For example, in certain embodiments, the nucleic acid lipid particle based on formulation D may comprise two different gRNA molecules, wherein a combination of the two different gRNA molecules is selected from any one of the combinations described herein. In certain other embodiments, the nucleic acid lipid particle based on formulation D may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation E includes the following components (wherein the percentage values of the components are mole percent): PEG-lipid (1.8%), cationic lipid (52.1%), phospholipid (7.5%), cholesterol (38.5%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (1.8%), the cationic lipid is (6Z,9Z,28Z,31Z)-heptatriaconta-6,9,28,31-tetraen-19-yl 4-(dimethylamino)butanoate) (Compound (7)) (52.1%), the phospholipid is DPPC (7.5%), and cholesterol is present at 38.5%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation E, which comprises one or more gRNA molecules described herein. For example, in certain embodiments, the nucleic acid lipid particle based on formulation E may comprise two different gRNA molecules, wherein a combination of the two different gRNA molecules is selected from any one of the combinations described herein. In certain other embodiments, the nucleic acid lipid particle based on formulation E may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary formulation F includes the following components (wherein the percentage values of the components are mole percent): PEG-lipid (0.9%), cationic lipid (57.1%), phospholipid (8.1%), cholesterol (33.8%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (0.9%), the cationic lipid is 1,2-dilinolenyloxy-N,N-dimethylaminopropane (DLenDMA), 1,2-di-γ-linolenyloxy-N,N-dimethylaminopropane (γ-DLenDMA; Compound (15)) (57.1%), the phospholipid is DSPC (8.1%), and cholesterol is present at 33.8%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation F, which comprises one or more gRNA molecules described herein. For example, in certain embodiments, the nucleic acid lipid particle based on formulation F may comprise two different gRNA molecules, wherein a combination of the two different gRNA molecules is selected from any one of the combinations described herein. In certain other embodiments, the nucleic acid lipid particle based on formulation F may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation G includes the following components (wherein the percentage values of the components are mole percent): PEG-lipid (1.7%), cationic lipid (61.6%), phospholipid (11.2%), cholesterol (25.5%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (1.7%), the cationic lipid is 1,2-dilinolenyloxy-N,N-dimethylaminopropane (DLenDMA), 1,2-di-γ-linolenyloxy-N,N-dimethylaminopropane (γ-DLenDMA; Compound (15)) (61.6%), the phospholipid is DPPC (11.2%), and cholesterol is present at 25.5%, wherein the actual amounts of the lipids present may vary by, e.g.,±5% (or e.g.,±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation G, which comprises one or more gRNA molecules described herein. For example, in certain embodiments, the nucleic acid lipid particle based on formulation G may comprise two different gRNA molecules, wherein a combination of the two different gRNA molecules is selected from any one of the combinations described herein. In certain other embodiments, the nucleic acid lipid particle based on formulation G may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation H includes the following components (wherein the percentage values of the components are mole percent): PEG-lipid (1.1%), cationic lipid (55.0%), phospholipid (11.0%), cholesterol (33.0%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (1.1%), the cationic lipid is (6Z,16Z)-12-((Z)-dec-4-enyl)docosa-6,16-dien-11-yl 5-(dimethylamino)pentanoate (Compound (13)) (55.0%), the phospholipid is DSPC (11.0%), and cholesterol is present at 33.0%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation H, which comprises one or more gRNA molecules described herein. For example, in certain embodiments, the nucleic acid lipid particle based on formulation H may comprise two different gRNA molecules wherein a combination of the two different gRNA molecules is selected from any one of the combinations described herein. In certain other embodiments, the nucleic acid lipid particle based on formulation H may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation I includes the following components (wherein the percentage values of the components are mole percent): PEG-lipid (2.6%), cationic lipid (53.1%), phospholipid (9.4%), cholesterol (35.0%), wherein the actual amounts of the lipids present may vary by, e.g., ±5% (or e.g., ±4 mol %, ±3 mol %, ±2 mol %, ±1 mol%, ±0.75 mol %, ±0.5 mol %, ±0.25 mol %, or ±0.1 mol %). For example, in one representative embodiment, the PEG-lipid is PEG-C-DMA (compound (66)) (2.6%), the cationic lipid is (6Z,16Z)-12-((Z)-dec-4-enyl)docosa-6,16-dien-11-yl 5-(dimethylamino)pentanoate (Compound (13)) (53.1%), the phospholipid is DSPC (9.4%), and cholesterol is present at 35.0%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation I, which comprises one or more gRNA molecules described herein. For example, in certain embodiments, the nucleic acid lipid particle based on formulation I may comprise two different gRNA molecules, wherein a combination of the two different gRNA molecules is selected from any one of the combinations described herein. In certain other embodiments, the nucleic acid lipid particle based on formulation I may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation J includes the following components (wherein the percentage values of the components are mole percent): PEG-lipid (0.6%), cationic lipid (59.4%), phospholipid (10.2%), cholesterol (29.8%), wherein the actual amounts of the lipids present may vary by by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (0.6%), the cationic lipid is 1,2-dilinoleyloxy-N,N-dimethylaminopropane (DLinDMA) (59.4%), the phospholipid is DPPC (10.2%), and cholesterol is present at 29.8%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation J, which comprises one or more gRNA molecules described herein. For example, in certain embodiments, the nucleic acid lipid particle based on formulation J may comprise two different gRNA molecules, wherein a combination of the two different gRNA molecules is selected from any one of the combinations described herein. In certain other embodiments, the nucleic acid lipid particle based on formulation J may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation K includes the following components (wherein the percentage values of the components are mole percent): PEG-lipid (0.5%), cationic lipid (56.7%), phospholipid (13.1%), cholesterol (29.7%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (0.5%), the cationic lipid is (6Z,9Z,28Z,31Z)-heptatriaconta-6,9,28,31-tetraen-19-yl 4-(dimethylamino)butanoate) (Compound (7)) (56.7%), the phospholipid is DSPC (13.1%), and cholesterol is present at 29.7%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation K, which comprises one or more gRNA molecules described herein. For example, in certain embodiments, the nucleic acid lipid particle based on formulation K may comprise two different gRNA molecules, wherein a combination of the two different gRNA molecules is selected from any one of the combinations described herein. In certain other embodiments, the nucleic acid lipid particle based on formulation K may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation L includes the following components (wherein the percentage values of the components are mole percent): PEG-lipid (2.2%), cationic lipid (52.0%), phospholipid (9.7%), cholesterol (36.2%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (2.2%), the cationic lipid is 1,2-di-γ-linolenyloxy-N,N-dimethylaminopropane (γ-DLenDMA; Compound (15)) (52.0%), the phospholipid is DSPC (9.7%), and cholesterol is present at 36.2%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation L, which comprises one or more gRNA molecules described herein. For example, in certain embodiments, the nucleic acid lipid particle based on formulation L may comprise two different gRNA molecules, wherein a combination of the two different gRNA molecules is selected from any one of the combinations described herein. In certain other embodiments, the nucleic acid lipid particle based on formulation L may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation M includes the following components (wherein the percentage values of the components are mole percent): PEG-lipid (2.7%), cationic lipid (58.4%), phospholipid (13.1%), cholesterol (25.7%), wherein the actual amounts of the lipids present may vary by by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (2.7%), the cationic lipid is 1,2-dilinoleyloxy-N,N-dimethylaminopropane (DLinDMA) (58.4%), the phospholipid is DPPC (13.1%), and cholesterol is present at 25.7%, wherein the actual amounts of the lipids present may vary by, e.g., ±5% (or e.g.,±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation M, which comprises one or more gRNA molecules described herein. For example, in certain embodiments, the nucleic acid lipid particle based on formulation M may comprise two different gRNA molecules, wherein a combination of the two different gRNA molecules is selected from any one of the combinations described herein. In certain other embodiments, the nucleic acid lipid particle based on formulation M may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation N includes the following components (wherein the percentage values of the components are mole percent): PEG-lipid (3.0%), cationic lipid (53.3%), phospholipid (12.1%), cholesterol (31.5%), wherein the actual amounts of the lipids present may vary by by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (3.0%), the cationic lipid is 1,2-dilinoleyloxy-N,N-dimethylaminopropane (DLinDMA) (53.3%), the phospholipid is DPPC (12.1%), and cholesterol is present at 31.5%, wherein the actual amounts of the lipids present may vary by, e.g.,±5% (or e.g.,±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation N, which comprises one or more gRNA molecules described herein. For example, in certain embodiments, the nucleic acid lipid particle based on formulation N may comprise two different gRNA molecules, wherein a combination of the two different gRNA molecules is selected from any one of the combinations described herein. In certain other embodiments, the nucleic acid lipid particle based on formulation N may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation 0 includes the following components (wherein the percentage values of the components are mole percent): PEG-lipid (1.5%), cationic lipid (56.2%), phospholipid (7.8%), cholesterol (34.7%), wherein the actual amounts of the lipids present may vary by by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (1.5%), the cationic lipid is 1,2-dilinoleyloxy-N,N-dimethylaminopropane (DLinDMA) (56.2%), the phospholipid is DPPC (7.8%), and cholesterol is present at 34.7%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation 0, which comprises one or more gRNA molecules described herein. For example, in certain embodiments, the nucleic acid lipid particle based on formulation O may comprise two different gRNA molecules, wherein a combination of the two different gRNA molecules is selected from any one of the combinations described herein. In certain other embodiments, the nucleic acid lipid particle based on formulation 0 may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation P includes the following components (wherein the percentage values of the components are mole percent): PEG-lipid (2.1%), cationic lipid (48.6%), phospholipid (15.5%), cholesterol (33.8%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (2.1%), the cationic lipid is 3-((6Z,9Z,28Z,31Z)-heptatriaconta-6,9,28,31-tetraen-19-yloxy)-N,N-dimethylpropan-1-amine (DLin-MP-DMA; Compound (8)) (48.6%), the phospholipid is DSPC (15.5%), and cholesterol is present at 33.8%, wherein the actual amounts of the lipids present may vary by, e.g., ±5% (or e.g,±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation P, which comprises one or more gRNA molecules described herein. For example, in certain embodiments, the nucleic acid lipid particle based on formulation P may comprise two different gRNA molecules, wherein a combination of the two different gRNA molecules is selected from any one of the combinations described herein. In certain other embodiments, the nucleic acid lipid particle based on formulation P may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation Q includes the following components (wherein the percentage values of the components are mole percent): PEG-lipid (2.5%), cationic lipid (57.9%), phospholipid (9.2%), cholesterol (30.3%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (2.5%), the cationic lipid is (6Z,16Z)-12-((Z)-dec-4-enyl)docosa-6,16-dien-11-yl 5-(dimethylamino)pentanoate (Compound (13)) (57.9%), the phospholipid is DSPC (9.2%), and cholesterol is present at 30.3%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation Q, which comprises one or more gRNA molecules described herein. For example, in certain embodiments, the nucleic acid lipid particle based on formulation Q may comprise two different gRNA molecules, wherein a combination of the two different gRNA molecules is selected from any one of the combinations described herein. In certain other embodiments, the nucleic acid lipid particle based on formulation Q may comprise three different gRNA molecules, wherein a combination of the two different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation R includes the following components (wherein the percentage values of the components are mole percent): PEG-lipid (1.6%), cationic lipid (54.6%), phospholipid (10.9%), cholesterol (32.8%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (1.6%), the cationic lipid is 3-((6Z,9Z,28Z,31Z)-heptatriaconta-6,9,28,31-tetraen-19-yloxy)-N,N-dimethylpropan-1-amine (Compound (8)) (54.6%), the phospholipid is DSPC (10.9%), and cholesterol is present at 32.8%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation R, which comprises one or more gRNA molecules described herein. For example, in certain embodiments, the nucleic acid lipid particle based on formulation R may comprise two different gRNA molecules, wherein a combination of the two different gRNA molecules is selected from any one of the combinations described herein. In certain other embodiments, the nucleic acid lipid particle based on formulation R may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation S includes the following components (wherein the percentage values of the components are mole percent): PEG-lipid (2.9%), cationic lipid (49.6%), phospholipid (16.3%), cholesterol (31.3%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (2.9%), the cationic lipid is (6Z,16Z)-12-((Z)-dec-4-enyl)docosa-6,16-dien-11-yl 5-(dimethylamino)pentanoate (Compound (13)) (49.6%), the phospholipid is DPPC (16.3%), and cholesterol is present at 31.3%, wherein the actual amounts of the lipids present may vary by, e.g.,±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation S, which comprises one or more gRNA molecules described herein. For example, in certain embodiments, the nucleic acid lipid particle based on formulation S may comprise two different gRNA molecules, wherein a combination of the two different gRNA molecules is selected from any one of the combinations described herein. In certain other embodiments, the nucleic acid lipid particle based on formulation S may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation T includes the following components (wherein the percentage values of the components are mole percent): PEG-lipid (0.7%), cationic lipid (50.5%), phospholipid (8.9%), cholesterol (40.0%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (0.7%), the cationic lipid is 1,2-dilinoleyloxy-N,N-dimethylaminopropane (DLinDMA) (50.5%), the phospholipid is DPPC (8.9%), and cholesterol is present at 40.0%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation T, which comprises one or more gRNA molecules described herein. For example, in certain embodiments, the nucleic acid lipid particle based on formulation T may comprise two different gRNA molecules, wherein a combination of the two different gRNA molecules is selected from any one of the combinations described herein. In certain other embodiments, the nucleic acid lipid particle based on formulation T may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation U includes the following components (wherein the percentage values of the components are mole percent): PEG-lipid (1.0%), cationic lipid (51.4%), phospholipid (15.0%), cholesterol (32.6%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (1.0%), the cationic lipid is 1,2-dilinoleyloxy-N,N-dimethylaminopropane (DLinDMA) (51.4%), the phospholipid is DSPC (15.0%), and cholesterol is present at 32.6%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation U, which comprises one or more gRNA molecules described herein. For example, in certain embodiments, the nucleic acid lipid particle based on formulation U may comprise two different gRNA molecules, wherein a combination of the two different gRNA molecules is selected from any one of the combinations described herein. In certain other embodiments, the nucleic acid lipid particle based on formulation U may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation V includes the following components (wherein the percentage values of the components are mole percent): PEG-lipid (1.3%), cationic lipid (60.0%), phospholipid (7.2%), cholesterol (31.5%), wherein the actual amounts of the lipids present may vary by, e.g,±5% (or e.g., +4 mol %, +3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (1.3%), the cationic lipid is 1,2-dilinolenyloxy-N,N-dimethylaminopropane (DLenDMA) (60.0%), the phospholipid is DSPC (7.2%), and cholesterol is present at 31.5%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation V, which comprises one or more gRNA molecules described herein. For example, in certain embodiments, the nucleic acid lipid particle based on formulation V may comprise two different gRNA molecules, wherein a combination of the two different gRNA molecules is selected from any one of the combinations described herein. In certain other embodiments, the nucleic acid lipid particle based on formulation V may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation W includes the following components (wherein the percentage values of the components are mole percent): PEG-lipid (1.8%), cationic lipid (51.6%), phospholipid (8.4%), cholesterol (38.3%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (1.8%), the cationic lipid is 1,2-dilinoleyloxy-N,N-dimethylaminopropane (DLinDMA) (51.6%), the phospholipid is DSPC (8.4%), and cholesterol is present at 38.3%, wherein the actual amounts of the lipids present may vary by, e.g., ±5% (or e.g., ±4 mol %, ±3 mol %, ±2 mol %, ±1 mol %, ±0.75 mol %, ±0.5 mol %, 0.25 mol %, or ±0.1 mol %). Thus, certain embodiments of the invention provide a nucleic acid-lipid particle based on formulation W, which comprises one or more gRNA molecules described herein. For example, in certain embodiments, the nucleic acid lipid particle based on formulation W may comprise two different gRNA molecules, wherein a combination of the two different gRNA molecules is selected from any one of the combinations described herein. In certain other embodiments, the nucleic acid lipid particle based on formulation W may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation X includes the following components (wherein the percentage values of the components are mole percent): PEG-lipid (2.4%), cationic lipid (48.5%), phospholipid (10.0%), cholesterol (39.2%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (2.4%), the cationic lipid is 1,2-di-γ-linolenyloxy-N,N-dimethylaminopropane (γ-DLenDMA; Compound (15)) (48.5%), the phospholipid is DPPC (10.0%), and cholesterol is present at 39.2%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation X, which comprises one or more gRNA molecules described herein. For example, in certain embodiments, the nucleic acid lipid particle based on formulation X may comprise two different gRNA molecules, wherein a combination of the two different gRNA molecules is selected from any one of the combinations described herein. In certain other embodiments, the nucleic acid lipid particle based on formulation X may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation Y includes the following components (wherein the percentage values of the components are mole percent): PEG-lipid (2.6%), cationic lipid (61.2%), phospholipid (7.1%), cholesterol (29.2%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DMA (compound (66)) (2.6%), the cationic lipid is (6Z,16Z)-12-((Z)-dec-4-enyl)docosa-6,16-dien-11-yl 5-(dimethylamino)pentanoate (Compound (13)) (61.2%), the phospholipid is DSPC (7.1%), and cholesterol is present at 29.2%, wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation Y, which comprises one or more gRNA molecules described herein. For example, in certain embodiments, the nucleic acid lipid particle based on formulation Y may comprise two different gRNA molecules, wherein a combination of the two different gRNA molecules is selected from any one of the combinations described herein. In certain other embodiments, the nucleic acid lipid particle based on formulation Y may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Exemplary lipid formulation Z includes the following components (wherein the percentage values of the components are mole percent): PEG-lipid (2.2%), cationic lipid (49.7%), phospholipid (12.1%), cholesterol (36.0%), wherein the actual amounts of the lipids present may vary by, e.g., ±5% (or e.g., ±4 mol %, ±3 mol %, ±2 mol %, ±1 mol %, ±0.75 mol %, ±0.5 mol %, ±0.25 mol %, or ±0.1 mol %). For example, in one representative embodiment, the PEG-lipid is PEG-C-DOMG (compound (67)) (2.2%), the cationic lipid is (6Z,9Z,28Z,31Z)-heptatriaconta-6,9,28,31-tetraen-19-yl 4-(dimethylamino)butanoate) (Compound (7)) (49.7%), the phospholipid is DPPC (12.1%), and cholesterol is present at 36.0%, wherein the actual amounts of the lipids present may vary by, e.g., +5% (or e.g., ±4 mol %, ±3 mol %, ±2 mol %, ±1 mol %, ±0.75 mol %, ±0.5 mol %, ±0.25 mol %, or ±0.1 mol %). Thus, certain embodiments of the invention provide a nucleic acid-lipid particle based on formulation Z, which comprises one or more gRNA molecules described herein. For example, in certain embodiments, the nucleic acid lipid particle based on formulation Z may comprise two different gRNA molecules, wherein a combination of the two different gRNA molecules is selected from any one of the combinations described herein. In certain other embodiments, the nucleic acid lipid particle based on formulation Z may comprise three different gRNA molecules, wherein a combination of the three different gRNA molecules is selected from any one of the combinations described herein. In certain embodiments, the nucleic acid-lipid particle has a total lipid:gRNA mass ratio of from about 5:1 to about 15:1, or about 5:1, 6:1, 7:1, 8:1, 9:1, 10:1, 11:1, 12:1, 13:1, 14:1, or 15:1, or any fraction thereof or range therein. In certain embodiments, the nucleic acid-lipid particle has a total lipid:gRNA mass ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

Accordingly, certain embodiments of the invention provide a nucleic acid-lipid particle described herein, wherein the lipids are formulated as described in any one of formulations A, B, C, D, E, F, G, H, I, J, K, L, M, N, O, P, Q, R, S, T, U, V, W, X, Y or Z.

The present invention also provides pharmaceutical compositions comprising a nucleic acid-lipid particle and a pharmaceutically acceptable carrier.

The nucleic acid-lipid particles of the present invention are useful, for example, for the therapeutic delivery of gRNAs that silence the expression of one or more HBV genes. In some embodiments, a cocktail of gRNAs that target different regions (e.g., overlapping and/or non-overlapping sequences) of an HBV gene or transcript is formulated into the same or different nucleic acid-lipid particles, and the particles are administered to a mammal (e.g., a human) requiring such treatment. In certain instances, a therapeutically effective amount of the nucleic acid-lipid particles can be administered to the mammal, e.g., for treating HBV and/or HDV infection in a human.

In certain embodiments, the present invention provides a method for introducing one or more gRNA molecules described herein into a cell by contacting the cell with a nucleic acid-lipid particle described herein.

In certain embodiments, the present invention provides a method for introducing one or more gRNA molecules that silence expression of a Hepatitis B virus gene into a cell by contacting the cell with a nucleic acid-lipid particle described herein under conditions whereby the gRNA enters the cell and silences the expression of the Hepatitis B virus gene within the cell. In certain embodiments, the cell is in a mammal, such as a human. In certain embodiments, the human has been diagnosed with a Hepatitis B virus infection or a Hepatitis B virus/Hepatitis D virus infection. In certain embodiments, silencing of the Hepatitis B virus gene expression reduces Hepatitis B virus and/or Hepatitis D virus particle load in the mammal by at least about 50% (e.g., about 55, 60, 65, 70, 75, 80, 85, 90, 95, 96, 97, 98, 99 or 100%) relative to Hepatitis B virus and/or Hepatitis D virus particle load in the absence of the nucleic acid-lipid particle.

In certain embodiments, the present invention provides a method for silencing expression of a Hepatitis B virus gene in a cell, the method comprising the step of contacting a cell comprising an expressed Hepatitis B virus gene with a nucleic acid-lipid particle or a composition (e.g., a pharmaceutical composition) described herein under conditions whereby the gRNA enters the cell and silences the expression of the Hepatitis B virus gene within the cell. In certain embodiments, the cell is in a mammal, such as a human. In certain embodiments, the human has been diagnosed with a Hepatitis B virus infection or a Hepatitis B virus/Hepatitis D virus infection. In certain embodiments, the human has been diagnosed with liver disease caused by a Hepatitis B virus infection or a Hepatitis B virus/Hepatitis D virus infection. In certain embodiments, silencing of the Hepatitis B virus gene expression reduces Hepatitis B virus and/or Hepatitis D virus particle load in the mammal by at least about 50% (e.g., about 55, 60, 65, 70, 75, 80, 85, 90, 95, 96, 97, 98, 99 or 100%) relative to Hepatitis B virus and/or Hepatitis D virus particle load in the absence of the nucleic acid-lipid particle.

In some embodiments, the nucleic acid-lipid particles or compositions (e.g., a pharmaceutical composition) described herein are administered by one of the following routes of administration: oral, intranasal, intravenous, intraperitoneal, intramuscular, intra-articular, intralesional, intratracheal, subcutaneous, and intradermal. In particular embodiments, the nucleic acid-lipid particles are administered systemically, e.g., via enteral or parenteral routes of administration.

In certain aspects, the present invention provides methods for silencing HBV gene expression in a mammal (e.g., human) in need thereof, the method comprising administering to the mammal a therapeutically effective amount of a nucleic acid-lipid particle comprising one or more gRNAs described herein. In some embodiments, administration of nucleic acid-lipid particles comprising one or more gRNAs described herein reduces HBV RNA levels by at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% (or any range therein) relative to HBV RNA levels detected in the absence of the gRNA (e.g., buffer control or irrelevant non-HBV targeting gRNA control). In other embodiments, administration of nucleic acid-lipid particles comprising one or more HBV-targeting gRNAs reduces HBV RNA levels for at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 days or more (or any range therein) relative to a negative control such as, e.g., a buffer control or an irrelevant non-HBV targeting gRNA control.

In other aspects, the present invention provides methods for silencing HBV gene expression in a mammal (e.g., human) in need thereof, the method comprising administering to the mammal a therapeutically effective amount of a nucleic acid-lipid particle comprising one or more gRNAs described herein. In some embodiments, administration of nucleic acid-lipid particles comprising one or more HBV gRNAs reduces HBV mRNA levels by at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% (or any range therein) relative to HBV mRNA levels detected in the absence of the gRNA (e.g., buffer control or irrelevant non-HBV targeting gRNA control). In other embodiments, administration of nucleic acid-lipid particles comprising one or more HBV-targeting gRNAs reduces HBV mRNA levels for at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 days or more (or any range therein) relative to a negative control such as, e.g., a buffer control or an irrelevant non-HBV targeting gRNA control.

Certain embodiments of the invention provide a nucleic acid-lipid particle or a composition (e.g., a pharmaceutical composition) described herein for use in silencing expression of a Hepatitis B virus gene in a cell in a mammal (e.g., a human).

Certain embodiments of the invention provide the use of a nucleic acid-lipid particle or a composition (e.g., a pharmaceutical composition) described herein to prepare a medicament for silencing expression of a Hepatitis B virus gene in a cell in a mammal (e.g., a human).

In other aspects, the present invention provides methods for treating, preventing, reducing the risk or likelihood of developing (e.g., reducing the susceptibility to), delaying the onset of, and/or ameliorating one or more symptoms associated with HBV and/or HDV infection in a mammal (e.g., human) in need thereof, the method comprising administering to the mammal a therapeutically effective amount of a nucleic acid-lipid particle comprising one or more gRNA molecules described herein that target HBV gene expression. Examples of symptoms associated with HBV and/or HDV infection in a human include fever, abdominal pain, dark urine, joint pain, loss of appetite, nausea, vomiting, weakness, fatigue and yellowing of the skin (jaundice).

Certain embodiments of the invention provide a method for treating a Hepatitis B virus and/or Hepatitis D virus infection in a mammal, the method comprising the step of administering to the mammal a therapeutically effective amount of a nucleic acid-lipid particle or a composition (e.g., a pharmaceutical composition) as described herein.

Certain embodiments of the invention provide a nucleic acid-lipid particle or a composition (e.g., a pharmaceutical composition) for use in treating a Hepatitis B virus and/or Hepatitis D virus infection in a mammal (e.g., a human).

Certain embodiments of the invention provide the use of a nucleic acid-lipid particle or a composition (e.g., a pharmaceutical composition) to prepare a medicament for treating a Hepatitis B virus and/or Hepatitis D virus infection in a mammal (e.g., a human).

Certain embodiments of the invention provide a method for ameliorating one or more symptoms associated with Hepatitis B virus and/or Hepatitis D virus infection in a mammal, the method comprising the step of administering to the mammal a therapeutically effective amount of a nucleic acid-lipid particle or composition (e.g., a pharmaceutical composition) described herein, comprising one or more gRNA molecules described herein. In certain embodiments, the particle is administered via a systemic route. In certain embodiments, the gRNA of the nucleic acid-lipid particle inhibits expression of a Hepatitis B virus gene in the mammal. In certain embodiments, the mammal is a human. In certain embodiments, the human has liver disease.

Certain embodiments of the invention provide a nucleic acid-lipid particle or a composition (e.g., a pharmaceutical composition) as described herein for use in ameliorating one or more symptoms associated with a Hepatitis B virus and/or Hepatitis D virus infection in a mammal (e.g., a human).

Certain embodiments of the invention provide the use of a nucleic acid-lipid particle or a composition (e.g., a pharmaceutical composition) as described herein to prepare a medicament for ameliorating one or more symptoms associated with a Hepatitis B virus and/or Hepatitis D virus infection in a mammal (e.g., a human).

Certain embodiments of the present invention provide a method for inhibiting the replication of HDV and/or ameliorating one or more symptoms of HDV infection in a mammal (e.g., a human), the method comprising the step of administering a therapeutically effective amount of a nucleic acid-lipid particle or a composition (e.g., a pharmaceutical composition) as described herein to the mammal, wherein the nucleic acid-lipid particle or composition inhibits the synthesis of HBV surface antigen.

Certain embodiments of the invention provide a nucleic acid-lipid particle or a composition (e.g., a pharmaceutical composition) as described herein for use in inhibiting the replication of HDV and/or ameliorating one or more symptoms of HDV infection in a mammal (e.g., a human), wherein the nucleic acid-lipid particle or composition inhibits the synthesis of HBV surface antigen.

Certain embodiments of the invention provide the use of a nucleic acid-lipid particle or a composition (e.g., a pharmaceutical composition) as described herein to prepare a medicament for inhibiting the replication of HDV and/or ameliorating one or more symptoms of HDV infection in a mammal (e.g., a human), wherein the nucleic acid-lipid particle or composition inhibits the synthesis of HBV surface antigen.

Certain embodiments of the invention provide a nucleic acid-lipid particle or a composition (e.g., a pharmaceutical composition) as described herein for use in medical therapy.

In further aspects, the present invention provides a method for inactivating HBV and/or HDV in a mammal (e.g., human) in need thereof (e.g., a human suffering from HBV infection or HBV/HDV infection), the method comprising administering to the mammal a therapeutically effective amount of a nucleic acid-lipid particle comprising one or more gRNAs described herein that target HBV gene expression. In some embodiments, administration of nucleic acid-lipid particles comprising one or more HBV-targeting gRNAs lowers, reduces, or decreases HBV protein levels (e.g., HBV surface antigen protein) by at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% (or any range therein) relative to the HBV protein levels detected in the absence of the gRNA (e.g., buffer control or irrelevant non-HBV targeting gRNA control).

By way of example, IIBV mRNA can be measured using a branched DNA assay (QuantiGene®; Affymetrix). The branched DNA assay is a sandwich nucleic acid hybridization method that uses bDNA molecules to amplify signal from captured target RNA.

In addition to its utility in silencing the expression of any of the HBV genes for therapeutic purposes, the gRNA described herein are also useful in research and development applications as well as diagnostic, prophylactic, prognostic, clinical, and other healthcare applications. As a non-limiting example, the gRNA can be used in target validation studies directed at testing whether a specific member of the HBV gene family has the potential to be a therapeutic target.

Generating gRNA Molecules

In some embodiments, gRNA may be produced enzymatically or by partial/total organic synthesis, and modified ribonucleotides can be introduced by in vitro enzymatic or organic synthesis. In certain instances, the gRNA is prepared chemically. Methods of synthesizing nucleic acid molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Preferably, gRNA are chemically synthesized. The oligonucleotides that comprise the gRNA molecules of the invention can be synthesized using any of a variety of techniques known in the art, such as those described in Usman et aL, J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, CA). However, a larger or smaller scale of synthesis is also within the scope of this invention. Suitable reagents for oligonucleotide synthesis, methods for RNA deprotection, and methods for RNA purification are known to those of skill in the art.

Carrier Systems Containing Therapeutic Nucleic Acids

### A. Lipid Particles

In certain aspects, the present invention provides lipid particles comprising one or more gRNA molecules and one or more of cationic (amino) lipids or salts thereof. In some embodiments, the lipid particles of the invention further comprise one or more non-cationic lipids. In other embodiments, the lipid particles further comprise one or more conjugated lipids capable of reducing or inhibiting particle aggregation.

The lipid particles of the invention may comprise one or more gRNA, a cationic lipid, a non-cationic lipid, and a conjugated lipid that inhibits aggregation of particles. In some embodiments, the gRNA molecule is fully encapsulated within the lipid portion of the lipid particle such that the gRNA molecule in the lipid particle is resistant in aqueous solution to nuclease degradation. In other embodiments, the lipid particles described herein are substantially non-toxic to mammals such as humans. The lipid particles of the invention typically have a mean diameter of from about 30 mm to about 150 nm, from about 40 nm to about 150 nm, from about 50 nm to about 150 nm, from about 60 nm to about 130 nm, from about 70 nm to about 110 nm, or from about 70 to about 90 nm. In certain embodiments, the lipid particles of the invention have a median diameter of from about 30 nm to about 150 nm. The lipid particles of the invention also typically have a lipid:nucleic acid ratio (e.g., a lipid:gRNA ratio) (mass/mass ratio) of from about 1:1 to about 100:1, from about 1:1 to about 50:1, from about 2:1 to about 25:1, from about 3:1 to about 20:1, from about 5:1 to about 15:1, or from about 5:1 to about 10:1. In certain embodiments, the nucleic acid-lipid particle has a lipid:gRNA mass ratio of from about 5:1 to about 15:1.

In preferred embodiments, the lipid particles of the invention are serum-stable nucleic acid-lipid particles which comprise one or more gRNA molecules, a cationic lipid (e.g., one or more cationic lipids of Formula I-III or salts thereof as set forth herein), a non-cationic lipid (e.g., mixtures of one or more phospholipids and cholesterol), and a conjugated lipid that inhibits aggregation of the particles (e.g., one or more PEG-lipid conjugates). The lipid particle may comprise at least 1, 2, 3, 4, 5, 6, 7, 8, 9, 10, or more gRNA molecules that target one or more of the genes described herein. Nucleic acid-lipid particles and their method of preparation are described in, e.g., U.S. Pat. Nos. 5,753,613; 5,785,992; 5,705,385; 5,976,567; 5,981,501; 6,110,745; and 6,320,017; and PCT Publication No. WO 96/40964, the disclosures of which are each herein incorporated by reference in their entirety for all purposes.

In the nucleic acid-lipid particles of the invention, the one or more gRNA molecules may be fully encapsulated within the lipid portion of the particle, thereby protecting the gRNA from nuclease degradation. In certain instances, the gRNA in the nucleic acid-lipid particle is not substantially degraded after exposure of the particle to a nuclease at 37° C. for at least about 20, 30, 45, or 60 minutes. In certain other instances, the gRNA in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the gRNA is complexed with the lipid portion of the particle. One of the benefits of the formulations of the present invention is that the nucleic acid-lipid particle compositions are substantially non-toxic to mammals such as humans.

The term “fully encapsulated” indicates that the gRNA in the nucleic acid-lipid particle is not significantly degraded after exposure to serum or a nuclease assay that would significantly degrade free DNA or RNA. In a fully encapsulated system, preferably less than about 25% of the gRNA in the particle is degraded in a treatment that would normally degrade 100% of free gRNA, more preferably less than about 10%, and most preferably less than about 5% of the gRNA in the particle is degraded. “Fully encapsulated” also indicates that the nucleic acid-lipid particles are serum-stable, that is, that they do not rapidly decompose into their component parts upon in vivo administration.

In the context of nucleic acids, full encapsulation may be determined by performing a membrane-impermeable fluorescent dye exclusion assay, which uses a dye that has enhanced fluorescence when associated with nucleic acid. Specific dyes such as OliGreen® and RiboGreen® (Invitrogen Corp.; Carlsbad, Calif.) are available for the quantitative determination of plasmid DNA, single-stranded deoxyribonucleotides, and/or single- or double-stranded ribonucleotides. Encapsulation is determined by adding the dye to a liposomal formulation, measuring the resulting fluorescence, and comparing it to the fluorescence observed upon addition of a small amount of nonionic detergent. Detergent-mediated disruption of the liposomal bilayer releases the encapsulated nucleic acid, allowing it to interact with the membrane-impermeable dye. Nucleic acid encapsulation may be calculated as E=(Iₒ−I)/Iₒ, where I and Iₒ refer to the fluorescence intensities before and after the addition of detergent (see, Wheeler et al., Gene Ther., 6:271-281 (1999)).

In other embodiments, the present invention provides a nucleic acid-lipid particle composition comprising a plurality of nucleic acid-lipid particles.

In some instances, the nucleic acid-lipid particle composition comprises a gRNA molecule that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have the gRNA encapsulated therein.

In other instances, the nucleic acid-lipid particle composition comprises gRNA that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the input gRNA is encapsulated in the particles.

Depending on the intended use of the lipid particles of the invention, the proportions of the components can be varied and the delivery efficiency of a particular formulation can be measured using, e.g., an endosomal release parameter (ERP) assay.

### 1. Cationic Lipids

Any of a variety of cationic lipids or salts thereof may be used in the lipid particles of the present invention either alone or in combination with one or more other cationic lipid species or non-cationic lipid species. The cationic lipids include the (R) and/or (S) enantiomers thereof.

In one aspect of the invention, the cationic lipid is a dialkyl lipid. For example, dialkyl lipids may include lipids that comprise two saturated or unsaturated alkyl chains, wherein each of the alkyl chains may be substituted or unsubstituted. In certain embodiments, each of the two alkyl chains comprise at least, e.g., 8 carbon atoms, 10 carbon atoms, 12 carbon atoms, 14 carbon atoms, 16 carbon atoms, 18 carbon atoms, 20 carbon atoms, 22 carbon atoms or 24 carbon atoms.

In one aspect of the invention, the cationic lipid is a trialkyl lipid. For example, trialkyl lipids may include lipids that comprise three saturated or unsaturated alkyl chains, wherein each of the alkyl chains may be substituted or unsubstituted. In certain embodiments, each of the three alkyl chains comprise at least, e.g., 8 carbon atoms, 10 carbon atoms, 12 carbon atoms, 14 carbon atoms, 16 carbon atoms, 18 carbon atoms, 20 carbon atoms, 22 carbon atoms or 24 carbon atoms.

In one aspect, cationic lipids of Formula I having the following structure are useful in the present invention:

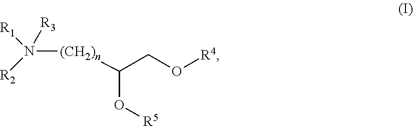

or salts thereof, wherein:

R¹ and R² are either the same or different and are independently hydrogen (H) or an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof;

R³ is either absent or is hydrogen (H) or a C₁-C₆ alkyl to provide a quaternary amine;

R⁴ and R⁵ are either the same or different and are independently an optionally substituted C₁₀-C₂₄ alkyl, C₁₀-C₂₄ alkenyl, C₁₀-C₂₄ alkynyl, or C₁₀-C₂₄ acyl, wherein at least one of R⁴ and R⁵ comprises at least two sites of unsaturation; and

n is 0, 1, 2, 3, or 4.

In some embodiments, R^{l} and R² are independently an optionally substituted C₁-C₄ alkyl, C₂-C₄ alkenyl, or C₂-C₄ alkynyl. In one preferred embodiment, R¹ and R² are both methyl groups. In other preferred embodiments, n is 1 or 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl, wherein at least one of R⁴ and R⁵ comprises at least two sites of unsaturation.

In certain embodiments, R⁴ and R⁵ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, an arachidonyl moiety, and a docosahexaenoyl moiety, as well as acyl derivatives thereof (e.g., linoleoyl, linolenoyl, γ-linolenoyl, etc.). In some instances, one of R⁴ and R⁵ comprises a branched alkyl group (e.g., a phytanyl moiety) or an acyl derivative thereof (e.g., a phytanoyl moiety). In certain instances, the octadecadienyl moiety is a linoleyl moiety. In certain other instances, the octadecatrienyl moiety is a linolenyl moiety or a γ-linolenyl moiety. In certain embodiments, R⁴ and R⁵ are both linoleyl moieties, linolenyl moieties, or y-linolenyl moieties. In particular embodiments, the cationic lipid of Formula I is 1,2-dilinoleyloxy-N,N-dimethylaminopropane (DLinDMA), 1,2-dilinolenyloxy-N,N-dimethylaminopropane (DLenDMA), 1,2-dilinoleyloxy-(N,N-dimethyl)-butyl-4-amine (C2-DLinDMA), 1,2-dilinoleoyloxy-(N,N-dimethyl)-butyl -4-amine (C2-DLinDAP), or mixtures thereof.

In some embodiments, the cationic lipid of Formula I forms a salt (preferably a crystalline salt) with one or more anions. In one particular embodiment, the cationic lipid of Formula I is the oxalate (e.g., hemioxalate) salt thereof, which is preferably a crystalline salt.

The synthesis of cationic lipids such as DLinDMA and DLenDMA, as well as additional cationic lipids, is described in U.S. Patent Publication No. 20060083780, the disclosure of which is herein incorporated by reference in its entirety for all purposes. The synthesis of cationic lipids such as C2-DLinDMA and C2-DLinDAP, as well as additional cationic lipids, is described in international patent application number W02011/000106 the disclosure of which is herein incorporated by reference in its entirety for all purposes.

In another aspect, cationic lipids of Formula II having the following structure (or salts thereof) are useful in the present invention:

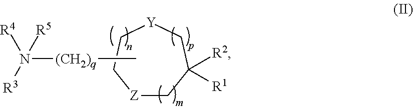

wherein Rand R² are either the same or different and are independently an optionally substituted C₁₂-C₂₄ alkyl, C₁₂-C₂₄ alkenyl, C₁₂-C₂₄ alkynyl, or C₁₂-C₂₄ acyl; R³ and R⁴ are either the same or different and are independently an optionally substituted Cᵢ-C₆ alkyl, C₂-C₆ alkenyl, or C₂-C₆ alkynyl, or R³ and R⁴ may join to form an optionally substituted heterocyclic ring of 4 to 6 carbon atoms and 1 or 2 heteroatoms chosen from nitrogen and oxygen; R⁵ is either absent or is hydrogen (H) or a C₁-C₆ alkyl to provide a quaternary amine; m, n, and p are either the same or different and are independently either 0, 1, or 2, with the proviso that m, n, and p are not simultaneously 0; q is 0, 1, 2, 3, or 4; and Y and Z are either the same or different and are independently O, S, or NH. In a preferred embodiment, q is 2.

In some embodiments, the cationic lipid of Formula II is 2,2-dilinoleyl-4-(2-dimethylaminoethyl)[1,3]-dioxolane (DLin-K-C2-DMA; “XTC2” or “C2K”), 2,2-dilinoleyl-4-(3-dimethyl aminopropyl)-[1,3]-dioxolane (DLin-K-C3-DMA; “C3K”), 2,2-dilinoleyl-4-(4-dimethylaminobutyl)-[1,3]-dioxolane (DLin-K-C4-DMA; “C4K”), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 2,2-dioleoyl-4-dimethylaminomethyl-[1,3]-dioxolane (DO-K-DMA), 2,2-distearoyl-4-dimethylaminomethyl-[1,3]-dioxolane (DS-K-DMA), 2,2-dilinoleyl-4-N-morpholino-[1,3]-dioxolane (DLin-K-MA), 2,2-Dilinoleyl-4-trimethylamino-[1,3]-dioxolane chloride (DLin-K-TMA.C1), 2,2-dilinoleyl-4,5-bis(dimethylaminomethyl)[1,3]-dioxolane (DLin-K²-DMA), 2,2-dilinoleyl-4-methylpiperzine-[1,3]-dioxolane (D-Lin-K-N-methylpiperzine), or mixtures thereof. In preferred embodiments, the cationic lipid of Formula II is DLin-K-C2-DMA.

In some embodiments, the cationic lipid of Formula II forms a salt (preferably a crystalline salt) with one or more anions. In one particular embodiment, the cationic lipid of Formula II is the oxalate (e.g., hemioxalate) salt thereof, which is preferably a crystalline salt.

The synthesis of cationic lipids such as DLin-K-DMA, as well as additional cationic lipids, is described in PCT Publication No. WO 09/086558, the disclosure of which is herein incorporated by reference in its entirety for all purposes. The synthesis of cationic lipids such as DLin-K-C2-DMA, DLin-K-C3-DMA, DLin-K-C4-DMA, DLin-K6-DMA, DLin-K-MPZ, DO-K-DMA, DS-K-DMA, DLin-K-MA, DLin-K-TMA.C1, DLin-K²-DMA, and D-Lin-K-N-methylpiperzine, as well as additional cationic lipids, is described in PCT Application No. PCT/US2009/060251, entitled “Improved Amino Lipids and Methods for the Delivery of Nucleic Acids,” filed Oct. 9, 2009, the disclosure of which is incorporated herein by reference in its entirety for all purposes.

In a further aspect, cationic lipids of Formula III having the following structure are useful in the present invention:

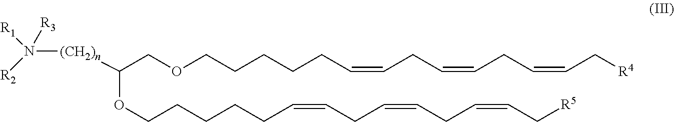

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either absent or present and when present are either the same or different and are independently an optionally substituted C₁-C₁₀ alkyl or C₂-Cᵢₒ alkenyl; and n is 0, 1, 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, R⁴ and R⁵ are both butyl groups. In yet another preferred embodiment, n is 1. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₂-C₆ or C₂-C₄ alkyl or C₂-C₆ or C₂-C₄ alkenyl.

In an alternative embodiment, the cationic lipid of Formula III comprises ester linkages between the amino head group and one or both of the alkyl chains. In some embodiments, the cationic lipid of Formula III forms a salt (preferably a crystalline salt) with one or more anions. In one particular embodiment, the cationic lipid of Formula III is the oxalate (e.g., hemioxalate) salt thereof, which is preferably a crystalline salt.

Although each of the alkyl chains in Formula III contains cis double bonds at positions 6, 9, and 12 (i.e., cis,cis,cis-Δ⁶, Δ⁹, Δ¹²), in an alternative embodiment, one, two, or three of these double bonds in one or both alkyl chains may be in the trans configuration.

In a particularly preferred embodiment, the cationic lipid of Formula III has the structure:

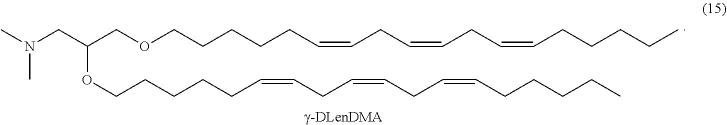 [SMILES: CCCCC/C=C\C/C=C\C/C=C\CCCCCOCC(CN(C)C)OCCCCC/C=C\C/C=C\C/C=C\CCCCC]

The synthesis of cationic lipids such as y-DLenDMA (15), as well as additional cationic lipids, is described in U.S. Provisional Application No. 61/222,462, entitled “Improved Cationic Lipids and Methods for the Delivery of Nucleic Acids,” filed Jul. 1, 2009, the disclosure of which is herein incorporated by reference in its entirety for all purposes.

The synthesis of cationic lipids such as DLin-M-C3-DMA (“MC3”), as well as additional cationic lipids (e.g., certain analogs of MC3), is described in U.S. Provisional Application No. 61/185,800, entitled “Novel Lipids and Compositions for the Delivery of Therapeutics,” filed Jun. 10, 2009, and U.S. Provisional Application No. 61/287,995, entitled “Methods and Compositions for Delivery of Nucleic Acids,” filed Dec. 18, 2009, the disclosures of which are herein incorporated by reference in their entirety for all purposes.

Examples of other cationic lipids or salts thereof which may be included in the lipid particles of the present invention include, but are not limited to, cationic lipids such as those described in WO2011/000106, the disclosure of which is herein incorporated by reference in its entirety for all purposes, as well as cationic lipids such as N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtri fluoro acetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′ -(cholest-5-en-3-beta-oxy)-3′-oxapentoxy)-3-dimethy-1-(cis,ci s-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethyl amino)etho x yprop ane (DLin-EG-DMA), 1,2-dioeylcarbamoyloxy-3-dimethylaminopropane (DO-C-DAP), 1,2-dimyristoleoyl-3-dimethylaminopropane (DMDAP), 1,2-dioleoyl-3-trimethylaminopropane chloride (DOTAP.C1), dilinoleylmethyl-3-dimethylaminopropionate (DLin-M-C2-DMA; also known as DLin-M-K-DMA or DLin-M-DMA), and mixtures thereof. Additional cationic lipids or salts thereof which may be included in the lipid particles of the present invention are described in U.S. Patent Publication No. 20090023673, the disclosure of which is herein incorporated by reference in its entirety for all purposes.

The synthesis of cationic lipids such as CLinDMA, as well as additional cationic lipids, is described in U.S. Patent Publication No. 20060240554, the disclosure of which is herein incorporated by reference in its entirety for all purposes. The synthesis of cationic lipids such as DLin-C-DAP, DLinDAC, DLinMA, DLinDAP, DLin-S-DMA, DLin-2-DMAP, DLinTMA.C1, DLinTAP.CI, DLinMPZ, DLinAP, DOAP, and DLin-EG-DMA, as well as additional cationic lipids, is described in PCT Publication No. WO 09/086558, the disclosure of which is herein incorporated by reference in its entirety for all purposes. The synthesis of cationic lipids such as DO-C-DAP, DMDAP, DOTAP.C1, DLin-M-C2-DMA, as well as additional cationic lipids, is described in PCT Application No. PCT/US2009/060251, entitled “Improved Amino Lipids and Methods for the Delivery of Nucleic Acids,” filed Oct. 9, 2009, the disclosure of which is incorporated herein by reference in its entirety for all purposes. The synthesis of a number of other cationic lipids and related analogs has been described in U.S. Pat. Nos. 5,208,036; 5,264,618; 5,279,833; 5,283,185; 5,753,613; and 5,785,992; and PCT Publication No. WO 96/10390, the disclosures of which are each herein incorporated by reference in their entirety for all purposes. Additionally, a number of commercial preparations of cationic lipids can be used, such as, e.g., LIPOFECTIN® (including DOTMA and DOPE, available from Invitrogen); LIPOFECTAMINE® (including DOSPA and DOPE, available from Invitrogen); and TRANSFECTAM® (including DOGS, available from Promega Corp.).

In some embodiments, the cationic lipid comprises from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, from about 50 mol % to about 60 mol %, from about 55 mol % to about 65 mol %, or from about 55 mol % to about 70 mol % (or any fraction thereof or range therein) of the total lipid present in the particle. In particular embodiments, the cationic lipid comprises about 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, or 65 mol % (or any fraction thereof) of the total lipid present in the particle.

In other embodiments, the cationic lipid comprises from about 2 mol % to about 60 mol %, from about 5 mol % to about 50 mol %, from about 10 mol % to about 50 mol %, from about 20 mol % to about 50 mol %, from about 20 mol % to about 40 mol %, from about 30 mol % to about 40 mol %, or about 40 mol % (or any fraction thereof or range therein) of the total lipid present in the particle.

Additional percentages and ranges of cationic lipids suitable for use in the lipid particles of the present invention are described in PCT Publication No. WO 09/127060, U.S. Published Application No. US 2011/0071208, PCT Publication No. W02011/000106, and U.S. Published Application No. US 2011/0076335, the disclosures of which are herein incorporated by reference in their entirety for all purposes.

It should be understood that the percentage of cationic lipid present in the lipid particles of the invention is a target amount, and that the actual amount of cationic lipid present in the formulation may vary, for example, by ±5 mol %. For example, in one exemplary lipid particle formulation, the target amount of cationic lipid is 57.1 mol %, but the actual amount of cationic lipid may be ±5 mol %, ±4 mol %, ±3 mol %, ±2 mol %, ±1 mol %, ±0.75 mol %, ±0.5 mol %, ±0.25 mol %, or ±0.1 mol % of that target amount, with the balance of the formulation being made up of other lipid components (adding up to 100 mol % of total lipids present in the particle; however, one skilled in the art will understand that the total mol % may deviate slightly from 100% due to rounding, for example, 99.9 mol % or 100.1 mol %.).

Further examples of cationic lipids useful for inclusion in lipid particles used in the present invention are shown below:

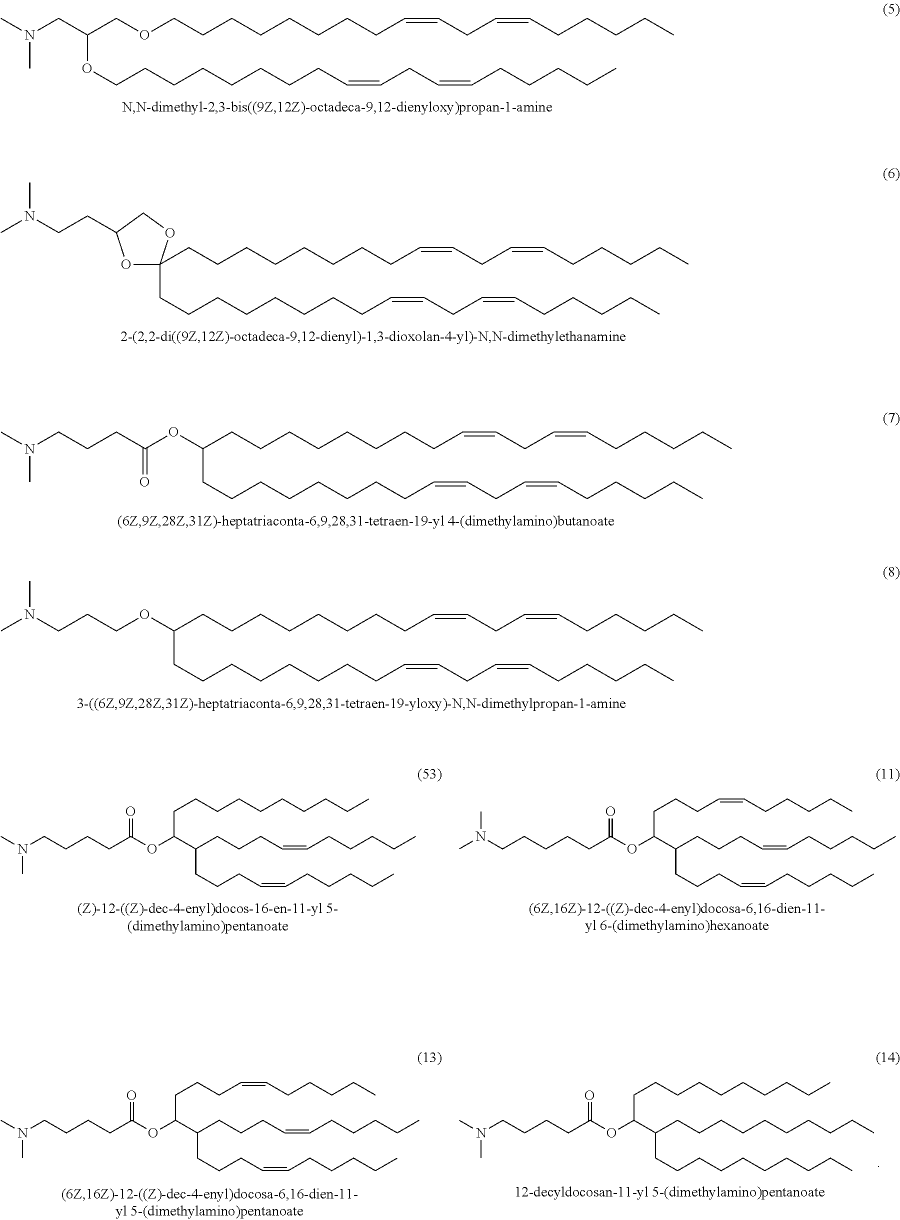

### 2. Non-Cationic Lipids

The non-cationic lipids used in the lipid particles of the invention can be any of a variety of neutral uncharged, zwitterionic, or anionic lipids capable of producing a stable complex.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), di oleoylpho sphatidyl gl ycerol (DOPG), di palmitoylpho sphatidyl glyc erol (DPP G), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In preferred embodiments, the cholesterol derivative is a polar analogue such as cholesteryl-(4′-hydroxy)-butyl ether. The synthesis of cholesteryl-(2′-hydroxy)-ethyl ether is described in PCT Publication No. WO 09/127060, the disclosure of which is herein incorporated by reference in its entirety for all purposes.

In some embodiments, the non-cationic lipid present in the lipid particles comprises or consists of a mixture of one or more phospholipids and cholesterol or a derivative thereof. In other embodiments, the non-cationic lipid present in the lipid particles comprises or consists of one or more phospholipids, e.g., a cholesterol-free lipid particle formulation. In yet other embodiments, the non-cationic lipid present in the lipid particles comprises or consists of cholesterol or a derivative thereof, e.g., a phospholipid-free lipid particle formulation.

Other examples of non-cationic lipids suitable for use in the present invention include nonphosphorous containing lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid comprises from about 10 mol % to about 60 mol %, from about 20 mol % to about 55 mol %, from about 20 mol % to about 45 mol %, from about 20 mol % to about 40 mol %, from about 25 mol % to about 50 mol %, from about 25 mol % to about 45 mol %, from about 30 mol % to about 50 mol %, from about 30 mol % to about 45 mol %, from about 30 mol % to about 40 mol %, from about 35 mol % to about 45 mol %, from about 37 mol % to about 45 mol %, or about 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipid present in the particle.

In embodiments where the lipid particles contain a mixture of phospholipid and cholesterol or a cholesterol derivative, the mixture may comprise up to about 40 mol %, 45 mol %, 50 mol %, 55 mol %, or 60 mol % of the total lipid present in the particle.

In some embodiments, the phospholipid component in the mixture may comprise from about 2 mol % to about 20 mol %, from about 2 mol % to about 15 mol %, from about 2 mol % to about 12 mol %, from about 4 mol % to about 15 mol %, or from about 4 mol % to about 10 mol % (or any fraction thereof or range therein) of the total lipid present in the particle. In an certain embodiments, the phospholipid component in the mixture comprises from about 5 mol % to about 17 mol %, from about 7 mol % to about 17 mol %, from about 7 mol % to about 15 mol %, from about 8 mol % to about 15 mol %, or about 8 mol %, 9 mol %, 10 mol %, 11 mol %, 12 mol %, 13 mol %, 14 mol %, or 15 mol % (or any fraction thereof or range therein) of the total lipid present in the particle. As a non-limiting example, a lipid particle formulation comprising a mixture of phospholipid and cholesterol may comprise a phospholipid such as DPPC or DSPC at about 7 mol % (or any fraction thereof), e.g., in a mixture with cholesterol or a cholesterol derivative at about 34 mol % (or any fraction thereof) of the total lipid present in the particle. As another non-limiting example, a lipid particle formulation comprising a mixture of phospholipid and cholesterol may comprise a phospholipid such as DPPC or DSPC at about 7 mol % (or any fraction thereof), e.g., in a mixture with cholesterol or a cholesterol derivative at about 32 mol % (or any fraction thereof) of the total lipid present in the particle.

By way of further example, a lipid formulation useful in the practice of the invention has a lipid to drug (e.g., gRNA) ratio of about 10:1 (e.g., a lipid:drug ratio of from 9.5:1 to 11:1, or from 9.9:1 to 11:1, or from 10:1 to 10.9:1). In certain other embodiments, a lipid formulation useful in the practice of the invention has a lipid to drug (e.g., gRNA) ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

In other embodiments, the cholesterol component in the mixture may comprise from about 25 mol % to about 45 mol %, from about 25 mol % to about 40 mol %, from about 30 mol % to about 45 mol %, from about 30 mol % to about 40 mol %, from about 27 mol % to about 37 mol %, from about 25 mol % to about 30 mol %, or from about 35 mol % to about 40 mol % (or any fraction thereof or range therein) of the total lipid present in the particle. In certain preferred embodiments, the cholesterol component in the mixture comprises from about 25 mol % to about 35 mol %, from about 27 mol % to about 35 mol %, from about 29 mol % to about 35 mol %, from about 30 mol % to about 35 mol %, from about 30 mol % to about 34 mol %, from about 31 mol % to about 33 mol %, or about 30 mol %, 31 mol %, 32 mol %, 33 mol %, 34 mol %, or 35 mol % (or any fraction thereof or range therein) of the total lipid present in the particle.

In embodiments where the lipid particles are phospholipid-free, the cholesterol or derivative thereof may comprise up to about 25 mol %, 30 mol %, 35 mol %, 40 mol %, 45 mol %, 50 mol %, 55 mol %, or 60 mol % of the total lipid present in the particle.

In some embodiments, the cholesterol or derivative thereof in the phospholipid-free lipid particle formulation may comprise from about 25 mol % to about 45 mol %, from about 25 mol % to about 40 mol %, from about 30 mol % to about 45 mol %, from about 30 mol % to about 40 mol %, from about 31 mol % to about 39 mol %, from about 32 mol % to about 38 mol %, from about 33 mol % to about 37 mol %, from about 35 mol % to about 45 mol %, from about 30 mol % to about 35 mol %, from about 35 mol % to about 40 mol %, or about 30 mol %, 31 mol %, 32 mol %, 33 mol %, 34 mol %, 35 mol %, 36 mol %, 37 mol %, 38 mol %, 39 mol %, or 40 mol % (or any fraction thereof or range therein) of the total lipid present in the particle. As a non-limiting example, a lipid particle formulation may comprise cholesterol at about 37 mol % (or any fraction thereof) of the total lipid present in the particle. As another non-limiting example, a lipid particle formulation may comprise cholesterol at about 35 mol % (or any fraction thereof) of the total lipid present in the particle.

In other embodiments, the non-cationic lipid comprises from about 5 mol % to about 90 mol %, from about 10 mol % to about 85 mol %, from about 20 mol % to about 80 mol %, about 10 mol % (e.g., phospholipid only), or about 60 mol % (e.g., phospholipid and cholesterol or derivative thereof) (or any fraction thereof or range therein) of the total lipid present in the particle.

Additional percentages and ranges of non-cationic lipids suitable for use in the lipid particles of the present invention are described in PCT Publication No. WO 09/127060, U.S. Published Application No. US 2011/0071208, PCT Publication No. WO2011/000106, and U.S. Published Application No. US 2011/0076335, the disclosures of which are herein incorporated by reference in their entirety for all purposes.

It should be understood that the percentage of non-cationic lipid present in the lipid particles of the invention is a target amount, and that the actual amount of non-cationic lipid present in the formulation may vary, for example, by ±5 mol %, ±4 mol %, ±3 mol %, ±2 mol %, ±1 mol %, ±0.75 mol %, ±0.5 mol %, ±0.25 mol %, or ±0.1 mol %.

### 3. Lipid Conjugates

In addition to cationic and non-cationic lipids, the lipid particles of the invention may further comprise a lipid conjugate. The conjugated lipid is useful in that it prevents the aggregation of particles. Suitable conjugated lipids include, but are not limited to, PEG-lipid conjugates, POZ-lipid conjugates, ATTA-lipid conjugates, cationic-polymer-lipid conjugates (CPLs), and mixtures thereof. In certain embodiments, the particles comprise either a PEG-lipid conjugate or an ATTA-lipid conjugate together with a CPL.

In a preferred embodiment, the lipid conjugate is a PEG-lipid. Examples of PEG-lipids include, but are not limited to, PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides as described in, e.g., U.S. Pat. No. 5,885,613, PEG conjugated to cholesterol or a derivative thereof, and mixtures thereof. The disclosures of these patent documents are herein incorporated by reference in their entirety for all purposes.

Additional PEG-lipids suitable for use in the invention include, without limitation, mPEG2000-1,2-di-O-alkyl-sn3-carbomoylglyceride (PEG-C-DOMG). The synthesis of PEG-C-DOMG is described in PCT Publication No. WO 09/086558, the disclosure of which is herein incorporated by reference in its entirety for all purposes. Yet additional suitable PEG-lipid conjugates include, without limitation, 1-[8′-(1,2-dimyristoyl-3-propanoxy)-carboxamido-3′,6′-dioxaoctanyl]carbamoyl-ω-methyl-poly(ethylene glycol) (2KPEG-DMG). The synthesis of 2KPEG-DMG is described in U.S. Pat. No. 7,404,969, the disclosure of which is herein incorporated by reference in its entirety for all purposes.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, but are not limited to, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH₂, etc.). Other PEGs such as those described in U.S. Pat. Nos. 6,774,180 and 7,053,150 (e.g., mPEG (20 KDa) amine) are also useful for preparing the PEG-lipid conjugates of the present invention. The disclosures of these patents are herein incorporated by reference in their entirety for all purposes. In addition, monomethoxypolyethyleneglycol-acetic acid (MePEG-CH₂COOH) is particularly useful for preparing PEG-lipid conjugates including, e.g., PEG-DAA conjugates.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In preferred embodiments, the PEG moiety has an average molecular weight of about 2,000 daltons or about 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to the lipid. Suitable ester containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available, or can be isolated or synthesized using conventional techniques known to those of skill in the art. Phosphatidyl-ethanolamines containing saturated or unsaturated fatty acids with carbon chain lengths in the range of C ₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

The term “ATTA” or “polyamide” includes, without limitation, compounds described in U.S. Pat. Nos. 6,320,017 and 6,586,559, the disclosures of which are herein incorporated by reference in their entirety for all purposes. These compounds include a compound having the formula:

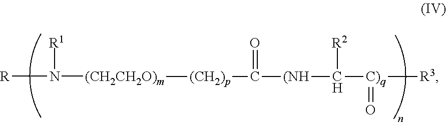

wherein R is a member selected from the group consisting of hydrogen, alkyl and acyl; R¹ is a member selected from the group consisting of hydrogen and alkyl; or optionally, R and R¹ and the nitrogen to which they are bound form an azido moiety; R² is a member of the group selected from hydrogen, optionally substituted alkyl, optionally substituted aryl and a side chain of an amino acid; R³ is a member selected from the group consisting of hydrogen, halogen, hydroxy, alkoxy, mercapto, hydrazino, amino and NR⁴R⁵, wherein R⁴ and R⁵ are independently hydrogen or alkyl; n is 4 to 80; m is 2 to 6; p is 1 to 4; and q is 0 or 1. It will be apparent to those of skill in the art that other polyamides can be used in the compounds of the present invention.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (C₁₈), and icosoyl (C₂₀). In preferred embodiments, R¹ and R² are the same, i.e., R¹ and R² are both myristoyl (i.e., dimyristoyl), R¹ and R² are both stearoyl (i.e., distearoyl), etc. Diacylglycerols have the following general formula:

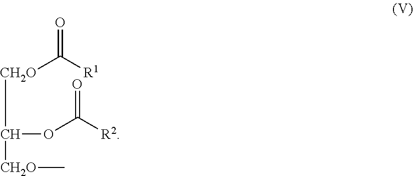

The term “dialkyloxypropyl” or “DAA” includes a compound having 2 alkyl chains, R¹ and R², both of which have independently between 2 and 30 carbons. The alkyl groups can be saturated or have varying degrees of unsaturation. Dialkyloxypropyls have the following general formula:

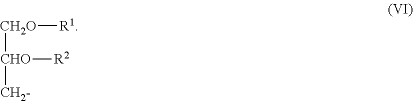

In a preferred embodiment, the PEG-lipid is a PEG-DAA conjugate having the following formula:

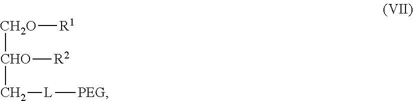

wherein R¹ and R² are independently selected and are long-chain alkyl groups having from about 10 to about 22 carbon atoms; PEG is a polyethyleneglycol; and L is a non-ester containing linker moiety or an ester containing linker moiety as described above. The long-chain alkyl groups can be saturated or unsaturated. Suitable alkyl groups include, but are not limited to, decyl (C₁₀), lauryl (C₁₂), myristyl (C₁₄), palmityl (C₁₆), stearyl (C₁₈), and icosyl (C₂₀). In preferred embodiments, R¹ and R² are the same, i.e., R¹ and R² are both myristyl (i.e., dimyristyl), re and R² are both stearyl (i.e., distearyl), etc.

In Formula VII above, the PEG has an average molecular weight ranging from about 550 daltons to about 10,000 daltons. In certain instances, the PEG has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In preferred embodiments, the PEG has an average molecular weight of about 2,000 daltons or about 750 daltons. The PEG can be optionally substituted with alkyl, alkoxy, acyl, or aryl groups. In certain embodiments, the terminal hydroxyl group is substituted with a methoxy or methyl group.

In a preferred embodiment, “L” is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a disulphide linker moiety, a succinamidyl linker moiety, and combinations thereof. In a preferred embodiment, the non-ester containing linker moiety is a carbamate linker moiety (i.e., a PEG-C-DAA conjugate). In another preferred embodiment, the non-ester containing linker moiety is an amido linker moiety (i.e., a PEG-A-DAA conjugate). In yet another preferred embodiment, the non-ester containing linker moiety is a succinamidyl linker moiety (i.e., a PEG-S-DAA conjugate).

In particular embodiments, the PEG-lipid conjugate is selected from:

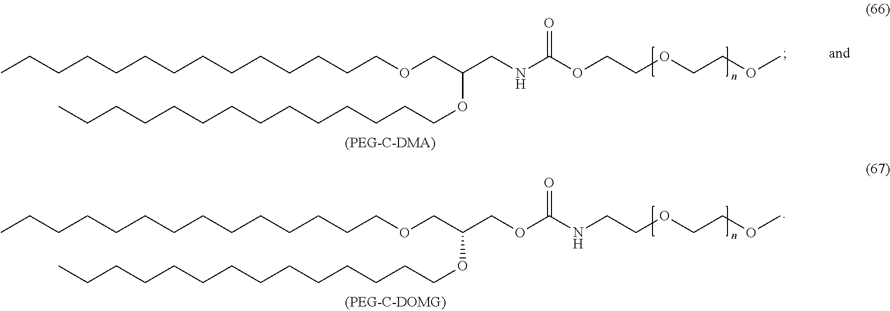

The PEG-DAA conjugates are synthesized using standard techniques and reagents known to those of skill in the art. It will be recognized that the PEG-DAA conjugates will contain various amide, amine, ether, thio, carbamate, and urea linkages. Those of skill in the art will recognize that methods and reagents for forming these bonds are well known and readily available. See, e.g., March, ADVANCED ORGANIC CHEMISTRY (Wiley 1992); Larock, COMPREHENSIVE ORGANIC TRANSFORMATIONS (VCH 1989); and Furniss, VOGEL′S TEXTBOOK OF PRACTICAL ORGANIC CHEMISTRY, 5th ed. (Longman 1989). It will also be appreciated that any functional groups present may require protection and deprotection at different points in the synthesis of the PEG-DAA conjugates. Those of skill in the art will recognize that such techniques are well known. See, e.g., Green and Wuts, PROTECTIVE GROUPS IN ORGANIC SYNTHESIS (Wiley 1991).

Preferably, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 or about 2,000 daltons. In one particularly preferred embodiment, the PEG-lipid conjugate comprises PEG2000-C-DMA, wherein the “2000” denotes the average molecular weight of the PEG, the “C” denotes a carbamate linker moiety, and the “DMA” denotes dimyristyloxypropyl. In another particularly preferred embodiment, the PEG-lipid conjugate comprises PEG750-C-DMA, wherein the “750” denotes the average molecular weight of the PEG, the “C” denotes a carbamate linker moiety, and the “DMA” denotes dimyristyloxypropyl. In particular embodiments, the terminal hydroxyl group of the PEG is substituted with a methyl group. Those of skill in the art will readily appreciate that other dialkyloxypropyls can be used in the PEG-DAA conjugates of the present invention.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the lipid particles of the present invention can further comprise cationic poly(ethylene glycol) (PEG) lipids or CPLs (see, e.g., Chen et al., Bioconj. Chem., 11:433-437 (2000); U.S. Pat. No. 6,852,334; PCT Publication No. WO 00/62813, the disclosures of which are herein incorporated by reference in their entirety for all purposes).

Suitable CPLs include compounds of Formula VIII:

A-W-Y (VIII),

wherein A, W, and Y are as described below.

With reference to Formula VIII, “A” is a lipid moiety such as an amphipathic lipid, a neutral lipid, or a hydrophobic lipid that acts as a lipid anchor. Suitable lipid examples include, but are not limited to, diacylglycerolyls, dialkylglycerolyls, N-N-dialkylaminos, 1,2-diacyloxy-3-aminopropanes, and 1,2-dialkyl-3-aminopropanes.

“W” is a polymer or an oligomer such as a hydrophilic polymer or oligomer. Preferably, the hydrophilic polymer is a biocompatable polymer that is nonimmunogenic or possesses low inherent immunogenicity. Alternatively, the hydrophilic polymer can be weakly antigenic if used with appropriate adjuvants. Suitable nonimmunogenic polymers include, but are not limited to, PEG, polyamides, polylactic acid, polyglycolic acid, polylactic acid/polyglycolic acid copolymers, and combinations thereof. In a preferred embodiment, the polymer has a molecular weight of from about 250 to about 7,000 daltons.

“Y” is a polycationic moiety. The term polycationic moiety refers to a compound, derivative, or functional group having a positive charge, preferably at least 2 positive charges at a selected pH, preferably physiological pH. Suitable polycationic moieties include basic amino acids and their derivatives such as arginine, asparagine, glutamine, lysine, and histidine; spermine; spermidine; cationic dendrimers; polyamines; polyamine sugars; and amino polysaccharides. The polycationic moieties can be linear, such as linear tetralysine, branched or dendrimeric in structure. Polycationic moieties have between about 2 to about 15 positive charges, preferably between about 2 to about 12 positive charges, and more preferably between about 2 to about 8 positive charges at selected pH values. The selection of which polycationic moiety to employ may be determined by the type of particle application which is desired.

The charges on the polycationic moieties can be either distributed around the entire particle moiety, or alternatively, they can be a discrete concentration of charge density in one particular area of the particle moiety e.g., a charge spike. If the charge density is distributed on the particle, the charge density can be equally distributed or unequally distributed. All variations of charge distribution of the polycationic moiety are encompassed by the present invention.

The lipid “A” and the nonimmunogenic polymer “W” can be attached by various methods and preferably by covalent attachment. Methods known to those of skill in the art can be used for the covalent attachment of “A” and “W.” Suitable linkages include, but are not limited to, amide, amine, carboxyl, carbonate, carbamate, ester, and hydrazone linkages. It will be apparent to those skilled in the art that “A” and “W” must have complementary functional groups to effectuate the linkage. The reaction of these two groups, one on the lipid and the other on the polymer, will provide the desired linkage. For example, when the lipid is a diacylglycerol and the terminal hydroxyl is activated, for instance with NHS and DCC, to form an active ester, and is then reacted with a polymer which contains an amino group, such as with a polyamide (see, e.g., U.S. Pat. Nos. 6,320,017 and 6,586,559, the disclosures of which are herein incorporated by reference in their entirety for all purposes), an amide bond will form between the two groups.

In certain instances, the polycationic moiety can have a ligand attached, such as a targeting ligand or a chelating moiety for complexing calcium. Preferably, after the ligand is attached, the cationic moiety maintains a positive charge. In certain instances, the ligand that is attached has a positive charge. Suitable ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

In some embodiments, the lipid conjugate (e.g., PEG-lipid) comprises from about 0.1 mol % to about 3 mol %, from about 0.5 mol % to about 3 mol %, or about 0.6 mol %, 0.7 mol %, 0.8 mol %, 0.9 mol %, 1.0 mol %, 1.1 mol %, 1.2 mol %, 1.3 mol %, 1.4 mol %, 1.5 mol %, 1.6 mol %, 1.7 mol %, 1.8 mol %, 1.9 mol %, 2.0 mol %, 2.1 mol%, 2.2 mol%, 2.3 mol %, 2.4 mol %, 2.5 mol %, 2.6 mol %, 2.7 mol %, 2.8 mol %, 2.9 mol % or 3 mol (or any fraction thereof or range therein) of the total lipid present in the particle.

In other embodiments, the lipid conjugate (e.g., PEG-lipid) comprises from about 0 mol % to about 20 mol %, from about 0.5 mol % to about 20 mol %, from about 2 mol % to about 20 mol %, from about 1.5 mol % to about 18 mol %, from about 2 mol % to about 15 mol %, from about 4 mol % to about 15 mol %, from about 2 mol % to about 12 mol %, from about 5 mol % to about 12 mol %, or about 2 mol % (or any fraction thereof or range therein) of the total lipid present in the particle.

In further embodiments, the lipid conjugate (e.g., PEG-lipid) comprises from about 4 mol % to about 10 mol %, from about 5 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle.

It should be understood that the percentage of lipid conjugate present in the lipid particles of the invention is a target amount, and that the actual amount of lipid conjugate present in the formulation may vary, for example, by ±5 mol %, ±4 mol %, ±3 mol %, ±2 mol %, ±1 mol %, ±0.75 mol %, ±0.5 mol %, ±0.25 mol %, or ±0.1 mol %.

Additional percentages and ranges of lipid conjugates suitable for use in the lipid particles of the present invention are described in PCT Publication No. WO 09/127060, U.S. Published Application No. US 2011/0071208, PCT Publication No. WO2011/000106, and U.S. Published Application No. US 2011/0076335, the disclosures of which are herein incorporated by reference in their entirety for all purposes.

One of ordinary skill in the art will appreciate that the concentration of the lipid conjugate can be varied depending on the lipid conjugate employed and the rate at which the lipid particle is to become fusogenic.

By controlling the composition and concentration of the lipid conjugate, one can control the rate at which the lipid conjugate exchanges out of the lipid particle and, in turn, the rate at which the lipid particle becomes fusogenic. For instance, when a PEG-DAA conjugate is used as the lipid conjugate, the rate at which the lipid particle becomes fusogenic can be varied, for example, by varying the concentration of the lipid conjugate, by varying the molecular weight of the PEG, or by varying the chain length and degree of saturation of the alkyl groups on the PEG-DAA conjugate. In addition, other variables including, for example, pH, temperature, ionic strength, etc. can be used to vary and/or control the rate at which the lipid particle becomes fusogenic. Other methods which can be used to control the rate at which the lipid particle becomes fusogenic will become apparent to those of skill in the art upon reading this disclosure. Also, by controlling the composition and concentration of the lipid conjugate, one can control the lipid particle size.

### B. Additional Carrier Systems

Non-limiting examples of additional lipid-based carrier systems suitable for use in the present invention include lipoplexes (see, e.g., U.S. Patent Publication No. 20030203865; and Zhang et al., J. Control Release, 100:165-180 (2004)), pH-sensitive lipoplexes (see, e.g., U.S. Patent Publication No. 20020192275), reversibly masked lipoplexes (see, e.g., U.S. Patent Publication Nos. 20030180950), cationic lipid-based compositions (see, e.g., U.S. Pat. No. 6,756,054; and U.S. Patent Publication No. 20050234232), cationic liposomes (see, e.g., U.S. Patent Publication Nos. 20030229040, 20020160038, and 20020012998; U.S. Pat. No. 5,908,635; and PCT Publication No. WO 01/72283), anionic liposomes (see, e.g., U.S. Patent Publication No. 20030026831), pH-sensitive liposomes (see, e.g., U.S. Patent Publication No. 20020192274; and AU 2003210303), antibody-coated liposomes (see, e.g., U.S. Patent Publication No. 20030108597; and PCT Publication No. WO 00/50008), cell-type specific liposomes (see, e.g., U.S. Patent Publication No. 20030198664), liposomes containing nucleic acid and peptides (see, e.g., U.S. Pat. No. 6,207,456), liposomes containing lipids derivatized with releasable hydrophilic polymers (see, e.g., U.S. Patent Publication No. 20030031704), lipid-entrapped nucleic acid (see, e.g., PCT Publication Nos. WO 03/057190 and WO 03/059322), lipid-encapsulated nucleic acid (see, e.g., U.S. Patent Publication No. 20030129221; and U.S. Pat. No. 5,756,122), other liposomal compositions (see, e.g., U.S. Patent Publication Nos. 20030035829 and 20030072794; and U.S. Pat. No. 6,200,599), stabilized mixtures of liposomes and emulsions (see, e.g., EP1304160), emulsion compositions (see, e.g., U.S. Pat. No. 6,747,014), and nucleic acid micro-emulsions (see, e.g., U.S. Patent Publication No. 20050037086).

Examples of polymer-based carrier systems suitable for use in the present invention include, but are not limited to, cationic polymer-nucleic acid complexes (i.e., polyplexes). To form a polyplex, a nucleic acid is typically complexed with a cationic polymer having a linear, branched, star, or dendritic polymeric structure that condenses the nucleic acid into positively charged particles capable of interacting with anionic proteoglycans at the cell surface and entering cells by endocytosis. In some embodiments, the polyplex comprises nucleic acid (e.g., a gRNA molecule) complexed with a cationic polymer such as polyethylenimine (PEI) (see, e.g., U.S. Pat. No. 6,013,240; commercially available from Qbiogene, Inc. (Carlsbad, Calif.) as In vivo jetPEI™, a linear form of PEI), polypropylenimine (PPI), polyvinylpyrrolidone (PVP), poly-L-lysine (PLL), diethylaminoethyl (DEAE)-dextran, poly((β-amino ester) (PAE) polymers (see, e.g., Lynn et al., J. Am. Chem. Soc., 123:8155-8156 (2001)), chitosan, polyamidoamine (PAMAM) dendrimers (see, e.g., Kukowska-Latallo et al., Proc. Natl. Acad. Sci. USA, 93:4897-4902 (1996)), porphyrin (see, e.g., U.S. Pat. No. 6,620,805), polyvinylether (see, e.g., U.S. Patent Publication No. 20040156909), polycyclic amidinium (see, e.g., U.S. Patent Publication No. 20030220289), other polymers comprising primary amine, imine, guanidine, and/or imidazole groups (see, e.g., U.S. Pat. No. 6,013,240; PCT Publication No. WO/9602655; PCT Publication No. WO95/21931; Zhang et al., J. Control Release, 100:165-180 (2004); and Tiera et al., Curr. Gene Ther., 6:59-71 (2006)), and a mixture thereof. In other embodiments, the polyplex comprises cationic polymer-nucleic acid complexes as described in U.S. Patent Publication Nos. 20060211643, 20050222064, 20030125281, and 20030185890, and PCT Publication No. WO 03/066069; biodegradable poly(I3-amino ester) polymer-nucleic acid complexes as described in U.S. Patent Publication No. 20040071654; microparticles containing polymeric matrices as described in U.S. Patent Publication No. 20040142475; other microparticle compositions as described in U.S. Patent Publication No. 20030157030; condensed nucleic acid complexes as described in U.S. Patent Publication No. 20050123600; and nanocapsule and microcapsule compositions as described in AU 2002358514 and PCT Publication No. WO 02/096551.

In certain instances, the gRNA may be complexed with cyclodextrin or a polymer thereof. Non-limiting examples of cyclodextrin-based carrier systems include the cyclodextrin-modified polymer-nucleic acid complexes described in U.S. Patent Publication No. 20040087024; the linear cyclodextrin copolymer-nucleic acid complexes described in U.S. Pat. Nos. 6,509,323, 6,884,789, and 7,091,192; and the cyclodextrin polymer-complexing agent-nucleic acid complexes described in U.S. Pat. No. 7,018,609. In certain other instances, the gRNA may be complexed with a peptide or polypeptide. An example of a protein-based carrier system includes, but is not limited to, the cationic oligopeptide-nucleic acid complex described in PCT Publication No. WO95/21931.

Preparation of Lipid Particles

The nucleic acid-lipid particles of the present invention, in which a nucleic acid (e.g., a gRNA) is entrapped within the lipid portion of the particle and is protected from degradation, can be formed by any method known in the art including, but not limited to, a continuous mixing method, a direct dilution process, and an in-line dilution process.

In particular embodiments, the cationic lipids may comprise lipids of Formula I-III or salts thereof, alone or in combination with other cationic lipids. In other embodiments, the non-cationic lipids are egg sphingomyelin (ESM), distearoylphosphatidylcholine (DSPC), dioleoylphosphatidylcholine (DOPC), 1-palmitoyl-2-oleoyl-phosphatidylcholine (POPC), dipalmitoyl-phosphatidylcholine (DPPC), monomethyl-phosphatidylethanolamine, dimethyl-phosphatidylethanolamine, 14:0 PE (1,2-dimyristoyl-phosphatidylethanolamine (DMPE)), 16:0 PE (1,2-dipalmitoyl-phosphatidylethanolamine (DPPE)), 18:0 PE (1,2-distearoyl-phosphatidylethanolamine (DSPE)), 18:1 PE (1,2-dioleoyl-phosphatidylethanolamine (DOPE)), 18:1 trans PE (1,2-dielaidoyl-phosphatidylethanolamine (DEPE)), 18:0-18:1 PE (1-stearoyl-2-oleoyl-phosphatidylethanolamine (SOPE)), 16:0-18:1 PE (1-palm itoyl-2-oleoyl-phosphatidylethanolamine (POPE)), polyethylene glycol-based polymers (e.g., PEG 2000, PEG 5000, PEG-modified diacylglycerols, or PEG-modified dialkyloxypropyls), cholesterol, derivatives thereof, or combinations thereof.

In certain embodiments, the present invention provides nucleic acid-lipid particles produced via a continuous mixing method, e.g., a process that includes providing an aqueous solution comprising a gRNA in a first reservoir, providing an organic lipid solution in a second reservoir (wherein the lipids present in the organic lipid solution are solubilized in an organic solvent, e.g., a lower alkanol such as ethanol), and mixing the aqueous solution with the organic lipid solution such that the organic lipid solution mixes with the aqueous solution so as to substantially instantaneously produce a lipid vesicle (e.g., liposome) encapsulating the gRNA within the lipid vesicle. This process and the apparatus for carrying out this process are described in detail in U.S. Patent Publication No. 20040142025, the disclosure of which is herein incorporated by reference in its entirety for all purposes.

The action of continuously introducing lipid and buffer solutions into a mixing environment, such as in a mixing chamber, causes a continuous dilution of the lipid solution with the buffer solution, thereby producing a lipid vesicle substantially instantaneously upon mixing. As used herein, the phrase “continuously diluting a lipid solution with a buffer solution” (and variations) generally means that the lipid solution is diluted sufficiently rapidly in a hydration process with sufficient force to effectuate vesicle generation. By mixing the aqueous solution comprising a nucleic acid with the organic lipid solution, the organic lipid solution undergoes a continuous stepwise dilution in the presence of the buffer solution (i.e., aqueous solution) to produce a nucleic acid-lipid particle.

The nucleic acid-lipid particles formed using the continuous mixing method typically have a size of from about 30 nm to about 150 nm, from about 40 nm to about 150 nm, from about 50 nm to about 150 nm, from about 60 rim to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 rim, 40 nm, 45 rim, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm (or any fraction thereof or range therein). The particles thus formed do not aggregate and are optionally sized to achieve a uniform particle size.

In another embodiment, the present invention provides nucleic acid-lipid particles produced via a direct dilution process that includes forming a lipid vesicle (e.g., liposome) solution and immediately and directly introducing the lipid vesicle solution into a collection vessel containing a controlled amount of dilution buffer. In preferred aspects, the collection vessel includes one or more elements configured to stir the contents of the collection vessel to facilitate dilution. In one aspect, the amount of dilution buffer present in the collection vessel is substantially equal to the volume of lipid vesicle solution introduced thereto. As a non-limiting example, a lipid vesicle solution in 45% ethanol when introduced into the collection vessel containing an equal volume of dilution buffer will advantageously yield smaller particles.

In yet another embodiment, the present invention provides nucleic acid-lipid particles produced via an in-line dilution process in which a third reservoir containing dilution buffer is fluidly coupled to a second mixing region. In this embodiment, the lipid vesicle (e.g., liposome) solution formed in a first mixing region is immediately and directly mixed with dilution buffer in the second mixing region. In preferred aspects, the second mixing region includes a T-connector arranged so that the lipid vesicle solution and the dilution buffer flows meet as opposing 180° flows; however, connectors providing shallower angles can be used, e.g., from about 27° to about 180° (e.g., about 90°). A pump mechanism delivers a controllable flow of buffer to the second mixing region. In one aspect, the flow rate of dilution buffer provided to the second mixing region is controlled to be substantially equal to the flow rate of lipid vesicle solution introduced thereto from the first mixing region. This embodiment advantageously allows for more control of the flow of dilution buffer mixing with the lipid vesicle solution in the second mixing region, and therefore also the concentration of lipid vesicle solution in buffer throughout the second mixing process. Such control of the dilution buffer flow rate advantageously allows for small particle size formation at reduced concentrations.

These processes and the apparatuses for carrying out these direct dilution and in-line dilution processes are described in detail in U.S. Patent Publication No. 20070042031, the disclosure of which is herein incorporated by reference in its entirety for all purposes.

The nucleic acid-lipid particles formed using the direct dilution and in-line dilution processes typically have a size of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm (or any fraction thereof or range therein). The particles thus formed do not aggregate and are optionally sized to achieve a uniform particle size.

If needed, the lipid particles of the invention can be sized by any of the methods available for sizing liposomes. The sizing may be conducted in order to achieve a desired size range and relatively narrow distribution of particle sizes.

Several techniques are available for sizing the particles to a desired size. One sizing method, used for liposomes and equally applicable to the present particles, is described in U.S. Pat. No. 4,737,323, the disclosure of which is herein incorporated by reference in its entirety for all purposes. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and about 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In some embodiments, the nucleic acids present in the particles (e.g., the gRNA molecules) are precondensed as described in, e.g., U.S. patent application Ser. No. 09/744,103, the disclosure of which is herein incorporated by reference in its entirety for all purposes.

In other embodiments, the methods may further comprise adding non-lipid polycations which are useful to effect the lipofection of cells using the present compositions. Examples of suitable non-lipid polycations include, hexadimethrine bromide (sold under the brand name POLYBRENE®, from Aldrich Chemical Co., Milwaukee, Wisconsin, USA) or other salts of hexadimethrine. Other suitable polycations include, for example, salts of poly-L-ornithine, poly-L-arginine, poly-L-lysine, poly-D-lysine, polyallylamine, and polyethyleneimine. Addition of these salts is preferably after the particles have been formed.

In some embodiments, the nucleic acid (e.g., gRNA) to lipid ratios (mass/mass ratios) in a formed nucleic acid-lipid particle will range from about 0.01 to about 0.2, from about 0.05 to about 0.2, from about 0.02 to about 0.1, from about 0.03 to about 0.1, or from about 0.01 to about 0.08. The ratio of the starting materials (input) also falls within this range. In other embodiments, the particle preparation uses about 400 μg nucleic acid per 10 mg total lipid or a nucleic acid to lipid mass ratio of about 0.01 to about 0.08 and, more preferably, about 0.04, which corresponds to 1.25 mg of total lipid per 50 μg of nucleic acid. In other preferred embodiments, the particle has a nucleic acid:lipid mass ratio of about 0.08.

In other embodiments, the lipid to nucleic acid (e.g., gRNA) ratios (mass/mass ratios) in a formed nucleic acid-lipid particle will range from about 1 (1:1) to about 100 (100:1), from about 5 (5:1) to about 100 (100:1), from about 1 (1:1) to about 50 (50:1), from about 2 (2:1) to about 50 (50:1), from about 3 (3:1) to about 50 (50:1), from about 4 (4:1) to about 50 (50:1), from about 5 (5:1) to about 50 (50:1), from about 1 (1:1) to about 25 (25:1), from about 2 (2:1) to about 25 (25:1), from about 3 (3:1) to about 25 (25:1), from about 4 (4:1) to about 25 (25:1), from about 5 (5:1) to about 25 (25:1), from about 5 (5:1) to about 20 (20:1), from about 5 (5:1) to about 15 (15:1), from about 5 (5:1) to about 10 (10:1), or about 5 (5:1), 6 (6:1), 7 (7:1), 8 (8:1), 9 (9:1), 10 (10:1), 11 (11:1), 12 (12:1), 13 (13:1), 14 (14:1), 15 (15:1), 16 (16:1), 17 (17:1), 18 (18:1), 19 (19:1), 20 (20:1), 21 (21:1), 22 (22:1), 23 (23:1), 24 (24:1), or 25 (25:1), or any fraction thereof or range therein. The ratio of the starting materials (input) also falls within this range.

As previously discussed, the conjugated lipid may further include a CPL. A variety of general methods for making lipid particle-CPLs (CPL-containing lipid particles) are discussed herein. Two general techniques include the “post-insertion” technique, that is, insertion of a CPL into, for example, a pre-formed lipid particle, and the “standard” technique, wherein the CPL is included in the lipid mixture during, for example, the lipid particle formation steps. The post-insertion technique results in lipid particles having CPLs mainly in the external face of the lipid particle bilayer membrane, whereas standard techniques provide lipid particles having CPLs on both internal and external faces. The method is especially useful for vesicles made from phospholipids (which can contain cholesterol) and also for vesicles containing PEG-lipids (such as PEG-DAAs and PEG-DAGs). Methods of making lipid particle-CPLs are taught, for example, in U.S. Pat. Nos. 5,705,385; 6,586,410; 5,981,501; 6,534,484; and 6,852,334; U.S. Patent Publication No. 20020072121; and PCT Publication No. WO 00/62813, the disclosures of which are herein incorporated by reference in their entirety for all purposes.

Kits

The present invention also provides lipid particles in kit form. In some embodiments, the kit comprises a container which is compartmentalized for holding the various elements of the lipid particles (e.g., the active agents, such as gRNA molecules and the individual lipid components of the particles). Preferably, the kit comprises a container (e.g., a vial or ampoule) which holds the lipid particles of the invention, wherein the particles are produced by one of the processes set forth herein. In certain embodiments, the kit may further comprise an endosomal membrane destabilizer (e.g., calcium ions). The kit typically contains the particle compositions of the invention, either as a suspension in a pharmaceutically acceptable carrier or in dehydrated form, with instructions for their rehydration (if lyophilized) and administration.

The formulations of the present invention can be tailored to preferentially target particular cells, tissues, or organs of interest. Preferential targeting of a nucleic acid-lipid particle may be carried out by controlling the composition of the lipid particle itself. In particular embodiments, the kits of the invention comprise these lipid particles, wherein the particles are present in a container as a suspension or in dehydrated form.

In certain instances, it may be desirable to have a targeting moiety attached to the surface of the lipid particle to further enhance the targeting of the particle. Methods of attaching targeting moieties (e.g., antibodies, proteins, etc.) to lipids (such as those used in the present particles) are known to those of skill in the art.

Administration of Lipid Particles

Once formed, the lipid particles of the invention are particularly useful for the introduction of a gRNA molecule into cells. Accordingly, the present invention also provides methods for introducing a gRNA molecule into a cell in combination with an mRNA encoding a Cas9. In particular embodiments, the gRNA molecule and mRNA encoding a Cas9 are introduced into an infected cell. The methods may be carried out in vitro or in vivo by first forming the particles as described above and then contacting the particles with the cells for a period of time sufficient for delivery of gRNA to the cells to occur.

The lipid particles of the invention (e.g., a nucleic-acid lipid particle) can be adsorbed to almost any cell type with which they are mixed or contacted. Once adsorbed, the particles can either be endocytosed by a portion of the cells, exchange lipids with cell membranes, or fuse with the cells. Transfer or incorporation of the gRNA portion of the particle can take place via any one of these pathways. In particular, when fusion takes place, the particle membrane is integrated into the cell membrane and the contents of the particle combine with the intracellular fluid.

The lipid particles of the invention (e.g., nucleic acid-lipid particles) can be administered either alone or in a mixture with a pharmaceutically acceptable carrier (e.g., physiological saline or phosphate buffer) selected in accordance with the route of administration and standard pharmaceutical practice. Generally, normal buffered saline (e.g., 135-150 mM NaCl) will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. Additional suitable carriers are described in, e.g., REMINGTON′S PHARMACEUTICAL SCIENCES, Mack Publishing Company, Philadelphia, Pa., 17th ed. (1985). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The phrase “pharmaceutically acceptable” refers to molecular entities and compositions that do not produce an allergic or similar untoward reaction when administered to a human.

The pharmaceutically acceptable carrier is generally added following lipid particle formation. Thus, after the lipid particle is formed, the particle can be diluted into pharmaceutically acceptable carriers such as normal buffered saline.

The concentration of particles in the pharmaceutical formulations can vary widely, i.e., from less than about 0.05%, usually at or at least about 2 to 5%, to as much as about 10 to 90% by weight, and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

The pharmaceutical compositions of the present invention may be sterilized by conventional, well-known sterilization techniques. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol, and water-soluble iron-specific chelators, such as ferrioxamine, are suitable.

In some embodiments, the lipid particles of the invention are particularly useful in methods for the therapeutic delivery of one or more gRNA molecules. In particular, it is an object of this invention to provide in vivo methods for treatment of HBV and/or HDV infection in humans by downregulating or silencing the transcription and/or translation of one or more HBV genes.

A. In vivo Administration

Systemic delivery for in vivo therapy, e.g., delivery of a gRNA molecule described herein, to a distal target cell via body systems such as the circulation, has been achieved using nucleic acid-lipid particles such as those described in PCT Publication Nos. WO 05/007196, WO 05/121348, WO 05/120152, and WO 04/002453, the disclosures of which are herein incorporated by reference in their entirety for all purposes. The present invention also provides fully encapsulated lipid particles that protect the gRNA from nuclease degradation in serum, are non-immunogenic, are small in size, and are suitable for repeat dosing. Additionally, the one or more gRNA molecules may be administered alone in the lipid particles of the invention, or in combination (e.g., co-administered) with lipid particles comprising peptides, polypeptides, or small molecules such as conventional drugs.

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol., 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993). Still other methods of administering lipid-based therapeutics are described in, for example, U.S. Pat. Nos. 3,993,754; 4,145,410; 4,235,871; 4,224,179; 4,522,803; and 4,588,578. The lipid particles can be administered by direct injection at the site of disease or by injection at a site distal from the site of disease (see, e.g., Culver, HUMAN GENE THERAPY, MaryAnn Liebert, Inc., Publishers, New York. pp.70-'71(1994)). The disclosures of the above-described references are herein incorporated by reference in their entirety for all purposes.

In embodiments where the lipid particles of the present invention are administered intravenously, at least about 5%, 10%, 15%, 20%, or 25% of the total injected dose of the particles is present in plasma about 8, 12, 24, 36, or 48 hours after injection. In other embodiments, more than about 20%, 30%, 40% and as much as about 60%, 70% or 80% of the total injected dose of the lipid particles is present in plasma about 8, 12, 24, 36, or 48 hours after injection. In certain instances, more than about 10% of a plurality of the particles is present in the plasma of a mammal about 1 hour after administration. In certain other instances, the presence of the lipid particles is detectable at least about 1 hour after administration of the particle. In some embodiments, the presence of a gRNA molecule is detectable in cells at about 8, 12, 24, 36, 48, 60, 72 or 96 hours after administration. In other embodiments, downregulation of expression of a target sequence, such as a viral or host sequence, by a gRNA molecule is detectable at about 8, 12, 24, 36, 48, 60, 72 or 96 hours after administration. In yet other embodiments, downregulation of expression of a target sequence, such as a viral or host sequence, by a gRNA molecule occurs preferentially in infected cells and/or cells capable of being infected. In further embodiments, the presence or effect of a gRNA molecule in cells at a site proximal or distal to the site of administration is detectable at about 12, 24, 48, 72, or 96 hours, or at about 6, 8, 10, 12, 14, 16, 18, 19, 20, 22, 24, 26, or 28 days after administration. In additional embodiments, the lipid particles of the invention are administered parenterally or intraperitoneally.

The compositions of the present invention, either alone or in combination with other suitable components, can be made into aerosol formulations (i.e., they can be “nebulized”) to be administered via inhalation (e.g., intranasally or intratracheally) (see, Brigham et al., Am. J. Sci., 298:278 (1989)). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. 5,725,871) are also well-known in the pharmaceutical arts. Similarly, transmucosal drug delivery in the form of a polytetrafluoroetheylene support matrix is described in U.S. Pat. No. 5,780,045. The disclosures of the above-described patents are herein incorporated by reference in their entirety for all purposes.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions are preferably administered, for example, by intravenous infusion, orally, topically, intraperitoneally, intravesically, or intrathecally.

Generally, when administered intravenously, the lipid particle formulations are formulated with a suitable pharmaceutical carrier. Many pharmaceutically acceptable carriers may be employed in the compositions and methods of the present invention. Suitable formulations for use in the present invention are found, for example, in REMINGTON'S PHARMACEUTICAL SCIENCES, Mack Publishing Company, Philadelphia, Pa., 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

Generally, lipid particles will not be delivered orally. However, in certain applications, the lipid particles disclosed herein may be delivered via oral administration to the individual. The particles may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451, the disclosures of which are herein incorporated by reference in their entirety for all purposes). These oral dosage forms may also contain the following: binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

Typically, these oral formulations may contain at least about 0.1% of the lipid particles or more, although the percentage of the particles may, of course, be varied and may conveniently be between about 1% or 2% and about 60% or 70% or more of the weight or volume of the total formulation. Naturally, the amount of particles in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Formulations suitable for oral administration can consist of: (a) liquid solutions, such as an effective amount of a packaged gRNA molecule suspended in diluents such as water, saline, or PEG 400; (b) capsules, sachets, or tablets, each containing a predetermined amount of a gRNA molecule, as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a gRNA molecule in a flavor, e.g., sucrose, as well as pastilles comprising the therapeutic nucleic acid in an inert base, such as gelatin and glycerin or sucrose and acacia emulsions, gels, and the like containing, in addition to the gRNA molecule, carriers known in the art.

In another example of their use, lipid particles can be incorporated into a broad range of topical dosage forms. For instance, a suspension containing nucleic acid-lipid particles can be formulated and administered as gels, oils, emulsions, topical creams, pastes, ointments, lotions, foams, mousses, and the like.

When preparing pharmaceutical preparations of the lipid particles of the invention, it is preferable to use quantities of the particles which have been purified to reduce or eliminate empty particles or particles with therapeutic agents such as gRNA associated with the external surface.

The methods of the present invention may be practiced in a variety of hosts. Preferred hosts include mammalian species, such as primates (e.g., humans and chimpanzees as well as other nonhuman primates), canines, felines, equines, bovines, ovines, caprines, rodents (e.g., rats and mice), lagomorphs, and swine.

The amount of particles administered will depend upon the ratio of gRNA molecules to lipid, the particular gRNA used, the strain of HBV being treated, the age, weight, and condition of the patient, and the judgment of the clinician, but will generally be between about 0.01 and about 50 mg per kilogram of body weight, preferably between about 0.1 and about 5 mg/kg of body weight, or about 10⁸-10¹⁰ particles per administration (e.g., injection).

B. In vitro Administration

For in vitro applications, the delivery of gRNA molecules can be to any cell grown in culture. In preferred embodiments, the cells are animal cells, more preferably mammalian cells, and most preferably human cells.

Contact between the cells and the lipid particles, when carried out in vitro, takes place in a biologically compatible medium. The concentration of particles varies widely depending on the particular application, but is generally between about 1 mol and about 10 mmol. Treatment of the cells with the lipid particles is generally carried out at physiological temperatures (about 37° C.) for periods of time of from about 1 to 48 hours, preferably of from about 2 to 4 hours.

In one group of preferred embodiments, a lipid particle suspension is added to 60-80% confluent plated cells having a cell density of from about 10³ to about 10⁵ cells/ml, more preferably about 2×10⁴ cells/ml. The concentration of the suspension added to the cells is preferably of from about 0.01 to 0.2 μg/ml, more preferably about 0.1 μg/ml.

To the extent that tissue culture of cells may be required, it is well-known in the art. For example, Freshney, Culture of Animal Cells, a Manual of Basic Technique, 3rd Ed., Wiley-Liss, New York (1994), Kuchler et al., Biochemical Methods in Cell Culture and Virology, Dowden, Hutchinson and Ross, Inc. (1977), and the references cited therein provide a general guide to the culture of cells. Cultured cell systems often will be in the form of monolayers of cells, although cell suspensions are also used.

Using an Endosomal Release Parameter (ERP) assay, the delivery efficiency of a nucleic acid-lipid particle of the invention can be optimized. An ERP assay is described in detail in U.S. Patent Publication No. 20030077829, the disclosure of which is herein incorporated by reference in its entirety for all purposes. More particularly, the purpose of an ERP assay is to distinguish the effect of various cationic lipids and helper lipid components of the lipid particle based on their relative effect on binding/uptake or fusion with/destabilization of the endosomal membrane. This assay allows one to determine quantitatively how each component of the lipid particle affects delivery efficiency, thereby optimizing the lipid particle. Usually, an ERP assay measures expression of a reporter protein (e.g., luciferase, β-galactosidase, green fluorescent protein (GFP), etc.), and in some instances, a lipid particle formulation optimized for an expression plasmid will also be appropriate for encapsulating a gRNA. In other instances, an ERP assay can be adapted to measure downregulation of transcription or translation of a target sequence in the presence or absence of a gRNA. By comparing the ERPs for each of the various lipid particles, one can readily determine the optimized system, e.g., the lipid particle that has the greatest uptake in the cell.

### C. Detection of Lipid Particles

In some embodiments, the lipid particles of the present invention are detectable in the subject at about 1, 2, 3, 4, 5, 6, 7, 8 or more hours. In other embodiments, the lipid particles of the present invention are detectable in the subject at about 8, 12, 24, 48, 60, 72, or 96 hours, or about 6, 8, 10, 12, 14, 16, 18, 19, 22, 24, 25, or 28 days after administration of the particles. The presence of the particles can be detected in the cells, tissues, or other biological samples from the subject. The particles may be detected, e.g, by direct detection of the particles, detection of a gRNA sequence, detection of the target sequence of interest (i.e., by detecting expression or reduced expression of the sequence of interest), detection of a compound modulated by an EBOV protein (e.g., interferon), detection of viral load in the subject, or a combination thereof.

### 1. Detection of Particles

Lipid particles of the invention can be detected using any method known in the art. For example, a label can be coupled directly or indirectly to a component of the lipid particle using methods well-known in the art. A wide variety of labels can be used, with the choice of label depending on sensitivity required, ease of conjugation with the lipid particle component, stability requirements, and available instrumentation and disposal provisions. Suitable labels include, but are not limited to, spectral labels such as fluorescent dyes (e.g., fluorescein and derivatives, such as fluorescein isothiocyanate (FITC) and Oregon Green™; rhodamine and derivatives such Texas red, tetrarhodimine isothiocynate (TRITC), etc., digoxigenin, biotin, phycoerythrin, AMCA, CyDyes™, and the like; radiolabels such as ³H, ¹²⁵I, ³⁵S, ¹⁴C, ³²P, ³³P, etc.; enzymes such as horse radish peroxidase, alkaline phosphatase, etc.; spectral colorimetric labels such as colloidal gold or colored glass or plastic beads such as polystyrene, polypropylene, latex, etc. The label can be detected using any means known in the art.

### 2. Detection of Nucleic Acids

Nucleic acids (e.g., gRNA molecules) are detected and quantified herein by any of a number of means well-known to those of skill in the art. The detection of nucleic acids may proceed by well-known methods such as Southern analysis, Northern analysis, gel electrophoresis, PCR, radiolabeling, scintillation counting, and affinity chromatography. Additional analytic biochemical methods such as spectrophotometry, radiography, electrophoresis, capillary electrophoresis, high performance liquid chromatography (HPLC), thin layer chromatography (TLC), and hyperdiffusion chromatography may also be employed.

The selection of a nucleic acid hybridization format is not critical. A variety of nucleic acid hybridization formats are known to those skilled in the art. For example, common formats include sandwich assays and competition or displacement assays. Hybridization techniques are generally described in, e.g., “Nucleic Acid Hybridization, A Practical Approach,” Eds. Hames and Higgins, IRL Press (1985).

The sensitivity of the hybridization assays may be enhanced through the use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR), the ligase chain reaction (LCR), Q3-replicase amplification, and other RNA polymerase mediated techniques (e.g., NASBA™) are found in Sambrook et al., In Molecular Cloning: A Laboratory Manual, Cold Spring Harbor Laboratory Press (2000); and Ausubel et al., SHORT PROTOCOLS IN MOLECULAR BIOLOGY, eds., Current Protocols, Greene Publishing Associates, Inc. and John Wiley & Sons, Inc. (2002); as well as U.S. Pat. No. 4,683,202; PCR Protocols, A Guide to Methods and Applications (Innis et al. eds.) Academic Press Inc. San Diego, Calif. (1990); Arnheim & Levinson (Oct. 1, 1990), C&EN 36; The Journal Of NIH Research, 3:81 (1991); Kwoh et al., Proc. Natl. Acad. Sci. USA, 86:1173 (1989); Guatelli et al., Proc. Natl. Acad. Sci. USA, 87:1874 (1990); Lomell et al., J Clin. Chem., 35:1826 (1989); Landegren et al., Science, 241:1077 (1988); Van Brunt, Biotechnology, 8:291 (1990); Wu and Wallace, Gene, 4:560 (1989); Barringer et al., Gene, 89:117 (1990); and Sooknanan and Malek, Biotechnology, 13:563 (1995). Improved methods of cloning in vitro amplified nucleic acids are described in U.S. Pat. No. 5,426,039. Other methods described in the art are the nucleic acid sequence based amplification (NASBA™, Cangene, Mississauga, Ontario) and Qβ-replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation. The disclosures of the above-described references are herein incorporated by reference in their entirety for all purposes.

Nucleic acids for use as probes, e.g., in in vitro amplification methods, for use as gene probes, or as inhibitor components are typically synthesized chemically according to the solid phase phosphoramidite triester method described by Beaucage et al., Tetrahedron Letts., 22:1859 1862 (1981), e. g. , using an automated synthesizer, as described in Needham VanDevanter et al., Nucleic Acids Res., 12:6159 (1984). Purification of polynucleotides, where necessary, is typically performed by either native acrylamide gel electrophoresis or by anion exchange HPLC as described in Pearson et al., J. Chrom., 255:137 149 (1983). The sequence of the synthetic polynucleotides can be verified using the chemical degradation method of Maxam and Gilbert (1980) in Grossman and Moldave (eds.) Academic Press, New York, Methods in Enzymology, 65:499.

An alternative means for determining the level of transcription is in situ hybridization. In situ hybridization assays are well-known and are generally described in Angerer et al., Methods Enzymol., 152:649 (1987). In an in situ hybridization assay, cells are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

This Example describes the identification and description of CRISPR target sites contained in HBV. Representative sequences from four HBV genotypes (A-D) were used in this Example. A total of 1966 sites for the four different Cas9 variants, having different PAM motifs, were identified. Among the target sites, there are certain conserved sites across all 4 of the HBV sequences searched (about 40 in total; please refer to FIGS. 1-4 for a description of the targets and the conserved targets). These identified target sites can be used to prepare gRNA sequences that target HBV, which, when used in combination with Cas9, can be used to treat HBV infections in patients in need thereof. In certain embodiments, the gRNA are designed using the conserved target sequences.

TABLE 1
Summary of the Results Presented in FIGS. 1-4.

| | Size | PAM | Target sites in 4 |
|---|---|---|---|
| Species | (ORF) | Sequence | HBVs (A-D) |
| Streptococcus | 4104 nt | NGG | 1456 |
| pyogenes (SP)* | | | |
| Neisseria | 3249 nt | NNNNGATT | 90 |
| meningitidis (NM) | | | |
| Streptococcus | 3360 nt | NNAGAA(W) | 59 |
| thermophilus (ST) | | | |
| Streptococcus | 3159 nt | NNGRR(T) | 361 |
| aureus (SA) | | | |
| | | | 1966 |

### Example 2

This example describes CRISPR/Cas9-induced gene editing of an endogenous gene following delivery of messenger RNA (mRNA) and single guide RNA (gRNA) to a mouse liver in vivo via lipid nanoparticles (LNP).

Mice were injected intravenously with a LNP formulation of mRNA for the Cas9 protein (2 mg/kg body weight) and a LNP formulation of a gRNA (0.42 mg/kg) containing a target sequence within the mouse Pcsk9 gene. The gRNA contained the 20 base-pair target site GGCTGATGAGGCCGCACATG (SEQ ID NO:6), which lies within exon 1 of mouse Pcsk9.

Two days post-treatment, the animal livers were harvested and hepatic genomic DNA was isolated. This DNA isolate was used as a template for polymerase chain reaction to generate an amplicon of 415 base pairs in length containing the CRISPR target cut site 291 base pairs from the amplicon end. Surveyor assay (Guschin et al., Methods Mol. BioL, 649, 247 (2010)) was run to detect small insertion/deletion (indel) mutations at the target cut site, and 100% (6/6) of treated animals showed a positive result for gene editing, while 0% (0/9) of the negative control animals (Saline-treated or LNP-mRNA only-treated) showed a positive result.

This positive Surveyor result from the harvested mouse livers provided evidence that LNP can be utilized to deliver Cas9 mRNA and gRNA to the liver in vivo and that the intended activity of the CRISPR reagents, i.e., target site-specific DNA cleavage followed by imprecise repair and mutagenesis, can be achieved.

### Example 3

This example describes CRISPR/Cas9-induced gene editing of an exogenously introduced hepatitis B virus (HBV) genome following delivery of messenger RNA (mRNA) and single guide RNA (gRNA) to a mouse liver in vivo via lipid nanoparticles (LNP).

HBV DNA was delivered into the livers of NOD-SCID mice via hydrodynamic injection (HDI) in the tail vein with a 1.3-overlength HBV plasmid (10 μg/mouse in 1.6 mL saline in <5 sec). HBV plasmid HDI results in a stable pool of HBV DNA in the mouse liver and stable expression of HBV antigens.

Seven days post-HDI, the mice were injected intravenously with a LNP formulation of mRNA for the Cas9 protein (2 mg/kg body weight) and a LNP formulation of a gRNA (0.44 mg/kg) containing a target sequence within the HBV RT gene. The gRNA contained the 20 base-pair target site TTTCAGTTATATGGATGATG (SEQ ID NO:7), which lies in the HBV RT gene (Genbank ID: V01460; 2437-2456).

Two days post-treatment, the animal livers were harvested and hepatic DNA was isolated. This DNA isolate was used as a template for polymerase chain reaction to generate an amplicon of 562 base pairs in length containing the CRISPR target cut site 447 base pairs from the amplicon end. Surveyor assay (Guschin et al., Methods Mol. Biol., 649, 247 (2010)) was run to detect small insertion/deletion (indel) mutations at the target cut site, and 100% (8/8) of treated animals showed a positive result for gene editing, while 0% (0/14) of the negative control animals (Saline-treated, LNP-mRNA only-treated, or entecavir-treated) showed a positive result.

This positive Surveyor result from the harvested mouse livers provided evidence that LNP can be utilized to deliver Cas9 mRNA and gRNA to the liver in vivo and that the intended activity of the CRISPR reagents, i.e., target site-specific DNA cleavage followed by imprecise repair and mutagenesis, can be achieved, specifically the targeting of exogenous HBV DNA.

1. A guide RNA (gRNA) sequence comprising a first sequence that corresponds to a target sequence described in FIG. 1, FIG. 2, FIG. 3 or FIG. 4 and a second sequence that is a tracer RNA sequence located 3′ of the first sequence.

2. The gRNA of claim 1, wherein the target sequence is a conserved target sequence described in FIG. 1, FIG. 2, FIG. 3 or FIG. 4.

3. The gRNA of claim 1, wherein the tracer RNA sequence is 5′-GUUUUAGAGCUAGAAAUAGCAAGUUAAAAUAAGGCUAGUCCGUUAUCAACUUGA AAAAGUGGCACCGAGUCGGUGCUUUU-3′ (SEQ ID NO: 8).

4. (canceled)

5. (canceled)

6. (canceled)

7. (canceled)

8. (canceled)

9. (canceled)

10. (canceled)

11. A nucleic acid-lipid particle comprising:
(a) one or more gRNA of claim 1;
(b) a cationic lipid; and
(c) a non-cationic lipid.

12. The nucleic acid-lipid particle of claim 11, which further comprises a mRNA sequence encoding a CRISPR associated protein 9 (Cas9).

13. The nucleic acid-lipid particle of claim 12, wherein the mRNA sequence encoding the Cas9 further comprises a sequence encoding a nuclear localization signal (NLS).

14. The nucleic acid-lipid particle of claim 13, wherein the NLS is PKKKRKV (SEQ ID NO:1).

15. The nucleic acid-lipid particle of claim 12, wherein the mRNA sequence encoding the Cas9 further comprises a polyA tail, a 5′ untranslated region (UTR) and/or a 3′ UTR.

16. (canceled)

17. (canceled)

18. (canceled)

19. (canceled)

20. (canceled)

21. (canceled)

22. (canceled)

23. (canceled)

24. The nucleic acid-lipid particle of claim 11, wherein the gRNA is fully encapsulated in the particle.

25. The nucleic acid-lipid particle of claim 11, wherein the particle has a total lipid:gRNA mass ratio of from about 5:1 to about 15:1.

26. The nucleic acid-lipid particle of claim 11, wherein the particle has a median diameter of from about 30 nm to about 150 nm.

27. The nucleic acid-lipid particle of claim 11, wherein the particle has an electron dense core.

28. (canceled)

29. (canceled)

30. (canceled)

31. (canceled)

32. The nucleic acid-lipid particle of claim 11 comprising two different gRNA sequences.

33. The nucleic acid-lipid particle of claim 11 comprising three different gRNA sequences.

34. (canceled)

35. (canceled)

36. A pharmaceutical composition comprising a nucleic acid-lipid particle of claim 11 and a pharmaceutically acceptable carrier, which composition optionally comprises a second nucleic acid-lipid particle comprising a mRNA sequence encoding a Cas9, which second nucleic acid-lipid particle does not comprise the gRNA described in claim 11.

37. A method for silencing expression of a Hepatitis B virus gene in a cell, the method comprising the step of contacting a cell comprising an expressed Hepatitis B virus gene with the composition of claim 36 under conditions to silence the expression of the Hepatitis B virus gene within the cell.

38. (canceled)

39. (canceled)

40. (canceled)

41. (canceled)

42. (canceled)

43. (canceled)

44. (canceled)

45. A method for ameliorating one or more symptoms associated with Hepatitis B virus and/or Hepatitis D virus infection in a mammal, the method comprising the step of administering to the mammal a therapeutically effective amount of a nucleic acid-lipid particle of claim 11.

46. (canceled)

47. (canceled)

48. (canceled)

49. (canceled)

50. (canceled)

51. (canceled)

52. A method for treating a Hepatitis B virus and/or Hepatitis D virus infection in a mammal, the method comprising the step of administering to the mammal a therapeutically effective amount of a nucleic acid-lipid particle of claim 11.

53-77. (canceled)

78. A method for ameliorating one or more symptoms associated with Hepatitis B virus and/or Hepatitis D virus infection in a mammal, the method comprising the step of administering to the mammal a therapeutically effective amount of the pharmaceutical composition of claim 36.

79. A method for treating a Hepatitis B virus and/or Hepatitis D virus infection in a mammal, the method comprising the step of administering to the mammal a therapeutically effective amount of the pharmaceutical composition of claim 36.


SEQUENCE LISTING (2017 sequences)

SEQ ID NO: 1 - protein, 7 aa, Artificial Sequence
  Description of Artificial Sequence Synthetic peptide
PKKKRKV

SEQ ID NO: 2 - DNA, 4140 bp, Streptococcus pyogenes
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 3 - RNA, 100 nt, Artificial Sequence
  Description of Artificial Sequence Synthetic polynucleotide
  modified_base (1)..(20): a, c, u, g, unknown or other
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 4 - RNA, 80 nt, Artificial Sequence
  Description of Artificial Sequence Synthetic oligonucleotide
guuuuaguacucuggaaacagaaucuacuaaaacaaggcaaaaugccguguuuaucucgucaacuuguuggcgagauuuu

SEQ ID NO: 5 - RNA, 80 nt, Artificial Sequence
  Description of Artificial Sequence Synthetic oligonucleotide
guuuuuguacucgaaagaagcuacaaagauaaggcuucaugccgaaaucaacacccugucauuuuauggcaggguguuuu

SEQ ID NO: 6 - DNA, 20 bp, Mus sp.
ggctgatgaggccgcacatg

SEQ ID NO: 7 - DNA, 20 bp, Hepatitis B virus
tttcagttatatggatgatg

SEQ ID NO: 8 - RNA, 80 nt, Artificial Sequence
  Description of Artificial Sequence Synthetic oligonucleotide
guuuuagagcuagaaauagcaaguuaaaauaaggcuaguccguuaucaacuugaaaaaguggcaccgagucggugcuuuu

SEQ ID NO: 9 - DNA, 23 bp, Streptococcus pyogenes
ctgcaagatcccagagtgagagg

SEQ ID NO: 10 - DNA, 23 bp, Streptococcus pyogenes
agaggcctgtatttccctgctgg

SEQ ID NO: 11 - DNA, 23 bp, Streptococcus pyogenes
ggcctgtatttccctgctggtgg

SEQ ID NO: 12 - DNA, 23 bp, Streptococcus pyogenes
cctgctggtggctccagttcagg

SEQ ID NO: 13 - DNA, 23 bp, Streptococcus pyogenes
ccttatcgtcaatcttctcgagg

SEQ ID NO: 14 - DNA, 23 bp, Streptococcus pyogenes
tcgtcaatcttctcgaggattgg

SEQ ID NO: 15 - DNA, 23 bp, Streptococcus pyogenes
cgtcaatcttctcgaggattggg

SEQ ID NO: 16 - DNA, 23 bp, Streptococcus pyogenes
gtcaatcttctcgaggattgggg

SEQ ID NO: 17 - DNA, 23 bp, Streptococcus pyogenes
tggggaccctgcgctgaacatgg

SEQ ID NO: 18 - DNA, 23 bp, Streptococcus pyogenes
aacatggagaacatcacatcagg

SEQ ID NO: 19 - DNA, 23 bp, Streptococcus pyogenes
aacatcacatcaggattcctagg

SEQ ID NO: 20 - DNA, 23 bp, Streptococcus pyogenes
aggaccccttctcgtgttacagg

SEQ ID NO: 21 - DNA, 23 bp, Streptococcus pyogenes
accccttctcgtgttacaggcgg

SEQ ID NO: 22 - DNA, 23 bp, Streptococcus pyogenes
ccccttctcgtgttacaggcggg

SEQ ID NO: 23 - DNA, 23 bp, Streptococcus pyogenes
cccttctcgtgttacaggcgggg

SEQ ID NO: 24 - DNA, 23 bp, Streptococcus pyogenes
taccgcagagtctagactcgtgg

SEQ ID NO: 25 - DNA, 23 bp, Streptococcus pyogenes
cgcagagtctagactcgtggtgg

SEQ ID NO: 26 - DNA, 23 bp, Streptococcus pyogenes
tggacttctctcaattttctagg

SEQ ID NO: 27 - DNA, 23 bp, Streptococcus pyogenes
ggacttctctcaattttctaggg

SEQ ID NO: 28 - DNA, 23 bp, Streptococcus pyogenes
gacttctctcaattttctagggg

SEQ ID NO: 29 - DNA, 23 bp, Streptococcus pyogenes
acttctctcaattttctaggggg

SEQ ID NO: 30 - DNA, 23 bp, Streptococcus pyogenes
gggggaactaccgtgtgtcttgg

SEQ ID NO: 31 - DNA, 23 bp, Streptococcus pyogenes
tcttgtcctccaacttgtcctgg

SEQ ID NO: 32 - DNA, 23 bp, Streptococcus pyogenes
caacttgtcctggttatcgctgg

SEQ ID NO: 33 - DNA, 23 bp, Streptococcus pyogenes
gttatcgctggatgtgtctgcgg

SEQ ID NO: 34 - DNA, 23 bp, Streptococcus pyogenes
gctatgcctcatcttcttgttgg

SEQ ID NO: 35 - DNA, 23 bp, Streptococcus pyogenes
catcttcttgttggttcttctgg

SEQ ID NO: 36 - DNA, 23 bp, Streptococcus pyogenes
ttggttcttctggactatcaagg

SEQ ID NO: 37 - DNA, 23 bp, Streptococcus pyogenes
cccgtttgtcctctaattccagg

SEQ ID NO: 38 - DNA, 23 bp, Streptococcus pyogenes
aggatcctcaacaaccagcacgg

SEQ ID NO: 39 - DNA, 23 bp, Streptococcus pyogenes
ggatcctcaacaaccagcacggg

SEQ ID NO: 40 - DNA, 23 bp, Streptococcus pyogenes
caaccagcacgggaccatgccgg

SEQ ID NO: 41 - DNA, 23 bp, Streptococcus pyogenes
acctgcatgactactgctcaagg

SEQ ID NO: 42 - DNA, 23 bp, Streptococcus pyogenes
ctgttgctgtaccaaaccttcgg

SEQ ID NO: 43 - DNA, 23 bp, Streptococcus pyogenes
tgctgtaccaaaccttcggacgg

SEQ ID NO: 44 - DNA, 23 bp, Streptococcus pyogenes
gtattcccatcccatcatcctgg

SEQ ID NO: 45 - DNA, 23 bp, Streptococcus pyogenes
tattcccatcccatcatcctggg

SEQ ID NO: 46 - DNA, 23 bp, Streptococcus pyogenes
atcccatcatcctgggctttcgg

SEQ ID NO: 47 - DNA, 23 bp, Streptococcus pyogenes
gggctttcggaaaattcctatgg

SEQ ID NO: 48 - DNA, 23 bp, Streptococcus pyogenes
ggctttcggaaaattcctatggg

SEQ ID NO: 49 - DNA, 23 bp, Streptococcus pyogenes
tcggaaaattcctatgggagtgg

SEQ ID NO: 50 - DNA, 23 bp, Streptococcus pyogenes
cggaaaattcctatgggagtggg

SEQ ID NO: 51 - DNA, 23 bp, Streptococcus pyogenes
gggcctcagcccgtttctcctgg

SEQ ID NO: 52 - DNA, 23 bp, Streptococcus pyogenes
tactagtgccatttgttcagtgg

SEQ ID NO: 53 - DNA, 23 bp, Streptococcus pyogenes
ccatttgttcagtggttcgtagg

SEQ ID NO: 54 - DNA, 23 bp, Streptococcus pyogenes
catttgttcagtggttcgtaggg

SEQ ID NO: 55 - DNA, 23 bp, Streptococcus pyogenes
tagggctttcccccactgtttgg

SEQ ID NO: 56 - DNA, 23 bp, Streptococcus pyogenes
ctgtttggctttcagttatatgg

SEQ ID NO: 57 - DNA, 23 bp, Streptococcus pyogenes
tttcagttatatggatgatgtgg

SEQ ID NO: 58 - DNA, 23 bp, Streptococcus pyogenes
ttatatggatgatgtggtattgg

SEQ ID NO: 59 - DNA, 23 bp, Streptococcus pyogenes
tatatggatgatgtggtattggg

SEQ ID NO: 60 - DNA, 23 bp, Streptococcus pyogenes
atatggatgatgtggtattgggg

SEQ ID NO: 61 - DNA, 23 bp, Streptococcus pyogenes
tatggatgatgtggtattggggg

SEQ ID NO: 62 - DNA, 23 bp, Streptococcus pyogenes
taccaattttcttttgtctttgg

SEQ ID NO: 63 - DNA, 23 bp, Streptococcus pyogenes
accaattttcttttgtctttggg

SEQ ID NO: 64 - DNA, 23 bp, Streptococcus pyogenes
accctaacaaaacaaagagatgg

SEQ ID NO: 65 - DNA, 23 bp, Streptococcus pyogenes
ccctaacaaaacaaagagatggg

SEQ ID NO: 66 - DNA, 23 bp, Streptococcus pyogenes
cctaacaaaacaaagagatgggg

SEQ ID NO: 67 - DNA, 23 bp, Streptococcus pyogenes
gggttactctctaaattttatgg

SEQ ID NO: 68 - DNA, 23 bp, Streptococcus pyogenes
ggttactctctaaattttatggg

SEQ ID NO: 69 - DNA, 23 bp, Streptococcus pyogenes
aattttatgggttatgtcattgg

SEQ ID NO: 70 - DNA, 23 bp, Streptococcus pyogenes
ggttatgtcattggatgttatgg

SEQ ID NO: 71 - DNA, 23 bp, Streptococcus pyogenes
gttatgtcattggatgttatggg

SEQ ID NO: 72 - DNA, 23 bp, Streptococcus pyogenes
ttagaaaacttcctattaacagg

SEQ ID NO: 73 - DNA, 23 bp, Streptococcus pyogenes
ctattaacaggcctattgattgg

SEQ ID NO: 74 - DNA, 23 bp, Streptococcus pyogenes
gaaagtatgtcaacgaattgtgg

SEQ ID NO: 75 - DNA, 23 bp, Streptococcus pyogenes
aaagtatgtcaacgaattgtggg

SEQ ID NO: 76 - DNA, 23 bp, Streptococcus pyogenes
tcaacgaattgtgggtcttttgg

SEQ ID NO: 77 - DNA, 23 bp, Streptococcus pyogenes
caacgaattgtgggtcttttggg

SEQ ID NO: 78 - DNA, 23 bp, Streptococcus pyogenes
gctgccccttttacacaatgtgg

SEQ ID NO: 79 - DNA, 23 bp, Streptococcus pyogenes
tgcatgtattcaatctaagcagg

SEQ ID NO: 80 - DNA, 23 bp, Streptococcus pyogenes
cactttctcgccaacttacaagg

SEQ ID NO: 81 - DNA, 23 bp, Streptococcus pyogenes
tgaacctttaccccgttgcccgg

SEQ ID NO: 82 - DNA, 23 bp, Streptococcus pyogenes
tttaccccgttgcccggcaacgg

SEQ ID NO: 83 - DNA, 23 bp, Streptococcus pyogenes
cccgttgcccggcaacggccagg

SEQ ID NO: 84 - DNA, 23 bp, Streptococcus pyogenes
tttgctgacgcaacccccactgg

SEQ ID NO: 85 - DNA, 23 bp, Streptococcus pyogenes
ctgacgcaacccccactggctgg

SEQ ID NO: 86 - DNA, 23 bp, Streptococcus pyogenes
tgacgcaacccccactggctggg

SEQ ID NO: 87 - DNA, 23 bp, Streptococcus pyogenes
gacgcaacccccactggctgggg

SEQ ID NO: 88 - DNA, 23 bp, Streptococcus pyogenes
aacccccactggctggggcttgg

SEQ ID NO: 89 - DNA, 23 bp, Streptococcus pyogenes
cactggctggggcttggtcatgg

SEQ ID NO: 90 - DNA, 23 bp, Streptococcus pyogenes
actggctggggcttggtcatggg

SEQ ID NO: 91 - DNA, 23 bp, Streptococcus pyogenes
atgggccatcagcgcatgcgtgg

SEQ ID NO: 92 - DNA, 23 bp, Streptococcus pyogenes
gcgcatgcgtggaaccttttcgg

SEQ ID NO: 93 - DNA, 23 bp, Streptococcus pyogenes
tcctctgccgatccatactgcgg

SEQ ID NO: 94 - DNA, 23 bp, Streptococcus pyogenes
ccgcttgttttgctcgcagcagg

SEQ ID NO: 95 - DNA, 23 bp, Streptococcus pyogenes
tgttttgctcgcagcaggtctgg

SEQ ID NO: 96 - DNA, 23 bp, Streptococcus pyogenes
aggtctggagcaaacattatcgg

SEQ ID NO: 97 - DNA, 23 bp, Streptococcus pyogenes
ggtctggagcaaacattatcggg

SEQ ID NO: 98 - DNA, 23 bp, Streptococcus pyogenes
gcaaatatacatcgtttccatgg

SEQ ID NO: 99 - DNA, 23 bp, Streptococcus pyogenes
acatcgtttccatggctgctagg

SEQ ID NO: 100 - DNA, 23 bp, Streptococcus pyogenes
tgctaggctgtgctgccaactgg

SEQ ID NO: 101 - DNA, 23 bp, Streptococcus pyogenes
gctgccaactggatcctgcgcgg

SEQ ID NO: 102 - DNA, 23 bp, Streptococcus pyogenes
ctgccaactggatcctgcgcggg

SEQ ID NO: 103 - DNA, 23 bp, Streptococcus pyogenes
gtcctttgtttacgtcccgtcgg

SEQ ID NO: 104 - DNA, 23 bp, Streptococcus pyogenes
cccgtcggcgctgaatcctgcgg

SEQ ID NO: 105 - DNA, 23 bp, Streptococcus pyogenes
atcctgcggacgacccttctcgg

SEQ ID NO: 106 - DNA, 23 bp, Streptococcus pyogenes
tcctgcggacgacccttctcggg

SEQ ID NO: 107 - DNA, 23 bp, Streptococcus pyogenes
cctgcggacgacccttctcgggg

SEQ ID NO: 108 - DNA, 23 bp, Streptococcus pyogenes
cgacccttctcggggtcgcttgg

SEQ ID NO: 109 - DNA, 23 bp, Streptococcus pyogenes
gacccttctcggggtcgcttggg

SEQ ID NO: 110 - DNA, 23 bp, Streptococcus pyogenes
tctgccgttccgaccgaccacgg

SEQ ID NO: 111 - DNA, 23 bp, Streptococcus pyogenes
ctgccgttccgaccgaccacggg

SEQ ID NO: 112 - DNA, 23 bp, Streptococcus pyogenes
tgccgttccgaccgaccacgggg

SEQ ID NO: 113 - DNA, 23 bp, Streptococcus pyogenes
ggggcgcacctctctttacgcgg

SEQ ID NO: 114 - DNA, 23 bp, Streptococcus pyogenes
gtctgtgccttctcatctgccgg

SEQ ID NO: 115 - DNA, 23 bp, Streptococcus pyogenes
cttcacctctgcacgtcgcatgg

SEQ ID NO: 116 - DNA, 23 bp, Streptococcus pyogenes
cgcccaccaaatattgcccaagg

SEQ ID NO: 117 - DNA, 23 bp, Streptococcus pyogenes
tgcccaaggtcttacataagagg

SEQ ID NO: 118 - DNA, 23 bp, Streptococcus pyogenes
gtcttacataagaggactcttgg

SEQ ID NO: 119 - DNA, 23 bp, Streptococcus pyogenes
aatgtcaacgaccgaccttgagg

SEQ ID NO: 120 - DNA, 23 bp, Streptococcus pyogenes
aagactgtttgtttaaagactgg

SEQ ID NO: 121 - DNA, 23 bp, Streptococcus pyogenes
agactgtttgtttaaagactggg

SEQ ID NO: 122 - DNA, 23 bp, Streptococcus pyogenes
ctgtttgtttaaagactgggagg

SEQ ID NO: 123 - DNA, 23 bp, Streptococcus pyogenes
gtttaaagactgggaggagttgg

SEQ ID NO: 124 - DNA, 23 bp, Streptococcus pyogenes
tttaaagactgggaggagttggg

SEQ ID NO: 125 - DNA, 23 bp, Streptococcus pyogenes
ttaaagactgggaggagttgggg

SEQ ID NO: 126 - DNA, 23 bp, Streptococcus pyogenes
taaagactgggaggagttggggg

SEQ ID NO: 127 - DNA, 23 bp, Streptococcus pyogenes
agactgggaggagttgggggagg

SEQ ID NO: 128 - DNA, 23 bp, Streptococcus pyogenes
aggagttgggggaggagattagg

SEQ ID NO: 129 - DNA, 23 bp, Streptococcus pyogenes
gggggaggagattaggttaaagg

SEQ ID NO: 130 - DNA, 23 bp, Streptococcus pyogenes
aggttaaaggtctttgtactagg

SEQ ID NO: 131 - DNA, 23 bp, Streptococcus pyogenes
ttaaaggtctttgtactaggagg

SEQ ID NO: 132 - DNA, 23 bp, Streptococcus pyogenes
tctttgtactaggaggctgtagg

SEQ ID NO: 133 - DNA, 23 bp, Streptococcus pyogenes
aggaggctgtaggcataaattgg

SEQ ID NO: 134 - DNA, 23 bp, Streptococcus pyogenes
caagcctccaagctgtgccttgg

SEQ ID NO: 135 - DNA, 23 bp, Streptococcus pyogenes
aagcctccaagctgtgccttggg

SEQ ID NO: 136 - DNA, 23 bp, Streptococcus pyogenes
cctccaagctgtgccttgggtgg

SEQ ID NO: 137 - DNA, 23 bp, Streptococcus pyogenes
agctgtgccttgggtggctttgg

SEQ ID NO: 138 - DNA, 23 bp, Streptococcus pyogenes
gctgtgccttgggtggctttggg

SEQ ID NO: 139 - DNA, 23 bp, Streptococcus pyogenes
ctgtgccttgggtggctttgggg

SEQ ID NO: 140 - DNA, 23 bp, Streptococcus pyogenes
ccttgggtggctttggggcatgg

SEQ ID NO: 141 - DNA, 23 bp, Streptococcus pyogenes
atcgacccttataaagaatttgg

SEQ ID NO: 142 - DNA, 23 bp, Streptococcus pyogenes
taaagaatttggagctactgtgg

SEQ ID NO: 143 - DNA, 23 bp, Streptococcus pyogenes
ataccgcctcagctctgtatcgg

SEQ ID NO: 144 - DNA, 23 bp, Streptococcus pyogenes
taccgcctcagctctgtatcggg

SEQ ID NO: 145 - DNA, 23 bp, Streptococcus pyogenes
cacctcaccatactgcactcagg

SEQ ID NO: 146 - DNA, 23 bp, Streptococcus pyogenes
tcaggcaagcaattctttgctgg

SEQ ID NO: 147 - DNA, 23 bp, Streptococcus pyogenes
caggcaagcaattctttgctggg

SEQ ID NO: 148 - DNA, 23 bp, Streptococcus pyogenes
aggcaagcaattctttgctgggg

SEQ ID NO: 149 - DNA, 23 bp, Streptococcus pyogenes
ggcaagcaattctttgctggggg

SEQ ID NO: 150 - DNA, 23 bp, Streptococcus pyogenes
gcaagcaattctttgctgggggg

SEQ ID NO: 151 - DNA, 23 bp, Streptococcus pyogenes
aactaatgactctagctacctgg

SEQ ID NO: 152 - DNA, 23 bp, Streptococcus pyogenes
actaatgactctagctacctggg

SEQ ID NO: 153 - DNA, 23 bp, Streptococcus pyogenes
aatgactctagctacctgggtgg

SEQ ID NO: 154 - DNA, 23 bp, Streptococcus pyogenes
atgactctagctacctgggtggg

SEQ ID NO: 155 - DNA, 23 bp, Streptococcus pyogenes
tacctgggtgggtgttaatttgg

SEQ ID NO: 156 - DNA, 23 bp, Streptococcus pyogenes
cagttatgtcaacactaatatgg

SEQ ID NO: 157 - DNA, 23 bp, Streptococcus pyogenes
agttatgtcaacactaatatggg

SEQ ID NO: 158 - DNA, 23 bp, Streptococcus pyogenes
ctaatatgggcctaaagttcagg

SEQ ID NO: 159 - DNA, 23 bp, Streptococcus pyogenes
taaagttcaggcaactcttgtgg

SEQ ID NO: 160 - DNA, 23 bp, Streptococcus pyogenes
cacatttcttgtctcacttttgg

SEQ ID NO: 161 - DNA, 23 bp, Streptococcus pyogenes
agaaacagttatagagtatttgg

SEQ ID NO: 162 - DNA, 23 bp, Streptococcus pyogenes
atagagtatttggtgtctttcgg

SEQ ID NO: 163 - DNA, 23 bp, Streptococcus pyogenes
atttggtgtctttcggagtgtgg

SEQ ID NO: 164 - DNA, 23 bp, Streptococcus pyogenes
ccctatcctatcaacacttccgg

SEQ ID NO: 165 - DNA, 23 bp, Streptococcus pyogenes
actactgttgttagacgacgagg

SEQ ID NO: 166 - DNA, 23 bp, Streptococcus pyogenes
ctgttgttagacgacgaggcagg

SEQ ID NO: 167 - DNA, 23 bp, Streptococcus pyogenes
ctccctcgcctcgcagacgaagg

SEQ ID NO: 168 - DNA, 23 bp, Streptococcus pyogenes
gtcgcagaagatctcaatctcgg

SEQ ID NO: 169 - DNA, 23 bp, Streptococcus pyogenes
tcgcagaagatctcaatctcggg

SEQ ID NO: 170 - DNA, 23 bp, Streptococcus pyogenes
atctcaatgttagtattccttgg

SEQ ID NO: 171 - DNA, 23 bp, Streptococcus pyogenes
tagtattccttggactcataagg

SEQ ID NO: 172 - DNA, 23 bp, Streptococcus pyogenes
tattccttggactcataaggtgg

SEQ ID NO: 173 - DNA, 23 bp, Streptococcus pyogenes
attccttggactcataaggtggg

SEQ ID NO: 174 - DNA, 23 bp, Streptococcus pyogenes
ttccttggactcataaggtgggg

SEQ ID NO: 175 - DNA, 23 bp, Streptococcus pyogenes
cataaggtggggaactttactgg

SEQ ID NO: 176 - DNA, 23 bp, Streptococcus pyogenes
ataaggtggggaactttactggg

SEQ ID NO: 177 - DNA, 23 bp, Streptococcus pyogenes
tacctgtctttaatcctcattgg

SEQ ID NO: 178 - DNA, 23 bp, Streptococcus pyogenes
aaaaaatgtgaacagtttgtagg

SEQ ID NO: 179 - DNA, 23 bp, Streptococcus pyogenes
tgcaattgattatgcctgccagg

SEQ ID NO: 180 - DNA, 23 bp, Streptococcus pyogenes
gcctgccaggttttatccaaagg

SEQ ID NO: 181 - DNA, 23 bp, Streptococcus pyogenes
ggttaccaaatatttaccattgg

SEQ ID NO: 182 - DNA, 23 bp, Streptococcus pyogenes
caaatatttaccattggataagg

SEQ ID NO: 183 - DNA, 23 bp, Streptococcus pyogenes
aaatatttaccattggataaggg

SEQ ID NO: 184 - DNA, 23 bp, Streptococcus pyogenes
gacactatttacacactctatgg

SEQ ID NO: 185 - DNA, 23 bp, Streptococcus pyogenes
ctatttacacactctatggaagg

SEQ ID NO: 186 - DNA, 23 bp, Streptococcus pyogenes
tttacacactctatggaaggcgg

SEQ ID NO: 187 - DNA, 23 bp, Streptococcus pyogenes
ttacacactctatggaaggcggg

SEQ ID NO: 188 - DNA, 23 bp, Streptococcus pyogenes
acacatagcgcctcattttgtgg

SEQ ID NO: 189 - DNA, 23 bp, Streptococcus pyogenes
cacatagcgcctcattttgtggg

SEQ ID NO: 190 - DNA, 23 bp, Streptococcus pyogenes
tttgtgggtcaccatattcttgg

SEQ ID NO: 191 - DNA, 23 bp, Streptococcus pyogenes
ttgtgggtcaccatattcttggg

SEQ ID NO: 192 - DNA, 23 bp, Streptococcus pyogenes
tgggaacaagatctacagcatgg

SEQ ID NO: 193 - DNA, 23 bp, Streptococcus pyogenes
gggaacaagatctacagcatggg

SEQ ID NO: 194 - DNA, 23 bp, Streptococcus pyogenes
ggaacaagatctacagcatgggg

SEQ ID NO: 195 - DNA, 23 bp, Streptococcus pyogenes
tctttccaccagcaatcctctgg

SEQ ID NO: 196 - DNA, 23 bp, Streptococcus pyogenes
ctttccaccagcaatcctctggg

SEQ ID NO: 197 - DNA, 23 bp, Streptococcus pyogenes
attctttcccgaccaccagttgg

SEQ ID NO: 198 - DNA, 23 bp, Streptococcus pyogenes
caaacaccgcaaatccagattgg

SEQ ID NO: 199 - DNA, 23 bp, Streptococcus pyogenes
aaacaccgcaaatccagattggg

SEQ ID NO: 200 - DNA, 23 bp, Streptococcus pyogenes
ttgggacttcaatcccaacaagg

SEQ ID NO: 201 - DNA, 23 bp, Streptococcus pyogenes
tcaatcccaacaaggacacctgg

SEQ ID NO: 202 - DNA, 23 bp, Streptococcus pyogenes
cacctggccagacgccaacaagg

SEQ ID NO: 203 - DNA, 23 bp, Streptococcus pyogenes
tggccagacgccaacaaggtagg

SEQ ID NO: 204 - DNA, 23 bp, Streptococcus pyogenes
gacgccaacaaggtaggagctgg

SEQ ID NO: 205 - DNA, 23 bp, Streptococcus pyogenes
aaggtaggagctggagcattcgg

SEQ ID NO: 206 - DNA, 23 bp, Streptococcus pyogenes
aggtaggagctggagcattcggg

SEQ ID NO: 207 - DNA, 23 bp, Streptococcus pyogenes
aggagctggagcattcgggctgg

SEQ ID NO: 208 - DNA, 23 bp, Streptococcus pyogenes
ggagctggagcattcgggctggg

SEQ ID NO: 209 - DNA, 23 bp, Streptococcus pyogenes
ctgggtttcaccccaccgcacgg

SEQ ID NO: 210 - DNA, 23 bp, Streptococcus pyogenes
ggtttcaccccaccgcacggagg

SEQ ID NO: 211 - DNA, 23 bp, Streptococcus pyogenes
cccaccgcacggaggccttttgg

SEQ ID NO: 212 - DNA, 23 bp, Streptococcus pyogenes
ccaccgcacggaggccttttggg

SEQ ID NO: 213 - DNA, 23 bp, Streptococcus pyogenes
caccgcacggaggccttttgggg

SEQ ID NO: 214 - DNA, 23 bp, Streptococcus pyogenes
cgcacggaggccttttggggtgg

SEQ ID NO: 215 - DNA, 23 bp, Streptococcus pyogenes
ccttttggggtggagccctcagg

SEQ ID NO: 216 - DNA, 23 bp, Streptococcus pyogenes
ggggtggagccctcaggctcagg

SEQ ID NO: 217 - DNA, 23 bp, Streptococcus pyogenes
gggtggagccctcaggctcaggg

SEQ ID NO: 218 - DNA, 23 bp, Streptococcus pyogenes
gcctccaccaatcgccagtcagg

SEQ ID NO: 219 - DNA, 23 bp, Streptococcus pyogenes
ccaccaatcgccagtcaggaagg

SEQ ID NO: 220 - DNA, 23 bp, Streptococcus pyogenes
tttgagaaacactcatcctcagg

SEQ ID NO: 221 - DNA, 23 bp, Streptococcus pyogenes
ctcatcctcaggccatgcagtgg

SEQ ID NO: 222 - DNA, 23 bp, Streptococcus pyogenes
tgaggatgagtgtttctcaaagg

SEQ ID NO: 223 - DNA, 23 bp, Streptococcus pyogenes
ggatgagtgtttctcaaaggtgg

SEQ ID NO: 224 - DNA, 23 bp, Streptococcus pyogenes
tttctcaaaggtggagacagcgg

SEQ ID NO: 225 - DNA, 23 bp, Streptococcus pyogenes
ttctcaaaggtggagacagcggg

SEQ ID NO: 226 - DNA, 23 bp, Streptococcus pyogenes
tctcaaaggtggagacagcgggg

SEQ ID NO: 227 - DNA, 23 bp, Streptococcus pyogenes
aaaggtggagacagcggggtagg

SEQ ID NO: 228 - DNA, 23 bp, Streptococcus pyogenes
gggtaggctgccttcctgactgg

SEQ ID NO: 229 - DNA, 23 bp, Streptococcus pyogenes
ctgccttcctgactggcgattgg

SEQ ID NO: 230 - DNA, 23 bp, Streptococcus pyogenes
ccttcctgactggcgattggtgg

SEQ ID NO: 231 - DNA, 23 bp, Streptococcus pyogenes
tcctgactggcgattggtggagg

SEQ ID NO: 232 - DNA, 23 bp, Streptococcus pyogenes
gactggcgattggtggaggcagg

SEQ ID NO: 233 - DNA, 23 bp, Streptococcus pyogenes
tggcgattggtggaggcaggagg

SEQ ID NO: 234 - DNA, 23 bp, Streptococcus pyogenes
cgattggtggaggcaggaggcgg

SEQ ID NO: 235 - DNA, 23 bp, Streptococcus pyogenes
gaggcaggaggcggatttgctgg

SEQ ID NO: 236 - DNA, 23 bp, Streptococcus pyogenes
tgtagtatgccctgagcctgagg

SEQ ID NO: 237 - DNA, 23 bp, Streptococcus pyogenes
gtagtatgccctgagcctgaggg

SEQ ID NO: 238 - DNA, 23 bp, Streptococcus pyogenes
cctgagggctccaccccaaaagg

SEQ ID NO: 239 - DNA, 23 bp, Streptococcus pyogenes
caccccaaaaggcctccgtgcgg

SEQ ID NO: 240 - DNA, 23 bp, Streptococcus pyogenes
cccaaaaggcctccgtgcggtgg

SEQ ID NO: 241 - DNA, 23 bp, Streptococcus pyogenes
ccaaaaggcctccgtgcggtggg

SEQ ID NO: 242 - DNA, 23 bp, Streptococcus pyogenes
caaaaggcctccgtgcggtgggg

SEQ ID NO: 243 - DNA, 23 bp, Streptococcus pyogenes
tgctccagctcctaccttgttgg

SEQ ID NO: 244 - DNA, 23 bp, Streptococcus pyogenes
gctcctaccttgttggcgtctgg

SEQ ID NO: 245 - DNA, 23 bp, Streptococcus pyogenes
taccttgttggcgtctggccagg

SEQ ID NO: 246 - DNA, 23 bp, Streptococcus pyogenes
gtctggccaggtgtccttgttgg

SEQ ID NO: 247 - DNA, 23 bp, Streptococcus pyogenes
tctggccaggtgtccttgttggg

SEQ ID NO: 248 - DNA, 23 bp, Streptococcus pyogenes
ttgggattgaagtcccaatctgg

SEQ ID NO: 249 - DNA, 23 bp, Streptococcus pyogenes
gaagtcccaatctggatttgcgg

SEQ ID NO: 250 - DNA, 23 bp, Streptococcus pyogenes
atttgcggtgtttgctctgaagg

SEQ ID NO: 251 - DNA, 23 bp, Streptococcus pyogenes
gcggtgtttgctctgaaggctgg

SEQ ID NO: 252 - DNA, 23 bp, Streptococcus pyogenes
ctctgaaggctggatccaactgg

SEQ ID NO: 253 - DNA, 23 bp, Streptococcus pyogenes
tgaaggctggatccaactggtgg

SEQ ID NO: 254 - DNA, 23 bp, Streptococcus pyogenes
ggctggatccaactggtggtcgg

SEQ ID NO: 255 - DNA, 23 bp, Streptococcus pyogenes
gctggatccaactggtggtcggg

SEQ ID NO: 256 - DNA, 23 bp, Streptococcus pyogenes
tggtcgggaaagaatcccagagg

SEQ ID NO: 257 - DNA, 23 bp, Streptococcus pyogenes
aaagaatcccagaggattgctgg

SEQ ID NO: 258 - DNA, 23 bp, Streptococcus pyogenes
gaatcccagaggattgctggtgg

SEQ ID NO: 259 - DNA, 23 bp, Streptococcus pyogenes
agatcttgttcccaagaatatgg

SEQ ID NO: 260 - DNA, 23 bp, Streptococcus pyogenes
atatggtgacccacaaaatgagg

SEQ ID NO: 261 - DNA, 23 bp, Streptococcus pyogenes
tgtgtaaatagtgtctagtttgg

SEQ ID NO: 262 - DNA, 23 bp, Streptococcus pyogenes
taatgattaactagatgttctgg

SEQ ID NO: 263 - DNA, 23 bp, Streptococcus pyogenes
actagatgttctggataataagg

SEQ ID NO: 264 - DNA, 23 bp, Streptococcus pyogenes
ggtttaatacccttatccaatgg

SEQ ID NO: 265 - DNA, 23 bp, Streptococcus pyogenes
cttatccaatggtaaatatttgg

SEQ ID NO: 266 - DNA, 23 bp, Streptococcus pyogenes
ggtaaatatttggtaacctttgg

SEQ ID NO: 267 - DNA, 23 bp, Streptococcus pyogenes
ggtaacctttggataaaacctgg

SEQ ID NO: 268 - DNA, 23 bp, Streptococcus pyogenes
acctttggataaaacctggcagg

SEQ ID NO: 269 - DNA, 23 bp, Streptococcus pyogenes
cttttctcattaactgtgagtgg

SEQ ID NO: 270 - DNA, 23 bp, Streptococcus pyogenes
ttttctcattaactgtgagtggg

SEQ ID NO: 271 - DNA, 23 bp, Streptococcus pyogenes
cacattttttgataatgtcttgg

SEQ ID NO: 272 - DNA, 23 bp, Streptococcus pyogenes
tcttggtgtaaatgtatattagg

SEQ ID NO: 273 - DNA, 23 bp, Streptococcus pyogenes
aaatgtatattaggaaaagatgg

SEQ ID NO: 274 - DNA, 23 bp, Streptococcus pyogenes
aaagatggtgttttccaatgagg

SEQ ID NO: 275 - DNA, 23 bp, Streptococcus pyogenes
ttccaatgaggattaaagacagg

SEQ ID NO: 276 - DNA, 23 bp, Streptococcus pyogenes
ttccccaccttatgagtccaagg

SEQ ID NO: 277 - DNA, 23 bp, Streptococcus pyogenes
gattgagatcttctgcgacgcgg

SEQ ID NO: 278 - DNA, 23 bp, Streptococcus pyogenes
gattgagaccttcgtctgcgagg

SEQ ID NO: 279 - DNA, 23 bp, Streptococcus pyogenes
agaccttcgtctgcgaggcgagg

SEQ ID NO: 280 - DNA, 23 bp, Streptococcus pyogenes
gaccttcgtctgcgaggcgaggg

SEQ ID NO: 281 - DNA, 23 bp, Streptococcus pyogenes
ggcgagggagttcttcttctagg

SEQ ID NO: 282 - DNA, 23 bp, Streptococcus pyogenes
gcgagggagttcttcttctaggg

SEQ ID NO: 283 - DNA, 23 bp, Streptococcus pyogenes
cgagggagttcttcttctagggg

SEQ ID NO: 284 - DNA, 23 bp, Streptococcus pyogenes
cgtctaacaacagtagtctccgg

SEQ ID NO: 285 - DNA, 23 bp, Streptococcus pyogenes
tagtctccggaagtgttgatagg

SEQ ID NO: 286 - DNA, 23 bp, Streptococcus pyogenes
tccggaagtgttgataggatagg

SEQ ID NO: 287 - DNA, 23 bp, Streptococcus pyogenes
ccggaagtgttgataggataggg

SEQ ID NO: 288 - DNA, 23 bp, Streptococcus pyogenes
cggaagtgttgataggatagggg

SEQ ID NO: 289 - DNA, 23 bp, Streptococcus pyogenes
gttgataggataggggcatttgg

SEQ ID NO: 290 - DNA, 23 bp, Streptococcus pyogenes
gataggataggggcatttggtgg

SEQ ID NO: 291 - DNA, 23 bp, Streptococcus pyogenes
gcatttggtggtctataagctgg

SEQ ID NO: 292 - DNA, 23 bp, Streptococcus pyogenes
tttggtggtctataagctggagg

SEQ ID NO: 293 - DNA, 23 bp, Streptococcus pyogenes
acaagagttgcctgaactttagg

SEQ ID NO: 294 - DNA, 23 bp, Streptococcus pyogenes
tgttgacataactgactactagg

SEQ ID NO: 295 - DNA, 23 bp, Streptococcus pyogenes
actactaggtctctagacgctgg

SEQ ID NO: 296 - DNA, 23 bp, Streptococcus pyogenes
ttccaaattaacacccacccagg

SEQ ID NO: 297 - DNA, 23 bp, Streptococcus pyogenes
ttgcttgcctgagtgcagtatgg

SEQ ID NO: 298 - DNA, 23 bp, Streptococcus pyogenes
tgcctgagtgcagtatggtgagg

SEQ ID NO: 299 - DNA, 23 bp, Streptococcus pyogenes
tggtgaggtgaacaatgctcagg

SEQ ID NO: 300 - DNA, 23 bp, Streptococcus pyogenes
acaatgctcaggagactctaagg

SEQ ID NO: 301 - DNA, 23 bp, Streptococcus pyogenes
ggcttcccgatacagagctgagg

SEQ ID NO: 302 - DNA, 23 bp, Streptococcus pyogenes
ttcccgatacagagctgaggcgg

SEQ ID NO: 303 - DNA, 23 bp, Streptococcus pyogenes
tctagaagatctcgtactgaagg

SEQ ID NO: 304 - DNA, 23 bp, Streptococcus pyogenes
actgaaggaaagaagtcagaagg

SEQ ID NO: 305 - DNA, 23 bp, Streptococcus pyogenes
gtagctccaaattctttataagg

SEQ ID NO: 306 - DNA, 23 bp, Streptococcus pyogenes
tagctccaaattctttataaggg

SEQ ID NO: 307 - DNA, 23 bp, Streptococcus pyogenes
ccatgccccaaagccacccaagg

SEQ ID NO: 308 - DNA, 23 bp, Streptococcus pyogenes
aagccacccaaggcacagcttgg

SEQ ID NO: 309 - DNA, 23 bp, Streptococcus pyogenes
ccacccaaggcacagcttggagg

SEQ ID NO: 310 - DNA, 23 bp, Streptococcus pyogenes
agcttggaggcttgaacagtagg

SEQ ID NO: 311 - DNA, 23 bp, Streptococcus pyogenes
gacatgaacaagagatgattagg

SEQ ID NO: 312 - DNA, 23 bp, Streptococcus pyogenes
aacaagagatgattaggcagagg

SEQ ID NO: 313 - DNA, 23 bp, Streptococcus pyogenes
gcagaggtgaaaaagttgcatgg

SEQ ID NO: 314 - DNA, 23 bp, Streptococcus pyogenes
gtgaaaaagttgcatggtgctgg

SEQ ID NO: 315 - DNA, 23 bp, Streptococcus pyogenes
agtctttgaagtatgcctcaagg

SEQ ID NO: 316 - DNA, 23 bp, Streptococcus pyogenes
tttgaagtatgcctcaaggtcgg

SEQ ID NO: 317 - DNA, 23 bp, Streptococcus pyogenes
agtcctcttatgtaagaccttgg

SEQ ID NO: 318 - DNA, 23 bp, Streptococcus pyogenes
gtcctcttatgtaagaccttggg

SEQ ID NO: 319 - DNA, 23 bp, Streptococcus pyogenes
gtaagaccttgggcaatatttgg

SEQ ID NO: 320 - DNA, 23 bp, Streptococcus pyogenes
agaccttgggcaatatttggtgg

SEQ ID NO: 321 - DNA, 23 bp, Streptococcus pyogenes
gaccttgggcaatatttggtggg

SEQ ID NO: 322 - DNA, 23 bp, Streptococcus pyogenes
caatatttggtgggcgttcacgg

SEQ ID NO: 323 - DNA, 23 bp, Streptococcus pyogenes
tatttggtgggcgttcacggtgg

SEQ ID NO: 324 - DNA, 23 bp, Streptococcus pyogenes
ggtctccatgcgacgtgcagagg

SEQ ID NO: 325 - DNA, 23 bp, Streptococcus pyogenes
gaggtgaagcgaagtgcacacgg

SEQ ID NO: 326 - DNA, 23 bp, Streptococcus pyogenes
gaagcgaagtgcacacggtccgg

SEQ ID NO: 327 - DNA, 23 bp, Streptococcus pyogenes
acacggtccggcagatgagaagg

SEQ ID NO: 328 - DNA, 23 bp, Streptococcus pyogenes
ggcagatgagaaggcacagacgg

SEQ ID NO: 329 - DNA, 23 bp, Streptococcus pyogenes
gcagatgagaaggcacagacggg

SEQ ID NO: 330 - DNA, 23 bp, Streptococcus pyogenes
cagatgagaaggcacagacgggg

SEQ ID NO: 331 - DNA, 23 bp, Streptococcus pyogenes
cggggagtccgcgtaaagagagg

SEQ ID NO: 332 - DNA, 23 bp, Streptococcus pyogenes
gtaaagagaggtgcgccccgtgg

SEQ ID NO: 333 - DNA, 23 bp, Streptococcus pyogenes
agagaggtgcgccccgtggtcgg

SEQ ID NO: 334 - DNA, 23 bp, Streptococcus pyogenes
aggtgcgccccgtggtcggtcgg

SEQ ID NO: 335 - DNA, 23 bp, Streptococcus pyogenes
cgccccgtggtcggtcggaacgg

SEQ ID NO: 336 - DNA, 23 bp, Streptococcus pyogenes
tggtcggtcggaacggcagacgg

SEQ ID NO: 337 - DNA, 23 bp, Streptococcus pyogenes
gtcggaacggcagacggagaagg

SEQ ID NO: 338 - DNA, 23 bp, Streptococcus pyogenes
tcggaacggcagacggagaaggg

SEQ ID NO: 339 - DNA, 23 bp, Streptococcus pyogenes
cggaacggcagacggagaagggg

SEQ ID NO: 340 - DNA, 23 bp, Streptococcus pyogenes
agtcccaagcgaccccgagaagg

SEQ ID NO: 341 - DNA, 23 bp, Streptococcus pyogenes
gtcccaagcgaccccgagaaggg

SEQ ID NO: 342 - DNA, 23 bp, Streptococcus pyogenes
ccccgagaagggtcgtccgcagg

SEQ ID NO: 343 - DNA, 23 bp, Streptococcus pyogenes
tccgcaggattcagcgccgacgg

SEQ ID NO: 344 - DNA, 23 bp, Streptococcus pyogenes
ccgcaggattcagcgccgacggg

SEQ ID NO: 345 - DNA, 23 bp, Streptococcus pyogenes
cgccgacgggacgtaaacaaagg

SEQ ID NO: 346 - DNA, 23 bp, Streptococcus pyogenes
aaacaaaggacgtcccgcgcagg

SEQ ID NO: 347 - DNA, 23 bp, Streptococcus pyogenes
cgtcccgcgcaggatccagttgg

SEQ ID NO: 348 - DNA, 23 bp, Streptococcus pyogenes
gcagcacagcctagcagccatgg

SEQ ID NO: 349 - DNA, 23 bp, Streptococcus pyogenes
atggaaacgatgtatatttgcgg

SEQ ID NO: 350 - DNA, 23 bp, Streptococcus pyogenes
tggaaacgatgtatatttgcggg

SEQ ID NO: 351 - DNA, 23 bp, Streptococcus pyogenes
acgatgtatatttgcgggatagg

SEQ ID NO: 352 - DNA, 23 bp, Streptococcus pyogenes
cctgctgcgagcaaaacaagcgg

SEQ ID NO: 353 - DNA, 23 bp, Streptococcus pyogenes
tgcgagcaaaacaagcggctagg

SEQ ID NO: 354 - DNA, 23 bp, Streptococcus pyogenes
cggctaggagttccgcagtatgg

SEQ ID NO: 355 - DNA, 23 bp, Streptococcus pyogenes
aggagttccgcagtatggatcgg

SEQ ID NO: 356 - DNA, 23 bp, Streptococcus pyogenes
tccgcagtatggatcggcagagg

SEQ ID NO: 357 - DNA, 23 bp, Streptococcus pyogenes
gatcggcagaggagccgaaaagg

SEQ ID NO: 358 - DNA, 23 bp, Streptococcus pyogenes
aggttccacgcatgcgctgatgg

SEQ ID NO: 359 - DNA, 23 bp, Streptococcus pyogenes
catgaccaagccccagccagtgg

SEQ ID NO: 360 - DNA, 23 bp, Streptococcus pyogenes
atgaccaagccccagccagtggg

SEQ ID NO: 361 - DNA, 23 bp, Streptococcus pyogenes
tgaccaagccccagccagtgggg

SEQ ID NO: 362 - DNA, 23 bp, Streptococcus pyogenes
gaccaagccccagccagtggggg

SEQ ID NO: 363 - DNA, 23 bp, Streptococcus pyogenes
gggttgcgtcagcaaacacttgg

SEQ ID NO: 364 - DNA, 23 bp, Streptococcus pyogenes
gcaaacacttggcacagacctgg

SEQ ID NO: 365 - DNA, 23 bp, Streptococcus pyogenes
ggcacagacctggccgttgccgg

SEQ ID NO: 366 - DNA, 23 bp, Streptococcus pyogenes
gcacagacctggccgttgccggg

SEQ ID NO: 367 - DNA, 23 bp, Streptococcus pyogenes
acctggccgttgccgggcaacgg

SEQ ID NO: 368 - DNA, 23 bp, Streptococcus pyogenes
cctggccgttgccgggcaacggg

SEQ ID NO: 369 - DNA, 23 bp, Streptococcus pyogenes
ctggccgttgccgggcaacgggg

SEQ ID NO: 370 - DNA, 23 bp, Streptococcus pyogenes
gttgccgggcaacggggtaaagg

SEQ ID NO: 371 - DNA, 23 bp, Streptococcus pyogenes
gggcaacggggtaaaggttcagg

SEQ ID NO: 372 - DNA, 23 bp, Streptococcus pyogenes
caggtattgtttacacagaaagg

SEQ ID NO: 373 - DNA, 23 bp, Streptococcus pyogenes
cacagaaaggccttgtaagttgg

SEQ ID NO: 374 - DNA, 23 bp, Streptococcus pyogenes
gattgaatacatgcatacaaagg

SEQ ID NO: 375 - DNA, 23 bp, Streptococcus pyogenes
gcatacaaaggcatcaacgcagg

SEQ ID NO: 376 - DNA, 23 bp, Streptococcus pyogenes
ggataaccacattgtgtaaaagg

SEQ ID NO: 377 - DNA, 23 bp, Streptococcus pyogenes
gataaccacattgtgtaaaaggg

SEQ ID NO: 378 - DNA, 23 bp, Streptococcus pyogenes
ataaccacattgtgtaaaagggg

SEQ ID NO: 379 - DNA, 23 bp, Streptococcus pyogenes
tgacatactttccaatcaatagg

SEQ ID NO: 380 - DNA, 23 bp, Streptococcus pyogenes
caatcaataggcctgttaatagg

SEQ ID NO: 381 - DNA, 23 bp, Streptococcus pyogenes
ttttgtatgatgtgttcttgtgg

SEQ ID NO: 382 - DNA, 23 bp, Streptococcus pyogenes
tatgatgtgttcttgtggcaagg

SEQ ID NO: 383 - DNA, 23 bp, Streptococcus pyogenes
ccccatctctttgttttgttagg

SEQ ID NO: 384 - DNA, 23 bp, Streptococcus pyogenes
cccatctctttgttttgttaggg

SEQ ID NO: 385 - DNA, 23 bp, Streptococcus pyogenes
acccaaagacaaaagaaaattgg

SEQ ID NO: 386 - DNA, 23 bp, Streptococcus pyogenes
caaaagaaaattggtaacagcgg

SEQ ID NO: 387 - DNA, 23 bp, Streptococcus pyogenes
aattggtaacagcggtaaaaagg

SEQ ID NO: 388 - DNA, 23 bp, Streptococcus pyogenes
attggtaacagcggtaaaaaggg

SEQ ID NO: 389 - DNA, 23 bp, Streptococcus pyogenes
ctcaagatgctgtacagacttgg

SEQ ID NO: 390 - DNA, 23 bp, Streptococcus pyogenes
tataactgaaagccaaacagtgg

SEQ ID NO: 391 - DNA, 23 bp, Streptococcus pyogenes
ataactgaaagccaaacagtggg

SEQ ID NO: 392 - DNA, 23 bp, Streptococcus pyogenes
taactgaaagccaaacagtgggg

SEQ ID NO: 393 - DNA, 23 bp, Streptococcus pyogenes
aactgaaagccaaacagtggggg

SEQ ID NO: 394 - DNA, 23 bp, Streptococcus pyogenes
cctacgaaccactgaacaaatgg

SEQ ID NO: 395 - DNA, 23 bp, Streptococcus pyogenes
tggcactagtaaactgagccagg

SEQ ID NO: 396 - DNA, 23 bp, Streptococcus pyogenes
gtaaactgagccaggagaaacgg

SEQ ID NO: 397 - DNA, 23 bp, Streptococcus pyogenes
taaactgagccaggagaaacggg

SEQ ID NO: 398 - DNA, 23 bp, Streptococcus pyogenes
gagccaggagaaacgggctgagg

SEQ ID NO: 399 - DNA, 23 bp, Streptococcus pyogenes
gggctgaggcccactcccatagg

SEQ ID NO: 400 - DNA, 23 bp, Streptococcus pyogenes
taggaattttccgaaagcccagg

SEQ ID NO: 401 - DNA, 23 bp, Streptococcus pyogenes
tttccgaaagcccaggatgatgg

SEQ ID NO: 402 - DNA, 23 bp, Streptococcus pyogenes
ttccgaaagcccaggatgatggg

SEQ ID NO: 403 - DNA, 23 bp, Streptococcus pyogenes
gaaagcccaggatgatgggatgg

SEQ ID NO: 404 - DNA, 23 bp, Streptococcus pyogenes
aaagcccaggatgatgggatggg

SEQ ID NO: 405 - DNA, 23 bp, Streptococcus pyogenes
ggatgatgggatgggaatacagg

SEQ ID NO: 406 - DNA, 23 bp, Streptococcus pyogenes
caggtgcaatttccgtccgaagg

SEQ ID NO: 407 - DNA, 23 bp, Streptococcus pyogenes
gcaatttccgtccgaaggtttgg

SEQ ID NO: 408 - DNA, 23 bp, Streptococcus pyogenes
cgaaggtttggtacagcaacagg

SEQ ID NO: 409 - DNA, 23 bp, Streptococcus pyogenes
aggtttggtacagcaacaggagg

SEQ ID NO: 410 - DNA, 23 bp, Streptococcus pyogenes
ggtttggtacagcaacaggaggg

SEQ ID NO: 411 - DNA, 23 bp, Streptococcus pyogenes
gcaacaggagggatacatagagg

SEQ ID NO: 412 - DNA, 23 bp, Streptococcus pyogenes
tccttgagcagtagtcatgcagg

SEQ ID NO: 413 - DNA, 23 bp, Streptococcus pyogenes
gagcagtagtcatgcaggtccgg

SEQ ID NO: 414 - DNA, 23 bp, Streptococcus pyogenes
gtagtcatgcaggtccggcatgg

SEQ ID NO: 415 - DNA, 23 bp, Streptococcus pyogenes
ggtccggcatggtcccgtgctgg

SEQ ID NO: 416 - DNA, 23 bp, Streptococcus pyogenes
tggtcccgtgctggttgttgagg

SEQ ID NO: 417 - DNA, 23 bp, Streptococcus pyogenes
gtgctggttgttgaggatcctgg

SEQ ID NO: 418 - DNA, 23 bp, Streptococcus pyogenes
gttgaggatcctggaattagagg

SEQ ID NO: 419 - DNA, 23 bp, Streptococcus pyogenes
tcctggaattagaggacaaacgg

SEQ ID NO: 420 - DNA, 23 bp, Streptococcus pyogenes
cctggaattagaggacaaacggg

SEQ ID NO: 421 - DNA, 23 bp, Streptococcus pyogenes
gaagaaccaacaagaagatgagg

SEQ ID NO: 422 - DNA, 23 bp, Streptococcus pyogenes
agaagatgaggcatagcagcagg

SEQ ID NO: 423 - DNA, 23 bp, Streptococcus pyogenes
ggcatagcagcaggatgaagagg

SEQ ID NO: 424 - DNA, 23 bp, Streptococcus pyogenes
agacacatccagcgataaccagg

SEQ ID NO: 425 - DNA, 23 bp, Streptococcus pyogenes
cagcgataaccaggacaagttgg

SEQ ID NO: 426 - DNA, 23 bp, Streptococcus pyogenes
cgataaccaggacaagttggagg

SEQ ID NO: 427 - DNA, 23 bp, Streptococcus pyogenes
aggacaagttggaggacaagagg

SEQ ID NO: 428 - DNA, 23 bp, Streptococcus pyogenes
caagttggaggacaagaggttgg

SEQ ID NO: 429 - DNA, 23 bp, Streptococcus pyogenes
acaagaggttggtgagtgattgg

SEQ ID NO: 430 - DNA, 23 bp, Streptococcus pyogenes
agaggttggtgagtgattggagg

SEQ ID NO: 431 - DNA, 23 bp, Streptococcus pyogenes
gttggtgagtgattggaggttgg

SEQ ID NO: 432 - DNA, 23 bp, Streptococcus pyogenes
ttggtgagtgattggaggttggg

SEQ ID NO: 433 - DNA, 23 bp, Streptococcus pyogenes
tggtgagtgattggaggttgggg

SEQ ID NO: 434 - DNA, 23 bp, Streptococcus pyogenes
aggttggggactgcgaattttgg

SEQ ID NO: 435 - DNA, 23 bp, Streptococcus pyogenes
cgaattttggccaagacacacgg

SEQ ID NO: 436 - DNA, 23 bp, Streptococcus pyogenes
caccacgagtctagactctgcgg

SEQ ID NO: 437 - DNA, 23 bp, Streptococcus pyogenes
ctagactctgcggtattgtgagg

SEQ ID NO: 438 - DNA, 23 bp, Streptococcus pyogenes
accccgcctgtaacacgagaagg

SEQ ID NO: 439 - DNA, 23 bp, Streptococcus pyogenes
ccccgcctgtaacacgagaaggg

SEQ ID NO: 440 - DNA, 23 bp, Streptococcus pyogenes
cccgcctgtaacacgagaagggg

SEQ ID NO: 441 - DNA, 23 bp, Streptococcus pyogenes
gtaacacgagaaggggtcctagg

SEQ ID NO: 442 - DNA, 23 bp, Streptococcus pyogenes
atgttctccatgttcagcgcagg

SEQ ID NO: 443 - DNA, 23 bp, Streptococcus pyogenes
tgttctccatgttcagcgcaggg

SEQ ID NO: 444 - DNA, 23 bp, Streptococcus pyogenes
cctcgagaagattgacgataagg

SEQ ID NO: 445 - DNA, 23 bp, Streptococcus pyogenes
ctcgagaagattgacgataaggg

SEQ ID NO: 446 - DNA, 23 bp, Streptococcus pyogenes
gaagattgacgataagggagagg

SEQ ID NO: 447 - DNA, 23 bp, Streptococcus pyogenes
ggagaggcagtagtcagaacagg

SEQ ID NO: 448 - DNA, 23 bp, Streptococcus pyogenes
gagaggcagtagtcagaacaggg

SEQ ID NO: 449 - DNA, 23 bp, Streptococcus pyogenes
agggtttactgttcctgaactgg

SEQ ID NO: 450 - DNA, 23 bp, Streptococcus pyogenes
cctgaactggagccaccagcagg

SEQ ID NO: 451 - DNA, 23 bp, Streptococcus pyogenes
ctgaactggagccaccagcaggg

SEQ ID NO: 452 - DNA, 23 bp, Streptococcus pyogenes
agccaccagcagggaaatacagg

SEQ ID NO: 453 - DNA, 23 bp, Streptococcus pyogenes
gaaatacaggcctctcactctgg

SEQ ID NO: 454 - DNA, 23 bp, Streptococcus pyogenes
aaatacaggcctctcactctggg

SEQ ID NO: 455 - DNA, 23 bp, Streptococcus pyogenes
ctctgggatcttgcagagtttgg

SEQ ID NO: 456 - DNA, 23 bp, Streptococcus pyogenes
tgggatcttgcagagtttggtgg

SEQ ID NO: 457 - DNA, 23 bp, Streptococcus pyogenes
atcttgcagagtttggtggaagg

SEQ ID NO: 458 - DNA, 23 bp, Streptococcus pyogenes
cagagtttggtggaaggttgtgg

SEQ ID NO: 459 - DNA, 23 bp, Streptococcus pyogenes
ggttgtggaattccactgcatgg

SEQ ID NO: 460 - DNA, 23 bp, Streptococcus pyogenes
gaattccactgcatggcctgagg

SEQ ID NO: 461 - DNA, 23 bp, Streptococcus pyogenes
tgccttccaccaagctctgcagg

SEQ ID NO: 462 - DNA, 23 bp, Streptococcus pyogenes
ctctgcaggatcccagagtcagg

SEQ ID NO: 463 - DNA, 23 bp, Streptococcus pyogenes
tctgcaggatcccagagtcaggg

SEQ ID NO: 464 - DNA, 23 bp, Streptococcus pyogenes
ctgcaggatcccagagtcagggg

SEQ ID NO: 465 - DNA, 23 bp, Streptococcus pyogenes
aggggtctgtattttcctgctgg

SEQ ID NO: 466 - DNA, 23 bp, Streptococcus pyogenes
ggtctgtattttcctgctggtgg

SEQ ID NO: 467 - DNA, 23 bp, Streptococcus pyogenes
acatctcgtcaatctccgcgagg

SEQ ID NO: 468 - DNA, 23 bp, Streptococcus pyogenes
tcgtcaatctccgcgaggactgg

SEQ ID NO: 469 - DNA, 23 bp, Streptococcus pyogenes
cgtcaatctccgcgaggactggg

SEQ ID NO: 470 - DNA, 23 bp, Streptococcus pyogenes
gtcaatctccgcgaggactgggg

SEQ ID NO: 471 - DNA, 23 bp, Streptococcus pyogenes
tggggaccctgtgacgaacatgg

SEQ ID NO: 472 - DNA, 23 bp, Streptococcus pyogenes
aggacccctgctcgtgttacagg

SEQ ID NO: 473 - DNA, 23 bp, Streptococcus pyogenes
acccctgctcgtgttacaggcgg

SEQ ID NO: 474 - DNA, 23 bp, Streptococcus pyogenes
cccctgctcgtgttacaggcggg

SEQ ID NO: 475 - DNA, 23 bp, Streptococcus pyogenes
ccctgctcgtgttacaggcgggg

SEQ ID NO: 476 - DNA, 23 bp, Streptococcus pyogenes
gggggatcacccgtgtgtcttgg

SEQ ID NO: 477 - DNA, 23 bp, Streptococcus pyogenes
tcctgtcctccaatttgtcctgg

SEQ ID NO: 478 - DNA, 23 bp, Streptococcus pyogenes
caatttgtcctggttatcgctgg

SEQ ID NO: 479 - DNA, 23 bp, Streptococcus pyogenes
gctatgcctcatcttcttattgg

SEQ ID NO: 480 - DNA, 23 bp, Streptococcus pyogenes
catcttcttattggttcttctgg

SEQ ID NO: 481 - DNA, 23 bp, Streptococcus pyogenes
ttggttcttctggattatcaagg

SEQ ID NO: 482 - DNA, 23 bp, Streptococcus pyogenes
aggatcaacaacaaccagtacgg

SEQ ID NO: 483 - DNA, 23 bp, Streptococcus pyogenes
ggatcaacaacaaccagtacggg

SEQ ID NO: 484 - DNA, 23 bp, Streptococcus pyogenes
acctgcacgactcctgctcaagg

SEQ ID NO: 485 - DNA, 23 bp, Streptococcus pyogenes
atgttgctgtacaaaacctacgg

SEQ ID NO: 486 - DNA, 23 bp, Streptococcus pyogenes
tgctgtacaaaacctacggatgg

SEQ ID NO: 487 - DNA, 23 bp, Streptococcus pyogenes
gtattcccatcccatcgtcctgg

SEQ ID NO: 488 - DNA, 23 bp, Streptococcus pyogenes
tattcccatcccatcgtcctggg

SEQ ID NO: 489 - DNA, 23 bp, Streptococcus pyogenes
gggctttcgcaaaatacctatgg

SEQ ID NO: 490 - DNA, 23 bp, Streptococcus pyogenes
ggctttcgcaaaatacctatggg

SEQ ID NO: 491 - DNA, 23 bp, Streptococcus pyogenes
tcgcaaaatacctatgggagtgg

SEQ ID NO: 492 - DNA, 23 bp, Streptococcus pyogenes
cgcaaaatacctatgggagtggg

SEQ ID NO: 493 - DNA, 23 bp, Streptococcus pyogenes
gggcctcagtccgtttctcttgg

SEQ ID NO: 494 - DNA, 23 bp, Streptococcus pyogenes
ctgtttggctttcagctatatgg

SEQ ID NO: 495 - DNA, 23 bp, Streptococcus pyogenes
tttcagctatatggatgatgtgg

SEQ ID NO: 496 - DNA, 23 bp, Streptococcus pyogenes
ctatatggatgatgtggtattgg

SEQ ID NO: 497 - DNA, 23 bp, Streptococcus pyogenes
taccaattttcttttgtctctgg

SEQ ID NO: 498 - DNA, 23 bp, Streptococcus pyogenes
accaattttcttttgtctctggg

SEQ ID NO: 499 - DNA, 23 bp, Streptococcus pyogenes
accctaacaaaacaaaaagatgg

SEQ ID NO: 500 - DNA, 23 bp, Streptococcus pyogenes
ccctaacaaaacaaaaagatggg

SEQ ID NO: 501 - DNA, 23 bp, Streptococcus pyogenes
cctaacaaaacaaaaagatgggg

SEQ ID NO: 502 - DNA, 23 bp, Streptococcus pyogenes
gggttattccctaaacttcatgg

SEQ ID NO: 503 - DNA, 23 bp, Streptococcus pyogenes
ggttattccctaaacttcatggg

SEQ ID NO: 504 - DNA, 23 bp, Streptococcus pyogenes
aacttcatgggttacataattgg

SEQ ID NO: 505 - DNA, 23 bp, Streptococcus pyogenes
tgggttacataattggaagttgg

SEQ ID NO: 506 - DNA, 23 bp, Streptococcus pyogenes
gggttacataattggaagttggg

SEQ ID NO: 507 - DNA, 23 bp, Streptococcus pyogenes
ggttacataattggaagttgggg

SEQ ID NO: 508 - DNA, 23 bp, Streptococcus pyogenes
aagttggggaactttgccacagg

SEQ ID NO: 509 - DNA, 23 bp, Streptococcus pyogenes
tcagaaaacttcctgttaacagg

SEQ ID NO: 510 - DNA, 23 bp, Streptococcus pyogenes
ctgttaacaggcctattgattgg

SEQ ID NO: 511 - DNA, 23 bp, Streptococcus pyogenes
gaaagtatgtcaaagaattgtgg

SEQ ID NO: 512 - DNA, 23 bp, Streptococcus pyogenes
aaagtatgtcaaagaattgtggg

SEQ ID NO: 513 - DNA, 23 bp, Streptococcus pyogenes
tcaaagaattgtgggtcttttgg

SEQ ID NO: 514 - DNA, 23 bp, Streptococcus pyogenes
caaagaattgtgggtcttttggg

SEQ ID NO: 515 - DNA, 23 bp, Streptococcus pyogenes
gctgctccatttacacaatgtgg

SEQ ID NO: 516 - DNA, 23 bp, Streptococcus pyogenes
tgcatgtatacaagctaaacagg

SEQ ID NO: 517 - DNA, 23 bp, Streptococcus pyogenes
tgaacctttaccccgttgctcgg

SEQ ID NO: 518 - DNA, 23 bp, Streptococcus pyogenes
tttaccccgttgctcggcaacgg

SEQ ID NO: 519 - DNA, 23 bp, Streptococcus pyogenes
cccgttgctcggcaacggcctgg

SEQ ID NO: 520 - DNA, 23 bp, Streptococcus pyogenes
actggctggggcttggccatagg

SEQ ID NO: 521 - DNA, 23 bp, Streptococcus pyogenes
ataggccatcagcgcatgcgtgg

SEQ ID NO: 522 - DNA, 23 bp, Streptococcus pyogenes
gcgcatgcgtggaacctttgtgg

SEQ ID NO: 523 - DNA, 23 bp, Streptococcus pyogenes
ccgcttgttttgctcgcagccgg

SEQ ID NO: 524 - DNA, 23 bp, Streptococcus pyogenes
tgttttgctcgcagccggtctgg

SEQ ID NO: 525 - DNA, 23 bp, Streptococcus pyogenes
cggtctggagcaaagctcatcgg

SEQ ID NO: 526 - DNA, 23 bp, Streptococcus pyogenes
acaattctgtcgtcctctcgcgg

SEQ ID NO: 527 - DNA, 23 bp, Streptococcus pyogenes
ggaaatatacatcgtttccatgg

SEQ ID NO: 528 - DNA, 23 bp, Streptococcus pyogenes
tgctaggctgtactgccaactgg

SEQ ID NO: 529 - DNA, 23 bp, Streptococcus pyogenes
actgccaactggatccttcgcgg

SEQ ID NO: 530 - DNA, 23 bp, Streptococcus pyogenes
ctgccaactggatccttcgcggg

SEQ ID NO: 531 - DNA, 23 bp, Streptococcus pyogenes
gtcctttgtctacgtcccgtcgg

SEQ ID NO: 532 - DNA, 23 bp, Streptococcus pyogenes
cccgtcggcgctgaatcccgcgg

SEQ ID NO: 533 - DNA, 23 bp, Streptococcus pyogenes
atcccgcggacgacccctcgcgg

SEQ ID NO: 534 - DNA, 23 bp, Streptococcus pyogenes
tcccgcggacgacccctcgcggg

SEQ ID NO: 535 - DNA, 23 bp, Streptococcus pyogenes
cccgcggacgacccctcgcgggg

SEQ ID NO: 536 - DNA, 23 bp, Streptococcus pyogenes
cgacccctcgcggggccgcttgg

SEQ ID NO: 537 - DNA, 23 bp, Streptococcus pyogenes
gacccctcgcggggccgcttggg

SEQ ID NO: 538 - DNA, 23 bp, Streptococcus pyogenes
tctgccgttccagccgaccacgg

SEQ ID NO: 539 - DNA, 23 bp, Streptococcus pyogenes
ctgccgttccagccgaccacggg

SEQ ID NO: 540 - DNA, 23 bp, Streptococcus pyogenes
tgccgttccagccgaccacgggg

SEQ ID NO: 541 - DNA, 23 bp, Streptococcus pyogenes
cttcacctctgcacgttgcatgg

SEQ ID NO: 542 - DNA, 23 bp, Streptococcus pyogenes
cgcccatcagatcctgcccaagg

SEQ ID NO: 543 - DNA, 23 bp, Streptococcus pyogenes
cttcaaagactgtgtgtttaagg

SEQ ID NO: 544 - DNA, 23 bp, Streptococcus pyogenes
aagactgtgtgtttaaggactgg

SEQ ID NO: 545 - DNA, 23 bp, Streptococcus pyogenes
agactgtgtgtttaaggactggg

SEQ ID NO: 546 - DNA, 23 bp, Streptococcus pyogenes
ctgtgtgtttaaggactgggagg

SEQ ID NO: 547 - DNA, 23 bp, Streptococcus pyogenes
gtttaaggactgggaggagctgg

SEQ ID NO: 548 - DNA, 23 bp, Streptococcus pyogenes
tttaaggactgggaggagctggg

SEQ ID NO: 549 - DNA, 23 bp, Streptococcus pyogenes
ttaaggactgggaggagctgggg

SEQ ID NO: 550 - DNA, 23 bp, Streptococcus pyogenes
taaggactgggaggagctggggg

SEQ ID NO: 551 - DNA, 23 bp, Streptococcus pyogenes
ggactgggaggagctgggggagg

SEQ ID NO: 552 - DNA, 23 bp, Streptococcus pyogenes
aggagctgggggaggagattagg

SEQ ID NO: 553 - DNA, 23 bp, Streptococcus pyogenes
aggttaaaggtctttgtattagg

SEQ ID NO: 554 - DNA, 23 bp, Streptococcus pyogenes
ttaaaggtctttgtattaggagg

SEQ ID NO: 555 - DNA, 23 bp, Streptococcus pyogenes
tctttgtattaggaggctgtagg

SEQ ID NO: 556 - DNA, 23 bp, Streptococcus pyogenes
attgacccttataaagaatttgg

SEQ ID NO: 557 - DNA, 23 bp, Streptococcus pyogenes
tcaggcaagccattctctgctgg

SEQ ID NO: 558 - DNA, 23 bp, Streptococcus pyogenes
caggcaagccattctctgctggg

SEQ ID NO: 559 - DNA, 23 bp, Streptococcus pyogenes
aggcaagccattctctgctgggg

SEQ ID NO: 560 - DNA, 23 bp, Streptococcus pyogenes
ggcaagccattctctgctggggg

SEQ ID NO: 561 - DNA, 23 bp, Streptococcus pyogenes
gcaagccattctctgctgggggg

SEQ ID NO: 562 - DNA, 23 bp, Streptococcus pyogenes
aattgatgactctagctacctgg

SEQ ID NO: 563 - DNA, 23 bp, Streptococcus pyogenes
attgatgactctagctacctggg

SEQ ID NO: 564 - DNA, 23 bp, Streptococcus pyogenes
gatgactctagctacctgggtgg

SEQ ID NO: 565 - DNA, 23 bp, Streptococcus pyogenes
tacctgggtgggtaataatttgg

SEQ ID NO: 566 - DNA, 23 bp, Streptococcus pyogenes
atttggaagatccagcatccagg

SEQ ID NO: 567 - DNA, 23 bp, Streptococcus pyogenes
tttggaagatccagcatccaggg

SEQ ID NO: 568 - DNA, 23 bp, Streptococcus pyogenes
caattatgttaatactaacatgg

SEQ ID NO: 569 - DNA, 23 bp, Streptococcus pyogenes
aattatgttaatactaacatggg

SEQ ID NO: 570 - DNA, 23 bp, Streptococcus pyogenes
ctaacatgggtttaaagatcagg

SEQ ID NO: 571 - DNA, 23 bp, Streptococcus pyogenes
taaagatcaggcaactattgtgg

SEQ ID NO: 572 - DNA, 23 bp, Streptococcus pyogenes
catatatcttgccttacttttgg

SEQ ID NO: 573 - DNA, 23 bp, Streptococcus pyogenes
agagactgtacttgaatatttgg

SEQ ID NO: 574 - DNA, 23 bp, Streptococcus pyogenes
cttgaatatttggtctctttcgg

SEQ ID NO: 575 - DNA, 23 bp, Streptococcus pyogenes
atttggtctctttcggagtgtgg

SEQ ID NO: 576 - DNA, 23 bp, Streptococcus pyogenes
ccctatcttatcaacacttccgg

SEQ ID NO: 577 - DNA, 23 bp, Streptococcus pyogenes
aaactactgttgttagacgacgg

SEQ ID NO: 578 - DNA, 23 bp, Streptococcus pyogenes
aactactgttgttagacgacggg

SEQ ID NO: 579 - DNA, 23 bp, Streptococcus pyogenes
gttgttagacgacgggaccgagg

SEQ ID NO: 580 - DNA, 23 bp, Streptococcus pyogenes
ttagacgacgggaccgaggcagg

SEQ ID NO: 581 - DNA, 23 bp, Streptococcus pyogenes
tcataaggtgggaaactttacgg

SEQ ID NO: 582 - DNA, 23 bp, Streptococcus pyogenes
cataaggtgggaaactttacggg

SEQ ID NO: 583 - DNA, 23 bp, Streptococcus pyogenes
ataaggtgggaaactttacgggg

SEQ ID NO: 584 - DNA, 23 bp, Streptococcus pyogenes
tacctatctttaatcctgaatgg

SEQ ID NO: 585 - DNA, 23 bp, Streptococcus pyogenes
tcctaagattcatttacaagagg

SEQ ID NO: 586 - DNA, 23 bp, Streptococcus pyogenes
tacaagaggacattattaatagg

SEQ ID NO: 587 - DNA, 23 bp, Streptococcus pyogenes
taataggtgtcaacaatttgtgg

SEQ ID NO: 588 - DNA, 23 bp, Streptococcus pyogenes
aataggtgtcaacaatttgtggg

SEQ ID NO: 589 - DNA, 23 bp, Streptococcus pyogenes
aaatatttgcccttagacaaagg

SEQ ID NO: 590 - DNA, 23 bp, Streptococcus pyogenes
taaaccttattatccagatcagg

SEQ ID NO: 591 - DNA, 23 bp, Streptococcus pyogenes
gacattatttacatactctttgg

SEQ ID NO: 592 - DNA, 23 bp, Streptococcus pyogenes
ttatttacatactctttggaagg

SEQ ID NO: 593 - DNA, 23 bp, Streptococcus pyogenes
ttacatactctttggaaggctgg

SEQ ID NO: 594 - DNA, 23 bp, Streptococcus pyogenes
aggctggtattctatataagagg

SEQ ID NO: 595 - DNA, 23 bp, Streptococcus pyogenes
ggctggtattctatataagaggg

SEQ ID NO: 596 - DNA, 23 bp, Streptococcus pyogenes
acacgtagcgcatcattttgcgg

SEQ ID NO: 597 - DNA, 23 bp, Streptococcus pyogenes
cacgtagcgcatcattttgcggg

SEQ ID NO: 598 - DNA, 23 bp, Streptococcus pyogenes
tttgcgggtcaccatattcttgg

SEQ ID NO: 599 - DNA, 23 bp, Streptococcus pyogenes
ttgcgggtcaccatattcttggg

SEQ ID NO: 600 - DNA, 23 bp, Streptococcus pyogenes
tgggaacaagagctacagcatgg

SEQ ID NO: 601 - DNA, 23 bp, Streptococcus pyogenes
gggaacaagagctacagcatggg

SEQ ID NO: 602 - DNA, 23 bp, Streptococcus pyogenes
aacaagagctacagcatgggagg

SEQ ID NO: 603 - DNA, 23 bp, Streptococcus pyogenes
agagctacagcatgggaggttgg

SEQ ID NO: 604 - DNA, 23 bp, Streptococcus pyogenes
tggtcatcaaaacctcgcaaagg

SEQ ID NO: 605 - DNA, 23 bp, Streptococcus pyogenes
atcaaaacctcgcaaaggcatgg

SEQ ID NO: 606 - DNA, 23 bp, Streptococcus pyogenes
tcaaaacctcgcaaaggcatggg

SEQ ID NO: 607 - DNA, 23 bp, Streptococcus pyogenes
caaaacctcgcaaaggcatgggg

SEQ ID NO: 608 - DNA, 23 bp, Streptococcus pyogenes
tctttctgttcccaaccctctgg

SEQ ID NO: 609 - DNA, 23 bp, Streptococcus pyogenes
ctttctgttcccaaccctctggg

SEQ ID NO: 610 - DNA, 23 bp, Streptococcus pyogenes
attctttcccgatcatcagttgg

SEQ ID NO: 611 - DNA, 23 bp, Streptococcus pyogenes
catcagttggaccctgcattcgg

SEQ ID NO: 612 - DNA, 23 bp, Streptococcus pyogenes
ccaactcaaacaatccagattgg

SEQ ID NO: 613 - DNA, 23 bp, Streptococcus pyogenes
caactcaaacaatccagattggg

SEQ ID NO: 614 - DNA, 23 bp, Streptococcus pyogenes
ttgggacttcaaccccatcaagg

SEQ ID NO: 615 - DNA, 23 bp, Streptococcus pyogenes
tcaaccccatcaaggaccactgg

SEQ ID NO: 616 - DNA, 23 bp, Streptococcus pyogenes
ccactggccagcagccaaccagg

SEQ ID NO: 617 - DNA, 23 bp, Streptococcus pyogenes
tggccagcagccaaccaggtagg

SEQ ID NO: 618 - DNA, 23 bp, Streptococcus pyogenes
agcagccaaccaggtaggagtgg

SEQ ID NO: 619 - DNA, 23 bp, Streptococcus pyogenes
gcagccaaccaggtaggagtggg

SEQ ID NO: 620 - DNA, 23 bp, Streptococcus pyogenes
caggtaggagtgggagcattcgg

SEQ ID NO: 621 - DNA, 23 bp, Streptococcus pyogenes
aggtaggagtgggagcattcggg

SEQ ID NO: 622 - DNA, 23 bp, Streptococcus pyogenes
ggagtgggagcattcgggccagg

SEQ ID NO: 623 - DNA, 23 bp, Streptococcus pyogenes
gagtgggagcattcgggccaggg

SEQ ID NO: 624 - DNA, 23 bp, Streptococcus pyogenes
ccagggctcacccctccacacgg

SEQ ID NO: 625 - DNA, 23 bp, Streptococcus pyogenes
gggctcacccctccacacggcgg

SEQ ID NO: 626 - DNA, 23 bp, Streptococcus pyogenes
ccctccacacggcggtattttgg

SEQ ID NO: 627 - DNA, 23 bp, Streptococcus pyogenes
cctccacacggcggtattttggg

SEQ ID NO: 628 - DNA, 23 bp, Streptococcus pyogenes
ctccacacggcggtattttgggg

SEQ ID NO: 629 - DNA, 23 bp, Streptococcus pyogenes
cacacggcggtattttggggtgg

SEQ ID NO: 630 - DNA, 23 bp, Streptococcus pyogenes
tattttggggtggagccctcagg

SEQ ID NO: 631 - DNA, 23 bp, Streptococcus pyogenes
ctcctcctgcctccaccaatcgg

SEQ ID NO: 632 - DNA, 23 bp, Streptococcus pyogenes
gcctccaccaatcggcagtcagg

SEQ ID NO: 633 - DNA, 23 bp, Streptococcus pyogenes
ccaccaatcggcagtcaggaagg

SEQ ID NO: 634 - DNA, 23 bp, Streptococcus pyogenes
tctaagagacagtcatcctcagg

SEQ ID NO: 635 - DNA, 23 bp, Streptococcus pyogenes
gtcatcctcaggccatgcagtgg

SEQ ID NO: 636 - DNA, 23 bp, Streptococcus pyogenes
tgaggatgactgtctcttagagg

SEQ ID NO: 637 - DNA, 23 bp, Streptococcus pyogenes
ggatgactgtctcttagaggtgg

SEQ ID NO: 638 - DNA, 23 bp, Streptococcus pyogenes
gtctcttagaggtggagagatgg

SEQ ID NO: 639 - DNA, 23 bp, Streptococcus pyogenes
tctcttagaggtggagagatggg

SEQ ID NO: 640 - DNA, 23 bp, Streptococcus pyogenes
agaggtggagagatgggagtagg

SEQ ID NO: 641 - DNA, 23 bp, Streptococcus pyogenes
ctgccttcctgactgccgattgg

SEQ ID NO: 642 - DNA, 23 bp, Streptococcus pyogenes
ccttcctgactgccgattggtgg

SEQ ID NO: 643 - DNA, 23 bp, Streptococcus pyogenes
tcctgactgccgattggtggagg

SEQ ID NO: 644 - DNA, 23 bp, Streptococcus pyogenes
gactgccgattggtggaggcagg

SEQ ID NO: 645 - DNA, 23 bp, Streptococcus pyogenes
tgccgattggtggaggcaggagg

SEQ ID NO: 646 - DNA, 23 bp, Streptococcus pyogenes
cgattggtggaggcaggaggagg

SEQ ID NO: 647 - DNA, 23 bp, Streptococcus pyogenes
aggaggaattgttgacactgtgg

SEQ ID NO: 648 - DNA, 23 bp, Streptococcus pyogenes
gtcaatatgccctgagcctgagg

SEQ ID NO: 649 - DNA, 23 bp, Streptococcus pyogenes
tcaatatgccctgagcctgaggg

SEQ ID NO: 650 - DNA, 23 bp, Streptococcus pyogenes
caccccaaaataccgccgtgtgg

SEQ ID NO: 651 - DNA, 23 bp, Streptococcus pyogenes
cccaaaataccgccgtgtggagg

SEQ ID NO: 652 - DNA, 23 bp, Streptococcus pyogenes
ccaaaataccgccgtgtggaggg

SEQ ID NO: 653 - DNA, 23 bp, Streptococcus pyogenes
caaaataccgccgtgtggagggg

SEQ ID NO: 654 - DNA, 23 bp, Streptococcus pyogenes
ccgtgtggaggggtgagccctgg

SEQ ID NO: 655 - DNA, 23 bp, Streptococcus pyogenes
cgaatgctcccactcctacctgg

SEQ ID NO: 656 - DNA, 23 bp, Streptococcus pyogenes
tgctcccactcctacctggttgg

SEQ ID NO: 657 - DNA, 23 bp, Streptococcus pyogenes
actcctacctggttggctgctgg

SEQ ID NO: 658 - DNA, 23 bp, Streptococcus pyogenes
cctggttggctgctggccagtgg

SEQ ID NO: 659 - DNA, 23 bp, Streptococcus pyogenes
tgctggccagtggtccttgatgg

SEQ ID NO: 660 - DNA, 23 bp, Streptococcus pyogenes
gctggccagtggtccttgatggg

SEQ ID NO: 661 - DNA, 23 bp, Streptococcus pyogenes
ctggccagtggtccttgatgggg

SEQ ID NO: 662 - DNA, 23 bp, Streptococcus pyogenes
atggggttgaagtcccaatctgg

SEQ ID NO: 663 - DNA, 23 bp, Streptococcus pyogenes
ccaatctggattgtttgagttgg

SEQ ID NO: 664 - DNA, 23 bp, Streptococcus pyogenes
tttgagttggctccgaatgcagg

SEQ ID NO: 665 - DNA, 23 bp, Streptococcus pyogenes
ttgagttggctccgaatgcaggg

SEQ ID NO: 666 - DNA, 23 bp, Streptococcus pyogenes
tgcagggtccaactgatgatcgg

SEQ ID NO: 667 - DNA, 23 bp, Streptococcus pyogenes
gcagggtccaactgatgatcggg

SEQ ID NO: 668 - DNA, 23 bp, Streptococcus pyogenes
tgatcgggaaagaatcccagagg

SEQ ID NO: 669 - DNA, 23 bp, Streptococcus pyogenes
gatcgggaaagaatcccagaggg

SEQ ID NO: 670 - DNA, 23 bp, Streptococcus pyogenes
gggaaagaatcccagagggttgg

SEQ ID NO: 671 - DNA, 23 bp, Streptococcus pyogenes
ggaaagaatcccagagggttggg

SEQ ID NO: 672 - DNA, 23 bp, Streptococcus pyogenes
ttcgtccccatgcctttgcgagg

SEQ ID NO: 673 - DNA, 23 bp, Streptococcus pyogenes
agctcttgttcccaagaatatgg

SEQ ID NO: 674 - DNA, 23 bp, Streptococcus pyogenes
gcaaaatgatgcgctacgtgtgg

SEQ ID NO: 675 - DNA, 23 bp, Streptococcus pyogenes
aagagtatgtaaataatgtctgg

SEQ ID NO: 676 - DNA, 23 bp, Streptococcus pyogenes
tatgtaaataatgtctggtttgg

SEQ ID NO: 677 - DNA, 23 bp, Streptococcus pyogenes
taatgattaactacctgatctgg

SEQ ID NO: 678 - DNA, 23 bp, Streptococcus pyogenes
actacctgatctggataataagg

SEQ ID NO: 679 - DNA, 23 bp, Streptococcus pyogenes
aggtttaattcctttgtctaagg

SEQ ID NO: 680 - DNA, 23 bp, Streptococcus pyogenes
ggtttaattcctttgtctaaggg

SEQ ID NO: 681 - DNA, 23 bp, Streptococcus pyogenes
ctaagggcaaatatttagtgtgg

SEQ ID NO: 682 - DNA, 23 bp, Streptococcus pyogenes
taagggcaaatatttagtgtggg

SEQ ID NO: 683 - DNA, 23 bp, Streptococcus pyogenes
ggcaaatatttagtgtgggtagg

SEQ ID NO: 684 - DNA, 23 bp, Streptococcus pyogenes
tgggtaggataaaatctagcagg

SEQ ID NO: 685 - DNA, 23 bp, Streptococcus pyogenes
ctcttttcatttacagtgagagg

SEQ ID NO: 686 - DNA, 23 bp, Streptococcus pyogenes
tcttttcatttacagtgagaggg

SEQ ID NO: 687 - DNA, 23 bp, Streptococcus pyogenes
tcctcttgtaaatgaatcttagg

SEQ ID NO: 688 - DNA, 23 bp, Streptococcus pyogenes
ttgtaaatgaatcttaggaaagg

SEQ ID NO: 689 - DNA, 23 bp, Streptococcus pyogenes
aaatgaatcttaggaaaggaagg

SEQ ID NO: 690 - DNA, 23 bp, Streptococcus pyogenes
aaggaaggagtttgccattcagg

SEQ ID NO: 691 - DNA, 23 bp, Streptococcus pyogenes
tgccattcaggattaaagatagg

SEQ ID NO: 692 - DNA, 23 bp, Streptococcus pyogenes
attaaagataggtactgtagagg

SEQ ID NO: 693 - DNA, 23 bp, Streptococcus pyogenes
tttcccaccttatgagtccaagg

SEQ ID NO: 694 - DNA, 23 bp, Streptococcus pyogenes
gattgagatctgcgtctgcgagg

SEQ ID NO: 695 - DNA, 23 bp, Streptococcus pyogenes
agatctgcgtctgcgaggcgagg

SEQ ID NO: 696 - DNA, 23 bp, Streptococcus pyogenes
gatctgcgtctgcgaggcgaggg

SEQ ID NO: 697 - DNA, 23 bp, Streptococcus pyogenes
ttcttctaggggacctgcctcgg

SEQ ID NO: 698 - DNA, 23 bp, Streptococcus pyogenes
cgtctaacaacagtagtttccgg

SEQ ID NO: 699 - DNA, 23 bp, Streptococcus pyogenes
tccggaagtgttgataagatagg

SEQ ID NO: 700 - DNA, 23 bp, Streptococcus pyogenes
ccggaagtgttgataagataggg

SEQ ID NO: 701 - DNA, 23 bp, Streptococcus pyogenes
cggaagtgttgataagatagggg

SEQ ID NO: 702 - DNA, 23 bp, Streptococcus pyogenes
gttgataagataggggcatttgg

SEQ ID NO: 703 - DNA, 23 bp, Streptococcus pyogenes
gataagataggggcatttggtgg

SEQ ID NO: 704 - DNA, 23 bp, Streptococcus pyogenes
aggggcatttggtggtctatagg

SEQ ID NO: 705 - DNA, 23 bp, Streptococcus pyogenes
gcatttggtggtctataggctgg

SEQ ID NO: 706 - DNA, 23 bp, Streptococcus pyogenes
tttggtggtctataggctggagg

SEQ ID NO: 707 - DNA, 23 bp, Streptococcus pyogenes
cagtctctcttccaaaagtaagg

SEQ ID NO: 708 - DNA, 23 bp, Streptococcus pyogenes
ataattgactactagatccctgg

SEQ ID NO: 709 - DNA, 23 bp, Streptococcus pyogenes
actactagatccctggatgctgg

SEQ ID NO: 710 - DNA, 23 bp, Streptococcus pyogenes
ttccaaattattacccacccagg

SEQ ID NO: 711 - DNA, 23 bp, Streptococcus pyogenes
caattccccccagcagagaatgg

SEQ ID NO: 712 - DNA, 23 bp, Streptococcus pyogenes
tggcttgcctgagtgcagtatgg

SEQ ID NO: 713 - DNA, 23 bp, Streptococcus pyogenes
tggtgaggtgagcaatgctcagg

SEQ ID NO: 714 - DNA, 23 bp, Streptococcus pyogenes
gcaatgctcaggagactctaagg

SEQ ID NO: 715 - DNA, 23 bp, Streptococcus pyogenes
ggcttctcgatacagagctgagg

SEQ ID NO: 716 - DNA, 23 bp, Streptococcus pyogenes
ttctcgatacagagctgaggcgg

SEQ ID NO: 717 - DNA, 23 bp, Streptococcus pyogenes
acagagctgaggcggtgtctagg

SEQ ID NO: 718 - DNA, 23 bp, Streptococcus pyogenes
ggtgtctaggagatctctgacgg

SEQ ID NO: 719 - DNA, 23 bp, Streptococcus pyogenes
tctaggagatctctgacggaagg

SEQ ID NO: 720 - DNA, 23 bp, Streptococcus pyogenes
acggaaggaaagaagtcagaagg

SEQ ID NO: 721 - DNA, 23 bp, Streptococcus pyogenes
cagcttggaggcttgaacagtgg

SEQ ID NO: 722 - DNA, 23 bp, Streptococcus pyogenes
agcttggaggcttgaacagtggg

SEQ ID NO: 723 - DNA, 23 bp, Streptococcus pyogenes
gacatgtacaagagatgattagg

SEQ ID NO: 724 - DNA, 23 bp, Streptococcus pyogenes
tacaagagatgattaggcagagg

SEQ ID NO: 725 - DNA, 23 bp, Streptococcus pyogenes
aaacacacagtctttgaagtagg

SEQ ID NO: 726 - DNA, 23 bp, Streptococcus pyogenes
agtctttgaagtaggcctcaagg

SEQ ID NO: 727 - DNA, 23 bp, Streptococcus pyogenes
tttgaagtaggcctcaaggtcgg

SEQ ID NO: 728 - DNA, 23 bp, Streptococcus pyogenes
aggtcggtcgttgacattgctgg

SEQ ID NO: 729 - DNA, 23 bp, Streptococcus pyogenes
ggtcggtcgttgacattgctggg

SEQ ID NO: 730 - DNA, 23 bp, Streptococcus pyogenes
tcttatgtaagaccttgggcagg

SEQ ID NO: 731 - DNA, 23 bp, Streptococcus pyogenes
agaccttgggcaggatctgatgg

SEQ ID NO: 732 - DNA, 23 bp, Streptococcus pyogenes
gaccttgggcaggatctgatggg

SEQ ID NO: 733 - DNA, 23 bp, Streptococcus pyogenes
caggatctgatgggcgttcacgg

SEQ ID NO: 734 - DNA, 23 bp, Streptococcus pyogenes
gatctgatgggcgttcacggtgg

SEQ ID NO: 735 - DNA, 23 bp, Streptococcus pyogenes
ggtcgccatgcaacgtgcagagg

SEQ ID NO: 736 - DNA, 23 bp, Streptococcus pyogenes
gaagcgaagtgcacacggaccgg

SEQ ID NO: 737 - DNA, 23 bp, Streptococcus pyogenes
acacggaccggcagatgagaagg

SEQ ID NO: 738 - DNA, 23 bp, Streptococcus pyogenes
cggggagaccgcgtaaagagagg

SEQ ID NO: 739 - DNA, 23 bp, Streptococcus pyogenes
aggtgcgccccgtggtcggctgg

SEQ ID NO: 740 - DNA, 23 bp, Streptococcus pyogenes
cgccccgtggtcggctggaacgg

SEQ ID NO: 741 - DNA, 23 bp, Streptococcus pyogenes
tggtcggctggaacggcagacgg

SEQ ID NO: 742 - DNA, 23 bp, Streptococcus pyogenes
gctggaacggcagacggagaagg

SEQ ID NO: 743 - DNA, 23 bp, Streptococcus pyogenes
ctggaacggcagacggagaaggg

SEQ ID NO: 744 - DNA, 23 bp, Streptococcus pyogenes
tggaacggcagacggagaagggg

SEQ ID NO: 745 - DNA, 23 bp, Streptococcus pyogenes
gacggagaaggggacgagatagg

SEQ ID NO: 746 - DNA, 23 bp, Streptococcus pyogenes
ggggacgagataggcccaagcgg

SEQ ID NO: 747 - DNA, 23 bp, Streptococcus pyogenes
taggcccaagcggccccgcgagg

SEQ ID NO: 748 - DNA, 23 bp, Streptococcus pyogenes
aggcccaagcggccccgcgaggg

SEQ ID NO: 749 - DNA, 23 bp, Streptococcus pyogenes
ggcccaagcggccccgcgagggg

SEQ ID NO: 750 - DNA, 23 bp, Streptococcus pyogenes
gccccgcgaggggtcgtccgcgg

SEQ ID NO: 751 - DNA, 23 bp, Streptococcus pyogenes
ccccgcgaggggtcgtccgcggg

SEQ ID NO: 752 - DNA, 23 bp, Streptococcus pyogenes
tccgcgggattcagcgccgacgg

SEQ ID NO: 753 - DNA, 23 bp, Streptococcus pyogenes
ccgcgggattcagcgccgacggg

SEQ ID NO: 754 - DNA, 23 bp, Streptococcus pyogenes
cgccgacgggacgtagacaaagg

SEQ ID NO: 755 - DNA, 23 bp, Streptococcus pyogenes
agacaaaggacgtcccgcgaagg

SEQ ID NO: 756 - DNA, 23 bp, Streptococcus pyogenes
cgtcccgcgaaggatccagttgg

SEQ ID NO: 757 - DNA, 23 bp, Streptococcus pyogenes
gcagtacagcctagcagccatgg

SEQ ID NO: 758 - DNA, 23 bp, Streptococcus pyogenes
acgatgtatatttccgcgagagg

SEQ ID NO: 759 - DNA, 23 bp, Streptococcus pyogenes
cgatgagctttgctccagaccgg

SEQ ID NO: 760 - DNA, 23 bp, Streptococcus pyogenes
ccggctgcgagcaaaacaagcgg

SEQ ID NO: 761 - DNA, 23 bp, Streptococcus pyogenes
gatcggcagaggagccacaaagg

SEQ ID NO: 762 - DNA, 23 bp, Streptococcus pyogenes
acgcatgcgctgatggcctatgg

SEQ ID NO: 763 - DNA, 23 bp, Streptococcus pyogenes
tatggccaagccccagccagtgg

SEQ ID NO: 764 - DNA, 23 bp, Streptococcus pyogenes
atggccaagccccagccagtggg

SEQ ID NO: 765 - DNA, 23 bp, Streptococcus pyogenes
tggccaagccccagccagtgggg

SEQ ID NO: 766 - DNA, 23 bp, Streptococcus pyogenes
ggccaagccccagccagtggggg

SEQ ID NO: 767 - DNA, 23 bp, Streptococcus pyogenes
gcaaacacttggcacagaccagg

SEQ ID NO: 768 - DNA, 23 bp, Streptococcus pyogenes
accaggccgttgccgagcaacgg

SEQ ID NO: 769 - DNA, 23 bp, Streptococcus pyogenes
ccaggccgttgccgagcaacggg

SEQ ID NO: 770 - DNA, 23 bp, Streptococcus pyogenes
caggccgttgccgagcaacgggg

SEQ ID NO: 771 - DNA, 23 bp, Streptococcus pyogenes
gttgccgagcaacggggtaaagg

SEQ ID NO: 772 - DNA, 23 bp, Streptococcus pyogenes
catgtactgtttacttagaaagg

SEQ ID NO: 773 - DNA, 23 bp, Streptococcus pyogenes
cttagaaaggccttgtaagttgg

SEQ ID NO: 774 - DNA, 23 bp, Streptococcus pyogenes
gcttgtatacatgcatacaaagg

SEQ ID NO: 775 - DNA, 23 bp, Streptococcus pyogenes
acatgcatacaaaggcattaagg

SEQ ID NO: 776 - DNA, 23 bp, Streptococcus pyogenes
gcatacaaaggcattaaggcagg

SEQ ID NO: 777 - DNA, 23 bp, Streptococcus pyogenes
ggatatccacattgtgtaaatgg

SEQ ID NO: 778 - DNA, 23 bp, Streptococcus pyogenes
caatcaataggcctgttaacagg

SEQ ID NO: 779 - DNA, 23 bp, Streptococcus pyogenes
ttttgtacaatatgatcctgtgg

SEQ ID NO: 780 - DNA, 23 bp, Streptococcus pyogenes
ttatgtaacccatgaagtttagg

SEQ ID NO: 781 - DNA, 23 bp, Streptococcus pyogenes
tatgtaacccatgaagtttaggg

SEQ ID NO: 782 - DNA, 23 bp, Streptococcus pyogenes
ccccatctttttgttttgttagg

SEQ ID NO: 783 - DNA, 23 bp, Streptococcus pyogenes
cccatctttttgttttgttaggg

SEQ ID NO: 784 - DNA, 23 bp, Streptococcus pyogenes
acccagagacaaaagaaaattgg

SEQ ID NO: 785 - DNA, 23 bp, Streptococcus pyogenes
aattggtaacagcggtataaagg

SEQ ID NO: 786 - DNA, 23 bp, Streptococcus pyogenes
attggtaacagcggtataaaggg

SEQ ID NO: 787 - DNA, 23 bp, Streptococcus pyogenes
ctcacgatgctgtacagacttgg

SEQ ID NO: 788 - DNA, 23 bp, Streptococcus pyogenes
tatagctgaaagccaaacagtgg

SEQ ID NO: 789 - DNA, 23 bp, Streptococcus pyogenes
atagctgaaagccaaacagtggg

SEQ ID NO: 790 - DNA, 23 bp, Streptococcus pyogenes
tagctgaaagccaaacagtgggg

SEQ ID NO: 791 - DNA, 23 bp, Streptococcus pyogenes
agctgaaagccaaacagtggggg

SEQ ID NO: 792 - DNA, 23 bp, Streptococcus pyogenes
gtaaactgagccaagagaaacgg

SEQ ID NO: 793 - DNA, 23 bp, Streptococcus pyogenes
gagccaagagaaacggactgagg

SEQ ID NO: 794 - DNA, 23 bp, Streptococcus pyogenes
ggactgaggcccactcccatagg

SEQ ID NO: 795 - DNA, 23 bp, Streptococcus pyogenes
taggtattttgcgaaagcccagg

SEQ ID NO: 796 - DNA, 23 bp, Streptococcus pyogenes
tttgcgaaagcccaggacgatgg

SEQ ID NO: 797 - DNA, 23 bp, Streptococcus pyogenes
ttgcgaaagcccaggacgatggg

SEQ ID NO: 798 - DNA, 23 bp, Streptococcus pyogenes
gaaagcccaggacgatgggatgg

SEQ ID NO: 799 - DNA, 23 bp, Streptococcus pyogenes
aaagcccaggacgatgggatggg

SEQ ID NO: 800 - DNA, 23 bp, Streptococcus pyogenes
ggacgatgggatgggaatacagg

SEQ ID NO: 801 - DNA, 23 bp, Streptococcus pyogenes
caggtgcaatttccatccgtagg

SEQ ID NO: 802 - DNA, 23 bp, Streptococcus pyogenes
aggttttgtacagcaacatgagg

SEQ ID NO: 803 - DNA, 23 bp, Streptococcus pyogenes
ggttttgtacagcaacatgaggg

SEQ ID NO: 804 - DNA, 23 bp, Streptococcus pyogenes
aacatagagttgccttgagcagg

SEQ ID NO: 805 - DNA, 23 bp, Streptococcus pyogenes
gccttgagcaggagtcgtgcagg

SEQ ID NO: 806 - DNA, 23 bp, Streptococcus pyogenes
ggagtcgtgcaggttttgcatgg

SEQ ID NO: 807 - DNA, 23 bp, Streptococcus pyogenes
ggttttgcatggtcccgtactgg

SEQ ID NO: 808 - DNA, 23 bp, Streptococcus pyogenes
gtactggttgttgttgatcctgg

SEQ ID NO: 809 - DNA, 23 bp, Streptococcus pyogenes
gttgttgatcctggaattagagg

SEQ ID NO: 810 - DNA, 23 bp, Streptococcus pyogenes
gaagaaccaataagaagatgagg

SEQ ID NO: 811 - DNA, 23 bp, Streptococcus pyogenes
cagcgataaccaggacaaattgg

SEQ ID NO: 812 - DNA, 23 bp, Streptococcus pyogenes
cgataaccaggacaaattggagg

SEQ ID NO: 813 - DNA, 23 bp, Streptococcus pyogenes
accaggacaaattggaggacagg

SEQ ID NO: 814 - DNA, 23 bp, Streptococcus pyogenes
aggacaaattggaggacaggagg

SEQ ID NO: 815 - DNA, 23 bp, Streptococcus pyogenes
caaattggaggacaggaggttgg

SEQ ID NO: 816 - DNA, 23 bp, Streptococcus pyogenes
acaggaggttggtgagtgattgg

SEQ ID NO: 817 - DNA, 23 bp, Streptococcus pyogenes
ggaggttggtgagtgattggagg

SEQ ID NO: 818 - DNA, 23 bp, Streptococcus pyogenes
gaattttggccaagacacacggg

SEQ ID NO: 819 - DNA, 23 bp, Streptococcus pyogenes
accccgcctgtaacacgagcagg

SEQ ID NO: 820 - DNA, 23 bp, Streptococcus pyogenes
ccccgcctgtaacacgagcaggg

SEQ ID NO: 821 - DNA, 23 bp, Streptococcus pyogenes
cccgcctgtaacacgagcagggg

SEQ ID NO: 822 - DNA, 23 bp, Streptococcus pyogenes
gtaacacgagcaggggtcctagg

SEQ ID NO: 823 - DNA, 23 bp, Streptococcus pyogenes
atgttctccatgttcgtcacagg

SEQ ID NO: 824 - DNA, 23 bp, Streptococcus pyogenes
tgttctccatgttcgtcacaggg

SEQ ID NO: 825 - DNA, 23 bp, Streptococcus pyogenes
cacagggtccccagtcctcgcgg

SEQ ID NO: 826 - DNA, 23 bp, Streptococcus pyogenes
ggagattgacgagatgtgagagg

SEQ ID NO: 827 - DNA, 23 bp, Streptococcus pyogenes
gagatgtgagaggcaatattcgg

SEQ ID NO: 828 - DNA, 23 bp, Streptococcus pyogenes
tgagaggcaatattcggagcagg

SEQ ID NO: 829 - DNA, 23 bp, Streptococcus pyogenes
gagaggcaatattcggagcaggg

SEQ ID NO: 830 - DNA, 23 bp, Streptococcus pyogenes
aaaatacagacccctgactctgg

SEQ ID NO: 831 - DNA, 23 bp, Streptococcus pyogenes
aaatacagacccctgactctggg

SEQ ID NO: 832 - DNA, 23 bp, Streptococcus pyogenes
ctctgggatcctgcagagcttgg

SEQ ID NO: 833 - DNA, 23 bp, Streptococcus pyogenes
tgggatcctgcagagcttggtgg

SEQ ID NO: 834 - DNA, 23 bp, Streptococcus pyogenes
atcctgcagagcttggtggaagg

SEQ ID NO: 835 - DNA, 23 bp, Streptococcus pyogenes
cagagcttggtggaaggcagtgg

SEQ ID NO: 836 - DNA, 23 bp, Streptococcus pyogenes
ggcagtggaattccactgcatgg

SEQ ID NO: 837 - DNA, 23 bp, Streptococcus pyogenes
ctcttcaagatcccagagtcagg

SEQ ID NO: 838 - DNA, 23 bp, Streptococcus pyogenes
tcttcaagatcccagagtcaggg

SEQ ID NO: 839 - DNA, 23 bp, Streptococcus pyogenes
agggctctgtattttcctgctgg

SEQ ID NO: 840 - DNA, 23 bp, Streptococcus pyogenes
gctctgtattttcctgctggtgg

SEQ ID NO: 841 - DNA, 23 bp, Streptococcus pyogenes
tcgtcaatcttatcgaagactgg

SEQ ID NO: 842 - DNA, 23 bp, Streptococcus pyogenes
cgtcaatcttatcgaagactggg

SEQ ID NO: 843 - DNA, 23 bp, Streptococcus pyogenes
gtcaatcttatcgaagactgggg

SEQ ID NO: 844 - DNA, 23 bp, Streptococcus pyogenes
tggggaccctgtgccgaacatgg

SEQ ID NO: 845 - DNA, 23 bp, Streptococcus pyogenes
aacatggagaacatcgcatcagg

SEQ ID NO: 846 - DNA, 23 bp, Streptococcus pyogenes
aacatcgcatcaggactcctagg

SEQ ID NO: 847 - DNA, 23 bp, Streptococcus pyogenes
taccacagagtctagactcgtgg

SEQ ID NO: 848 - DNA, 23 bp, Streptococcus pyogenes
cacagagtctagactcgtggtgg

SEQ ID NO: 849 - DNA, 23 bp, Streptococcus pyogenes
gggggaacacccgtgtgtcttgg

SEQ ID NO: 850 - DNA, 23 bp, Streptococcus pyogenes
tgttgtcctccaatttgtcctgg

SEQ ID NO: 851 - DNA, 23 bp, Streptococcus pyogenes
aggatcatcaaccaccagcacgg

SEQ ID NO: 852 - DNA, 23 bp, Streptococcus pyogenes
ggatcatcaaccaccagcacggg

SEQ ID NO: 853 - DNA, 23 bp, Streptococcus pyogenes
acctgcacaactcctgctcaagg

SEQ ID NO: 854 - DNA, 23 bp, Streptococcus pyogenes
gtattcccatcccatcatcttgg

SEQ ID NO: 855 - DNA, 23 bp, Streptococcus pyogenes
tattcccatcccatcatcttggg

SEQ ID NO: 856 - DNA, 23 bp, Streptococcus pyogenes
tagggctttcccccactgtctgg

SEQ ID NO: 857 - DNA, 23 bp, Streptococcus pyogenes
ctgtctggctttcagttatatgg

SEQ ID NO: 858 - DNA, 23 bp, Streptococcus pyogenes
accctcacaaaacaaaaagatgg

SEQ ID NO: 859 - DNA, 23 bp, Streptococcus pyogenes
ccctcacaaaacaaaaagatggg

SEQ ID NO: 860 - DNA, 23 bp, Streptococcus pyogenes
cctcacaaaacaaaaagatgggg

SEQ ID NO: 861 - DNA, 23 bp, Streptococcus pyogenes
gggatactcccttaacttcatgg

SEQ ID NO: 862 - DNA, 23 bp, Streptococcus pyogenes
ggatactcccttaacttcatggg

SEQ ID NO: 863 - DNA, 23 bp, Streptococcus pyogenes
aacttcatgggatatgtaattgg

SEQ ID NO: 864 - DNA, 23 bp, Streptococcus pyogenes
acttcatgggatatgtaattggg

SEQ ID NO: 865 - DNA, 23 bp, Streptococcus pyogenes
tgggatatgtaattgggagttgg

SEQ ID NO: 866 - DNA, 23 bp, Streptococcus pyogenes
gggatatgtaattgggagttggg

SEQ ID NO: 867 - DNA, 23 bp, Streptococcus pyogenes
ggatatgtaattgggagttgggg

SEQ ID NO: 868 - DNA, 23 bp, Streptococcus pyogenes
gagttggggaacattgccacagg

SEQ ID NO: 869 - DNA, 23 bp, Streptococcus pyogenes
ttagaaaacttcctgtaaacagg

SEQ ID NO: 870 - DNA, 23 bp, Streptococcus pyogenes
ctgtaaacaggcctattgattgg

SEQ ID NO: 871 - DNA, 23 bp, Streptococcus pyogenes
gaaagtatgtcagagaattgtgg

SEQ ID NO: 872 - DNA, 23 bp, Streptococcus pyogenes
aaagtatgtcagagaattgtggg

SEQ ID NO: 873 - DNA, 23 bp, Streptococcus pyogenes
tcagagaattgtgggtcttttgg

SEQ ID NO: 874 - DNA, 23 bp, Streptococcus pyogenes
cagagaattgtgggtcttttggg

SEQ ID NO: 875 - DNA, 23 bp, Streptococcus pyogenes
agagaattgtgggtcttttgggg

SEQ ID NO: 876 - DNA, 23 bp, Streptococcus pyogenes
tgcatgtatacaagcaaaacagg

SEQ ID NO: 877 - DNA, 23 bp, Streptococcus pyogenes
tactttctcgccaacttacaagg

SEQ ID NO: 878 - DNA, 23 bp, Streptococcus pyogenes
tgaccctttaccccgttgctcgg

SEQ ID NO: 879 - DNA, 23 bp, Streptococcus pyogenes
ctgacgcaacccccactggttgg

SEQ ID NO: 880 - DNA, 23 bp, Streptococcus pyogenes
tgacgcaacccccactggttggg

SEQ ID NO: 881 - DNA, 23 bp, Streptococcus pyogenes
gacgcaacccccactggttgggg

SEQ ID NO: 882 - DNA, 23 bp, Streptococcus pyogenes
aacccccactggttggggcttgg

SEQ ID NO: 883 - DNA, 23 bp, Streptococcus pyogenes
actggttggggcttggccatagg

SEQ ID NO: 884 - DNA, 23 bp, Streptococcus pyogenes
aggtctggagcgaaactcatcgg

SEQ ID NO: 885 - DNA, 23 bp, Streptococcus pyogenes
ggtctggagcgaaactcatcggg

SEQ ID NO: 886 - DNA, 23 bp, Streptococcus pyogenes
gcaagtatacatcatttccatgg

SEQ ID NO: 887 - DNA, 23 bp, Streptococcus pyogenes
acatcatttccatggctgctagg

SEQ ID NO: 888 - DNA, 23 bp, Streptococcus pyogenes
atcccgcggacgacccctcccgg

SEQ ID NO: 889 - DNA, 23 bp, Streptococcus pyogenes
tcccgcggacgacccctcccggg

SEQ ID NO: 890 - DNA, 23 bp, Streptococcus pyogenes
cccgcggacgacccctcccgggg

SEQ ID NO: 891 - DNA, 23 bp, Streptococcus pyogenes
cgacccctcccggggccgcttgg

SEQ ID NO: 892 - DNA, 23 bp, Streptococcus pyogenes
gacccctcccggggccgcttggg

SEQ ID NO: 893 - DNA, 23 bp, Streptococcus pyogenes
acccctcccggggccgcttgggg

SEQ ID NO: 894 - DNA, 23 bp, Streptococcus pyogenes
tctgccgtaccgaccgtccacgg

SEQ ID NO: 895 - DNA, 23 bp, Streptococcus pyogenes
ctgccgtaccgaccgtccacggg

SEQ ID NO: 896 - DNA, 23 bp, Streptococcus pyogenes
tgccgtaccgaccgtccacgggg

SEQ ID NO: 897 - DNA, 23 bp, Streptococcus pyogenes
gtctgtgccttctcgtctgccgg

SEQ ID NO: 898 - DNA, 23 bp, Streptococcus pyogenes
cttcacctctgcatgtcgcatgg

SEQ ID NO: 899 - DNA, 23 bp, Streptococcus pyogenes
agaccaccgtgaacgcccaccgg

SEQ ID NO: 900 - DNA, 23 bp, Streptococcus pyogenes
cgcccaccggaacctgcccaagg

SEQ ID NO: 901 - DNA, 23 bp, Streptococcus pyogenes
tgcccaaggtcttgcataagagg

SEQ ID NO: 902 - DNA, 23 bp, Streptococcus pyogenes
gtcttgcataagaggactcttgg

SEQ ID NO: 903 - DNA, 23 bp, Streptococcus pyogenes
aagactgtgtgtttactgagtgg

SEQ ID NO: 904 - DNA, 23 bp, Streptococcus pyogenes
agactgtgtgtttactgagtggg

SEQ ID NO: 905 - DNA, 23 bp, Streptococcus pyogenes
ctgtgtgtttactgagtgggagg

SEQ ID NO: 906 - DNA, 23 bp, Streptococcus pyogenes
gtttactgagtgggaggagctgg

SEQ ID NO: 907 - DNA, 23 bp, Streptococcus pyogenes
tttactgagtgggaggagctggg

SEQ ID NO: 908 - DNA, 23 bp, Streptococcus pyogenes
ttactgagtgggaggagctgggg

SEQ ID NO: 909 - DNA, 23 bp, Streptococcus pyogenes
tactgagtgggaggagctggggg

SEQ ID NO: 910 - DNA, 23 bp, Streptococcus pyogenes
tgagtgggaggagctgggggagg

SEQ ID NO: 911 - DNA, 23 bp, Streptococcus pyogenes
aggagctgggggaggagatgagg

SEQ ID NO: 912 - DNA, 23 bp, Streptococcus pyogenes
gggggaggagatgaggttaaagg

SEQ ID NO: 913 - DNA, 23 bp, Streptococcus pyogenes
gccttctgacttctttccgtcgg

SEQ ID NO: 914 - DNA, 23 bp, Streptococcus pyogenes
ataccgctgctgctctgtatcgg

SEQ ID NO: 915 - DNA, 23 bp, Streptococcus pyogenes
taccgctgctgctctgtatcggg

SEQ ID NO: 916 - DNA, 23 bp, Streptococcus pyogenes
cacctcaccacacagcactcagg

SEQ ID NO: 917 - DNA, 23 bp, Streptococcus pyogenes
tcaggcaagctattctgtgctgg

SEQ ID NO: 918 - DNA, 23 bp, Streptococcus pyogenes
caggcaagctattctgtgctggg

SEQ ID NO: 919 - DNA, 23 bp, Streptococcus pyogenes
aggcaagctattctgtgctgggg

SEQ ID NO: 920 - DNA, 23 bp, Streptococcus pyogenes
ggcaagctattctgtgctggggg

SEQ ID NO: 921 - DNA, 23 bp, Streptococcus pyogenes
gcaagctattctgtgctgggggg

SEQ ID NO: 922 - DNA, 23 bp, Streptococcus pyogenes
aattaatgactctagctacctgg

SEQ ID NO: 923 - DNA, 23 bp, Streptococcus pyogenes
attaatgactctagctacctggg

SEQ ID NO: 924 - DNA, 23 bp, Streptococcus pyogenes
atttagaagatccagcctccagg

SEQ ID NO: 925 - DNA, 23 bp, Streptococcus pyogenes
tttagaagatccagcctccaggg

SEQ ID NO: 926 - DNA, 23 bp, Streptococcus pyogenes
caattatgttaacactaatatgg

SEQ ID NO: 927 - DNA, 23 bp, Streptococcus pyogenes
aattatgttaacactaatatggg

SEQ ID NO: 928 - DNA, 23 bp, Streptococcus pyogenes
ctaatatgggcctaaagatcagg

SEQ ID NO: 929 - DNA, 23 bp, Streptococcus pyogenes
taaagatcaggcaattattgtgg

SEQ ID NO: 930 - DNA, 23 bp, Streptococcus pyogenes
cacatttcttgtcttacttttgg

SEQ ID NO: 931 - DNA, 23 bp, Streptococcus pyogenes
agaaactgttcttgaatatttgg

SEQ ID NO: 932 - DNA, 23 bp, Streptococcus pyogenes
cttgaatatttggtgtcttttgg

SEQ ID NO: 933 - DNA, 23 bp, Streptococcus pyogenes
atttggtgtcttttggagtgtgg

SEQ ID NO: 934 - DNA, 23 bp, Streptococcus pyogenes
atcccaatgttagtatcccttgg

SEQ ID NO: 935 - DNA, 23 bp, Streptococcus pyogenes
tagtatcccttggactcataagg

SEQ ID NO: 936 - DNA, 23 bp, Streptococcus pyogenes
tatcccttggactcataaggtgg

SEQ ID NO: 937 - DNA, 23 bp, Streptococcus pyogenes
atcccttggactcataaggtggg

SEQ ID NO: 938 - DNA, 23 bp, Streptococcus pyogenes
tacctgtctttaatcctgaatgg

SEQ ID NO: 939 - DNA, 23 bp, Streptococcus pyogenes
ttttccagacattcatttgcagg

SEQ ID NO: 940 - DNA, 23 bp, Streptococcus pyogenes
tccagacattcatttgcaggagg

SEQ ID NO: 941 - DNA, 23 bp, Streptococcus pyogenes
tgatagatgtaaacaatttgtgg

SEQ ID NO: 942 - DNA, 23 bp, Streptococcus pyogenes
gatagatgtaaacaatttgtggg

SEQ ID NO: 943 - DNA, 23 bp, Streptococcus pyogenes
cccttacagtaaatgaaaccagg

SEQ ID NO: 944 - DNA, 23 bp, Streptococcus pyogenes
aaatatttgcccttagataaagg

SEQ ID NO: 945 - DNA, 23 bp, Streptococcus pyogenes
aatatttgcccttagataaaggg

SEQ ID NO: 946 - DNA, 23 bp, Streptococcus pyogenes
caaaccttattatccagagcagg

SEQ ID NO: 947 - DNA, 23 bp, Streptococcus pyogenes
gacattatttgcatactctttgg

SEQ ID NO: 948 - DNA, 23 bp, Streptococcus pyogenes
ttatttgcatactctttggaagg

SEQ ID NO: 949 - DNA, 23 bp, Streptococcus pyogenes
tttgcatactctttggaaggcgg

SEQ ID NO: 950 - DNA, 23 bp, Streptococcus pyogenes
ttgcatactctttggaaggcggg

SEQ ID NO: 951 - DNA, 23 bp, Streptococcus pyogenes
acacatagcgcctcattttgcgg

SEQ ID NO: 952 - DNA, 23 bp, Streptococcus pyogenes
cacatagcgcctcattttgcggg

SEQ ID NO: 953 - DNA, 23 bp, Streptococcus pyogenes
aacaagatctacagcatgggagg

SEQ ID NO: 954 - DNA, 23 bp, Streptococcus pyogenes
agatctacagcatgggaggttgg

SEQ ID NO: 955 - DNA, 23 bp, Streptococcus pyogenes
tggtcttccaaacctcgaaaagg

SEQ ID NO: 956 - DNA, 23 bp, Streptococcus pyogenes
ttccaaacctcgaaaaggcatgg

SEQ ID NO: 957 - DNA, 23 bp, Streptococcus pyogenes
tccaaacctcgaaaaggcatggg

SEQ ID NO: 958 - DNA, 23 bp, Streptococcus pyogenes
ccaaacctcgaaaaggcatgggg

SEQ ID NO: 959 - DNA, 23 bp, Streptococcus pyogenes
tctttctgtccccaatcccctgg

SEQ ID NO: 960 - DNA, 23 bp, Streptococcus pyogenes
ctttctgtccccaatcccctggg

SEQ ID NO: 961 - DNA, 23 bp, Streptococcus pyogenes
attcttccccgatcatcagttgg

SEQ ID NO: 962 - DNA, 23 bp, Streptococcus pyogenes
ccaactcagaaaatccagattgg

SEQ ID NO: 963 - DNA, 23 bp, Streptococcus pyogenes
caactcagaaaatccagattggg

SEQ ID NO: 964 - DNA, 23 bp, Streptococcus pyogenes
ttgggacctcaacccacacaagg

SEQ ID NO: 965 - DNA, 23 bp, Streptococcus pyogenes
tcaacccacacaaggacaactgg

SEQ ID NO: 966 - DNA, 23 bp, Streptococcus pyogenes
cccacacaaggacaactggccgg

SEQ ID NO: 967 - DNA, 23 bp, Streptococcus pyogenes
caactggccggacgcccacaagg

SEQ ID NO: 968 - DNA, 23 bp, Streptococcus pyogenes
ctggccggacgcccacaaggtgg

SEQ ID NO: 969 - DNA, 23 bp, Streptococcus pyogenes
tggccggacgcccacaaggtggg

SEQ ID NO: 970 - DNA, 23 bp, Streptococcus pyogenes
ggacgcccacaaggtgggagtgg

SEQ ID NO: 971 - DNA, 23 bp, Streptococcus pyogenes
gacgcccacaaggtgggagtggg

SEQ ID NO: 972 - DNA, 23 bp, Streptococcus pyogenes
aaggtgggagtgggagcattcgg

SEQ ID NO: 973 - DNA, 23 bp, Streptococcus pyogenes
aggtgggagtgggagcattcggg

SEQ ID NO: 974 - DNA, 23 bp, Streptococcus pyogenes
ccagggttcacccctccccatgg

SEQ ID NO: 975 - DNA, 23 bp, Streptococcus pyogenes
cagggttcacccctccccatggg

SEQ ID NO: 976 - DNA, 23 bp, Streptococcus pyogenes
agggttcacccctccccatgggg

SEQ ID NO: 977 - DNA, 23 bp, Streptococcus pyogenes
gggttcacccctccccatggggg

SEQ ID NO: 978 - DNA, 23 bp, Streptococcus pyogenes
ccctccccatgggggactgttgg

SEQ ID NO: 979 - DNA, 23 bp, Streptococcus pyogenes
cctccccatgggggactgttggg

SEQ ID NO: 980 - DNA, 23 bp, Streptococcus pyogenes
ctccccatgggggactgttgggg

SEQ ID NO: 981 - DNA, 23 bp, Streptococcus pyogenes
cccatgggggactgttggggtgg

SEQ ID NO: 982 - DNA, 23 bp, Streptococcus pyogenes
actgttggggtggagccctcagg

SEQ ID NO: 983 - DNA, 23 bp, Streptococcus pyogenes
ctcccttatctccacctctaagg

SEQ ID NO: 984 - DNA, 23 bp, Streptococcus pyogenes
tcccttatctccacctctaaggg

SEQ ID NO: 985 - DNA, 23 bp, Streptococcus pyogenes
tctaagggacactcatcctcagg

SEQ ID NO: 986 - DNA, 23 bp, Streptococcus pyogenes
tgaggatgagtgtcccttagagg

SEQ ID NO: 987 - DNA, 23 bp, Streptococcus pyogenes
ggatgagtgtcccttagaggtgg

SEQ ID NO: 988 - DNA, 23 bp, Streptococcus pyogenes
gtcccttagaggtggagataagg

SEQ ID NO: 989 - DNA, 23 bp, Streptococcus pyogenes
tcccttagaggtggagataaggg

SEQ ID NO: 990 - DNA, 23 bp, Streptococcus pyogenes
agaggtggagataagggagttgg

SEQ ID NO: 991 - DNA, 23 bp, Streptococcus pyogenes
ttgccttcctgactgccgattgg

SEQ ID NO: 992 - DNA, 23 bp, Streptococcus pyogenes
gaggcaggaggaggagctgctgg

SEQ ID NO: 993 - DNA, 23 bp, Streptococcus pyogenes
gtgagtatgccctgagcctgagg

SEQ ID NO: 994 - DNA, 23 bp, Streptococcus pyogenes
tgagtatgccctgagcctgaggg

SEQ ID NO: 995 - DNA, 23 bp, Streptococcus pyogenes
tccaccccaacagtcccccatgg

SEQ ID NO: 996 - DNA, 23 bp, Streptococcus pyogenes
ccaccccaacagtcccccatggg

SEQ ID NO: 997 - DNA, 23 bp, Streptococcus pyogenes
caccccaacagtcccccatgggg

SEQ ID NO: 998 - DNA, 23 bp, Streptococcus pyogenes
cccaacagtcccccatggggagg

SEQ ID NO: 999 - DNA, 23 bp, Streptococcus pyogenes
ccaacagtcccccatggggaggg

SEQ ID NO: 1000 - DNA, 23 bp, Streptococcus pyogenes
caacagtcccccatggggagggg

SEQ ID NO: 1001 - DNA, 23 bp, Streptococcus pyogenes
ccatggggaggggtgaaccctgg

SEQ ID NO: 1002 - DNA, 23 bp, Streptococcus pyogenes
atgctcccactcccaccttgtgg

SEQ ID NO: 1003 - DNA, 23 bp, Streptococcus pyogenes
tgctcccactcccaccttgtggg

SEQ ID NO: 1004 - DNA, 23 bp, Streptococcus pyogenes
actcccaccttgtgggcgtccgg

SEQ ID NO: 1005 - DNA, 23 bp, Streptococcus pyogenes
tccggccagttgtccttgtgtgg

SEQ ID NO: 1006 - DNA, 23 bp, Streptococcus pyogenes
ccggccagttgtccttgtgtggg

SEQ ID NO: 1007 - DNA, 23 bp, Streptococcus pyogenes
agttgtccttgtgtgggttgagg

SEQ ID NO: 1008 - DNA, 23 bp, Streptococcus pyogenes
tgtgggttgaggtcccaatctgg

SEQ ID NO: 1009 - DNA, 23 bp, Streptococcus pyogenes
ccaatctggattttctgagttgg

SEQ ID NO: 1010 - DNA, 23 bp, Streptococcus pyogenes
tctgagttggctttgaatgcagg

SEQ ID NO: 1011 - DNA, 23 bp, Streptococcus pyogenes
ctgagttggctttgaatgcaggg

SEQ ID NO: 1012 - DNA, 23 bp, Streptococcus pyogenes
cagggtccaactgatgatcgggg

SEQ ID NO: 1013 - DNA, 23 bp, Streptococcus pyogenes
gatgatcggggaagaatcccagg

SEQ ID NO: 1014 - DNA, 23 bp, Streptococcus pyogenes
atgatcggggaagaatcccaggg

SEQ ID NO: 1015 - DNA, 23 bp, Streptococcus pyogenes
tgatcggggaagaatcccagggg

SEQ ID NO: 1016 - DNA, 23 bp, Streptococcus pyogenes
ggggaagaatcccaggggattgg

SEQ ID NO: 1017 - DNA, 23 bp, Streptococcus pyogenes
gggaagaatcccaggggattggg

SEQ ID NO: 1018 - DNA, 23 bp, Streptococcus pyogenes
ggaagaatcccaggggattgggg

SEQ ID NO: 1019 - DNA, 23 bp, Streptococcus pyogenes
tttgtccccatgccttttcgagg

SEQ ID NO: 1020 - DNA, 23 bp, Streptococcus pyogenes
ccccatgccttttcgaggtttgg

SEQ ID NO: 1021 - DNA, 23 bp, Streptococcus pyogenes
atatggtgacccgcaaaatgagg

SEQ ID NO: 1022 - DNA, 23 bp, Streptococcus pyogenes
tatgcaaataatgtctcgtctgg

SEQ ID NO: 1023 - DNA, 23 bp, Streptococcus pyogenes
taatgattaactacctgctctgg

SEQ ID NO: 1024 - DNA, 23 bp, Streptococcus pyogenes
actacctgctctggataataagg

SEQ ID NO: 1025 - DNA, 23 bp, Streptococcus pyogenes
aggtttgatccctttatctaagg

SEQ ID NO: 1026 - DNA, 23 bp, Streptococcus pyogenes
ggtttgatccctttatctaaggg

SEQ ID NO: 1027 - DNA, 23 bp, Streptococcus pyogenes
tttatctaagggcaaatatttgg

SEQ ID NO: 1028 - DNA, 23 bp, Streptococcus pyogenes
ggcaaatatttggtaacattagg

SEQ ID NO: 1029 - DNA, 23 bp, Streptococcus pyogenes
acattaggataaaatctagcagg

SEQ ID NO: 1030 - DNA, 23 bp, Streptococcus pyogenes
cattattaattttagtctcctgg

SEQ ID NO: 1031 - DNA, 23 bp, Streptococcus pyogenes
tcctggtttcatttactgtaagg

SEQ ID NO: 1032 - DNA, 23 bp, Streptococcus pyogenes
cctggtttcatttactgtaaggg

SEQ ID NO: 1033 - DNA, 23 bp, Streptococcus pyogenes
ctggtttcatttactgtaagggg

SEQ ID NO: 1034 - DNA, 23 bp, Streptococcus pyogenes
tcctcctgcaaatgaatgtctgg

SEQ ID NO: 1035 - DNA, 23 bp, Streptococcus pyogenes
aaatgaatgtctggaaaagaagg

SEQ ID NO: 1036 - DNA, 23 bp, Streptococcus pyogenes
aaagaaggagtttgccattcagg

SEQ ID NO: 1037 - DNA, 23 bp, Streptococcus pyogenes
tgccattcaggattaaagacagg

SEQ ID NO: 1038 - DNA, 23 bp, Streptococcus pyogenes
ttcccaccttatgagtccaaggg

SEQ ID NO: 1039 - DNA, 23 bp, Streptococcus pyogenes
agtccaagggatactaacattgg

SEQ ID NO: 1040 - DNA, 23 bp, Streptococcus pyogenes
gtccaagggatactaacattggg

SEQ ID NO: 1041 - DNA, 23 bp, Streptococcus pyogenes
aggggcatttggtggtctgtagg

SEQ ID NO: 1042 - DNA, 23 bp, Streptococcus pyogenes
gcatttggtggtctgtaggcagg

SEQ ID NO: 1043 - DNA, 23 bp, Streptococcus pyogenes
tttggtggtctgtaggcaggagg

SEQ ID NO: 1044 - DNA, 23 bp, Streptococcus pyogenes
acaataattgcctgatctttagg

SEQ ID NO: 1045 - DNA, 23 bp, Streptococcus pyogenes
attgactactagatccctggagg

SEQ ID NO: 1046 - DNA, 23 bp, Streptococcus pyogenes
actactagatccctggaggctgg

SEQ ID NO: 1047 - DNA, 23 bp, Streptococcus pyogenes
ttctaaattattacccacccagg

SEQ ID NO: 1048 - DNA, 23 bp, Streptococcus pyogenes
tagcttgcctgagtgctgtgtgg

SEQ ID NO: 1049 - DNA, 23 bp, Streptococcus pyogenes
tgcctgagtgctgtgtggtgagg

SEQ ID NO: 1050 - DNA, 23 bp, Streptococcus pyogenes
tggtgaggtgagcaatgttcagg

SEQ ID NO: 1051 - DNA, 23 bp, Streptococcus pyogenes
gcaatgttcaggagattctaagg

SEQ ID NO: 1052 - DNA, 23 bp, Streptococcus pyogenes
ttcccgatacagagcagcagcgg

SEQ ID NO: 1053 - DNA, 23 bp, Streptococcus pyogenes
acagagcagcagcggtatctagg

SEQ ID NO: 1054 - DNA, 23 bp, Streptococcus pyogenes
tctaggagatctcgcaccgacgg

SEQ ID NO: 1055 - DNA, 23 bp, Streptococcus pyogenes
accgacggaaagaagtcagaagg

SEQ ID NO: 1056 - DNA, 23 bp, Streptococcus pyogenes
gacatgaacaagagatgactagg

SEQ ID NO: 1057 - DNA, 23 bp, Streptococcus pyogenes
aacaagagatgactaggcagagg

SEQ ID NO: 1058 - DNA, 23 bp, Streptococcus pyogenes
agtcctcttatgcaagaccttgg

SEQ ID NO: 1059 - DNA, 23 bp, Streptococcus pyogenes
gtcctcttatgcaagaccttggg

SEQ ID NO: 1060 - DNA, 23 bp, Streptococcus pyogenes
tcttatgcaagaccttgggcagg

SEQ ID NO: 1061 - DNA, 23 bp, Streptococcus pyogenes
gcaagaccttgggcaggttccgg

SEQ ID NO: 1062 - DNA, 23 bp, Streptococcus pyogenes
agaccttgggcaggttccggtgg

SEQ ID NO: 1063 - DNA, 23 bp, Streptococcus pyogenes
gaccttgggcaggttccggtggg

SEQ ID NO: 1064 - DNA, 23 bp, Streptococcus pyogenes
caggttccggtgggcgttcacgg

SEQ ID NO: 1065 - DNA, 23 bp, Streptococcus pyogenes
gttccggtgggcgttcacggtgg

SEQ ID NO: 1066 - DNA, 23 bp, Streptococcus pyogenes
ggtctccatgcgacatgcagagg

SEQ ID NO: 1067 - DNA, 23 bp, Streptococcus pyogenes
acacggtccggcagacgagaagg

SEQ ID NO: 1068 - DNA, 23 bp, Streptococcus pyogenes
ggcagacgagaaggcacagacgg

SEQ ID NO: 1069 - DNA, 23 bp, Streptococcus pyogenes
gcagacgagaaggcacagacggg

SEQ ID NO: 1070 - DNA, 23 bp, Streptococcus pyogenes
cagacgagaaggcacagacgggg

SEQ ID NO: 1071 - DNA, 23 bp, Streptococcus pyogenes
agagaggtgcgccccgtggacgg

SEQ ID NO: 1072 - DNA, 23 bp, Streptococcus pyogenes
aggtgcgccccgtggacggtcgg

SEQ ID NO: 1073 - DNA, 23 bp, Streptococcus pyogenes
cgccccgtggacggtcggtacgg

SEQ ID NO: 1074 - DNA, 23 bp, Streptococcus pyogenes
tggacggtcggtacggcagacgg

SEQ ID NO: 1075 - DNA, 23 bp, Streptococcus pyogenes
cggtacggcagacggagaagcgg

SEQ ID NO: 1076 - DNA, 23 bp, Streptococcus pyogenes
ggtacggcagacggagaagcggg

SEQ ID NO: 1077 - DNA, 23 bp, Streptococcus pyogenes
acggcagacggagaagcgggcgg

SEQ ID NO: 1078 - DNA, 23 bp, Streptococcus pyogenes
gcgggcggtagagccccaagcgg

SEQ ID NO: 1079 - DNA, 23 bp, Streptococcus pyogenes
ggtagagccccaagcggccccgg

SEQ ID NO: 1080 - DNA, 23 bp, Streptococcus pyogenes
gtagagccccaagcggccccggg

SEQ ID NO: 1081 - DNA, 23 bp, Streptococcus pyogenes
gagccccaagcggccccgggagg

SEQ ID NO: 1082 - DNA, 23 bp, Streptococcus pyogenes
agccccaagcggccccgggaggg

SEQ ID NO: 1083 - DNA, 23 bp, Streptococcus pyogenes
gccccaagcggccccgggagggg

SEQ ID NO: 1084 - DNA, 23 bp, Streptococcus pyogenes
gccccgggaggggtcgtccgcgg

SEQ ID NO: 1085 - DNA, 23 bp, Streptococcus pyogenes
ccccgggaggggtcgtccgcggg

SEQ ID NO: 1086 - DNA, 23 bp, Streptococcus pyogenes
atggaaatgatgtatacttgcgg

SEQ ID NO: 1087 - DNA, 23 bp, Streptococcus pyogenes
tggaaatgatgtatacttgcggg

SEQ ID NO: 1088 - DNA, 23 bp, Streptococcus pyogenes
gatcggcagaggagacacaaagg

SEQ ID NO: 1089 - DNA, 23 bp, Streptococcus pyogenes
tatggccaagccccaaccagtgg

SEQ ID NO: 1090 - DNA, 23 bp, Streptococcus pyogenes
atggccaagccccaaccagtggg

SEQ ID NO: 1091 - DNA, 23 bp, Streptococcus pyogenes
tggccaagccccaaccagtgggg

SEQ ID NO: 1092 - DNA, 23 bp, Streptococcus pyogenes
ggccaagccccaaccagtggggg

SEQ ID NO: 1093 - DNA, 23 bp, Streptococcus pyogenes
ttgccgagcaacggggtaaaggg

SEQ ID NO: 1094 - DNA, 23 bp, Streptococcus pyogenes
cagatactgtttacttagaaagg

SEQ ID NO: 1095 - DNA, 23 bp, Streptococcus pyogenes
gcttgtatacatgcatataaagg

SEQ ID NO: 1096 - DNA, 23 bp, Streptococcus pyogenes
gcatataaaggcattaaagcagg

SEQ ID NO: 1097 - DNA, 23 bp, Streptococcus pyogenes
ggatatccacattgtgtaaaagg

SEQ ID NO: 1098 - DNA, 23 bp, Streptococcus pyogenes
gatatccacattgtgtaaaaggg

SEQ ID NO: 1099 - DNA, 23 bp, Streptococcus pyogenes
atatccacattgtgtaaaagggg

SEQ ID NO: 1100 - DNA, 23 bp, Streptococcus pyogenes
caatcaataggcctgtttacagg

SEQ ID NO: 1101 - DNA, 23 bp, Streptococcus pyogenes
ttttgtacaatatgttcctgtgg

SEQ ID NO: 1102 - DNA, 23 bp, Streptococcus pyogenes
ttacatatcccatgaagttaagg

SEQ ID NO: 1103 - DNA, 23 bp, Streptococcus pyogenes
tacatatcccatgaagttaaggg

SEQ ID NO: 1104 - DNA, 23 bp, Streptococcus pyogenes
ccccatctttttgttttgtgagg

SEQ ID NO: 1105 - DNA, 23 bp, Streptococcus pyogenes
cccatctttttgttttgtgaggg

SEQ ID NO: 1106 - DNA, 23 bp, Streptococcus pyogenes
aattggtaacagcggcataaagg

SEQ ID NO: 1107 - DNA, 23 bp, Streptococcus pyogenes
attggtaacagcggcataaaggg

SEQ ID NO: 1108 - DNA, 23 bp, Streptococcus pyogenes
ctcaagatgttgtacagacttgg

SEQ ID NO: 1109 - DNA, 23 bp, Streptococcus pyogenes
tataactgaaagccagacagtgg

SEQ ID NO: 1110 - DNA, 23 bp, Streptococcus pyogenes
ataactgaaagccagacagtggg

SEQ ID NO: 1111 - DNA, 23 bp, Streptococcus pyogenes
taactgaaagccagacagtgggg

SEQ ID NO: 1112 - DNA, 23 bp, Streptococcus pyogenes
aactgaaagccagacagtggggg

SEQ ID NO: 1113 - DNA, 23 bp, Streptococcus pyogenes
tttgcgaaagcccaagatgatgg

SEQ ID NO: 1114 - DNA, 23 bp, Streptococcus pyogenes
ttgcgaaagcccaagatgatggg

SEQ ID NO: 1115 - DNA, 23 bp, Streptococcus pyogenes
gaaagcccaagatgatgggatgg

SEQ ID NO: 1116 - DNA, 23 bp, Streptococcus pyogenes
aaagcccaagatgatgggatggg

SEQ ID NO: 1117 - DNA, 23 bp, Streptococcus pyogenes
agatgatgggatgggaatacagg

SEQ ID NO: 1118 - DNA, 23 bp, Streptococcus pyogenes
caggtgcagtttccatccgtagg

SEQ ID NO: 1119 - DNA, 23 bp, Streptococcus pyogenes
gcaacatgagggaaacatagagg

SEQ ID NO: 1120 - DNA, 23 bp, Streptococcus pyogenes
aacatagaggttccttgagcagg

SEQ ID NO: 1121 - DNA, 23 bp, Streptococcus pyogenes
tccttgagcaggagttgtgcagg

SEQ ID NO: 1122 - DNA, 23 bp, Streptococcus pyogenes
ggtcttgcatgatcccgtgctgg

SEQ ID NO: 1123 - DNA, 23 bp, Streptococcus pyogenes
cttgcatgatcccgtgctggtgg

SEQ ID NO: 1124 - DNA, 23 bp, Streptococcus pyogenes
gtgctggtggttgatgatcctgg

SEQ ID NO: 1125 - DNA, 23 bp, Streptococcus pyogenes
gttgatgatcctggaattagagg

SEQ ID NO: 1126 - DNA, 23 bp, Streptococcus pyogenes
ggcatagcagcaggatgcagagg

SEQ ID NO: 1127 - DNA, 23 bp, Streptococcus pyogenes
aggacaaattggaggacaacagg

SEQ ID NO: 1128 - DNA, 23 bp, Streptococcus pyogenes
caaattggaggacaacaggttgg

SEQ ID NO: 1129 - DNA, 23 bp, Streptococcus pyogenes
acaacaggttggtgagtgactgg

SEQ ID NO: 1130 - DNA, 23 bp, Streptococcus pyogenes
ttggtgagtgactggagatttgg

SEQ ID NO: 1131 - DNA, 23 bp, Streptococcus pyogenes
tggtgagtgactggagatttggg

SEQ ID NO: 1132 - DNA, 23 bp, Streptococcus pyogenes
agatttgggactgcgaattttgg

SEQ ID NO: 1133 - DNA, 23 bp, Streptococcus pyogenes
caccacgagtctagactctgtgg

SEQ ID NO: 1134 - DNA, 23 bp, Streptococcus pyogenes
ctagactctgtggtattgtgagg

SEQ ID NO: 1135 - DNA, 23 bp, Streptococcus pyogenes
gatgcgatgttctccatgttcgg

SEQ ID NO: 1136 - DNA, 23 bp, Streptococcus pyogenes
atgttctccatgttcggcacagg

SEQ ID NO: 1137 - DNA, 23 bp, Streptococcus pyogenes
tgttctccatgttcggcacaggg

SEQ ID NO: 1138 - DNA, 23 bp, Streptococcus pyogenes
cttcgataagattgacgatatgg

SEQ ID NO: 1139 - DNA, 23 bp, Streptococcus pyogenes
gcagagacagtattctgagcagg

SEQ ID NO: 1140 - DNA, 23 bp, Streptococcus pyogenes
cagagacagtattctgagcaggg

SEQ ID NO: 1141 - DNA, 23 bp, Streptococcus pyogenes
agggcttactgttcctgaactgg

SEQ ID NO: 1142 - DNA, 23 bp, Streptococcus pyogenes
aaaatacagagccctgactctgg

SEQ ID NO: 1143 - DNA, 23 bp, Streptococcus pyogenes
aaatacagagccctgactctggg

SEQ ID NO: 1144 - DNA, 23 bp, Streptococcus pyogenes
ctctgggatcttgaagagtttgg

SEQ ID NO: 1145 - DNA, 23 bp, Streptococcus pyogenes
tgggatcttgaagagtttggtgg

SEQ ID NO: 1146 - DNA, 23 bp, Streptococcus pyogenes
ttgaagagtttggtggaaagtgg

SEQ ID NO: 1147 - DNA, 23 bp, Streptococcus pyogenes
aagagtttggtggaaagtggtgg

SEQ ID NO: 1148 - DNA, 23 bp, Streptococcus pyogenes
agtggtggagttccactgcatgg

SEQ ID NO: 1149 - DNA, 23 bp, Streptococcus pyogenes
gagttccactgcatggcctgagg

SEQ ID NO: 1150 - DNA, 23 bp, Streptococcus pyogenes
ctctgctagaccccagagtgagg

SEQ ID NO: 1151 - DNA, 23 bp, Streptococcus pyogenes
tctgctagaccccagagtgaggg

SEQ ID NO: 1152 - DNA, 23 bp, Streptococcus pyogenes
ctgctagaccccagagtgagggg

SEQ ID NO: 1153 - DNA, 23 bp, Streptococcus pyogenes
aggggcctatatcttcctgctgg

SEQ ID NO: 1154 - DNA, 23 bp, Streptococcus pyogenes
ggcctatatcttcctgctggtgg

SEQ ID NO: 1155 - DNA, 23 bp, Streptococcus pyogenes
cctgctggtggctccagttccgg

SEQ ID NO: 1156 - DNA, 23 bp, Streptococcus pyogenes
ccatatcgtcaatcttctcgagg

SEQ ID NO: 1157 - DNA, 23 bp, Streptococcus pyogenes
tcgtcaatcttctcgaggactgg

SEQ ID NO: 1158 - DNA, 23 bp, Streptococcus pyogenes
cgtcaatcttctcgaggactggg

SEQ ID NO: 1159 - DNA, 23 bp, Streptococcus pyogenes
gtcaatcttctcgaggactgggg

SEQ ID NO: 1160 - DNA, 23 bp, Streptococcus pyogenes
tggggaccctgcaccgaacatgg

SEQ ID NO: 1161 - DNA, 23 bp, Streptococcus pyogenes
aacatggagaacacaacatcagg

SEQ ID NO: 1162 - DNA, 23 bp, Streptococcus pyogenes
aacacaacatcaggattcctagg

SEQ ID NO: 1163 - DNA, 23 bp, Streptococcus pyogenes
gggggagcacccacgtgtcctgg

SEQ ID NO: 1164 - DNA, 23 bp, Streptococcus pyogenes
tcttgtcctccaatttgtcctgg

SEQ ID NO: 1165 - DNA, 23 bp, Streptococcus pyogenes
caatttgtcctggctatcgctgg

SEQ ID NO: 1166 - DNA, 23 bp, Streptococcus pyogenes
gctatcgctggatgtgtctgcgg

SEQ ID NO: 1167 - DNA, 23 bp, Streptococcus pyogenes
ttggttcttctggactaccaagg

SEQ ID NO: 1168 - DNA, 23 bp, Streptococcus pyogenes
cccgtttgtcctctacttccagg

SEQ ID NO: 1169 - DNA, 23 bp, Streptococcus pyogenes
aggaacatcaactaccagcacgg

SEQ ID NO: 1170 - DNA, 23 bp, Streptococcus pyogenes
ggaacatcaactaccagcacggg

SEQ ID NO: 1171 - DNA, 23 bp, Streptococcus pyogenes
acctgcacgattcctgctcaagg

SEQ ID NO: 1172 - DNA, 23 bp, Streptococcus pyogenes
ttgttgctgtacaaaaccttcgg

SEQ ID NO: 1173 - DNA, 23 bp, Streptococcus pyogenes
tgctgtacaaaaccttcggacgg

SEQ ID NO: 1174 - DNA, 23 bp, Streptococcus pyogenes
gggctttcgcaagattcctatgg

SEQ ID NO: 1175 - DNA, 23 bp, Streptococcus pyogenes
ggctttcgcaagattcctatggg

SEQ ID NO: 1176 - DNA, 23 bp, Streptococcus pyogenes
tcgcaagattcctatgggagtgg

SEQ ID NO: 1177 - DNA, 23 bp, Streptococcus pyogenes
cgcaagattcctatgggagtggg

SEQ ID NO: 1178 - DNA, 23 bp, Streptococcus pyogenes
gggcctcagtccgtttctcctgg

SEQ ID NO: 1179 - DNA, 23 bp, Streptococcus pyogenes
accctaataaaaccaaacgttgg

SEQ ID NO: 1180 - DNA, 23 bp, Streptococcus pyogenes
ccctaataaaaccaaacgttggg

SEQ ID NO: 1181 - DNA, 23 bp, Streptococcus pyogenes
cctaataaaaccaaacgttgggg

SEQ ID NO: 1182 - DNA, 23 bp, Streptococcus pyogenes
gggctactcccttaacttcatgg

SEQ ID NO: 1183 - DNA, 23 bp, Streptococcus pyogenes
ggctactcccttaacttcatggg

SEQ ID NO: 1184 - DNA, 23 bp, Streptococcus pyogenes
tgggatatgtaattggatgttgg

SEQ ID NO: 1185 - DNA, 23 bp, Streptococcus pyogenes
gggatatgtaattggatgttggg

SEQ ID NO: 1186 - DNA, 23 bp, Streptococcus pyogenes
ggatatgtaattggatgttgggg

SEQ ID NO: 1187 - DNA, 23 bp, Streptococcus pyogenes
ctgtaaatagacctattgattgg

SEQ ID NO: 1188 - DNA, 23 bp, Streptococcus pyogenes
tgcatgtatacaatctaagcagg

SEQ ID NO: 1189 - DNA, 23 bp, Streptococcus pyogenes
cccgttgcccggcaacggtcagg

SEQ ID NO: 1190 - DNA, 23 bp, Streptococcus pyogenes
ctgacgcaacccccactggatgg

SEQ ID NO: 1191 - DNA, 23 bp, Streptococcus pyogenes
tgacgcaacccccactggatggg

SEQ ID NO: 1192 - DNA, 23 bp, Streptococcus pyogenes
gacgcaacccccactggatgggg

SEQ ID NO: 1193 - DNA, 23 bp, Streptococcus pyogenes
aacccccactggatggggcttgg

SEQ ID NO: 1194 - DNA, 23 bp, Streptococcus pyogenes
actggatggggcttggctatcgg

SEQ ID NO: 1195 - DNA, 23 bp, Streptococcus pyogenes
atcggccatcgccgcatgcgtgg

SEQ ID NO: 1196 - DNA, 23 bp, Streptococcus pyogenes
ccgcatgcgtggaacctttgtgg

SEQ ID NO: 1197 - DNA, 23 bp, Streptococcus pyogenes
tccgctgccgatccatactgcgg

SEQ ID NO: 1198 - DNA, 23 bp, Streptococcus pyogenes
cagcttgttttgctcgcagccgg

SEQ ID NO: 1199 - DNA, 23 bp, Streptococcus pyogenes
cggtctggagcgaaacttatcgg

SEQ ID NO: 1200 - DNA, 23 bp, Streptococcus pyogenes
acaactctgttgtcctctctcgg

SEQ ID NO: 1201 - DNA, 23 bp, Streptococcus pyogenes
ggaaatacacctcatttccatgg

SEQ ID NO: 1202 - DNA, 23 bp, Streptococcus pyogenes
acctcatttccatggctgctagg

SEQ ID NO: 1203 - DNA, 23 bp, Streptococcus pyogenes
cctcatttccatggctgctaggg

SEQ ID NO: 1204 - DNA, 23 bp, Streptococcus pyogenes
tgctagggtgtgctgccaactgg

SEQ ID NO: 1205 - DNA, 23 bp, Streptococcus pyogenes
atcccgcggacgacccgtctcgg

SEQ ID NO: 1206 - DNA, 23 bp, Streptococcus pyogenes
tcccgcggacgacccgtctcggg

SEQ ID NO: 1207 - DNA, 23 bp, Streptococcus pyogenes
cccgcggacgacccgtctcgggg

SEQ ID NO: 1208 - DNA, 23 bp, Streptococcus pyogenes
cgacccgtctcggggccgtttgg

SEQ ID NO: 1209 - DNA, 23 bp, Streptococcus pyogenes
gacccgtctcggggccgtttggg

SEQ ID NO: 1210 - DNA, 23 bp, Streptococcus pyogenes
cccttcttcatctgccgttccgg

SEQ ID NO: 1211 - DNA, 23 bp, Streptococcus pyogenes
tctgccgttccggccgaccacgg

SEQ ID NO: 1212 - DNA, 23 bp, Streptococcus pyogenes
ctgccgttccggccgaccacggg

SEQ ID NO: 1213 - DNA, 23 bp, Streptococcus pyogenes
tgccgttccggccgaccacgggg

SEQ ID NO: 1214 - DNA, 23 bp, Streptococcus pyogenes
aaccaccgtgaacgcccaccagg

SEQ ID NO: 1215 - DNA, 23 bp, Streptococcus pyogenes
cgcccaccaggtcttgcccaagg

SEQ ID NO: 1216 - DNA, 23 bp, Streptococcus pyogenes
tgcccaaggtcttatataagagg

SEQ ID NO: 1217 - DNA, 23 bp, Streptococcus pyogenes
gtcttatataagaggactcttgg

SEQ ID NO: 1218 - DNA, 23 bp, Streptococcus pyogenes
gatgtcaacgaccgaccttgagg

SEQ ID NO: 1219 - DNA, 23 bp, Streptococcus pyogenes
cttcaaagactgtttgtttaagg

SEQ ID NO: 1220 - DNA, 23 bp, Streptococcus pyogenes
aagactgtttgtttaaggactgg

SEQ ID NO: 1221 - DNA, 23 bp, Streptococcus pyogenes
agactgtttgtttaaggactggg

SEQ ID NO: 1222 - DNA, 23 bp, Streptococcus pyogenes
ctgtttgtttaaggactgggagg

SEQ ID NO: 1223 - DNA, 23 bp, Streptococcus pyogenes
gtttaaggactgggaggagttgg

SEQ ID NO: 1224 - DNA, 23 bp, Streptococcus pyogenes
tttaaggactgggaggagttggg

SEQ ID NO: 1225 - DNA, 23 bp, Streptococcus pyogenes
ttaaggactgggaggagttgggg

SEQ ID NO: 1226 - DNA, 23 bp, Streptococcus pyogenes
taaggactgggaggagttggggg

SEQ ID NO: 1227 - DNA, 23 bp, Streptococcus pyogenes
ggactgggaggagttgggggagg

SEQ ID NO: 1228 - DNA, 23 bp, Streptococcus pyogenes
aggttaaagatttttgtactagg

SEQ ID NO: 1229 - DNA, 23 bp, Streptococcus pyogenes
ttaaagatttttgtactaggagg

SEQ ID NO: 1230 - DNA, 23 bp, Streptococcus pyogenes
tttttgtactaggaggctgtagg

SEQ ID NO: 1231 - DNA, 23 bp, Streptococcus pyogenes
attgacccgtataaagaatttgg

SEQ ID NO: 1232 - DNA, 23 bp, Streptococcus pyogenes
taaagaatttggagcttctgtgg

SEQ ID NO: 1233 - DNA, 23 bp, Streptococcus pyogenes
acaccgcctcagctctgtatcgg

SEQ ID NO: 1234 - DNA, 23 bp, Streptococcus pyogenes
caccgcctcagctctgtatcggg

SEQ ID NO: 1235 - DNA, 23 bp, Streptococcus pyogenes
cgcctcagctctgtatcgggagg

SEQ ID NO: 1236 - DNA, 23 bp, Streptococcus pyogenes
tcgggaggccttagagtctccgg

SEQ ID NO: 1237 - DNA, 23 bp, Streptococcus pyogenes
ctcctcaccatacagcactcagg

SEQ ID NO: 1238 - DNA, 23 bp, Streptococcus pyogenes
tcaggcaagctattctgtgttgg

SEQ ID NO: 1239 - DNA, 23 bp, Streptococcus pyogenes
caggcaagctattctgtgttggg

SEQ ID NO: 1240 - DNA, 23 bp, Streptococcus pyogenes
aggcaagctattctgtgttgggg

SEQ ID NO: 1241 - DNA, 23 bp, Streptococcus pyogenes
ttggggtgagttgatgaatctgg

SEQ ID NO: 1242 - DNA, 23 bp, Streptococcus pyogenes
agttgatgaatctggccacctgg

SEQ ID NO: 1243 - DNA, 23 bp, Streptococcus pyogenes
gttgatgaatctggccacctggg

SEQ ID NO: 1244 - DNA, 23 bp, Streptococcus pyogenes
gatgaatctggccacctgggtgg

SEQ ID NO: 1245 - DNA, 23 bp, Streptococcus pyogenes
atgaatctggccacctgggtggg

SEQ ID NO: 1246 - DNA, 23 bp, Streptococcus pyogenes
cacctgggtgggaagtaatttgg

SEQ ID NO: 1247 - DNA, 23 bp, Streptococcus pyogenes
cagctatgtcaatgttaatatgg

SEQ ID NO: 1248 - DNA, 23 bp, Streptococcus pyogenes
agctatgtcaatgttaatatggg

SEQ ID NO: 1249 - DNA, 23 bp, Streptococcus pyogenes
taaaaatcagacaactactgtgg

SEQ ID NO: 1250 - DNA, 23 bp, Streptococcus pyogenes
cacatttcctgtcttacttttgg

SEQ ID NO: 1251 - DNA, 23 bp, Streptococcus pyogenes
agaaactgttcttgagtatttgg

SEQ ID NO: 1252 - DNA, 23 bp, Streptococcus pyogenes
cttgagtatttggtgtcttttgg

SEQ ID NO: 1253 - DNA, 23 bp, Streptococcus pyogenes
atctcaatgttagtatcccttgg

SEQ ID NO: 1254 - DNA, 23 bp, Streptococcus pyogenes
cataaggtgggaaactttactgg

SEQ ID NO: 1255 - DNA, 23 bp, Streptococcus pyogenes
ataaggtgggaaactttactggg

SEQ ID NO: 1256 - DNA, 23 bp, Streptococcus pyogenes
tacctgtctttaatcctgagtgg

SEQ ID NO: 1257 - DNA, 23 bp, Streptococcus pyogenes
ctttcctcacattcatttacagg

SEQ ID NO: 1258 - DNA, 23 bp, Streptococcus pyogenes
tcctcacattcatttacaggagg

SEQ ID NO: 1259 - DNA, 23 bp, Streptococcus pyogenes
taatagatgtcaacaatatgtgg

SEQ ID NO: 1260 - DNA, 23 bp, Streptococcus pyogenes
aatagatgtcaacaatatgtggg

SEQ ID NO: 1261 - DNA, 23 bp, Streptococcus pyogenes
ctcttacagttaatgaaaaaagg

SEQ ID NO: 1262 - DNA, 23 bp, Streptococcus pyogenes
taaaattaattatgcctgctagg

SEQ ID NO: 1263 - DNA, 23 bp, Streptococcus pyogenes
ccttaccaaatatttgccattgg

SEQ ID NO: 1264 - DNA, 23 bp, Streptococcus pyogenes
aaatatttgccattggacaaagg

SEQ ID NO: 1265 - DNA, 23 bp, Streptococcus pyogenes
ttaatcattacttcaaaactagg

SEQ ID NO: 1266 - DNA, 23 bp, Streptococcus pyogenes
ggcattatttacatactctgtgg

SEQ ID NO: 1267 - DNA, 23 bp, Streptococcus pyogenes
ttatttacatactctgtggaagg

SEQ ID NO: 1268 - DNA, 23 bp, Streptococcus pyogenes
tttacatactctgtggaaggcgg

SEQ ID NO: 1269 - DNA, 23 bp, Streptococcus pyogenes
ttacatactctgtggaaggcggg

SEQ ID NO: 1270 - DNA, 23 bp, Streptococcus pyogenes
acacgcagtgcctcattctgtgg

SEQ ID NO: 1271 - DNA, 23 bp, Streptococcus pyogenes
cacgcagtgcctcattctgtggg

SEQ ID NO: 1272 - DNA, 23 bp, Streptococcus pyogenes
tctgtgggtcaccatattcttgg

SEQ ID NO: 1273 - DNA, 23 bp, Streptococcus pyogenes
ctgtgggtcaccatattcttggg

SEQ ID NO: 1274 - DNA, 23 bp, Streptococcus pyogenes
tggtcttccaaacctcgacaagg

SEQ ID NO: 1275 - DNA, 23 bp, Streptococcus pyogenes
ttccaaacctcgacaaggcatgg

SEQ ID NO: 1276 - DNA, 23 bp, Streptococcus pyogenes
tccaaacctcgacaaggcatggg

SEQ ID NO: 1277 - DNA, 23 bp, Streptococcus pyogenes
ccaaacctcgacaaggcatgggg

SEQ ID NO: 1278 - DNA, 23 bp, Streptococcus pyogenes
tctttctgttcccaatcctctgg

SEQ ID NO: 1279 - DNA, 23 bp, Streptococcus pyogenes
ctttctgttcccaatcctctggg

SEQ ID NO: 1280 - DNA, 23 bp, Streptococcus pyogenes
attctttcccgatcaccagttgg

SEQ ID NO: 1281 - DNA, 23 bp, Streptococcus pyogenes
tcccgatcaccagttggacccgg

SEQ ID NO: 1282 - DNA, 23 bp, Streptococcus pyogenes
caccagttggacccggcgttcgg

SEQ ID NO: 1283 - DNA, 23 bp, Streptococcus pyogenes
ttgggacttcaaccccaacaagg

SEQ ID NO: 1284 - DNA, 23 bp, Streptococcus pyogenes
tcaaccccaacaaggatcattgg

SEQ ID NO: 1285 - DNA, 23 bp, Streptococcus pyogenes
caacaaggatcattggccagagg

SEQ ID NO: 1286 - DNA, 23 bp, Streptococcus pyogenes
tcattggccagaggcaaatcagg

SEQ ID NO: 1287 - DNA, 23 bp, Streptococcus pyogenes
tggccagaggcaaatcaggtagg

SEQ ID NO: 1288 - DNA, 23 bp, Streptococcus pyogenes
agaggcaaatcaggtaggagcgg

SEQ ID NO: 1289 - DNA, 23 bp, Streptococcus pyogenes
gaggcaaatcaggtaggagcggg

SEQ ID NO: 1290 - DNA, 23 bp, Streptococcus pyogenes
caggtaggagcgggagcattcgg

SEQ ID NO: 1291 - DNA, 23 bp, Streptococcus pyogenes
aggtaggagcgggagcattcggg

SEQ ID NO: 1292 - DNA, 23 bp, Streptococcus pyogenes
ggagcgggagcattcgggccagg

SEQ ID NO: 1293 - DNA, 23 bp, Streptococcus pyogenes
gagcgggagcattcgggccaggg

SEQ ID NO: 1294 - DNA, 23 bp, Streptococcus pyogenes
ccagggttcaccccaccacacgg

SEQ ID NO: 1295 - DNA, 23 bp, Streptococcus pyogenes
gggttcaccccaccacacggcgg

SEQ ID NO: 1296 - DNA, 23 bp, Streptococcus pyogenes
cccaccacacggcggtcttttgg

SEQ ID NO: 1297 - DNA, 23 bp, Streptococcus pyogenes
ccaccacacggcggtcttttggg

SEQ ID NO: 1298 - DNA, 23 bp, Streptococcus pyogenes
caccacacggcggtcttttgggg

SEQ ID NO: 1299 - DNA, 23 bp, Streptococcus pyogenes
cacacggcggtcttttggggtgg

SEQ ID NO: 1300 - DNA, 23 bp, Streptococcus pyogenes
tcttttggggtggagcccacagg

SEQ ID NO: 1301 - DNA, 23 bp, Streptococcus pyogenes
ggggtggagcccacaggcacagg

SEQ ID NO: 1302 - DNA, 23 bp, Streptococcus pyogenes
gggtggagcccacaggcacaggg

SEQ ID NO: 1303 - DNA, 23 bp, Streptococcus pyogenes
gtcatcctcaggccatgcaatgg

SEQ ID NO: 1304 - DNA, 23 bp, Streptococcus pyogenes
ctgtcttcctgactgccgattgg

SEQ ID NO: 1305 - DNA, 23 bp, Streptococcus pyogenes
tcttcctgactgccgattggtgg

SEQ ID NO: 1306 - DNA, 23 bp, Streptococcus pyogenes
gaggcaggaggaggtgctactgg

SEQ ID NO: 1307 - DNA, 23 bp, Streptococcus pyogenes
aggaggaggtgctactggcacgg

SEQ ID NO: 1308 - DNA, 23 bp, Streptococcus pyogenes
gtcaatacgccctgtgcctgtgg

SEQ ID NO: 1309 - DNA, 23 bp, Streptococcus pyogenes
tcaatacgccctgtgcctgtggg

SEQ ID NO: 1310 - DNA, 23 bp, Streptococcus pyogenes
caccccaaaagaccgccgtgtgg

SEQ ID NO: 1311 - DNA, 23 bp, Streptococcus pyogenes
cccaaaagaccgccgtgtggtgg

SEQ ID NO: 1312 - DNA, 23 bp, Streptococcus pyogenes
ccaaaagaccgccgtgtggtggg

SEQ ID NO: 1313 - DNA, 23 bp, Streptococcus pyogenes
caaaagaccgccgtgtggtgggg

SEQ ID NO: 1314 - DNA, 23 bp, Streptococcus pyogenes
ccgtgtggtggggtgaaccctgg

SEQ ID NO: 1315 - DNA, 23 bp, Streptococcus pyogenes
gctcctacctgatttgcctctgg

SEQ ID NO: 1316 - DNA, 23 bp, Streptococcus pyogenes
ctctggccaatgatccttgttgg

SEQ ID NO: 1317 - DNA, 23 bp, Streptococcus pyogenes
tctggccaatgatccttgttggg

SEQ ID NO: 1318 - DNA, 23 bp, Streptococcus pyogenes
ctggccaatgatccttgttgggg

SEQ ID NO: 1319 - DNA, 23 bp, Streptococcus pyogenes
ttggggttgaagtcccaatctgg

SEQ ID NO: 1320 - DNA, 23 bp, Streptococcus pyogenes
tttgagttggctccgaacgccgg

SEQ ID NO: 1321 - DNA, 23 bp, Streptococcus pyogenes
ttgagttggctccgaacgccggg

SEQ ID NO: 1322 - DNA, 23 bp, Streptococcus pyogenes
ctccgaacgccgggtccaactgg

SEQ ID NO: 1323 - DNA, 23 bp, Streptococcus pyogenes
cgccgggtccaactggtgatcgg

SEQ ID NO: 1324 - DNA, 23 bp, Streptococcus pyogenes
gccgggtccaactggtgatcggg

SEQ ID NO: 1325 - DNA, 23 bp, Streptococcus pyogenes
gggaaagaatcccagaggattgg

SEQ ID NO: 1326 - DNA, 23 bp, Streptococcus pyogenes
ggaaagaatcccagaggattggg

SEQ ID NO: 1327 - DNA, 23 bp, Streptococcus pyogenes
ttcgtccccatgccttgtcgagg

SEQ ID NO: 1328 - DNA, 23 bp, Streptococcus pyogenes
ccccatgccttgtcgaggtttgg

SEQ ID NO: 1329 - DNA, 23 bp, Streptococcus pyogenes
atatggtgacccacagaatgagg

SEQ ID NO: 1330 - DNA, 23 bp, Streptococcus pyogenes
taatgattaactgcatgttcagg

SEQ ID NO: 1331 - DNA, 23 bp, Streptococcus pyogenes
actgcatgttcaggataatatgg

SEQ ID NO: 1332 - DNA, 23 bp, Streptococcus pyogenes
ggtttaatgcctttgtccaatgg

SEQ ID NO: 1333 - DNA, 23 bp, Streptococcus pyogenes
tttgtccaatggcaaatatttgg

SEQ ID NO: 1334 - DNA, 23 bp, Streptococcus pyogenes
ccaatggcaaatatttggtaagg

SEQ ID NO: 1335 - DNA, 23 bp, Streptococcus pyogenes
ggcaaatatttggtaaggttagg

SEQ ID NO: 1336 - DNA, 23 bp, Streptococcus pyogenes
aggttaggatagaacctagcagg

SEQ ID NO: 1337 - DNA, 23 bp, Streptococcus pyogenes
cttttttcattaactgtaagagg

SEQ ID NO: 1338 - DNA, 23 bp, Streptococcus pyogenes
ttttttcattaactgtaagaggg

SEQ ID NO: 1339 - DNA, 23 bp, Streptococcus pyogenes
tcctcctgtaaatgaatgtgagg

SEQ ID NO: 1340 - DNA, 23 bp, Streptococcus pyogenes
ctgtaaatgaatgtgaggaaagg

SEQ ID NO: 1341 - DNA, 23 bp, Streptococcus pyogenes
taaatgaatgtgaggaaaggagg

SEQ ID NO: 1342 - DNA, 23 bp, Streptococcus pyogenes
aaatgaatgtgaggaaaggaggg

SEQ ID NO: 1343 - DNA, 23 bp, Streptococcus pyogenes
aaggagggagtttgccactcagg

SEQ ID NO: 1344 - DNA, 23 bp, Streptococcus pyogenes
tgccactcaggattaaagacagg

SEQ ID NO: 1345 - DNA, 23 bp, Streptococcus pyogenes
gcatttggtggtctgtaagcagg

SEQ ID NO: 1346 - DNA, 23 bp, Streptococcus pyogenes
tttggtggtctgtaagcaggagg

SEQ ID NO: 1347 - DNA, 23 bp, Streptococcus pyogenes
ttctcttccaaaagtaagacagg

SEQ ID NO: 1348 - DNA, 23 bp, Streptococcus pyogenes
acagtagttgtctgatttttagg

SEQ ID NO: 1349 - DNA, 23 bp, Streptococcus pyogenes
atagctgactactaattccctgg

SEQ ID NO: 1350 - DNA, 23 bp, Streptococcus pyogenes
actactaattccctggatgctgg

SEQ ID NO: 1351 - DNA, 23 bp, Streptococcus pyogenes
ttccaaattacttcccacccagg

SEQ ID NO: 1352 - DNA, 23 bp, Streptococcus pyogenes
caaattacttcccacccaggtgg

SEQ ID NO: 1353 - DNA, 23 bp, Streptococcus pyogenes
tagcttgcctgagtgctgtatgg

SEQ ID NO: 1354 - DNA, 23 bp, Streptococcus pyogenes
tgcctgagtgctgtatggtgagg

SEQ ID NO: 1355 - DNA, 23 bp, Streptococcus pyogenes
tggtgaggagaacaatgttccgg

SEQ ID NO: 1356 - DNA, 23 bp, Streptococcus pyogenes
acaatgttccggagactctaagg

SEQ ID NO: 1357 - DNA, 23 bp, Streptococcus pyogenes
ggcctcccgatacagagctgagg

SEQ ID NO: 1358 - DNA, 23 bp, Streptococcus pyogenes
ctcccgatacagagctgaggcgg

SEQ ID NO: 1359 - DNA, 23 bp, Streptococcus pyogenes
acagagctgaggcggtgtcgagg

SEQ ID NO: 1360 - DNA, 23 bp, Streptococcus pyogenes
tcgaggagatctcgaatagaagg

SEQ ID NO: 1361 - DNA, 23 bp, Streptococcus pyogenes
atagaaggaaagaagtcagaagg

SEQ ID NO: 1362 - DNA, 23 bp, Streptococcus pyogenes
gaagctccaaattctttatacgg

SEQ ID NO: 1363 - DNA, 23 bp, Streptococcus pyogenes
aagctccaaattctttatacggg

SEQ ID NO: 1364 - DNA, 23 bp, Streptococcus pyogenes
gacatgaacatgagatgattagg

SEQ ID NO: 1365 - DNA, 23 bp, Streptococcus pyogenes
aacatgagatgattaggcagagg

SEQ ID NO: 1366 - DNA, 23 bp, Streptococcus pyogenes
agtcctcttatataagaccttgg

SEQ ID NO: 1367 - DNA, 23 bp, Streptococcus pyogenes
gtcctcttatataagaccttggg

SEQ ID NO: 1368 - DNA, 23 bp, Streptococcus pyogenes
ataagaccttgggcaagacctgg

SEQ ID NO: 1369 - DNA, 23 bp, Streptococcus pyogenes
agaccttgggcaagacctggtgg

SEQ ID NO: 1370 - DNA, 23 bp, Streptococcus pyogenes
gaccttgggcaagacctggtggg

SEQ ID NO: 1371 - DNA, 23 bp, Streptococcus pyogenes
caagacctggtgggcgttcacgg

SEQ ID NO: 1372 - DNA, 23 bp, Streptococcus pyogenes
gacctggtgggcgttcacggtgg

SEQ ID NO: 1373 - DNA, 23 bp, Streptococcus pyogenes
ggtttccatgcgacgtgcagagg

SEQ ID NO: 1374 - DNA, 23 bp, Streptococcus pyogenes
aggtgcgccccgtggtcggccgg

SEQ ID NO: 1375 - DNA, 23 bp, Streptococcus pyogenes
cgccccgtggtcggccggaacgg

SEQ ID NO: 1376 - DNA, 23 bp, Streptococcus pyogenes
gccggaacggcagatgaagaagg

SEQ ID NO: 1377 - DNA, 23 bp, Streptococcus pyogenes
ccggaacggcagatgaagaaggg

SEQ ID NO: 1378 - DNA, 23 bp, Streptococcus pyogenes
cggaacggcagatgaagaagggg

SEQ ID NO: 1379 - DNA, 23 bp, Streptococcus pyogenes
ggggacgatagagtcccaaacgg

SEQ ID NO: 1380 - DNA, 23 bp, Streptococcus pyogenes
agtcccaaacggccccgagacgg

SEQ ID NO: 1381 - DNA, 23 bp, Streptococcus pyogenes
gtcccaaacggccccgagacggg

SEQ ID NO: 1382 - DNA, 23 bp, Streptococcus pyogenes
gccccgagacgggtcgtccgcgg

SEQ ID NO: 1383 - DNA, 23 bp, Streptococcus pyogenes
ccccgagacgggtcgtccgcggg

SEQ ID NO: 1384 - DNA, 23 bp, Streptococcus pyogenes
agacaaaggacgtcccgcgcagg

SEQ ID NO: 1385 - DNA, 23 bp, Streptococcus pyogenes
gcagcacaccctagcagccatgg

SEQ ID NO: 1386 - DNA, 23 bp, Streptococcus pyogenes
ccctagcagccatggaaatgagg

SEQ ID NO: 1387 - DNA, 23 bp, Streptococcus pyogenes
atgaggtgtatttccgagagagg

SEQ ID NO: 1388 - DNA, 23 bp, Streptococcus pyogenes
agagaggacaacagagttgtcgg

SEQ ID NO: 1389 - DNA, 23 bp, Streptococcus pyogenes
cgataagtttcgctccagaccgg

SEQ ID NO: 1390 - DNA, 23 bp, Streptococcus pyogenes
tgcgagcaaaacaagctgctagg

SEQ ID NO: 1391 - DNA, 23 bp, Streptococcus pyogenes
ctgctaggagttccgcagtatgg

SEQ ID NO: 1392 - DNA, 23 bp, Streptococcus pyogenes
tccgcagtatggatcggcagcgg

SEQ ID NO: 1393 - DNA, 23 bp, Streptococcus pyogenes
gatcggcagcggagccacaaagg

SEQ ID NO: 1394 - DNA, 23 bp, Streptococcus pyogenes
ccacaaaggttccacgcatgcgg

SEQ ID NO: 1395 - DNA, 23 bp, Streptococcus pyogenes
aggttccacgcatgcggcgatgg

SEQ ID NO: 1396 - DNA, 23 bp, Streptococcus pyogenes
gatagccaagccccatccagtgg

SEQ ID NO: 1397 - DNA, 23 bp, Streptococcus pyogenes
atagccaagccccatccagtggg

SEQ ID NO: 1398 - DNA, 23 bp, Streptococcus pyogenes
tagccaagccccatccagtgggg

SEQ ID NO: 1399 - DNA, 23 bp, Streptococcus pyogenes
agccaagccccatccagtggggg

SEQ ID NO: 1400 - DNA, 23 bp, Streptococcus pyogenes
ggcagagacctgaccgttgccgg

SEQ ID NO: 1401 - DNA, 23 bp, Streptococcus pyogenes
gcagagacctgaccgttgccggg

SEQ ID NO: 1402 - DNA, 23 bp, Streptococcus pyogenes
acctgaccgttgccgggcaacgg

SEQ ID NO: 1403 - DNA, 23 bp, Streptococcus pyogenes
cctgaccgttgccgggcaacggg

SEQ ID NO: 1404 - DNA, 23 bp, Streptococcus pyogenes
ctgaccgttgccgggcaacgggg

SEQ ID NO: 1405 - DNA, 23 bp, Streptococcus pyogenes
cagatattgtttacacagaaagg

SEQ ID NO: 1406 - DNA, 23 bp, Streptococcus pyogenes
gattgtatacatgcatataaagg

SEQ ID NO: 1407 - DNA, 23 bp, Streptococcus pyogenes
acatgcatataaaggcattaagg

SEQ ID NO: 1408 - DNA, 23 bp, Streptococcus pyogenes
gcatataaaggcattaaggcagg

SEQ ID NO: 1409 - DNA, 23 bp, Streptococcus pyogenes
ggatagccacattgtgtaaaagg

SEQ ID NO: 1410 - DNA, 23 bp, Streptococcus pyogenes
gatagccacattgtgtaaaaggg

SEQ ID NO: 1411 - DNA, 23 bp, Streptococcus pyogenes
atagccacattgtgtaaaagggg

SEQ ID NO: 1412 - DNA, 23 bp, Streptococcus pyogenes
caatcaataggtctatttacagg

SEQ ID NO: 1413 - DNA, 23 bp, Streptococcus pyogenes
tttagtacaatatgttcttgcgg

SEQ ID NO: 1414 - DNA, 23 bp, Streptococcus pyogenes
aagggagtagccccaacgtttgg

SEQ ID NO: 1415 - DNA, 23 bp, Streptococcus pyogenes
ccccaacgtttggttttattagg

SEQ ID NO: 1416 - DNA, 23 bp, Streptococcus pyogenes
cccaacgtttggttttattaggg

SEQ ID NO: 1417 - DNA, 23 bp, Streptococcus pyogenes
caaaagaaaattggtaatagagg

SEQ ID NO: 1418 - DNA, 23 bp, Streptococcus pyogenes
aattggtaatagaggtaaaaagg

SEQ ID NO: 1419 - DNA, 23 bp, Streptococcus pyogenes
attggtaatagaggtaaaaaggg

SEQ ID NO: 1420 - DNA, 23 bp, Streptococcus pyogenes
gagccaggagaaacggactgagg

SEQ ID NO: 1421 - DNA, 23 bp, Streptococcus pyogenes
taggaatcttgcgaaagcccagg

SEQ ID NO: 1422 - DNA, 23 bp, Streptococcus pyogenes
cttgcgaaagcccaggatgatgg

SEQ ID NO: 1423 - DNA, 23 bp, Streptococcus pyogenes
ttgcgaaagcccaggatgatggg

SEQ ID NO: 1424 - DNA, 23 bp, Streptococcus pyogenes
caagtgcagtttccgtccgaagg

SEQ ID NO: 1425 - DNA, 23 bp, Streptococcus pyogenes
aggttttgtacagcaacaagagg

SEQ ID NO: 1426 - DNA, 23 bp, Streptococcus pyogenes
ggttttgtacagcaacaagaggg

SEQ ID NO: 1427 - DNA, 23 bp, Streptococcus pyogenes
gcaacaagagggaaacatagagg

SEQ ID NO: 1428 - DNA, 23 bp, Streptococcus pyogenes
tccttgagcaggaatcgtgcagg

SEQ ID NO: 1429 - DNA, 23 bp, Streptococcus pyogenes
ggaatcgtgcaggtcttgcatgg

SEQ ID NO: 1430 - DNA, 23 bp, Streptococcus pyogenes
ggtcttgcatggtcccgtgctgg

SEQ ID NO: 1431 - DNA, 23 bp, Streptococcus pyogenes
gtgctggtagttgatgttcctgg

SEQ ID NO: 1432 - DNA, 23 bp, Streptococcus pyogenes
gttgatgttcctggaagtagagg

SEQ ID NO: 1433 - DNA, 23 bp, Streptococcus pyogenes
tcctggaagtagaggacaaacgg

SEQ ID NO: 1434 - DNA, 23 bp, Streptococcus pyogenes
cctggaagtagaggacaaacggg

SEQ ID NO: 1435 - DNA, 23 bp, Streptococcus pyogenes
gacaaacgggcaacataccttgg

SEQ ID NO: 1436 - DNA, 23 bp, Streptococcus pyogenes
agacacatccagcgatagccagg

SEQ ID NO: 1437 - DNA, 23 bp, Streptococcus pyogenes
cagcgatagccaggacaaattgg

SEQ ID NO: 1438 - DNA, 23 bp, Streptococcus pyogenes
cgatagccaggacaaattggagg

SEQ ID NO: 1439 - DNA, 23 bp, Streptococcus pyogenes
aggacaaattggaggacaagagg

SEQ ID NO: 1440 - DNA, 23 bp, Streptococcus pyogenes
caaattggaggacaagaggttgg

SEQ ID NO: 1441 - DNA, 23 bp, Streptococcus pyogenes
ggggactgcgaattttggccagg

SEQ ID NO: 1442 - DNA, 23 bp, Streptococcus pyogenes
cgaattttggccaggacacgtgg

SEQ ID NO: 1443 - DNA, 23 bp, Streptococcus pyogenes
gaattttggccaggacacgtggg

SEQ ID NO: 1444 - DNA, 23 bp, Streptococcus pyogenes
gatgttgtgttctccatgttcgg

SEQ ID NO: 1445 - DNA, 23 bp, Streptococcus pyogenes
gtgttctccatgttcggtgcagg

SEQ ID NO: 1446 - DNA, 23 bp, Streptococcus pyogenes
tgttctccatgttcggtgcaggg

SEQ ID NO: 1447 - DNA, 23 bp, Streptococcus pyogenes
cctcgagaagattgacgatatgg

SEQ ID NO: 1448 - DNA, 23 bp, Streptococcus pyogenes
ctcgagaagattgacgatatggg

SEQ ID NO: 1449 - DNA, 23 bp, Streptococcus pyogenes
gaagattgacgatatgggtgagg

SEQ ID NO: 1450 - DNA, 23 bp, Streptococcus pyogenes
gatatgggtgaggcagtagtcgg

SEQ ID NO: 1451 - DNA, 23 bp, Streptococcus pyogenes
ggtgaggcagtagtcggaacagg

SEQ ID NO: 1452 - DNA, 23 bp, Streptococcus pyogenes
gtgaggcagtagtcggaacaggg

SEQ ID NO: 1453 - DNA, 23 bp, Streptococcus pyogenes
cggaacagggtttactgttccgg

SEQ ID NO: 1454 - DNA, 23 bp, Streptococcus pyogenes
agggtttactgttccggaactgg

SEQ ID NO: 1455 - DNA, 23 bp, Streptococcus pyogenes
ccggaactggagccaccagcagg

SEQ ID NO: 1456 - DNA, 23 bp, Streptococcus pyogenes
agccaccagcaggaagatatagg

SEQ ID NO: 1457 - DNA, 23 bp, Streptococcus pyogenes
aagatataggcccctcactctgg

SEQ ID NO: 1458 - DNA, 23 bp, Streptococcus pyogenes
agatataggcccctcactctggg

SEQ ID NO: 1459 - DNA, 23 bp, Streptococcus pyogenes
gatataggcccctcactctgggg

SEQ ID NO: 1460 - DNA, 23 bp, Streptococcus pyogenes
ctctggggtctagcagagcttgg

SEQ ID NO: 1461 - DNA, 23 bp, Streptococcus pyogenes
tggggtctagcagagcttggtgg

SEQ ID NO: 1462 - DNA, 23 bp, Streptococcus pyogenes
cagagcttggtggaatgttgtgg

SEQ ID NO: 1463 - DNA, 23 bp, Streptococcus pyogenes
tgttgtggagttccattgcatgg

SEQ ID NO: 1464 - DNA, 23 bp, Streptococcus pyogenes
gagttccattgcatggcctgagg

SEQ ID NO: 1465 - DNA, 23 bp, Streptococcus pyogenes
tggacttctctcaattttctagg

SEQ ID NO: 1466 - DNA, 23 bp, Streptococcus pyogenes
ggacttctctcaattttctaggg

SEQ ID NO: 1467 - DNA, 23 bp, Streptococcus pyogenes
gacttctctcaattttctagggg

SEQ ID NO: 1468 - DNA, 23 bp, Streptococcus pyogenes
acttctctcaattttctaggggg

SEQ ID NO: 1469 - DNA, 23 bp, Streptococcus pyogenes
tactagtgccatttgttcagtgg

SEQ ID NO: 1470 - DNA, 23 bp, Streptococcus pyogenes
ccatttgttcagtggttcgtagg

SEQ ID NO: 1471 - DNA, 23 bp, Streptococcus pyogenes
catttgttcagtggttcgtaggg

SEQ ID NO: 1472 - DNA, 23 bp, Streptococcus pyogenes
tatatggatgatgtggtattggg

SEQ ID NO: 1473 - DNA, 23 bp, Streptococcus pyogenes
atatggatgatgtggtattgggg

SEQ ID NO: 1474 - DNA, 23 bp, Streptococcus pyogenes
tatggatgatgtggtattggggg

SEQ ID NO: 1475 - DNA, 23 bp, Streptococcus pyogenes
tttgctgacgcaacccccactgg

SEQ ID NO: 1476 - DNA, 23 bp, Streptococcus pyogenes
ggggcgcacctctctttacgcgg

SEQ ID NO: 1477 - DNA, 23 bp, Streptococcus pyogenes
aggaggctgtaggcataaattgg

SEQ ID NO: 1478 - DNA, 23 bp, Streptococcus pyogenes
caagcctccaagctgtgccttgg

SEQ ID NO: 1479 - DNA, 23 bp, Streptococcus pyogenes
aagcctccaagctgtgccttggg

SEQ ID NO: 1480 - DNA, 23 bp, Streptococcus pyogenes
cctccaagctgtgccttgggtgg

SEQ ID NO: 1481 - DNA, 23 bp, Streptococcus pyogenes
agctgtgccttgggtggctttgg

SEQ ID NO: 1482 - DNA, 23 bp, Streptococcus pyogenes
gctgtgccttgggtggctttggg

SEQ ID NO: 1483 - DNA, 23 bp, Streptococcus pyogenes
ctgtgccttgggtggctttgggg

SEQ ID NO: 1484 - DNA, 23 bp, Streptococcus pyogenes
ccttgggtggctttggggcatgg

SEQ ID NO: 1485 - DNA, 23 bp, Streptococcus pyogenes
gtcgcagaagatctcaatctcgg

SEQ ID NO: 1486 - DNA, 23 bp, Streptococcus pyogenes
tcgcagaagatctcaatctcggg

SEQ ID NO: 1487 - DNA, 23 bp, Streptococcus pyogenes
gattgagatcttctgcgacgcgg

SEQ ID NO: 1488 - DNA, 23 bp, Streptococcus pyogenes
ggcgagggagttcttcttctagg

SEQ ID NO: 1489 - DNA, 23 bp, Streptococcus pyogenes
gcgagggagttcttcttctaggg

SEQ ID NO: 1490 - DNA, 23 bp, Streptococcus pyogenes
cgagggagttcttcttctagggg

SEQ ID NO: 1491 - DNA, 23 bp, Streptococcus pyogenes
ccatgccccaaagccacccaagg

SEQ ID NO: 1492 - DNA, 23 bp, Streptococcus pyogenes
aagccacccaaggcacagcttgg

SEQ ID NO: 1493 - DNA, 23 bp, Streptococcus pyogenes
ccacccaaggcacagcttggagg

SEQ ID NO: 1494 - DNA, 23 bp, Streptococcus pyogenes
gcagaggtgaaaaagttgcatgg

SEQ ID NO: 1495 - DNA, 23 bp, Streptococcus pyogenes
gtgaaaaagttgcatggtgctgg

SEQ ID NO: 1496 - DNA, 23 bp, Streptococcus pyogenes
gaggtgaagcgaagtgcacacgg

SEQ ID NO: 1497 - DNA, 23 bp, Streptococcus pyogenes
gtaaagagaggtgcgccccgtgg

SEQ ID NO: 1498 - DNA, 23 bp, Streptococcus pyogenes
aggagttccgcagtatggatcgg

SEQ ID NO: 1499 - DNA, 23 bp, Streptococcus pyogenes
gggttgcgtcagcaaacacttgg

SEQ ID NO: 1500 - DNA, 23 bp, Streptococcus pyogenes
tgacatactttccaatcaatagg

SEQ ID NO: 1501 - DNA, 23 bp, Streptococcus pyogenes
cctacgaaccactgaacaaatgg

SEQ ID NO: 1502 - DNA, 23 bp, Streptococcus pyogenes
agaagatgaggcatagcagcagg

SEQ ID NO: 1503 - DNA, 28 bp, Neisseria meningitidis
tcccttatcgtcaatcttctcgaggatt

SEQ ID NO: 1504 - DNA, 28 bp, Neisseria meningitidis
tgaacatggagaacatcacatcaggatt

SEQ ID NO: 1505 - DNA, 28 bp, Neisseria meningitidis
aaacttcctattaacaggcctattgatt

SEQ ID NO: 1506 - DNA, 28 bp, Neisseria meningitidis
agactgggaggagttgggggaggagatt

SEQ ID NO: 1507 - DNA, 28 bp, Neisseria meningitidis
gtatttggtgtctttcggagtgtggatt

SEQ ID NO: 1508 - DNA, 28 bp, Neisseria meningitidis
cactcacagttaatgagaaaagaagatt

SEQ ID NO: 1509 - DNA, 28 bp, Neisseria meningitidis
taatgagaaaagaagattgcaattgatt

SEQ ID NO: 1510 - DNA, 28 bp, Neisseria meningitidis
atctttccaccagcaatcctctgggatt

SEQ ID NO: 1511 - DNA, 28 bp, Neisseria meningitidis
ttcagagcaaacaccgcaaatccagatt

SEQ ID NO: 1512 - DNA, 28 bp, Neisseria meningitidis
gggtaggctgccttcctgactggcgatt

SEQ ID NO: 1513 - DNA, 28 bp, Neisseria meningitidis
ggcgattggtggaggcaggaggcggatt

SEQ ID NO: 1514 - DNA, 28 bp, Neisseria meningitidis
cgtctggccaggtgtccttgttgggatt

SEQ ID NO: 1515 - DNA, 28 bp, Neisseria meningitidis
tgttgggattgaagtcccaatctggatt

SEQ ID NO: 1516 - DNA, 28 bp, Neisseria meningitidis
ggtggtcgggaaagaatcccagaggatt

SEQ ID NO: 1517 - DNA, 28 bp, Neisseria meningitidis
atcccagaggattgctggtggaaagatt

SEQ ID NO: 1518 - DNA, 28 bp, Neisseria meningitidis
aatagtgtctagtttggaagtaatgatt

SEQ ID NO: 1519 - DNA, 28 bp, Neisseria meningitidis
gaaaagatggtgttttccaatgaggatt

SEQ ID NO: 1520 - DNA, 28 bp, Neisseria meningitidis
gagtccaaggaatactaacattgagatt

SEQ ID NO: 1521 - DNA, 28 bp, Neisseria meningitidis
gaatactaacattgagattcccgagatt

SEQ ID NO: 1522 - DNA, 28 bp, Neisseria meningitidis
gattgagatcttctgcgacgcggcgatt

SEQ ID NO: 1523 - DNA, 28 bp, Neisseria meningitidis
aacagtaggacatgaacaagagatgatt

SEQ ID NO: 1524 - DNA, 28 bp, Neisseria meningitidis
gaccccgagaagggtcgtccgcaggatt

SEQ ID NO: 1525 - DNA, 28 bp, Neisseria meningitidis
ggcgagaaagtgaaagcctgcttagatt

SEQ ID NO: 1526 - DNA, 28 bp, Neisseria meningitidis
aggaagttttctaaaacattctttgatt

SEQ ID NO: 1527 - DNA, 28 bp, Neisseria meningitidis
ttggaggacaagaggttggtgagtgatt

SEQ ID NO: 1528 - DNA, 28 bp, Neisseria meningitidis
gtctagactctgcggtattgtgaggatt

SEQ ID NO: 1529 - DNA, 28 bp, Neisseria meningitidis
cgcagggtccccaatcctcgagaagatt

SEQ ID NO: 1530 - DNA, 28 bp, Neisseria meningitidis
cgaacatggagaacatcacatcaggatt

SEQ ID NO: 1531 - DNA, 28 bp, Neisseria meningitidis
ctcatcttcttattggttcttctggatt

SEQ ID NO: 1532 - DNA, 28 bp, Neisseria meningitidis
aaacttcctgttaacaggcctattgatt

SEQ ID NO: 1533 - DNA, 28 bp, Neisseria meningitidis
ggactgggaggagctgggggaggagatt

SEQ ID NO: 1534 - DNA, 28 bp, Neisseria meningitidis
atatttggtctctttcggagtgtggatt

SEQ ID NO: 1535 - DNA, 28 bp, Neisseria meningitidis
atggcaaactccttcctttcctaagatt

SEQ ID NO: 1536 - DNA, 28 bp, Neisseria meningitidis
ctctcactgtaaatgaaaagagaagatt

SEQ ID NO: 1537 - DNA, 28 bp, Neisseria meningitidis
gattgaaattaattatgcctgctagatt

SEQ ID NO: 1538 - DNA, 28 bp, Neisseria meningitidis
atctttctgttcccaaccctctgggatt

SEQ ID NO: 1539 - DNA, 28 bp, Neisseria meningitidis
ttcggagccaactcaaacaatccagatt

SEQ ID NO: 1540 - DNA, 28 bp, Neisseria meningitidis
gagtaggctgccttcctgactgccgatt

SEQ ID NO: 1541 - DNA, 28 bp, Neisseria meningitidis
tgatggggttgaagtcccaatctggatt

SEQ ID NO: 1542 - DNA, 28 bp, Neisseria meningitidis
atcccagagggttgggaacagaaagatt

SEQ ID NO: 1543 - DNA, 28 bp, Neisseria meningitidis
aataatgtctggtttggaagtaatgatt

SEQ ID NO: 1544 - DNA, 28 bp, Neisseria meningitidis
gaaaggaaggagtttgccattcaggatt

SEQ ID NO: 1545 - DNA, 28 bp, Neisseria meningitidis
aacagtgggacatgtacaagagatgatt

SEQ ID NO: 1546 - DNA, 28 bp, Neisseria meningitidis
ggccccgcgaggggtcgtccgcgggatt

SEQ ID NO: 1547 - DNA, 28 bp, Neisseria meningitidis
ttggaggacaggaggttggtgagtgatt

SEQ ID NO: 1548 - DNA, 28 bp, Neisseria meningitidis
cacagggtccccagtcctcgcggagatt

SEQ ID NO: 1549 - DNA, 28 bp, Neisseria meningitidis
aaacttcctgtaaacaggcctattgatt

SEQ ID NO: 1550 - DNA, 28 bp, Neisseria meningitidis
atatttggtgtcttttggagtgtggatt

SEQ ID NO: 1551 - DNA, 28 bp, Neisseria meningitidis
gactaaaattaataatgcctgctagatt

SEQ ID NO: 1552 - DNA, 28 bp, Neisseria meningitidis
atctttctgtccccaatcccctgggatt

SEQ ID NO: 1553 - DNA, 28 bp, Neisseria meningitidis
ttcaaagccaactcagaaaatccagatt

SEQ ID NO: 1554 - DNA, 28 bp, Neisseria meningitidis
gagttggttgccttcctgactgccgatt

SEQ ID NO: 1555 - DNA, 28 bp, Neisseria meningitidis
tgtgtgggttgaggtcccaatctggatt

SEQ ID NO: 1556 - DNA, 28 bp, Neisseria meningitidis
gatgatcggggaagaatcccaggggatt

SEQ ID NO: 1557 - DNA, 28 bp, Neisseria meningitidis
atcccaggggattggggacagaaagatt

SEQ ID NO: 1558 - DNA, 28 bp, Neisseria meningitidis
aataatgtctcgtctggaagtaatgatt

SEQ ID NO: 1559 - DNA, 28 bp, Neisseria meningitidis
gaaaagaaggagtttgccattcaggatt

SEQ ID NO: 1560 - DNA, 28 bp, Neisseria meningitidis
gagtccaagggatactaacattgggatt

SEQ ID NO: 1561 - DNA, 28 bp, Neisseria meningitidis
ggatactaacattgggattcccgagatt

SEQ ID NO: 1562 - DNA, 28 bp, Neisseria meningitidis
gattgagatcttctgcgacgcggtgatt

SEQ ID NO: 1563 - DNA, 28 bp, Neisseria meningitidis
tggtgaggtgagcaatgttcaggagatt

SEQ ID NO: 1564 - DNA, 28 bp, Neisseria meningitidis
ggccccgggaggggtcgtccgcgggatt

SEQ ID NO: 1565 - DNA, 28 bp, Neisseria meningitidis
aggaagttttctaaaacatagtttgatt

SEQ ID NO: 1566 - DNA, 28 bp, Neisseria meningitidis
acaacaggttggtgagtgactggagatt

SEQ ID NO: 1567 - DNA, 28 bp, Neisseria meningitidis
gtctagactctgtggtattgtgaggatt

SEQ ID NO: 1568 - DNA, 28 bp, Neisseria meningitidis
cacagggtccccagtcttcgataagatt

SEQ ID NO: 1569 - DNA, 28 bp, Neisseria meningitidis
cgaacatggagaacacaacatcaggatt

SEQ ID NO: 1570 - DNA, 28 bp, Neisseria meningitidis
cacgggaccatgcaagacctgcacgatt

SEQ ID NO: 1571 - DNA, 28 bp, Neisseria meningitidis
tcccatcatcctgggctttcgcaagatt

SEQ ID NO: 1572 - DNA, 28 bp, Neisseria meningitidis
aaactgcctgtaaatagacctattgatt

SEQ ID NO: 1573 - DNA, 28 bp, Neisseria meningitidis
ggactgggaggagttgggggaggagatt

SEQ ID NO: 1574 - DNA, 28 bp, Neisseria meningitidis
gttgggggaggagattaggttaaagatt

SEQ ID NO: 1575 - DNA, 28 bp, Neisseria meningitidis
gtatttggtgtcttttggagtgtggatt

SEQ ID NO: 1576 - DNA, 28 bp, Neisseria meningitidis
ctcttacagttaatgaaaaaaggagatt

SEQ ID NO: 1577 - DNA, 28 bp, Neisseria meningitidis
atctttctgttcccaatcctctgggatt

SEQ ID NO: 1578 - DNA, 28 bp, Neisseria meningitidis
gagtaggctgtcttcctgactgccgatt

SEQ ID NO: 1579 - DNA, 28 bp, Neisseria meningitidis
gcccgaatgctcccgctcctacctgatt

SEQ ID NO: 1580 - DNA, 28 bp, Neisseria meningitidis
tgttggggttgaagtcccaatctggatt

SEQ ID NO: 1581 - DNA, 28 bp, Neisseria meningitidis
ggtgatcgggaaagaatcccagaggatt

SEQ ID NO: 1582 - DNA, 28 bp, Neisseria meningitidis
atcccagaggattgggaacagaaagatt

SEQ ID NO: 1583 - DNA, 28 bp, Neisseria meningitidis
aataatgcctagttttgaagtaatgatt

SEQ ID NO: 1584 - DNA, 28 bp, Neisseria meningitidis
gaaaggagggagtttgccactcaggatt

SEQ ID NO: 1585 - DNA, 28 bp, Neisseria meningitidis
gagtccaagggatactaacattgagatt

SEQ ID NO: 1586 - DNA, 28 bp, Neisseria meningitidis
ggatactaacattgagattcccgagatt

SEQ ID NO: 1587 - DNA, 28 bp, Neisseria meningitidis
aatgtgaaaccacagtagttgtctgatt

SEQ ID NO: 1588 - DNA, 28 bp, Neisseria meningitidis
aattacttcccacccaggtggccagatt

SEQ ID NO: 1589 - DNA, 28 bp, Neisseria meningitidis
aacagtaggacatgaacatgagatgatt

SEQ ID NO: 1590 - DNA, 28 bp, Neisseria meningitidis
ggccccgagacgggtcgtccgcgggatt

SEQ ID NO: 1591 - DNA, 28 bp, Neisseria meningitidis
aggcagttttcgaaaacattgcttgatt

SEQ ID NO: 1592 - DNA, 28 bp, Neisseria meningitidis
tgcagggtccccagtcctcgagaagatt

SEQ ID NO: 1593 - DNA, 27 bp, Streptococcus thermophilus
ggcggggtttttcttgttgacaagaat

SEQ ID NO: 1594 - DNA, 27 bp, Streptococcus thermophilus
aacacatcatacaaaaaatcaaagaat

SEQ ID NO: 1595 - DNA, 27 bp, Streptococcus thermophilus
acaaaaaatcaaagaatgttttagaaa

SEQ ID NO: 1596 - DNA, 27 bp, Streptococcus thermophilus
gcatggacatcgacccttataaagaat

SEQ ID NO: 1597 - DNA, 27 bp, Streptococcus thermophilus
tttcttgtctcacttttggaagagaaa

SEQ ID NO: 1598 - DNA, 27 bp, Streptococcus thermophilus
gtaggcccactcacagttaatgagaaa

SEQ ID NO: 1599 - DNA, 27 bp, Streptococcus thermophilus
aggcgggtatattatataagagagaaa

SEQ ID NO: 1600 - DNA, 27 bp, Streptococcus thermophilus
aacaagatctacagcatggggcagaat

SEQ ID NO: 1601 - DNA, 27 bp, Streptococcus thermophilus
taccccgctgtctccacctttgagaaa

SEQ ID NO: 1602 - DNA, 27 bp, Streptococcus thermophilus
ggatccaactggtggtcgggaaagaat

SEQ ID NO: 1603 - DNA, 27 bp, Streptococcus thermophilus
catgctgtagatcttgttcccaagaat

SEQ ID NO: 1604 - DNA, 27 bp, Streptococcus thermophilus
gattaaagacaggtacagtagaagaat

SEQ ID NO: 1605 - DNA, 27 bp, Streptococcus thermophilus
tttctcttccaaaagtgagacaagaaa

SEQ ID NO: 1606 - DNA, 27 bp, Streptococcus thermophilus
agtcattagttccccccagcaaagaat

SEQ ID NO: 1607 - DNA, 27 bp, Streptococcus thermophilus
aaggttcaggtattgtttacacagaaa

SEQ ID NO: 1608 - DNA, 27 bp, Streptococcus thermophilus
agaaaggccttgtaagttggcgagaaa

SEQ ID NO: 1609 - DNA, 27 bp, Streptococcus thermophilus
taaatgtatacccaaagacaaaagaaa

SEQ ID NO: 1610 - DNA, 27 bp, Streptococcus thermophilus
ggcactagtaaactgagccaggagaaa

SEQ ID NO: 1611 - DNA, 27 bp, Streptococcus thermophilus
aagacacacggtagttccccctagaaa

SEQ ID NO: 1612 - DNA, 27 bp, Streptococcus thermophilus
attgtgaggattcttgtcaacaagaaa

SEQ ID NO: 1613 - DNA, 27 bp, Streptococcus thermophilus
acaaaagatcaaacactgtttcagaaa

SEQ ID NO: 1614 - DNA, 27 bp, Streptococcus thermophilus
tattgattggaaagtatgtcaaagaat

SEQ ID NO: 1615 - DNA, 27 bp, Streptococcus thermophilus
gcatggacattgacccttataaagaat

SEQ ID NO: 1616 - DNA, 27 bp, Streptococcus thermophilus
gggtccaactgatgatcgggaaagaat

SEQ ID NO: 1617 - DNA, 27 bp, Streptococcus thermophilus
agaatcccagagggttgggaacagaaa

SEQ ID NO: 1618 - DNA, 27 bp, Streptococcus thermophilus
catgctgtagctcttgttcccaagaat

SEQ ID NO: 1619 - DNA, 27 bp, Streptococcus thermophilus
acgtgtggtttccctcttatatagaat

SEQ ID NO: 1620 - DNA, 27 bp, Streptococcus thermophilus
agtcatcaattccccccagcagagaat

SEQ ID NO: 1621 - DNA, 27 bp, Streptococcus thermophilus
tatatttccgcgagaggacgacagaat

SEQ ID NO: 1622 - DNA, 27 bp, Streptococcus thermophilus
aaggttcatgtactgtttacttagaaa

SEQ ID NO: 1623 - DNA, 27 bp, Streptococcus thermophilus
taaatgtatacccagagacaaaagaaa

SEQ ID NO: 1624 - DNA, 27 bp, Streptococcus thermophilus
ggcactagtaaactgagccaagagaaa

SEQ ID NO: 1625 - DNA, 27 bp, Streptococcus thermophilus
aagacacacgggtgatccccctagaaa

SEQ ID NO: 1626 - DNA, 27 bp, Streptococcus thermophilus
tcaggaacagtaagccctgctcagaat

SEQ ID NO: 1627 - DNA, 27 bp, Streptococcus thermophilus
acaaaaaatcaaactatgttttagaaa

SEQ ID NO: 1628 - DNA, 27 bp, Streptococcus thermophilus
tattgattggaaagtatgtcagagaat

SEQ ID NO: 1629 - DNA, 27 bp, Streptococcus thermophilus
ctgctctgtatcgggaagccttagaat

SEQ ID NO: 1630 - DNA, 27 bp, Streptococcus thermophilus
tttcttgtcttacttttggaagagaaa

SEQ ID NO: 1631 - DNA, 27 bp, Streptococcus thermophilus
accctgcattcaaagccaactcagaaa

SEQ ID NO: 1632 - DNA, 27 bp, Streptococcus thermophilus
gggtccaactgatgatcggggaagaat

SEQ ID NO: 1633 - DNA, 27 bp, Streptococcus thermophilus
agaatcccaggggattggggacagaaa

SEQ ID NO: 1634 - DNA, 27 bp, Streptococcus thermophilus
gattaaagacaggtactgtagaagaat

SEQ ID NO: 1635 - DNA, 27 bp, Streptococcus thermophilus
tttctcttccaaaagtaagacaagaaa

SEQ ID NO: 1636 - DNA, 27 bp, Streptococcus thermophilus
agtcattaattccccccagcacagaat

SEQ ID NO: 1637 - DNA, 27 bp, Streptococcus thermophilus
tatacttgcgggagagcacgacagaat

SEQ ID NO: 1638 - DNA, 27 bp, Streptococcus thermophilus
aagggtcagatactgtttacttagaaa

SEQ ID NO: 1639 - DNA, 27 bp, Streptococcus thermophilus
aagacacacgggtgttccccctagaaa

SEQ ID NO: 1640 - DNA, 27 bp, Streptococcus thermophilus
attgtgaggatttttgtcaacaagaaa

SEQ ID NO: 1641 - DNA, 27 bp, Streptococcus thermophilus
gcatggacattgacccgtataaagaat

SEQ ID NO: 1642 - DNA, 27 bp, Streptococcus thermophilus
tttcctgtcttacttttggaagagaaa

SEQ ID NO: 1643 - DNA, 27 bp, Streptococcus thermophilus
aggcgggcattctatataagagagaaa

SEQ ID NO: 1644 - DNA, 27 bp, Streptococcus thermophilus
gggtccaactggtgatcgggaaagaat

SEQ ID NO: 1645 - DNA, 27 bp, Streptococcus thermophilus
agaatcccagaggattgggaacagaaa

SEQ ID NO: 1646 - DNA, 27 bp, Streptococcus thermophilus
tcccaagaatatggtgacccacagaat

SEQ ID NO: 1647 - DNA, 27 bp, Streptococcus thermophilus
gcgtgtagtttctctcttatatagaat

SEQ ID NO: 1648 - DNA, 27 bp, Streptococcus thermophilus
attcatcaactcaccccaacacagaat

SEQ ID NO: 1649 - DNA, 27 bp, Streptococcus thermophilus
aaggttcagatattgtttacacagaaa

SEQ ID NO: 1650 - DNA, 27 bp, Streptococcus thermophilus
caaatgtatacccaaagacaaaagaaa

SEQ ID NO: 1651 - DNA, 27 bp, Streptococcus thermophilus
aggacacgtgggtgctccccctagaaa

SEQ ID NO: 1652 - DNA, 27 bp, Streptococcus thermophilus
agaaaggccttgtaagttggcgagaaa

SEQ ID NO: 1653 - DNA, 26 bp, Streptococcus aureus
caccaaactctgcaagatcccagagt

SEQ ID NO: 1654 - DNA, 26 bp, Streptococcus aureus
cccttatcgtcaatcttctcgaggat

SEQ ID NO: 1655 - DNA, 26 bp, Streptococcus aureus
gaacatggagaacatcacatcaggat

SEQ ID NO: 1656 - DNA, 26 bp, Streptococcus aureus
accccttctcgtgttacaggcggggt

SEQ ID NO: 1657 - DNA, 26 bp, Streptococcus aureus
gcggggtttttcttgttgacaagaat

SEQ ID NO: 1658 - DNA, 26 bp, Streptococcus aureus
caagaatcctcacaataccgcagagt

SEQ ID NO: 1659 - DNA, 26 bp, Streptococcus aureus
ccaacttgtcctggttatcgctggat

SEQ ID NO: 1660 - DNA, 26 bp, Streptococcus aureus
gcccgtttgtcctctaattccaggat

SEQ ID NO: 1661 - DNA, 26 bp, Streptococcus aureus
ggctttcggaaaattcctatgggagt

SEQ ID NO: 1662 - DNA, 26 bp, Streptococcus aureus
actgtttggctttcagttatatggat

SEQ ID NO: 1663 - DNA, 26 bp, Streptococcus aureus
ggccaagtctgtacagcatcttgagt

SEQ ID NO: 1664 - DNA, 26 bp, Streptococcus aureus
ttaccaattttcttttgtctttgggt

SEQ ID NO: 1665 - DNA, 26 bp, Streptococcus aureus
accctaacaaaacaaagagatggggt

SEQ ID NO: 1666 - DNA, 26 bp, Streptococcus aureus
ggggttactctctaaattttatgggt

SEQ ID NO: 1667 - DNA, 26 bp, Streptococcus aureus
aaattttatgggttatgtcattggat

SEQ ID NO: 1668 - DNA, 26 bp, Streptococcus aureus
gggttatgtcattggatgttatgggt

SEQ ID NO: 1669 - DNA, 26 bp, Streptococcus aureus
acacatcatacaaaaaatcaaagaat

SEQ ID NO: 1670 - DNA, 26 bp, Streptococcus aureus
attgattggaaagtatgtcaacgaat

SEQ ID NO: 1671 - DNA, 26 bp, Streptococcus aureus
ggaaagtatgtcaacgaattgtgggt

SEQ ID NO: 1672 - DNA, 26 bp, Streptococcus aureus
gtcaacgaattgtgggtcttttgggt

SEQ ID NO: 1673 - DNA, 26 bp, Streptococcus aureus
ctgctaggctgtgctgccaactggat

SEQ ID NO: 1674 - DNA, 26 bp, Streptococcus aureus
ttgtttacgtcccgtcggcgctgaat

SEQ ID NO: 1675 - DNA, 26 bp, Streptococcus aureus
atcctgcggacgacccttctcggggt

SEQ ID NO: 1676 - DNA, 26 bp, Streptococcus aureus
ctgtttgtttaaagactgggaggagt

SEQ ID NO: 1677 - DNA, 26 bp, Streptococcus aureus
tcaagcctccaagctgtgccttgggt

SEQ ID NO: 1678 - DNA, 26 bp, Streptococcus aureus
catggacatcgacccttataaagaat

SEQ ID NO: 1679 - DNA, 26 bp, Streptococcus aureus
taaagaatttggagctactgtggagt

SEQ ID NO: 1680 - DNA, 26 bp, Streptococcus aureus
agctctgtatcgggaagccttagagt

SEQ ID NO: 1681 - DNA, 26 bp, Streptococcus aureus
gaactaatgactctagctacctgggt

SEQ ID NO: 1682 - DNA, 26 bp, Streptococcus aureus
taatgactctagctacctgggtgggt

SEQ ID NO: 1683 - DNA, 26 bp, Streptococcus aureus
ttttggaagagaaacagttatagagt

SEQ ID NO: 1684 - DNA, 26 bp, Streptococcus aureus
atagagtatttggtgtctttcggagt

SEQ ID NO: 1685 - DNA, 26 bp, Streptococcus aureus
tatttggtgtctttcggagtgtggat

SEQ ID NO: 1686 - DNA, 26 bp, Streptococcus aureus
tcgcagaagatctcaatctcgggaat

SEQ ID NO: 1687 - DNA, 26 bp, Streptococcus aureus
aggttaccaaatatttaccattggat

SEQ ID NO: 1688 - DNA, 26 bp, Streptococcus aureus
ccaaatatttaccattggataagggt

SEQ ID NO: 1689 - DNA, 26 bp, Streptococcus aureus
atttacacactctatggaaggcgggt

SEQ ID NO: 1690 - DNA, 26 bp, Streptococcus aureus
aacacatagcgcctcattttgtgggt

SEQ ID NO: 1691 - DNA, 26 bp, Streptococcus aureus
acaagatctacagcatggggcagaat

SEQ ID NO: 1692 - DNA, 26 bp, Streptococcus aureus
tctttccaccagcaatcctctgggat

SEQ ID NO: 1693 - DNA, 26 bp, Streptococcus aureus
gattctttcccgaccaccagttggat

SEQ ID NO: 1694 - DNA, 26 bp, Streptococcus aureus
taggagctggagcattcgggctgggt

SEQ ID NO: 1695 - DNA, 26 bp, Streptococcus aureus
cccaccgcacggaggccttttggggt

SEQ ID NO: 1696 - DNA, 26 bp, Streptococcus aureus
ttccactgcatggcctgaggatgagt

SEQ ID NO: 1697 - DNA, 26 bp, Streptococcus aureus
tttctcaaaggtggagacagcggggt

SEQ ID NO: 1698 - DNA, 26 bp, Streptococcus aureus
gcgattggtggaggcaggaggcggat

SEQ ID NO: 1699 - DNA, 26 bp, Streptococcus aureus
cccaaaaggcctccgtgcggtggggt

SEQ ID NO: 1700 - DNA, 26 bp, Streptococcus aureus
gcggtggggtgaaacccagcccgaat

SEQ ID NO: 1701 - DNA, 26 bp, Streptococcus aureus
gtctggccaggtgtccttgttgggat

SEQ ID NO: 1702 - DNA, 26 bp, Streptococcus aureus
gttgggattgaagtcccaatctggat

SEQ ID NO: 1703 - DNA, 26 bp, Streptococcus aureus
tgcggtgtttgctctgaaggctggat

SEQ ID NO: 1704 - DNA, 26 bp, Streptococcus aureus
gatccaactggtggtcgggaaagaat

SEQ ID NO: 1705 - DNA, 26 bp, Streptococcus aureus
gtggtcgggaaagaatcccagaggat

SEQ ID NO: 1706 - DNA, 26 bp, Streptococcus aureus
atgctgtagatcttgttcccaagaat

SEQ ID NO: 1707 - DNA, 26 bp, Streptococcus aureus
tataatatacccgccttccatagagt

SEQ ID NO: 1708 - DNA, 26 bp, Streptococcus aureus
gtaatgattaactagatgttctggat

SEQ ID NO: 1709 - DNA, 26 bp, Streptococcus aureus
tggtaaatatttggtaacctttggat

SEQ ID NO: 1710 - DNA, 26 bp, Streptococcus aureus
atcttcttttctcattaactgtgagt

SEQ ID NO: 1711 - DNA, 26 bp, Streptococcus aureus
aaaagatggtgttttccaatgaggat

SEQ ID NO: 1712 - DNA, 26 bp, Streptococcus aureus
attaaagacaggtacagtagaagaat

SEQ ID NO: 1713 - DNA, 26 bp, Streptococcus aureus
ccagtaaagttccccaccttatgagt

SEQ ID NO: 1714 - DNA, 26 bp, Streptococcus aureus
ttccccaccttatgagtccaaggaat

SEQ ID NO: 1715 - DNA, 26 bp, Streptococcus aureus
gaccttcgtctgcgaggcgagggagt

SEQ ID NO: 1716 - DNA, 26 bp, Streptococcus aureus
gtagtctccggaagtgttgataggat

SEQ ID NO: 1717 - DNA, 26 bp, Streptococcus aureus
tttggtggtctataagctggaggagt

SEQ ID NO: 1718 - DNA, 26 bp, Streptococcus aureus
ggtctataagctggaggagtgcgaat

SEQ ID NO: 1719 - DNA, 26 bp, Streptococcus aureus
gacaagaaatgtgaaaccacaagagt

SEQ ID NO: 1720 - DNA, 26 bp, Streptococcus aureus
gactactaggtctctagacgctggat

SEQ ID NO: 1721 - DNA, 26 bp, Streptococcus aureus
ttaacacccacccaggtagctagagt

SEQ ID NO: 1722 - DNA, 26 bp, Streptococcus aureus
gtcattagttccccccagcaaagaat

SEQ ID NO: 1723 - DNA, 26 bp, Streptococcus aureus
cccagcaaagaattgcttgcctgagt

SEQ ID NO: 1724 - DNA, 26 bp, Streptococcus aureus
agaagtcagaaggcaaaaacgagagt

SEQ ID NO: 1725 - DNA, 26 bp, Streptococcus aureus
agtagctccaaattctttataagggt

SEQ ID NO: 1726 - DNA, 26 bp, Streptococcus aureus
ggtcggtcgttgacattgctgagagt

SEQ ID NO: 1727 - DNA, 26 bp, Streptococcus aureus
gttgacattgctgagagtccaagagt

SEQ ID NO: 1728 - DNA, 26 bp, Streptococcus aureus
cagatgagaaggcacagacggggagt

SEQ ID NO: 1729 - DNA, 26 bp, Streptococcus aureus
gcagacggagaaggggacgagagagt

SEQ ID NO: 1730 - DNA, 26 bp, Streptococcus aureus
gagtcccaagcgaccccgagaagggt

SEQ ID NO: 1731 - DNA, 26 bp, Streptococcus aureus
accccgagaagggtcgtccgcaggat

SEQ ID NO: 1732 - DNA, 26 bp, Streptococcus aureus
taaacaaaggacgtcccgcgcaggat

SEQ ID NO: 1733 - DNA, 26 bp, Streptococcus aureus
atggaaacgatgtatatttgcgggat

SEQ ID NO: 1734 - DNA, 26 bp, Streptococcus aureus
atatttgcgggataggacaacagagt

SEQ ID NO: 1735 - DNA, 26 bp, Streptococcus aureus
tgcgagcaaaacaagcggctaggagt

SEQ ID NO: 1736 - DNA, 26 bp, Streptococcus aureus
gcggctaggagttccgcagtatggat

SEQ ID NO: 1737 - DNA, 26 bp, Streptococcus aureus
atgaccaagccccagccagtgggggt

SEQ ID NO: 1738 - DNA, 26 bp, Streptococcus aureus
acctggccgttgccgggcaacggggt

SEQ ID NO: 1739 - DNA, 26 bp, Streptococcus aureus
aaagtgaaagcctgcttagattgaat

SEQ ID NO: 1740 - DNA, 26 bp, Streptococcus aureus
tgcatacaaaggcatcaacgcaggat

SEQ ID NO: 1741 - DNA, 26 bp, Streptococcus aureus
gacataacccataaaatttagagagt

SEQ ID NO: 1742 - DNA, 26 bp, Streptococcus aureus
accccatctctttgttttgttagggt

SEQ ID NO: 1743 - DNA, 26 bp, Streptococcus aureus
gggctgaggcccactcccataggaat

SEQ ID NO: 1744 - DNA, 26 bp, Streptococcus aureus
ataggaattttccgaaagcccaggat

SEQ ID NO: 1745 - DNA, 26 bp, Streptococcus aureus
tttccgaaagcccaggatgatgggat

SEQ ID NO: 1746 - DNA, 26 bp, Streptococcus aureus
aaagcccaggatgatgggatgggaat

SEQ ID NO: 1747 - DNA, 26 bp, Streptococcus aureus
aggtttggtacagcaacaggagggat

SEQ ID NO: 1748 - DNA, 26 bp, Streptococcus aureus
atggtcccgtgctggttgttgaggat

SEQ ID NO: 1749 - DNA, 26 bp, Streptococcus aureus
gtgctggttgttgaggatcctggaat

SEQ ID NO: 1750 - DNA, 26 bp, Streptococcus aureus
aagaagatgaggcatagcagcaggat

SEQ ID NO: 1751 - DNA, 26 bp, Streptococcus aureus
agttggaggacaagaggttggtgagt

SEQ ID NO: 1752 - DNA, 26 bp, Streptococcus aureus
gtgattggaggttggggactgcgaat

SEQ ID NO: 1753 - DNA, 26 bp, Streptococcus aureus
aaaattgagagaagtccaccacgagt

SEQ ID NO: 1754 - DNA, 26 bp, Streptococcus aureus
tctagactctgcggtattgtgaggat

SEQ ID NO: 1755 - DNA, 26 bp, Streptococcus aureus
accccgcctgtaacacgagaaggggt

SEQ ID NO: 1756 - DNA, 26 bp, Streptococcus aureus
gtaacacgagaaggggtcctaggaat

SEQ ID NO: 1757 - DNA, 26 bp, Streptococcus aureus
gatgttctccatgttcagcgcagggt

SEQ ID NO: 1758 - DNA, 26 bp, Streptococcus aureus
gggagaggcagtagtcagaacagggt

SEQ ID NO: 1759 - DNA, 26 bp, Streptococcus aureus
gaaatacaggcctctcactctgggat

SEQ ID NO: 1760 - DNA, 26 bp, Streptococcus aureus
cctctcactctgggatcttgcagagt

SEQ ID NO: 1761 - DNA, 26 bp, Streptococcus aureus
ggaattccactgcatggcctgaggat

SEQ ID NO: 1762 - DNA, 26 bp, Streptococcus aureus
caccaagctctgctagaccccagagt

SEQ ID NO: 1763 - DNA, 26 bp, Streptococcus aureus
gaacatggagaacacaacatcaggat

SEQ ID NO: 1764 - DNA, 26 bp, Streptococcus aureus
acccctgctcgtgttacaggcggggt

SEQ ID NO: 1765 - DNA, 26 bp, Streptococcus aureus
caagaatcctcacaataccacagagt

SEQ ID NO: 1766 - DNA, 26 bp, Streptococcus aureus
ccaatttgtcctggctatcgctggat

SEQ ID NO: 1767 - DNA, 26 bp, Streptococcus aureus
ggctttcgcaagattcctatgggagt

SEQ ID NO: 1768 - DNA, 26 bp, Streptococcus aureus
ggccaagtctgtacaacatcttgagt

SEQ ID NO: 1769 - DNA, 26 bp, Streptococcus aureus
gggctactcccttaacttcatgggat

SEQ ID NO: 1770 - DNA, 26 bp, Streptococcus aureus
taacttcatgggatatgtaattggat

SEQ ID NO: 1771 - DNA, 26 bp, Streptococcus aureus
tgggatatgtaattggatgttggggt

SEQ ID NO: 1772 - DNA, 26 bp, Streptococcus aureus
attgattggaaagtatgtcagagaat

SEQ ID NO: 1773 - DNA, 26 bp, Streptococcus aureus
ggaaagtatgtcagagaattgtgggt

SEQ ID NO: 1774 - DNA, 26 bp, Streptococcus aureus
gtttgctgacgcaacccccactggat

SEQ ID NO: 1775 - DNA, 26 bp, Streptococcus aureus
cacctcatttccatggctgctagggt

SEQ ID NO: 1776 - DNA, 26 bp, Streptococcus aureus
ctgctagggtgtgctgccaactggat

SEQ ID NO: 1777 - DNA, 26 bp, Streptococcus aureus
ttgtctacgtcccgtcggcgctgaat

SEQ ID NO: 1778 - DNA, 26 bp, Streptococcus aureus
ctgtttgtttaaggactgggaggagt

SEQ ID NO: 1779 - DNA, 26 bp, Streptococcus aureus
catggacattgacccgtataaagaat

SEQ ID NO: 1780 - DNA, 26 bp, Streptococcus aureus
taaagaatttggagcttctgtggagt

SEQ ID NO: 1781 - DNA, 26 bp, Streptococcus aureus
agctctgtatcgggaggccttagagt

SEQ ID NO: 1782 - DNA, 26 bp, Streptococcus aureus
tcaggcaagctattctgtgttggggt

SEQ ID NO: 1783 - DNA, 26 bp, Streptococcus aureus
gcaagctattctgtgttggggtgagt

SEQ ID NO: 1784 - DNA, 26 bp, Streptococcus aureus
ttctgtgttggggtgagttgatgaat

SEQ ID NO: 1785 - DNA, 26 bp, Streptococcus aureus
gagttgatgaatctggccacctgggt

SEQ ID NO: 1786 - DNA, 26 bp, Streptococcus aureus
tttggaagatccagcatccagggaat

SEQ ID NO: 1787 - DNA, 26 bp, Streptococcus aureus
ttttggaagagaaactgttcttgagt

SEQ ID NO: 1788 - DNA, 26 bp, Streptococcus aureus
cttgagtatttggtgtcttttggagt

SEQ ID NO: 1789 - DNA, 26 bp, Streptococcus aureus
tatttggtgtcttttggagtgtggat

SEQ ID NO: 1790 - DNA, 26 bp, Streptococcus aureus
tactgtacctgtctttaatcctgagt

SEQ ID NO: 1791 - DNA, 26 bp, Streptococcus aureus
tacacgcagtgcctcattctgtgggt

SEQ ID NO: 1792 - DNA, 26 bp, Streptococcus aureus
aacctcgacaaggcatggggacgaat

SEQ ID NO: 1793 - DNA, 26 bp, Streptococcus aureus
tctttctgttcccaatcctctgggat

SEQ ID NO: 1794 - DNA, 26 bp, Streptococcus aureus
attgggacttcaaccccaacaaggat

SEQ ID NO: 1795 - DNA, 26 bp, Streptococcus aureus
aggagcgggagcattcgggccagggt

SEQ ID NO: 1796 - DNA, 26 bp, Streptococcus aureus
cccaccacacggcggtcttttggggt

SEQ ID NO: 1797 - DNA, 26 bp, Streptococcus aureus
tctcttagaggtggagagatgggagt

SEQ ID NO: 1798 - DNA, 26 bp, Streptococcus aureus
cccaaaagaccgccgtgtggtggggt

SEQ ID NO: 1799 - DNA, 26 bp, Streptococcus aureus
gtggtggggtgaaccctggcccgaat

SEQ ID NO: 1800 - DNA, 26 bp, Streptococcus aureus
ctctggccaatgatccttgttggggt

SEQ ID NO: 1801 - DNA, 26 bp, Streptococcus aureus
gttggggttgaagtcccaatctggat

SEQ ID NO: 1802 - DNA, 26 bp, Streptococcus aureus
gaagtcccaatctggattgtttgagt

SEQ ID NO: 1803 - DNA, 26 bp, Streptococcus aureus
gtttgagttggctccgaacgccgggt

SEQ ID NO: 1804 - DNA, 26 bp, Streptococcus aureus
ggtccaactggtgatcgggaaagaat

SEQ ID NO: 1805 - DNA, 26 bp, Streptococcus aureus
gtgatcgggaaagaatcccagaggat

SEQ ID NO: 1806 - DNA, 26 bp, Streptococcus aureus
atgctgtagctcttgttcccaagaat

SEQ ID NO: 1807 - DNA, 26 bp, Streptococcus aureus
cccaagaatatggtgacccacagaat

SEQ ID NO: 1808 - DNA, 26 bp, Streptococcus aureus
cgtgtagtttctctcttatatagaat

SEQ ID NO: 1809 - DNA, 26 bp, Streptococcus aureus
tatagaatgcccgccttccacagagt

SEQ ID NO: 1810 - DNA, 26 bp, Streptococcus aureus
gtaatgattaactgcatgttcaggat

SEQ ID NO: 1811 - DNA, 26 bp, Streptococcus aureus
tggcaaatatttggtaaggttaggat

SEQ ID NO: 1812 - DNA, 26 bp, Streptococcus aureus
ttaataatgtcctcctgtaaatgaat

SEQ ID NO: 1813 - DNA, 26 bp, Streptococcus aureus
aaatgaatgtgaggaaaggagggagt

SEQ ID NO: 1814 - DNA, 26 bp, Streptococcus aureus
aaaggagggagtttgccactcaggat

SEQ ID NO: 1815 - DNA, 26 bp, Streptococcus aureus
ccagtaaagtttcccaccttatgagt

SEQ ID NO: 1816 - DNA, 26 bp, Streptococcus aureus
tttcccaccttatgagtccaagggat

SEQ ID NO: 1817 - DNA, 26 bp, Streptococcus aureus
tttggtggtctgtaagcaggaggagt

SEQ ID NO: 1818 - DNA, 26 bp, Streptococcus aureus
ggtctgtaagcaggaggagtgcgaat

SEQ ID NO: 1819 - DNA, 26 bp, Streptococcus aureus
catagctgactactaattccctggat

SEQ ID NO: 1820 - DNA, 26 bp, Streptococcus aureus
gactactaattccctggatgctggat

SEQ ID NO: 1821 - DNA, 26 bp, Streptococcus aureus
ttcatcaactcaccccaacacagaat

SEQ ID NO: 1822 - DNA, 26 bp, Streptococcus aureus
cccaacacagaatagcttgcctgagt

SEQ ID NO: 1823 - DNA, 26 bp, Streptococcus aureus
gaggcggtgtcgaggagatctcgaat

SEQ ID NO: 1824 - DNA, 26 bp, Streptococcus aureus
agaagtcagaaggcaaaaaagagagt

SEQ ID NO: 1825 - DNA, 26 bp, Streptococcus aureus
agaagctccaaattctttatacgggt

SEQ ID NO: 1826 - DNA, 26 bp, Streptococcus aureus
ggtcggtcgttgacatcgctgagagt

SEQ ID NO: 1827 - DNA, 26 bp, Streptococcus aureus
gttgacatcgctgagagtccaagagt

SEQ ID NO: 1828 - DNA, 26 bp, Streptococcus aureus
gcagatgaagaaggggacgatagagt

SEQ ID NO: 1829 - DNA, 26 bp, Streptococcus aureus
gagtcccaaacggccccgagacgggt

SEQ ID NO: 1830 - DNA, 26 bp, Streptococcus aureus
gccccgagacgggtcgtccgcgggat

SEQ ID NO: 1831 - DNA, 26 bp, Streptococcus aureus
tagacaaaggacgtcccgcgcaggat

SEQ ID NO: 1832 - DNA, 26 bp, Streptococcus aureus
gtatttccgagagaggacaacagagt

SEQ ID NO: 1833 - DNA, 26 bp, Streptococcus aureus
tgcgagcaaaacaagctgctaggagt

SEQ ID NO: 1834 - DNA, 26 bp, Streptococcus aureus
gctgctaggagttccgcagtatggat

SEQ ID NO: 1835 - DNA, 26 bp, Streptococcus aureus
atagccaagccccatccagtgggggt

SEQ ID NO: 1836 - DNA, 26 bp, Streptococcus aureus
acctgaccgttgccgggcaacggggt

SEQ ID NO: 1837 - DNA, 26 bp, Streptococcus aureus
tgcatataaaggcattaaggcaggat

SEQ ID NO: 1838 - DNA, 26 bp, Streptococcus aureus
tacatatcccatgaagttaagggagt

SEQ ID NO: 1839 - DNA, 26 bp, Streptococcus aureus
gccccaacgtttggttttattagggt

SEQ ID NO: 1840 - DNA, 26 bp, Streptococcus aureus
ggactgaggcccactcccataggaat

SEQ ID NO: 1841 - DNA, 26 bp, Streptococcus aureus
ataggaatcttgcgaaagcccaggat

SEQ ID NO: 1842 - DNA, 26 bp, Streptococcus aureus
cttgcgaaagcccaggatgatgggat

SEQ ID NO: 1843 - DNA, 26 bp, Streptococcus aureus
aacatagaggttccttgagcaggaat

SEQ ID NO: 1844 - DNA, 26 bp, Streptococcus aureus
ggcatagcagcaggatgaagaggaat

SEQ ID NO: 1845 - DNA, 26 bp, Streptococcus aureus
aattggaggacaagaggttggtgagt

SEQ ID NO: 1846 - DNA, 26 bp, Streptococcus aureus
gcgaattttggccaggacacgtgggt

SEQ ID NO: 1847 - DNA, 26 bp, Streptococcus aureus
tctagactctgtggtattgtgaggat

SEQ ID NO: 1848 - DNA, 26 bp, Streptococcus aureus
accccgcctgtaacacgagcaggggt

SEQ ID NO: 1849 - DNA, 26 bp, Streptococcus aureus
gtaacacgagcaggggtcctaggaat

SEQ ID NO: 1850 - DNA, 26 bp, Streptococcus aureus
tgtgttctccatgttcggtgcagggt

SEQ ID NO: 1851 - DNA, 26 bp, Streptococcus aureus
tcctcgagaagattgacgatatgggt

SEQ ID NO: 1852 - DNA, 26 bp, Streptococcus aureus
gggtgaggcagtagtcggaacagggt

SEQ ID NO: 1853 - DNA, 26 bp, Streptococcus aureus
aagatataggcccctcactctggggt

SEQ ID NO: 1854 - DNA, 26 bp, Streptococcus aureus
tggggtctagcagagcttggtggaat

SEQ ID NO: 1855 - DNA, 26 bp, Streptococcus aureus
ggagttccattgcatggcctgaggat

SEQ ID NO: 1856 - DNA, 26 bp, Streptococcus aureus
ctgccttccaccaagctctgcaggat

SEQ ID NO: 1857 - DNA, 26 bp, Streptococcus aureus
caccaagctctgcaggatcccagagt

SEQ ID NO: 1858 - DNA, 26 bp, Streptococcus aureus
ctctgcaggatcccagagtcaggggt

SEQ ID NO: 1859 - DNA, 26 bp, Streptococcus aureus
caggaacagtaaaccctgctccgaat

SEQ ID NO: 1860 - DNA, 26 bp, Streptococcus aureus
gacttctctcaattttctagggggat

SEQ ID NO: 1861 - DNA, 26 bp, Streptococcus aureus
ccaatttgtcctggttatcgctggat

SEQ ID NO: 1862 - DNA, 26 bp, Streptococcus aureus
tcatcttcttattggttcttctggat

SEQ ID NO: 1863 - DNA, 26 bp, Streptococcus aureus
catgttgctgtacaaaacctacggat

SEQ ID NO: 1864 - DNA, 26 bp, Streptococcus aureus
ggctttcgcaaaatacctatgggagt

SEQ ID NO: 1865 - DNA, 26 bp, Streptococcus aureus
actgtttggctttcagctatatggat

SEQ ID NO: 1866 - DNA, 26 bp, Streptococcus aureus
ggccaagtctgtacagcatcgtgagt

SEQ ID NO: 1867 - DNA, 26 bp, Streptococcus aureus
ttaccaattttcttttgtctctgggt

SEQ ID NO: 1868 - DNA, 26 bp, Streptococcus aureus
accctaacaaaacaaaaagatggggt

SEQ ID NO: 1869 - DNA, 26 bp, Streptococcus aureus
ggggttattccctaaacttcatgggt

SEQ ID NO: 1870 - DNA, 26 bp, Streptococcus aureus
gaagttggggaactttgccacaggat

SEQ ID NO: 1871 - DNA, 26 bp, Streptococcus aureus
attgattggaaagtatgtcaaagaat

SEQ ID NO: 1872 - DNA, 26 bp, Streptococcus aureus
ggaaagtatgtcaaagaattgtgggt

SEQ ID NO: 1873 - DNA, 26 bp, Streptococcus aureus
tgctgctccatttacacaatgtggat

SEQ ID NO: 1874 - DNA, 26 bp, Streptococcus aureus
ctgctaggctgtactgccaactggat

SEQ ID NO: 1875 - DNA, 26 bp, Streptococcus aureus
catggacattgacccttataaagaat

SEQ ID NO: 1876 - DNA, 26 bp, Streptococcus aureus
agctctgtatcgagaagccttagagt

SEQ ID NO: 1877 - DNA, 26 bp, Streptococcus aureus
gcaagccattctctgctggggggaat

SEQ ID NO: 1878 - DNA, 26 bp, Streptococcus aureus
gaattgatgactctagctacctgggt

SEQ ID NO: 1879 - DNA, 26 bp, Streptococcus aureus
tgatgactctagctacctgggtgggt

SEQ ID NO: 1880 - DNA, 26 bp, Streptococcus aureus
atttggaagatccagcatccagggat

SEQ ID NO: 1881 - DNA, 26 bp, Streptococcus aureus
tcaattatgttaatactaacatgggt

SEQ ID NO: 1882 - DNA, 26 bp, Streptococcus aureus
ttttggaagagagactgtacttgaat

SEQ ID NO: 1883 - DNA, 26 bp, Streptococcus aureus
cttgaatatttggtctctttcggagt

SEQ ID NO: 1884 - DNA, 26 bp, Streptococcus aureus
tatttggtctctttcggagtgtggat

SEQ ID NO: 1885 - DNA, 26 bp, Streptococcus aureus
tacagtacctatctttaatcctgaat

SEQ ID NO: 1886 - DNA, 26 bp, Streptococcus aureus
aaatatttgcccttagacaaaggaat

SEQ ID NO: 1887 - DNA, 26 bp, Streptococcus aureus
cacacgtagcgcatcattttgcgggt

SEQ ID NO: 1888 - DNA, 26 bp, Streptococcus aureus
aacctcgcaaaggcatggggacgaat

SEQ ID NO: 1889 - DNA, 26 bp, Streptococcus aureus
tctttctgttcccaaccctctgggat

SEQ ID NO: 1890 - DNA, 26 bp, Streptococcus aureus
tggccagcagccaaccaggtaggagt

SEQ ID NO: 1891 - DNA, 26 bp, Streptococcus aureus
ccctccacacggcggtattttggggt

SEQ ID NO: 1892 - DNA, 26 bp, Streptococcus aureus
cgattggtggaggcaggaggaggaat

SEQ ID NO: 1893 - DNA, 26 bp, Streptococcus aureus
cccaaaataccgccgtgtggaggggt

SEQ ID NO: 1894 - DNA, 26 bp, Streptococcus aureus
gtggaggggtgagccctggcccgaat

SEQ ID NO: 1895 - DNA, 26 bp, Streptococcus aureus
tgctggccagtggtccttgatggggt

SEQ ID NO: 1896 - DNA, 26 bp, Streptococcus aureus
gatggggttgaagtcccaatctggat

SEQ ID NO: 1897 - DNA, 26 bp, Streptococcus aureus
ctggattgtttgagttggctccgaat

SEQ ID NO: 1898 - DNA, 26 bp, Streptococcus aureus
gtttgagttggctccgaatgcagggt

SEQ ID NO: 1899 - DNA, 26 bp, Streptococcus aureus
ggtccaactgatgatcgggaaagaat

SEQ ID NO: 1900 - DNA, 26 bp, Streptococcus aureus
atgatcgggaaagaatcccagagggt

SEQ ID NO: 1901 - DNA, 26 bp, Streptococcus aureus
cgtgtggtttccctcttatatagaat

SEQ ID NO: 1902 - DNA, 26 bp, Streptococcus aureus
tatagaataccagccttccaaagagt

SEQ ID NO: 1903 - DNA, 26 bp, Streptococcus aureus
gtaatgattaactacctgatctggat

SEQ ID NO: 1904 - DNA, 26 bp, Streptococcus aureus
tctaagggcaaatatttagtgtgggt

SEQ ID NO: 1905 - DNA, 26 bp, Streptococcus aureus
gggcaaatatttagtgtgggtaggat

SEQ ID NO: 1906 - DNA, 26 bp, Streptococcus aureus
ttaataatgtcctcttgtaaatgaat

SEQ ID NO: 1907 - DNA, 26 bp, Streptococcus aureus
aaatgaatcttaggaaaggaaggagt

SEQ ID NO: 1908 - DNA, 26 bp, Streptococcus aureus
aaaggaaggagtttgccattcaggat

SEQ ID NO: 1909 - DNA, 26 bp, Streptococcus aureus
attaaagataggtactgtagaggaat

SEQ ID NO: 1910 - DNA, 26 bp, Streptococcus aureus
cccgtaaagtttcccaccttatgagt

SEQ ID NO: 1911 - DNA, 26 bp, Streptococcus aureus
tttcccaccttatgagtccaaggaat

SEQ ID NO: 1912 - DNA, 26 bp, Streptococcus aureus
gatctgcgtctgcgaggcgagggagt

SEQ ID NO: 1913 - DNA, 26 bp, Streptococcus aureus
tttggtggtctataggctggaggagt

SEQ ID NO: 1914 - DNA, 26 bp, Streptococcus aureus
ggtctataggctggaggagtgcgaat

SEQ ID NO: 1915 - DNA, 26 bp, Streptococcus aureus
cataattgactactagatccctggat

SEQ ID NO: 1916 - DNA, 26 bp, Streptococcus aureus
gactactagatccctggatgctggat

SEQ ID NO: 1917 - DNA, 26 bp, Streptococcus aureus
ttattacccacccaggtagctagagt

SEQ ID NO: 1918 - DNA, 26 bp, Streptococcus aureus
gtcatcaattccccccagcagagaat

SEQ ID NO: 1919 - DNA, 26 bp, Streptococcus aureus
cccagcagagaatggcttgcctgagt

SEQ ID NO: 1920 - DNA, 26 bp, Streptococcus aureus
ggtcggtcgttgacattgctgggagt

SEQ ID NO: 1921 - DNA, 26 bp, Streptococcus aureus
gttgacattgctgggagtccaagagt

SEQ ID NO: 1922 - DNA, 26 bp, Streptococcus aureus
ctcttatgtaagaccttgggcaggat

SEQ ID NO: 1923 - DNA, 26 bp, Streptococcus aureus
taggcccaagcggccccgcgaggggt

SEQ ID NO: 1924 - DNA, 26 bp, Streptococcus aureus
gccccgcgaggggtcgtccgcgggat

SEQ ID NO: 1925 - DNA, 26 bp, Streptococcus aureus
tagacaaaggacgtcccgcgaaggat

SEQ ID NO: 1926 - DNA, 26 bp, Streptococcus aureus
atatttccgcgagaggacgacagaat

SEQ ID NO: 1927 - DNA, 26 bp, Streptococcus aureus
atggccaagccccagccagtgggggt

SEQ ID NO: 1928 - DNA, 26 bp, Streptococcus aureus
accaggccgttgccgagcaacggggt

SEQ ID NO: 1929 - DNA, 26 bp, Streptococcus aureus
tgcatacaaaggcattaaggcaggat

SEQ ID NO: 1930 - DNA, 26 bp, Streptococcus aureus
tatgtaacccatgaagtttagggaat

SEQ ID NO: 1931 - DNA, 26 bp, Streptococcus aureus
accccatctttttgttttgttagggt

SEQ ID NO: 1932 - DNA, 26 bp, Streptococcus aureus
tttgcgaaagcccaggacgatgggat

SEQ ID NO: 1933 - DNA, 26 bp, Streptococcus aureus
aaagcccaggacgatgggatgggaat

SEQ ID NO: 1934 - DNA, 26 bp, Streptococcus aureus
acagcaacatgagggaaacatagagt

SEQ ID NO: 1935 - DNA, 26 bp, Streptococcus aureus
aacatagagttgccttgagcaggagt

SEQ ID NO: 1936 - DNA, 26 bp, Streptococcus aureus
gtactggttgttgttgatcctggaat

SEQ ID NO: 1937 - DNA, 26 bp, Streptococcus aureus
aattggaggacaggaggttggtgagt

SEQ ID NO: 1938 - DNA, 26 bp, Streptococcus aureus
gcgaattttggccaagacacacgggt

SEQ ID NO: 1939 - DNA, 26 bp, Streptococcus aureus
gatgttctccatgttcgtcacagggt

SEQ ID NO: 1940 - DNA, 26 bp, Streptococcus aureus
gtgagaggcaatattcggagcagggt

SEQ ID NO: 1941 - DNA, 26 bp, Streptococcus aureus
aaaatacagacccctgactctgggat

SEQ ID NO: 1942 - DNA, 26 bp, Streptococcus aureus
caccaaactcttcaagatcccagagt

SEQ ID NO: 1943 - DNA, 26 bp, Streptococcus aureus
caggaacagtaagccctgctcagaat

SEQ ID NO: 1944 - DNA, 26 bp, Streptococcus aureus
caaaaatcctcacaataccacagagt

SEQ ID NO: 1945 - DNA, 26 bp, Streptococcus aureus
aggatcatcaaccaccagcacgggat

SEQ ID NO: 1946 - DNA, 26 bp, Streptococcus aureus
actgtctggctttcagttatatggat

SEQ ID NO: 1947 - DNA, 26 bp, Streptococcus aureus
ccctcacaaaacaaaaagatggggat

SEQ ID NO: 1948 - DNA, 26 bp, Streptococcus aureus
gggatactcccttaacttcatgggat

SEQ ID NO: 1949 - DNA, 26 bp, Streptococcus aureus
acttcatgggatatgtaattgggagt

SEQ ID NO: 1950 - DNA, 26 bp, Streptococcus aureus
tcagagaattgtgggtcttttggggt

SEQ ID NO: 1951 - DNA, 26 bp, Streptococcus aureus
tgctgccccttttacacaatgtggat

SEQ ID NO: 1952 - DNA, 26 bp, Streptococcus aureus
cttcaaagactgtgtgtttactgagt

SEQ ID NO: 1953 - DNA, 26 bp, Streptococcus aureus
tgctctgtatcgggaagccttagaat

SEQ ID NO: 1954 - DNA, 26 bp, Streptococcus aureus
gcaagctattctgtgctggggggaat

SEQ ID NO: 1955 - DNA, 26 bp, Streptococcus aureus
gaattaatgactctagctacctgggt

SEQ ID NO: 1956 - DNA, 26 bp, Streptococcus aureus
atttagaagatccagcctccagggat

SEQ ID NO: 1957 - DNA, 26 bp, Streptococcus aureus
ttttggaagagaaactgttcttgaat

SEQ ID NO: 1958 - DNA, 26 bp, Streptococcus aureus
cttgaatatttggtgtcttttggagt

SEQ ID NO: 1959 - DNA, 26 bp, Streptococcus aureus
tacagtacctgtctttaatcctgaat

SEQ ID NO: 1960 - DNA, 26 bp, Streptococcus aureus
aaatatttgcccttagataaagggat

SEQ ID NO: 1961 - DNA, 26 bp, Streptococcus aureus
atttgcatactctttggaaggcgggt

SEQ ID NO: 1962 - DNA, 26 bp, Streptococcus aureus
ggcgggtatcttatataaaagagagt

SEQ ID NO: 1963 - DNA, 26 bp, Streptococcus aureus
aacacatagcgcctcattttgcgggt

SEQ ID NO: 1964 - DNA, 26 bp, Streptococcus aureus
tctttctgtccccaatcccctgggat

SEQ ID NO: 1965 - DNA, 26 bp, Streptococcus aureus
tggccggacgcccacaaggtgggagt

SEQ ID NO: 1966 - DNA, 26 bp, Streptococcus aureus
gggagtgggagcattcgggccagggt

SEQ ID NO: 1967 - DNA, 26 bp, Streptococcus aureus
ccctccccatgggggactgttggggt

SEQ ID NO: 1968 - DNA, 26 bp, Streptococcus aureus
tcccttagaggtggagataagggagt

SEQ ID NO: 1969 - DNA, 26 bp, Streptococcus aureus
gaggagctgctggcacagatgtgagt

SEQ ID NO: 1970 - DNA, 26 bp, Streptococcus aureus
cccaacagtcccccatggggaggggt

SEQ ID NO: 1971 - DNA, 26 bp, Streptococcus aureus
ggggaggggtgaaccctggcccgaat

SEQ ID NO: 1972 - DNA, 26 bp, Streptococcus aureus
gtccggccagttgtccttgtgtgggt

SEQ ID NO: 1973 - DNA, 26 bp, Streptococcus aureus
gtgtgggttgaggtcccaatctggat

SEQ ID NO: 1974 - DNA, 26 bp, Streptococcus aureus
gaggtcccaatctggattttctgagt

SEQ ID NO: 1975 - DNA, 26 bp, Streptococcus aureus
ctggattttctgagttggctttgaat

SEQ ID NO: 1976 - DNA, 26 bp, Streptococcus aureus
ttctgagttggctttgaatgcagggt

SEQ ID NO: 1977 - DNA, 26 bp, Streptococcus aureus
ggtccaactgatgatcggggaagaat

SEQ ID NO: 1978 - DNA, 26 bp, Streptococcus aureus
atgatcggggaagaatcccaggggat

SEQ ID NO: 1979 - DNA, 26 bp, Streptococcus aureus
tataagatacccgccttccaaagagt

SEQ ID NO: 1980 - DNA, 26 bp, Streptococcus aureus
gtaatgattaactacctgctctggat

SEQ ID NO: 1981 - DNA, 26 bp, Streptococcus aureus
gggcaaatatttggtaacattaggat

SEQ ID NO: 1982 - DNA, 26 bp, Streptococcus aureus
tcctggtttcatttactgtaaggggt

SEQ ID NO: 1983 - DNA, 26 bp, Streptococcus aureus
tcaacaatgtcctcctgcaaatgaat

SEQ ID NO: 1984 - DNA, 26 bp, Streptococcus aureus
aaatgaatgtctggaaaagaaggagt

SEQ ID NO: 1985 - DNA, 26 bp, Streptococcus aureus
aaaagaaggagtttgccattcaggat

SEQ ID NO: 1986 - DNA, 26 bp, Streptococcus aureus
attaaagacaggtactgtagaagaat

SEQ ID NO: 1987 - DNA, 26 bp, Streptococcus aureus
agtccaagggatactaacattgggat

SEQ ID NO: 1988 - DNA, 26 bp, Streptococcus aureus
tttggtggtctgtaggcaggaggagt

SEQ ID NO: 1989 - DNA, 26 bp, Streptococcus aureus
ggtctgtaggcaggaggagtgcgaat

SEQ ID NO: 1990 - DNA, 26 bp, Streptococcus aureus
gactactagatccctggaggctggat

SEQ ID NO: 1991 - DNA, 26 bp, Streptococcus aureus
gtcattaattccccccagcacagaat

SEQ ID NO: 1992 - DNA, 26 bp, Streptococcus aureus
cccagcacagaatagcttgcctgagt

SEQ ID NO: 1993 - DNA, 26 bp, Streptococcus aureus
gttgacattgctgaaagtccaagagt

SEQ ID NO: 1994 - DNA, 26 bp, Streptococcus aureus
cagacgagaaggcacagacggggagt

SEQ ID NO: 1995 - DNA, 26 bp, Streptococcus aureus
gagccccaagcggccccgggaggggt

SEQ ID NO: 1996 - DNA, 26 bp, Streptococcus aureus
gccccgggaggggtcgtccgcgggat

SEQ ID NO: 1997 - DNA, 26 bp, Streptococcus aureus
atacttgcgggagagcacgacagaat

SEQ ID NO: 1998 - DNA, 26 bp, Streptococcus aureus
cgacagaattgtcagtcccgatgagt

SEQ ID NO: 1999 - DNA, 26 bp, Streptococcus aureus
atggccaagccccaaccagtgggggt

SEQ ID NO: 2000 - DNA, 26 bp, Streptococcus aureus
cgttgccgagcaacggggtaaagggt

SEQ ID NO: 2001 - DNA, 26 bp, Streptococcus aureus
tgcatataaaggcattaaagcaggat

SEQ ID NO: 2002 - DNA, 26 bp, Streptococcus aureus
tccccatctttttgttttgtgagggt

SEQ ID NO: 2003 - DNA, 26 bp, Streptococcus aureus
tttgcgaaagcccaagatgatgggat

SEQ ID NO: 2004 - DNA, 26 bp, Streptococcus aureus
aaagcccaagatgatgggatgggaat

SEQ ID NO: 2005 - DNA, 26 bp, Streptococcus aureus
aacatagaggttccttgagcaggagt

SEQ ID NO: 2006 - DNA, 26 bp, Streptococcus aureus
gtgctggtggttgatgatcctggaat

SEQ ID NO: 2007 - DNA, 26 bp, Streptococcus aureus
aattggaggacaacaggttggtgagt

SEQ ID NO: 2008 - DNA, 26 bp, Streptococcus aureus
gtgactggagatttgggactgcgaat

SEQ ID NO: 2009 - DNA, 26 bp, Streptococcus aureus
gtaacacgagcaggggtcctaggagt

SEQ ID NO: 2010 - DNA, 26 bp, Streptococcus aureus
gatgttctccatgttcggcacagggt

SEQ ID NO: 2011 - DNA, 26 bp, Streptococcus aureus
aaaatacagagccctgactctgggat

SEQ ID NO: 2012 - DNA, 26 bp, Streptococcus aureus
gccctgactctgggatcttgaagagt

SEQ ID NO: 2013 - DNA, 26 bp, Streptococcus aureus
ggagttccactgcatggcctgaggat

SEQ ID NO: 2014 - DNA, 26 bp, Streptococcus aureus
tcaagcctccaagctgtgccttgggt

SEQ ID NO: 2015 - DNA, 26 bp, Streptococcus aureus
tcgcagaagatctcaatctcgggaat

SEQ ID NO: 2016 - DNA, 26 bp, Streptococcus aureus
aagaagatgaggcatagcagcaggat

SEQ ID NO: 2017 - DNA, 26 bp, Streptococcus aureus
aaaattgagagaagtccaccacgagt